(19) United States
(12) Patent Application Publication
(10) Pub. No.: US 2024/0165154 A1
(43) Pub. Date: May 23, 2024

(54) METHODS AND AGENTS FOR MODULATING ADOPTIVE IMMUNOTHERAPY

(71) Applicant: The Regents of the University of California, Oakland, CA (US)

(72) Inventors: Manish J. BUTTE, Los Angeles, CA (US); Matthew L. MILLER, Los Angeles, CA (US)

(73) Assignee: The Regents of the University of California, Oakland, CA (US)

(21) Appl. No.: 18/283,575

(22) PCT Filed: Apr. 11, 2022
(86) PCT No.: PCT/US22/24298

§ 371 Date: Sep. 22, 2023

Related U.S. Application Data
(60) Provisional application No. 63/173,133, filed on Apr. 9, 2021.

(51) Int. Cl. A61K 35/17; A61K 31/7016; C12N 5/0783; C12N 9/10; C12N 9/42; C12N 15/86

(52) U.S. Cl. CPC A61K 35/17; A61K 31/7016; C12N 5/0636; C12N 9/1051; C12N 9/2445; C12N 15/86; C07K 2319/02; C07K 2319/42; C12N 2740/10043; C12Y 204/0102

(57) ABSTRACT

This disclosure relates to methods and agents for modulating adoptive immunotherapy to enable bioengineered immune cells to utilize xenobiotic fuel, e.g., in a low glucose environment. The immune cells may be used, e.g., for treatment of a tumor or cancer, such as part of a therapeutic treatment of cancer or for treatment of a bacterial, fungal, or viral infection, alone or in combination with a low glucose (e.g., ketogenic) diet. They may also be used to treat a tumor, a cancer, an infection, an autoimmune disease, or an inflammatory or neuroinflammatory disease or condition in a patient on a low glucose diet. The immune cells may be used in combination with a scaffold or platform or with a microparticle or nanoparticle for localization of treatment or xenobiotic nutrients or for controlled release, as well as for other therapeutic uses.

### CROSS REFERENCE TO RELATED APPLICATION

This application claims benefit of U.S. Provisional Patent Application No. 63/173,133, filed Apr. 9, 2021, which is incorporated by reference herein in its entirety.

### FIELD OF INTEREST

This disclosure relates to methods and agents for modulating adoptive immunotherapy to enable bioengineered immune cells to utilize xenobiotic fuel, e.g., in a low glucose environment. The immune cells may be used, e.g., for treatment of a tumor or cancer, such as part of a therapeutic treatment of cancer or for treatment of a bacterial, fungal, or viral infection, alone or in combination with a low glucose (e.g., ketogenic) diet. They may also be used to treat a tumor, a cancer, an infection, an autoimmune disease, or an inflammatory or neuroinflammatory disease or condition in a patient on a low glucose diet. The immune cells may be used in combination with a scaffold or platform or with a microparticle or nanoparticle for localization of treatment or xenobiotic nutrients or for controlled release, as well as for other therapeutic uses.

### BACKGROUND

Glucose is a critical fuel for cellular bioenergetics and a major source of biosynthetic precursors for anabolic pathways. The absence of glucose impairs cellular function, which for T cells includes cytokine production, proliferation, and cytotoxicity (Buck et al. (2015) J. Exp. Med. 212: 1345-1360). Abnormally low glucose concentrations are found in the microenvironment of solid tumors (TME) due to the glucose-avid nature of tumor metabolism, impeding the function of tumor infiltrating lymphocytes (TILs) that might otherwise control tumor growth (Chang et al. (2015) Cell 162: 1229-1241; Singer et al. (2011) Int. J. Cancer 128: 2085-2095). Infusion of additional glucose is not a viable solution, and in practice would feed only the tumor and further starve T cells. A solution to this problem requires a source of glucose that only T cells could utilize.

Cellobiose, a glucose disaccharide found abundantly in plant matter, has great potential to serve as a carbon and energy source but remains inert to catabolic processes in mammalian systems for two primary reasons. First, metazoan sugar transport is restricted to monosaccharides. Second, the β-1,4-glycosidic bond that joins glucose molecules in cellobiose is inefficiently hydrolyzed by mammalian glycoside hydrolases. These processes, that is the transport and hydrolyzation of cellobiose, are efficiently carried out in cellulolytic microbes and many of the relevant genes and proteins have been identified and characterized (Bischof et al. (2016) Microb. Cell Fact. 15:106; Lambertz et al. (2014) Biotechnol. Biofuels 7: 135).

Cellulose, the world's most abundant organic polymer, is an organic compound with the formula (C₆H₁₀O₅)ₙ, a polysaccharide consisting of a linear chain of hundreds to thousands of β(1→4) linked D-glucose units. Cellulose is an important structural component of the primary cell wall of green plants, many forms of algae and the oomycetes. Some species of bacteria secrete it to form biofilms.

Some animals (e.g., ruminants, termites) are able to digest cellulose with the assistance of symbiotic micro-organisms that live in their guts (e.g., Trichonympha). Some ruminants like cows and sheep contain certain symbiotic anaerobic bacteria (such as Cellulomonas and Ruminococcus) in the flora of the rumen, and these bacteria produce enzymes called cellulases that hydrolyze cellulose. The breakdown products are then used by the ruminant as an energy source and by the bacteria for proliferation. The bacterial mass is later digested by the ruminant in its digestive system (stomach and small intestine). Horses use cellulose in their diet by fermentation in their hindgut. However, in human nutrition, cellulose is a non-digestible constituent of insoluble dietary fiber.

The cellulolytic enzyme (cellulase) complex of white-rot Basidiomycota like Phanerochaete chrysosporium and Ascomycota-like Trichoderma reesei consists of a number of hydrolytic enzymes: endoglucanase, exoglucanase and cellobiase (a 0-glucosidase) which work synergistically and, in both bacteria and fungi, are organized into an extracellular multienzyme complex called a cellulosome. Endoglucanase digests cellulose at random, producing glucose, cellobiose (a disaccharide made up of two glucose molecules) and some cellotriose (a trisaccharide). Exoglucanase acts from the non-reducing end of the cellulose molecule, removing glucose units and may also include a cellobiohydrolase activity, thereby producing cellobiose by attacking the non-reducing end of the polymer. Cellobiase hydrolyzes cellobiose to glucose. Glucose, which is easily metabolized, is the end-product of cellulose breakdown by enzymatic hydrolysis.

Cellobiose is a disaccharide with the formula (C₆H₇(OH)₄O)₂O. It is classified as a reducing sugar. In terms of its chemical structure, it is derived from the condensation of a pair β-glucose molecules forging a β(1→4) bond. It can be hydrolyzed to glucose enzymatically or with acid. Cellobiose has eight free alcohol (OH) groups, one acetal linkage and one hemiacetal linkage, which give rise to strong inter- and intramolecular hydrogen bonds. Cellobiose can be obtained by enzymatic or acidic hydrolysis of cellulose and cellulose-rich materials. It is a white solid.

Neurospora crassa (red bread mold), a cellulolytic fungus, utilizes two cellobiose plasma membrane transporters-cellodextrin transporter-1 (cdt-1), which actively transports cellobiose through the cell membrane at the cost of one adenosine triphosphate (ATP) per cellobiose, and cellodextrin transporter-2 (cdt-2), an energy-independent facilitator (passive transporter) of cellobiose transport. Once inside the cell, cellobiose can be cleaved by hydrolysis with an intracellular β-glucosidase (GH1-1) or phosphorolysis with cellobiose phosphorylase (CBP). GH1-1 β-glucosidase is an enzyme capable of cleaving the β(1→4) bond in cellobiose to generate two units of glucose When cellobiose is cleaved by 0-glucosidase (GH1-1), two moles of glucose are generated and enter the glycolytic pathway, subsequently converted to two moles of glucose-6-phosphate by hexokinases with the expense of two moles of ATP. However, phosphorolysis generates one mole of glucose and one mole of glucose-1-phosphate, saving one mole of ATP as glucose-1-phosphate is isomerized to glucose-6-phosphate by phosphoglucomutase without expending ATP.

Tumor cells engage in high rates of glycolysis and deplete extracellular glucose from the tumor microenvironment. Cancer cells undergo changes in their metabolism, including increased uptake of glucose (via aerobic glycolysis, known as the Warburg effect), enhanced rates of glutaminolysis and fatty acids synthesis, and these metabolic shifts support tumor cell growth and survival.

Infections by many species of bacteria (Mycobacterium tuberculosis, Legionella pneumophila, Brucella abortus, Chlamydia trachomatis, Chlamydia pneumoniae, etc.), infectious fungal strains (e.g., Candida albicans, etc.) and strains of viruses (e.g., Vaccinia virus, Dengue virus, human cytomegalovirus, Kaposi's sarcoma-associated herpesvirus, Epstein-Barr virus, hepatitis C virus, SARS-CoV-2 virus, etc.) likewise display the Warburg effect, e.g., engaging in high rates of aerobic glycolysis, to support proliferation and survival of the infectious agent.

T cells are an important component of the immune response. Recently, tumor and cancer treatments have been developed based on T cells. However, T cells are also dependent on high rates of glycolysis to support the high energetic burden of proliferation and effector function.

Moreover, there are patients who, for medical or other reasons, are unable to consume a normal dietary intake of glucose (e.g., who are on a ketogenic or low-glucose diet). Examples include, but are not limited to, individuals with seizures, individuals on ketogenic diets to lose weight, individuals on ketogenic diets due to cancer, and individuals with glycostorage diseases. These individuals need to maintain a low-glucose diet, but a low glucose diet puts them at risk for a reduced ability to fight infection by glucose-consuming immune cells (e.g., T cells).

Thus, there remains an unmet need for compositions and methods of treatment of cancers and other tumors, for example, but not limited to, treatment of benign or malignant solid tumors or malignant cells. A major gap in treatment exists, wherein there is an inability to provide immunological treatments (e.g., utilizing glucose-dependent, T cell-based tumor or cancer therapies) to an extracellular glucose-depleted tumor microenvironment.

Similarly, there remains an unmet need for compositions and methods of treatment of bacterial, fungal, and viral infections. A major gap in treatment exists, wherein there is an inability to mount an immunological response by glucose-consuming immune cells (e.g., T cells) in competition with high glucose-consuming bacteria, fungi, or viruses in an extracellular glucose-depleted environment.

In addition, there remains an unmet need for compositions and methods of treatment for individuals who, for medical or other reasons, are unable to consume a normal dietary intake of glucose, such as individuals who are on a ketogenic or low-glucose diet (e.g., individuals with seizures). A major gap in treatment exists, wherein there is a need for alternative types of nutrition to meet their need to fight infection via glucose-consuming immune cells (e.g., T cells). A need exists to be able to activate T cells at a particular site and/or at a particular time or times, including in cycles, to effectively target cancers, infected cells, or other foci of interest.

### SUMMARY

It would be desirable to enable bioengineered immune cells to utilize xenobiotic fuel, e.g., to import and break down cellobiose into glucose. The xenobiotic fuel (e.g., cellobiose) could thus offer a source of glucose that can feed immune cells but would not feed cancer cells, bacteria, fungi, or glucose-fueled, viral infected cells. Immune cells were engineered to express an importer of cellobiose and the glucosidase enzyme that breaks cellobiose into glucose. It was demonstrated that these immune cells, starved of glucose, can make use of cellobiose. There are numerous diverse applications. Examples include, but are not limited to, 1) cancer immunotherapy—patients are given very low glucose and infused with engineered T cells plus cellobiose sugar; 2) infections—patients are treated with engineered T cells and starved of glucose; and 3) any other applications where ketogenic or low glucose diets are used (patients with seizures, etc.). Moreover, the vast majority of cellobiose is excreted in the urine after intravenous infusion.

In some aspects, disclosed herein are bioengineered cells modified to metabolize a xenobiotic fuel, the xenobiotic fuel not metabolized by a corresponding unmodified cell, the bioengineered cell comprising: (a) at least one foreign nucleic acid encoding at least one transporter protein or a functional fragment thereof for transport of the xenobiotic fuel into the bioengineered cell; (b) at least one foreign nucleic acid encoding at least one protein or a functional fragment thereof for enabling the metabolizing of the xenobiotic fuel in the bioengineered cell; or (c) a combination of (a) and (b).

In related aspects, disclosed herein are methods of modulating an immune response at a focus of interest in a subject in need thereof, the method comprising: administering a xenobiotic fuel-enabled bioengineered immune cell to said subject said bioengineered immune cell comprising: (a) at least one vector comprising at least one nucleic acid encoding at least one transporter protein or a functional fragment thereof for transport of the xenobiotic fuel into the bioengineered immune cell; (b) at least one vector comprising at least one nucleic acid encoding at least one protein or a functional fragment thereof for enabling the metabolizing of the xenobiotic fuel in the bioengineered immune cell; or (c) a combination of (a) and (b); administering the xenobiotic fuel to said subject; wherein said modulating the immune response comprises stimulating said immune response or suppressing said immune response.

In other related aspects, disclosed herein is a method of modulating an immune response at the site of a solid tumor or infection, said method comprising: administering a cellobiose-enabled bioengineered T cell to said subject adjacent to a solid tumor or infection, said cellobiose-enabled bioengineered T cell comprising a vector comprising a nucleic acid encoding a cellodextrin transporter protein or a functional fragment thereof and a vector comprising a nucleic acid encoding a beta-glucosidase protein or a functional fragment thereof; and administering cellobiose to said subject or implanting a scaffold that releases cellobiose adjacent to said solid tumor or infection, said modulating the immune response comprising increasing proliferation of cytotoxic T cells; increasing proliferation of helper T cells; maintaining the population of helper T cells at the site of said tumor; activating cytotoxic T cells at the site of said solid tumor or infection; or any combination thereof.

In still other related aspects, disclosed herein is a method of modulating an immune response at the site of a solid tumor or infection, said method comprising: administering a cellobiose-enabled bioengineered B cell to said subject adjacent to a solid tumor or infection, said cellobiose-enabled bioengineered B cell comprising a vector comprising a nucleic acid encoding a cellodextrin transporter protein or a functional fragment thereof and a vector comprising a nucleic acid encoding a beta-glucosidase protein or a functional fragment thereof; and administering cellobiose to said subject or implanting a scaffold that releases cellobiose adjacent to said solid tumor or infection, said modulating the immune response comprising increasing production of antibodies from the B cell; increasing isotype switching; increasing affinity maturation; or any combination thereof.

In yet other related aspects, disclosed herein is a method of modulating an immune response at a focus of interest of an autoimmune disease, an allergic reaction, a localized infection or an infectious disease, an injury or other damage, a transplant or other surgical site, or a symptom thereof, or a combination thereof, in a subject in need thereof, comprising administering to said subject a bioengineered T regulatory (Treg) cell, adjacent to said focus of interest, said cellobiose-enabled bioengineered Treg cell comprising a vector comprising a nucleic acid encoding a cellodextrin transporter protein or a functional fragment thereof and a vector comprising a nucleic acid encoding a beta-glucosidase protein or a functional fragment thereof; and administering cellobiose to said subject or implanting a scaffold that release said cellobiose adjacent to said focus of interest; wherein said regulating the immune response comprises decreasing proliferation of cytotoxic T cells; decreasing proliferation of helper T cells; suppressing cytotoxic T cells at the site of said focus of interest; or any combination thereof.

In related aspects, disclosed herein is a vector comprising at least one nucleic acid sequence encoding at least one protein for modifying a bioengineered cell to enable metabolism of a xenobiotic fuel in the cell, the xenobiotic fuel not metabolized by a corresponding unmodified cell, the vector comprising: (a) a promoter, the promoter operably linked to (i) a nucleic acid encoding a transporter protein or a functional fragment thereof for transport of the xenobiotic fuel into the bioengineered cell; (ii) a nucleic acid encoding a protein or a functional fragment thereof for enabling the metabolizing of the xenobiotic fuel in the bioengineered cell; or (iii) a combination of (i) and (ii); and (b) a selective marker.

In other related aspects, disclosed herein is a method of making a xenobiotic-enabled bioengineered cell, modified to metabolize a xenobiotic fuel, the xenobiotic fuel not metabolized by a corresponding unmodified cell, the method comprising: (a) selecting a xenobiotic fuel; (b) selecting a transporter protein or functional fragment thereof for transport of the xenobiotic fuel and obtaining a nucleic acid sequence encoding the same; (c) selecting a protein or functional fragment thereof for enabling the metabolizing of the xenobiotic fuel and obtaining a nucleic acid sequence encoding the same; (d) providing (i) a vector comprising a promoter, the promoter operably linked to a nucleic acid encoding a transporter protein or a functional fragment thereof for transport of the xenobiotic fuel into the bioengineered cell, and a selective marker; and (ii) a vector comprising a promoter, the promoter operably linked to a nucleic acid encoding a protein or a functional fragment thereof for metabolizing the xenobiotic fuel in the bioengineered cell, and a selective marker; (e) isolating a cell of interest from a subject; (f) transfecting or transducing the cell of interest with (i) the vector comprising a nucleic acid encoding a transporter protein or a functional fragment thereof for transport of the xenobiotic fuel into the bioengineered cell; and (ii) the vector comprising a nucleic acid encoding a protein or a functional fragment thereof for enabling the metabolizing of the xenobiotic fuel in the bioengineered cell.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows schematic maps of two vectors forming a vector pair for gene delivery into mouse T cells. The vector utilizes components of the mouse stem cell virus (MSCV), a retrovirus capable of delivery DNA cargo into a target genome. The MSCV system comprises long terminal repeats (LTRs) that serve both to integrate into host genome and to promote and suppress transcription of DNA cargo. The vector contains a murine embryonic stem cell virus psi (MESV Ψ) signal element that facilitates packaging of the viral RNA into capsids particles. The only difference between the vectors is the expression of different fluorescent markers, with folding reporter variant green fluorescent protein (frGFP) or mCherry being constitutively driven by the PGK promoter. (mCherry is a member of the mFruits family of monomeric red fluorescent proteins (mRFPs).) When these plasmids are transfected into the PLATINUM-E™ cell line, a derivative of 293T cell line that expresses the gag (group antigens polyprotein), pol (reverse transcriptase polymerase), and env (envelope) viral proteins, infectious viral particles are produced that can be used to transduce primary T cells. The envelope of the virus is ecotropic (i.e., can only infect mouse or rat cells). The MCS_PGK-GFP vector (top; SEQ ID NO: 7) includes the following elements from the 5′-end: 5′ long terminal repeats (5′ LTR), murine embryonic stem cell virus psi (MESV ψ), multiple cloning site (MCS), mouse phosphoglycerate kinase 1 promoter (PGK promoter), folding reporter green fluorescent protein (frGFP) as a marker, and 3′ long terminal repeats (3′ LTR). The MCS_PGK-mCherry vector (bottom; SEQ ID NO: 8) is identical expect that it utilizes mCherry, a member of the monomeric red fluorescent protein family, as a marker.

FIG. 2 shows schematic maps of vectors for genomic integration of DNA cargo, in this case, an optimized version of the gh1-1 gene, expressing BETA-GLUCOSIDASE (GH1-1; Neurospora crassa [strain ATCC 24698/74-OR23-1A/CBS 708.71/DSM 1257/FGSC 987]), and or an optimized version of the cdt-1 gene, expressing CELLODEXTRIN TRANSPORTER 1 (Neurospora crassa [strain ATCC 24698/74-OR23-1A/CBS 708.71/DSM 1257/FGSC 987]). Each expressed protein was designed to have an N-terminal hemagglutinin tag (HA) on either the GH1-1 or CDT-1 protein. HA-cdt-1 or HA-gh1-1 constructs were inserted into the MCS of the vectors shown in FIG. 1. As a result, gh1-1 (gh1-1-PGK GFP [above top; SEQ ID NO: 9]) utilizes folding reporter green fluorescent protein (frGFP) as a marker, while the other gene cdt-1 (cdt-1 PGK mCherry [bottom; SEQ ID NO: 11], utilizes mCherry as a marker. Cdt-1 was also placed into the frGFP backbone (cdt-1 PGK frGFP [below top; SEQ ID NO: 10]), prior to the availability of the mCherry vector.

FIG. 3 is a photograph of a PLATINUM-E™ (Plat-E) cell (CELL BIOLABS™) immunoblot gel ladder (M), MSCV mCherry control (1), MSCV cdt-1 PGK mCherry vector (2), MSCV GFP control (3), and MSCV gh1-1 GFP vector (4). The immunoblots utilize a PLATINUM-E™ cell lysate, a primary anti-hemagglutinin (anti-HA) tag antibody (1:1000), and a donkey anti-rabbit secondary antibody. The ladder (M) provides proteins with the sizes as indicated on the left of FIG. 3. FIG. 3 shows the immunoblot after a 2-second exposure at an infrared wavelength of 800 nanometers (nm) (IR800). The expected protein size for gh1-1 was 55.4 kDaltons (kDa), as shown. The expected protein size for cdt-1 was 64.3 kDa, but the bands appeared to be the incorrect size (primarily at approximately 45 kDa with smaller, fainter bands at approximately 36 kDa), possibly due to N-terminal HA tag negatively impacting protein sorting to specific cellular compartments.

FIG. 4 depicts two new vector constructs. In one vector (top; SEQ ID NO: 12), the construct includes the following elements from the 5′-end: 5′ LTR, MESV psi (MESV ψ), cdt-1 with C-terminal HA tag-encoding sequence inserted in the MCS, PGK promoter, mCherry, and 3′ LTR. In the other vector (bottom; SEQ ID NO: 13), the construct includes the following elements from the 5′-end: 5′ LTR MESV psi (MESV T), cdt-1 with HA tag (HA tag-encoding sequence expressed C-terminal to the cdt-1; SEQ ID NO: 20) and ERES (SEQ ID NO: 23, SEQ ID NO: 24, and SEQ ID NO: 25) discrete endoplasmic reticulum export signal-encoding sequence (ERES), PGK promoter, mCherry, and 3′ LTR.

FIG. 5 is a series of confocal micrographs showing PLATINUM-E™ (CELL BIOLABS™) immunocytochemistry of PLATINUM-E™ cells transfected with the vector constructs as shown (MSCV GFP control [top row; see FIG. 1—top for vector; SEQ ID NO: 7]; MSCV gh1-1 GFP [middle row; see FIG. 2—below top for vector; SEQ ID NO: 10]; MSCV HA-cdt-1 GFP [N-terminal HA tag] [bottom row; see FIG. 2—above top for vector; SEQ ID NO: 9]), then detected as indicated with, left to right: green fluorescent protein (GFP); 2-(4-amidinophenyl)-1H-indole-6-carboxamidine (4′,6-diamidino-2-phenylindole; DAPI); GFP+DAPI; hemagglutinin (HA); HA+DAPI. Detection of the HA tag indicates gh1-1 and cdt-1 protein expression. However, while cdt-1 seems to localize to plasma membrane, it is also present diffusely in cytosol. Although not full-length, the cdt-1 is functional.

FIG. 6 is a series of confocal micrographs showing PLATINUM-E™ (CELL BIOLABS™) immunocytochemistry of PLATINUM-E™ cells transfected with the constructs as shown (MSCV mCherry control [top row; see FIG. 1—bottom for vector; SEQ ID NO: 8]; MSCV cdt-1 HA [HA tag C-terminal to cdt-1 protein] [middle row; see FIG. 4—top for vector; SEQ ID NO: 12]; MSCV cdt-1 HA ERES mCherry [bottom row; see FIG. 4—bottom for vector; SEQ ID NO: 13]), then detected as indicated with, left to right: mCherry; DAPI; mCherry+DAPI; HA; HA+DAPI. Compared with the results in FIG. 5, FIG. 6 demonstrates that cdt with C-terminal amendments (C-terminal HA or C-terminal HA ERES) shows much more discrete localization only in plasma membrane.

FIG. 7 compares selected electron micrographs of FIG. 5 and FIG. 6 showing PLATINUM-E™ (CELL BIOLABS™) immunocytochemistry of HA detection in PLATINUM-E™ cells transfected with the constructs as shown (MSCV HA cdt-1 GFP [N-terminal HA tag] [left; see FIG. 2—above top for vector; SEQ ID NO: 9]; MSCV cdt-1 HA mCherry [C-terminal HA tag] [center; see FIG. 4—top for vector; SEQ D NO: 12]; MSCV cdt-1 HA ERES mCherry [C-terminal HA tag+ERES] [right; see FIG. 4—bottom for vector; SEQ ID NO: 13]), demonstrating that the addition of an HA tag and ERES peptide motif to the C-terminus of protein results in best protein localization (right).

FIGS. 8A-8B are schematics and graphs depicting a PLATINUM-E™ (CELL BIOLABS™) functional experimental method and results measuring cell proliferation with a combination of cellobiose and low glucose, as compared to high glucose and low glucose controls. PLATINUM-E™ 293 cells were transfected with genes of interest, plated in metabolic conditions, and their proliferation monitored. FIG. 8A shows a schematic timeline (top) of the experiment. Transfection took place on Day 0 (d0). PLATINUM-E™ cells were transfected with MSCV gh1-1 GFP vector (see FIG. 2—below top for vector; SEQ ID NO: 10) and MSCV cdt-1 mCherry vector (see FIG. 2—bottom for vector; SEQ ID NO: 11), as represented by the schematic (bottom left). On Day 2 (d2), cells were stained with CELLTRACE™ VIOLET (CTV) using the CELLTRACE™ VIOLET Cell Proliferation Kit (CTV; THERMOFISHER™ SCIENTIFIC C34557, then sorted via fluorescence-activated cell sorting (FACS) for GFP+/mCherry+ cells (bottom center graph,), and then plated in metabolic conditions as shown on the schematic (bottom right). Metabolic conditions were: 10 millimolar (mM) glucose (high glucose); 0.1 mM glucose (low glucose); or 0.1 mM glucose (low glucose)+10 mM cellobiose. On Day 4 (d4), cells were harvested, and proliferation under each metabolic condition was measured as a function of CTV. FIG. 8B, an expanded view of the corresponding graph in FIG. 8A bottom center, is a logarithmic graph depicting the results of flow cytometric measurement of the GFP (X-axis) and mCherry (y-axis) fluorescent signals of PLATINUM-E™ cells post-transfection. By looking at Quadrant 2 it can be seen that 81% of the cells were double-positive, and it was this population that was sorted for proliferation analysis. FIG. 8B is a representative result of the mCherry and GFP signal of the PLATINUM-E™ cells after transfection.

FIG. 9 is a graph depicting the results of the PLATINUM-E™ (CELL BIOLABS™) cell proliferation experiment of FIGS. 8A-B. FIG. 9 is a series of graphs that depict the analysis pipeline used to determine which cells underwent division. Forward (x-axis) and side (y-axis) scatter were used to determine viable cells (top left, gating on “size”). Within this population, the cells expressing the highest levels GFP (x-axis) and mCherry (y-axis) were gated for further analysis (top center, gating on “GFP+ mCherry+”). Within the GFP+ mCherry+ population, a histogram projection displays the level of CELLTRACE™ Violet fluorescent signal (bottom left). This analysis is transformed by changing the y-axis from counts to forward scatter (bottom center). Finally, the discrete population of cells that has had the fluorescent signal diluted in half, which is considered the population of cells that has undergone division, was gated and quantified. (bottom right, “% divided”). FIG. 9 is a representative example of how cells were gated for analysis.

FIG. 10 is a bar graph depicting the results of a cell proliferation study in PLATINUM-E™ cells (CELL BIOLABS™) that follows the pipeline illustrated in FIG. 8A above. To select viable, GFP+ mCherry+ cells, events with a particular forward and side scatter (“alive cells”) were selected for further analysis. Within this population, events that were GFP and mCherry positive were selected for further analysis. FIG. 10 shows the results of the percentage of the viable, GFP+ mCherry+ cells that underwent division when incubated in each of the three metabolic conditions (high glucose [gray]; low glucose [blue]; low glucose+cellobiose [green]) for PLATINUM-E™ cells transfected with the control vectors (left trio, FIG. 1 above and below; SEQ ID NO: 7 and SEQ ID NO: 8), PLATINUM-E™ cells transfected with gh1-1 and cdt-1 vectors (center trio, FIG. 2 below top [SEQ ID NO: 10]+FIG. 4 top [SEQ ID NO: 12]), and PLATINUM-E™ cells transfected with gh1-1 and cdt-1-ERES vectors (right trio, FIG. 2 below top [SEQ ID NO: 10]+FIG. 4 bottom [SEQ ID NO: 13]). A slightly higher fraction of cells co-transfected with cdt-1 and gh1-1 undergo cell division in the presence of cellobiose compared to control.

FIG. 11 is a bar graph depicting the results of a second cell proliferation study. The proliferation experiment was repeated with different basal metabolic conditions, with the base media containing 5× less dFBS and 10× less D-glutamine (with new final concentrations of 2% and 200 uM respectively). FIG. 11 shows the results of the percentage of the viable, GFP+ mCherry+ cells that underwent division when incubated in the three metabolic conditions (high glucose [gray]; low glucose [blue]; low glucose+cellobiose [green]) for PLATINUM-E™ cells (CELL BIOLABS™) transfected with a the control vectors (left trio, FIG. 1 top and bottom [SEQ ID NO: 7 and SEQ ID NO: 8]), PLATINUM-E™ cells transfected with gh1-1 and cdt-1 vectors (center trio, FIG. 2 below top [SEQ ID NO: 10]+FIG. 4 top [SEQ ID NO: 12]), and PLATINUM-E™ cells transfected with gh1-1 and cdt-1-ERES vectors (right trio, FIG. 2 below top [SEQ ID NO: 10]+FIG. 4 bottom [SEQ ID NO: 13]). The ability of cells expressing gh1-1 and cdt-1 to proliferate using cellobiose was more apparent.

FIGS. 12A-12C are a series of compound light micrographs of PLATINUM-E™ cells (CELL BIOLABS™) in culture. FIG. 12A is a series of compound light micrographs of PLATINUM-E™ cells transfected with both of the parent plasmids [FIG. 1—top and bottom [SEQ ID NO: 7 and SEQ ID NO: 8] under various metabolic conditions (high glucose [left]; low glucose [center]; low glucose+cellobiose [right]). Of note is cell morphology, with cell cultured in high glucose showing a larger size and cellular projections, and adherence to the surface. Cells cultured in low glucose and low glucose+cellobiose display smaller, spherical morphology and exist in suspension or loosely adhered to the cell plate. FIG. 12B is a series of compound light micrographs of PLATINUM-E™ cells transfected with the gh1-1 vector [FIG. 2 below top (SEQ ID NO: 10)] and the cdt-1 vector [FIG. 4 top (SEQ ID NO: 12) under various metabolic conditions (high glucose [left]; low glucose [center]; low glucose+cellobiose [right]). Of note is cell morphology and adherence to the surface, with gh1-1+cdt-1 expressing cells displaying rescued size, projections, and adherence to the culture surface in the presence of low glucose+cellobiose. FIG. 12C is a series of compound light micrographs of PLATINUM-E™ cells transfected with the gh1-1 vector [FIG. 2 below top (SEQ ID NO: 10)] and the cdt-1-ERES vector [FIG. 4 bottom (SEQ ID NO: 12)] under various metabolic conditions (high glucose [left]; low glucose [center]; low glucose+cellobiose [right]). Of note is cell morphology and adherence to the surface, again with gh1-1+cdt-1-ERES expressing cells displaying rescued size, projections, and adherence to the culture surface in the presence of low glucose+cellobiose.

FIGS. 13A-13D are a schematic timeline and graphs depicting a T cell functional experimental method and results measuring cell proliferation with a combination of cellobiose and low glucose, as compared to high glucose and low glucose controls. Transduced T cells are assessed for their ability to proliferate with cellobiose. T cells received genetic cargo in the form of MSCV virus and then expressed gh1-1 and cdt-1. They were put into metabolic conditions and then their proliferation assessed. FIG. 13A shows a schematic timeline (top) of the experiment. On Day 0 (d0), T cells were harvested from the spleen of a BL/6J mouse, stained with CELLTRACE™ VIOLET using the CELLTRACE™ VIOLET Cell Proliferation Kit (CTV; THERMOFISHER™ SCIENTIFIC C34557 and then activated. On Day 1 (d1), the stained, activated T cells were transduced by spinfection with MSCV virus containing empty control, cdt-1, or gh1-1 genetic cargo [empty controls=FIG. 1—top and bottom (SEQ ID NO: 7 and SEQ ID NO: 8), gh1-1=FIG. 2—below top (SEQ ID NO: 10), cdt-1=FIG. 4 top (SEQ ID NO: 12) or FIG. 4 bottom (SEQ ID NO: 13)]. On Day 2 (d2), measured for CTV, and plated in metabolic conditions. On Day 4 (d4) proliferation under each metabolic condition was measured as a function of CTV. FIG. 13B is an enlarged view of the graph of FIG. 13A (bottom left) depicting mCherry (y-axis) and GPF (x-axis) fluorescent signal of T cells one day post-transduction, demonstrating that a significant fraction of T cells are successfully co-transduced, based on the percentage of cells that are double-positive for mCherry and GFP. Instead of selection or sorting, a small aliquot of cells was run on the cytometer to measure transduction efficiency (cells double positive for GFP and mCherry) and to measure the state of CTV signal at the onset of the various metabolic incubations. FIG. 13C is an enlarged view of the graph of FIG. 13A (bottom center) depicting forward scatter (y-axis) and CELLTRACE™ VIOLET (x-axis) fluorescent signal on Day 2. Day 2 fluorescence (left) is measured to establish a baseline signal before cells are plated into metabolic conditions and allowed to continue to proliferate. Dilution of the signal over successive cellular generations can be seen and is used to assess proliferation.

FIGS. 14A-14B are graphs depicting the results of the T cell proliferation experiment of FIGS. 13A-13C. FIG. 14A is a series of graphs depicting forward scatter (y-axis) and CELLTRACE™ VIOLET (CTV) (x-axis) fluorescent signal of transduced T cells incubated for two days in high glucose, low glucose, or low glucose+cellobiose metabolic conditions. The percentage of the cells that have divided 4 or more times have been quantified and annotated as “CTV low”. FIG. 14B is a bar graph depicting the results of a T cell proliferation study and shows the results of the relative CTV low % with respect to each of the three samples (T cells transduced with a control virus [left trio]; T cells transduced with gh1-1 and cdt-1 virus [center trio]; T cells transduced with gh1-1 and cdt-1-ERES virus [right trio]) with respect to each of the three metabolic conditions (high glucose [gray, left bar of each trio], low glucose [green, center bar of each trio], and low glucose+cellobiose [blue, right bar of each trio]).

FIGS. 15A-15B are bar graphs depicting the results of a PLATINUM-E™ cell (CELL BIOLABS™) proliferation study following single-gene control transfections. FIG. 15A shows the results of the relative CTV low % with respect to PLATINUM-E™ cells transfected with a single control vector (FIG. 1—top [SEQ ID NO: 7]) [left trio]; gh1-1 GFP vector (FIG. 2—below top [SEQ ID NO: 10]) [right trio] with respect to each of the three metabolic conditions (high glucose [gray, left bar of each trio], low glucose [green, center bar of each trio], and low glucose+cellobiose [blue, right bar of each trio]). FIG. 15B shows the results of the relative CTV low % with respect to PLATINUM-E™ cells transfected with a single control vector (FIG. 1—bottom [SEQ ID NO: 8]) [left trio]; cdt mCherry vector (FIG. 4—top [SEQ ID NO: 12]) [center trio]; cdt-ERES mCherry vector (FIG. 4—bottom [SEQ ID NO: 13]) [right trio]) with respect to each of the three metabolic conditions (high glucose [gray, left bar of each trio], low glucose [green, center bar of each trio], and low glucose+cellobiose [blue, right bar of each trio]). These data indicate that expression of a single gene, either cdt-1 or gh1-1, is not sufficient to rescue proliferation with cellobiose.

FIGS. 16A-16B are schematic maps and expression analysis of MSCV vectors that were constructed to contain different codon optimized variants of the cdt-1 gene, each with an HA-tag sequence amended to the C-terminus. In FIG. 16A, the top vector is the same as in FIG. 4 [above] (SEQ ID NO: 12). The second vector (SEQ ID NO: 14) from the top includes a different cdt-1 DNA sequence generated by the IDT codon optimization tool, which is the same tool used to generate the cdt-1 sequence in the top vector. This tool is not deterministic and results in different outputs each time a sequence is entered. The third vector (SEQ ID NO: 15) from the top is a third cdt-1 DNA sequence generated using a codon optimization tool from BLUE HERON™ BIOTECH, and the bottom vector (SEQ ID NO: 16) is a fourth cdt-1 DNA sequence generated using a codon optimization tool from GENSCRIPT™ BIOTECH. FIG. 16B shows flow cytometric analysis of transfected PLATINUM-E™ cells (CELL BIOLABS™) stained with anti HA-tag antibody. These graphs depict the anti-HA tag signal within the mCherry+ positive populations, or the populations that were successfully transfected. The percent of the parent population is displayed (or the percent of mCherry+ cells that have a detectable HA-tag signal) as well as the mean fluorescence intensity (MFI) of the HA-tag signal within the entire mCherry+ population. These results indicate that the GenScript codon optimized variant [FIG. 16B—far right; SEQ ID NO: 16] results in the highest percentage of mCherry+ cells with a detectable HA-tag signal as well as the highest MFI, showing a 7-10-fold increase over the other variants.

FIG. 17 shows the results of another PLATINUM-E™ cell (CELL BIOLABS™) proliferation experiment, using the new codon optimized cdt-1 variant from GENSCRIPT™, compared to the previously constructed variants. The results display the CELLTRACE™ VIOLET signal of the GFP+ mCherry+ positive cells transfected with the various constructs (control [FIG. 1—top and bottom together (SEQ ID NO: 7 and SEQ ID NO: 8)], the remaining conditions use the gh1-1 vector [FIG. 2—below top (SEQ ID NO: 10)] together with cdt-1 [from FIG. 2—bottom (SEQ ID NO: 11) or FIG. 4—top (SEQ ID NO: 12) or FIG. 4—bottom (SEQ ID NO: 13) or FIG. 16A—bottom (SEQ ID NO: 16)]); and incubated in a basal condition, basal condition plus glucose, or basal condition plus cellobiose. The signals are normalized to the control (EV) cells in the basal condition. These results indicate that the cells co-transfected with the construct containing gh1-1 and the GENSCRIPT™ cdt-1 gene can proliferate using cellobiose at a comparable level to control cells growing in glucose, suggesting that total expression of cdt-1 is an important factor for utility of cellobiose as a fuel source.

FIGS. 18A-18B show the effects of engineering primary, BL/6J mouse T cells to express CDT-1 and GH1-1 (CG-T cells). As described in FIGS. 13A-13C and FIGS. 14A-14B, activated T cells were co-transduced with MSCV carrying either CDT-1 or GH1-1. Nearly 50% of cells were co-transduced, as assessed by the dual expression of fluorescent markers. The same data set for those figures was re-analyzed here. The analysis was slightly changed (gating), and the plot in FIG. 18B shows absolute percentages, rather than relative percentages. FIG. 18A shows flow cytometric analysis of T cells co-transduced with MSCV (Control [EV-mCh+EV-GFP; left]; CDT-1+GH1-1 [center]; CDT-1-ERES+GH1-1 [right]), resulting in dual expression of mCherry and GFP in approximately 50% of the population. CELLTRACE™ VIOLET-stained CG-T cells were incubated in high glucose (HG), low glucose (LG), and low glucose+cellobiose (LG+C) conditions for 48 hr, after which their fluorescent signals were measured. CG-T cells showed a boost in proliferation when cellobiose was added to the low glucose environment. FIG. 18B is a series of bar graphs showing the results of FIG. 18A (Control [EV-mCh+EV-GFP; left trio]; CDT-1+GH1-1 [center trio]; CDT-1-ERES+GH1-1 [right trio]) with respect to HG [left in each trio], LG [center in each trio], and LG+C [right in each trio]. In mCh+ GFP+ cells, cellobiose (+C) rescued T-cell proliferation in starvation conditions (low glucose, LG), approaching the high glucose (HG) state. Notably, there was a minor increase in wild-type (WT) T-cell proliferation in the low glucose environment when cellobiose was added, suggesting possible spontaneous or enzymatic hydrolysis, but the increase in proliferation of WT T-cells did not match the increase in proliferation of CG-T cells.

FIG. 19 shows flow cytometric analysis demonstrating that cellobiose is inert to tumors. The high glucose (HG), low glucose (LG), and low glucose+cellobiose (LG+C) in vitro studies of FIGS. 18A-18B were repeated using a B16 melanoma cell line constitutively expressing GFP. By using GFP positivity as a proxy for cell viability, the data demonstrated that cellobiose does not provide B16 melanoma any survival advantage in low glucose environments. Cellobiose (+C) did not promote B16 melanoma tumor survival in starvation (low glucose, LG) conditions. Tumors did not derive benefit from cellobiose.

FIG. 20 shows schematic maps of two additional MSCV vectors forming a vector pair for gene delivery into mouse T cells. The MSCV system comprises long terminal repeats (LTRs) that serve both to integrate into host genome and to promote and suppress transcription of DNA cargo. The vector contains a murine embryonic stem cell virus psi (MESV Ψ) signal element that facilitates packaging of the viral RNA into capsids particles. A T2A (2A) ribosomal skipping sequence was provided 3′ to the cloning site, and the T2A sequence was then followed in-frame by either the mCherry (top) or GFP (bottom) coding sequence. Additionally, the Woodchuck hepatitis virus post-transcriptional regulatory element (WPRE) was added downstream from the transgene. WPRE has been shown to increase transcript stability and leads to enhanced protein expression on transcripts where it is present. The only difference between the vectors is the expression of different fluorescent markers, with mCherry (top) or with green fluorescent protein (GFP) (bottom) being constitutively driven by the PGK promoter. The MSCV_PGK-2A-mCherry vector (top; SEQ ID NO: 26) includes the following elements from the 5′-end: 5′ long terminal repeats (5′ LTR), murine embryonic stem cell virus psi (MESV ψ), mouse phosphoglycerate kinase 1 promoter (PGK promoter), T2A (2A) ribosomal skipping sequence, mCherry as a marker, WPRE, and 3′ long terminal repeats (3′ LTR). The MSCV_PGK-2A-GFP vector (bottom; SEQ ID NO: 27) is identical expect that it utilizes folding reporter green fluorescent protein (frGFP; GFP as abbreviated herein) as a marker. Restriction enzyme sites NotI and BamH1 for cloning in the gene are shown in the figure.

FIG. 21 shows schematic maps of two additional MSCV vectors forming a vector pair for gene delivery into mouse T cells. The transgenes, cdt-1 and gh1-1, each with a hemagglutinin (HA) tag, were relocated under the control of the strong, constitutive promoter PGK. The 3′ ends of the genes were modified to contain a T2A ribosomal skipping sequence that was then followed in-frame by either the mCherry or GFP coding sequence. Additionally, the Woodchuck hepatitis virus post-transcriptional regulatory element (WPRE) was added downstream from the transgene to increase stability. The difference between the vectors is the location of the hemagglutinin (HA) tag and the expression of both different proteins and different fluorescent markers, with 3′-HA-tagged/3′-2A CDT-1 and mCherry (top) or with 5′-HA-tagged/3′-2A GH1-1 and green fluorescent protein (GFP) (bottom) being constitutively driven by the PGK promoter. The MSCV_PGK-cdt-1-2A-mCherry vector (top; SEQ ID NO: 28) includes the following elements from the 5′-end: 5′ long terminal repeats (5′ LTR), murine embryonic stem cell virus psi (MESV ψ), mouse phosphoglycerate kinase 1 promoter (PGK promoter), cdt-1 coding sequence, hemagglutinin (HA) tag, T2A (2A) ribosomal skipping sequence, mCherry as a marker, WPRE, and 3′ long terminal repeats (3′ LTR). The MSCV_PGK-gh1-1-2A-GFP vector (bottom; SEQ ID NO: 29) includes the following elements from the 5′-end: 5′ long terminal repeats (5′ LTR), murine embryonic stem cell virus psi (MESV T), mouse phosphoglycerate kinase 1 promoter (PGK promoter), hemagglutinin (HA) tag, gh1-1 coding sequence, T2A (2A) ribosomal skipping sequence, green fluorescent protein (GFP) as a marker, WPRE, and 3′ long terminal repeats (3′ LTR).

FIG. 22 shows graphs of comparative results of a glucose stress test and a cellobiose stress test, each performed with various vectors above by SEAHORSE EXTRACELLULAR FLUX ASSAY™ (AGILENT™) on a SEAHORSE EXTRACELLULAR FLUX ANALYZER™ (AGILENT™), comparing functional output in CG-HEK-293 cells. the first-generation transgene constructs (SEQ ID NO: 12 and SEQ ID NO: 10; FIG. 4/FIG. 16A and FIG. 2, respectively), the second-generation transgene constructs (SEQ ID NO: 28 and SEQ ID NO: 29; FIG. 21), the first-generation empty vectors (SEQ ID NO: 7 and SEQ ID NO: 8; FIG. 1), or the second-generation empty vectors (SEQ ID NO: 26 and SEQ ID NO: 27; FIG. 20) were transfected in pairs into PLATINUM-E™ cells (an HEK293 derivative). After two days, the transfected cells were assayed on a SEAHORSE EXTRACELLULAR FLUX ANALYZER™ (AGILENT™) to measure extracellular acidification rates (ECAR) using either glucose or cellobiose as a primary carbon source. The ECAR of cells in basal media (no glucose or cellobiose) was first measured to obtain a baseline rate. Next, glucose (Glucose Stress Test; left panel) or cellobiose (Cellobiose Stress Test; right panel) was injected into each well, and the relative increase in ECAR (%) was measured as a function of time (minutes). When glucose was injected into the wells, all four conditions responded with a rapid increase in ECAR, with the rates increasing and plateauing between 250-350% over basal ECAR (left panel). In contrast, when cellobiose was injected into each well, only the cells transfected with the generation 2 transgene vectors (SEQ ID NO: 28 and SEQ ID NO: 29; FIG. 21) showed an increase in ECAR (right panel), indicating that the transgene expression level was enhanced sufficiently to allow for the consumption of cellobiose to be measured in this format. Oligomycin and 2-deoxyglucose were also sequentially injected into the wells. Oligomycin blocks ATP synthase and measures maximal glycolytic capacity and 2-deoxyglucose competes with glucose as a substrate for hexokinase and measures the portion of ECAR that is attributable to glycolysis. (The black line that repeats in value at 100% represents the normalized value from measurement 3, to which all other measurements are compared [y-axis is percent change in ECAR value relative to ECAR at measurement 3].)

It will be appreciated that for simplicity and clarity of illustration, elements shown in the figures have not necessarily been drawn to scale. For example, the dimensions of some of the elements may be exaggerated relative to other elements for clarity.

### DETAILED DESCRIPTION

Obstacles persist in developing and applying effective methods for activating cytotoxic T cells or other immune cells for treatment of cancer or other immunotherapy. As described herein, significant improvements have been made in the response of immune cells to solid tumors despite their immunosuppressive and/or metabolically restrictive tumor environment. Glucose is known to be a potent promoter of cancer growth and metastasis and tumor growth. Similarly, glucose is known to be a potent promoter of the growth and activity of infectious agents (e.g., bacteria, fungi, and virus-infected cells). Immune cells require a microenvironment with glucose present at sufficient levels in order to proliferate and/or launch an effective immune response, but tumor or cancer microenvironments and the microenvironment of an infection are often glucose-depleted.

It would be desirable to enable T cells or other immune cells to import and break down cellobiose into glucose. Cellobiose could thus offer a source of glucose that can feed immune cells but would not feed cancer cells or certain bacteria, fungi, or virus-infected cells. Such bioengineered T cells could be effectively used in such glucose-limited environments, by providing cellobiose systemically or locally. Cellobiose is metabolically inert to non-engineered human cells and is excreted through the urine.

Importation and breakdown by immune cells of cellobiose provides the immune cells with a source of glucose that feeds the immune cells without feeding cancer cells or certain bacteria, fungi, or virus infected cells. Provided herein are bioengineered immune cells that express an importer of cellobiose, and a glucosidase and/or phosphorylase enzyme that hydrolyzes or phosphorylyses cellobiose into glucose and/or into glucose-1-phosphate. These immune cells, starved of glucose, can make use of cellobiose. They may also be targeted to the area of a cancer, tumor, infection, or other localized symptom, disease, or medical condition and used to treat the cancer, tumor, infection, or other localized symptom, disease, or medical condition. They may also be used to provide more effective treatments for patients, who are on a ketogenic or low-glucose diet. Also provided are vectors for expressing an importer of cellobiose and/or the glucosidase enzyme, methods of bioengineering the immune cells, and methods of using them. Also provided are methods of using the bioengineered cell to treat or alleviate cancer or another disease or aberrant physiological condition.

In the following detailed description, numerous specific details are set forth in order to provide a thorough understanding of methods and agents for modulating adoptive immunotherapy to enable bioengineered immune cells to utilize xenobiotic fuel, e.g., in a low glucose environment. However, it will be understood by those skilled in the art that the production of these bioengineered immune cells and uses thereof may be practiced without these specific details. In other instances, well-known methods, procedures, and components have not been described in detail so as not to obscure their description.

Described herein are compositions and methods for obtaining a cell line engineered to co-express the appropriate transporter and hydrolyzing enzyme utilizing cellobiose to yield free glucose within the cytosol that will subsequently replenish glycolytic intermediates depleted in low glucose environments, rescuing glucose deprivation.

In some aspects, disclosed herein are bioengineered cells modified to metabolize a xenobiotic fuel, the xenobiotic fuel not metabolized by a corresponding unmodified cell, the bioengineered cell comprising: (a) at least one foreign nucleic acid encoding at least one transporter protein or a functional fragment thereof for transport of the xenobiotic fuel into the bioengineered cell; (b) at least one foreign nucleic acid encoding at least one protein or a functional fragment thereof for enabling the metabolizing of the xenobiotic fuel in the bioengineered cell; or (c) a combination of (a) and (b).

In some embodiments, (a) the xenobiotic fuel comprises cellobiose; (b) the transporter protein comprises a cellodextrin transporter protein or a functional fragment thereof; (c) the protein for enabling the metabolizing of the xenobiotic fuel comprises a beta-glucosidase protein or a functional fragment thereof or a cellobiose phosphorylase protein or a functional fragment thereof. In some embodiments, the sequence of the nucleic acid encoding the transporter protein or a functional fragment thereof or the sequence of the nucleic acid encoding a protein or a functional fragment thereof for enabling the metabolizing of the xenobiotic fuel in the bioengineered cell is codon-optimized for the bioengineered cell. In some embodiments, (a) the nucleic acid sequence encoding the cellodextrin transporter protein or a functional fragment thereof encodes a protein at least 90% identical to SEQ ID NO: 32 or SEQ ID NO: 3; or (b) the nucleic acid sequence encoding the beta-glucosidase protein or a functional fragment thereof encodes a protein at least 90% identical to SEQ ID NO: 37 or SEQ ID NO: 6. In some embodiments, the nucleic acid sequence encoding the cellodextrin transporter protein or a functional fragment thereof is at least 90% identical to SEQ ID NO: 2, SEQ ID NO: 17, SEQ ID NO: 18, or SEQ ID NO: 19; or (b) the nucleic acid sequence encoding the beta-glucosidase protein or a functional fragment thereof is at least 90% identical to SEQ ID NO: 5.

In some embodiments, the bioengineered cell further comprises a nucleic acid sequence comprising a Woodchuck hepatitis virus post-transcriptional regulatory element (WPRE) operably linked to the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof. In some embodiments, the WPRE is downstream of the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof.

In some embodiments, the cellodextrin transporter protein or functional fragment thereof is operably linked to a signal peptide, the signal peptide translocating the cellodextrin transporter protein or functional fragment thereof to the membrane of the bioengineered cell. In some embodiments, the signal peptide comprises an endoplasmic reticulum export signal (ERES).

In some embodiments, the bioengineered cell further comprises a hemagglutinin (HA) tag operably linked to the cellodextrin transporter protein or functional fragment thereof, the beta-glucosidase protein or functional fragment thereof, or the cellobiose phosphorylase protein or functional fragment thereof.

In some embodiments, the bioengineered cell further comprises a 2A ribosomal skipping peptide (e.g., T2A) operably linked to the cellodextrin transporter protein or a functional fragment thereof, the beta-glucosidase protein or a functional fragment thereof, or the cellobiose phosphorylase protein or a functional fragment thereof. In some embodiments, the 2A ribosomal skipping peptide is C-terminal to the cellodextrin transporter protein or functional fragment thereof, is C-terminal to the beta-glucosidase protein or functional fragment thereof, or is C-terminal to the cellobiose phosphorylase protein or functional fragment thereof. In some embodiments, the 2A ribosomal skipping peptide comprises a T2A ribosomal skipping peptide, a P2A ribosomal skipping peptide, a E2A ribosomal skipping peptide, or a F2A ribosomal skipping peptide. In some embodiments, the 2A ribosomal skipping peptide comprises a T2A ribosomal skipping peptide.

In some embodiments, the vector comprises a retroviral vector, a viral vector, or a plasmid vector.

In some embodiments, the bioengineered cell comprises: (a) a vector comprising a nucleic acid sequence encoding the cellodextrin transporter protein or a functional fragment thereof, the nucleic acid sequence at least 90% identical to SEQ ID NO: 28; and/or (b) a vector comprising a nucleic acid sequence encoding the cellodextrin transporter protein or a functional fragment thereof, the nucleic acid sequence at least 90% identical to SEQ ID NO: 29.

In any embodiments described herein, any nuclease system that takes advantage of homologous recombination, such as but not limited to CRISPR, may be employed in the preparation of the bioengineered cells herein.

In some embodiments, the bioengineered cell is a bioengineered immune cell. In some embodiments, the bioengineered immune cell is a mammalian cell or an avian cell. In some embodiments, the bioengineered cell is a bioengineered immune cell comprising a T-cell, a regulatory T-cell (Treg), a B-cell, a dendritic cell, a macrophage, an M1 polarized macrophage, a B cell receptor (BCR)-stimulated B cell, a tumor-infiltrating lymphocyte (TIL), or a natural killer cell (NK). In some embodiments, the bioengineered immune cell comprises a chimeric antigen receptor (CAR)-T cell, a CAR-B cell, a CAR-T regulatory cell (CAR Treg), or a T-cell engineered to alter the specificity of the T-cell receptor (TCR).

In other embodiments, the bioengineered cell is a stromal cell, a neuron, or a cardiac cell. In some embodiments, cells from a cell line may be used to prepare bioengineered immune or other cells for the various purposes described herein. In some embodiments, such cell line may be HLA matched for a particular patient population or subject to be administered and/or treated by the methods described herein.

In related aspects, disclosed herein are methods of modulating an immune response at a focus of interest in a subject in need thereof, the method comprising: administering a xenobiotic fuel-enabled bioengineered immune cell to said subject said bioengineered immune cell comprising: (a) at least one vector comprising at least one nucleic acid encoding at least one transporter protein or a functional fragment thereof for transport of the xenobiotic fuel into the bioengineered immune cell; (b) at least one vector comprising at least one nucleic acid encoding at least one protein or a functional fragment thereof for enabling the metabolizing of the xenobiotic fuel in the bioengineered immune cell; or (c) a combination of (a) and (b); administering the xenobiotic fuel to said subject; wherein said modulating the immune response comprises stimulating said immune response or suppressing said immune response.

In some embodiments, administering the xenobiotic fuel-enabled bioengineered immune cell to said subject comprises administering the xenobiotic fuel-enabled bioengineered immune cell on or adjacent to said focus of interest; or administering the xenobiotic fuel to said subject comprises implanting a scaffold comprising releasable xenobiotic fuel on, adjacent to, or near said focus of interest.

In some embodiments, (a) the xenobiotic fuel comprises cellobiose; (b) the transporter protein comprises a cellodextrin transporter protein or a functional fragment thereof; (c) the protein for enabling the metabolizing of the xenobiotic fuel comprises a beta-glucosidase protein or a functional fragment thereof or a cellobiose phosphorylase protein or a functional fragment thereof.

In some embodiments, the sequence of the nucleic acid encoding the transporter protein or a functional fragment thereof or the sequence of the nucleic acid encoding a protein or a functional fragment thereof for enabling the metabolizing of the xenobiotic fuel in the bioengineered immune cell is codon-optimized for the bioengineered immune cell.

In some embodiments, (a) the nucleic acid sequence encoding the cellodextrin transporter protein or a functional fragment thereof encodes a protein at least 90% identical to SEQ ID NO: 32 or SEQ ID NO: 3; or (b) the nucleic acid sequence encoding the beta-glucosidase protein or a functional fragment thereof encodes a protein at least 90% identical to SEQ ID NO: 37 or SEQ ID NO: 6.

In some embodiments, the method comprises a nucleic acid sequence further comprising a Woodchuck hepatitis virus post-transcriptional regulatory element (WPRE) operably linked to the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof. In some embodiments, the WPRE is downstream of the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof.

In some embodiments, the cellodextrin transporter protein or functional fragment thereof is operably linked to a signal peptide, the signal peptide translocating the cellodextrin transporter protein or functional fragment thereof to the membrane of the bioengineered immune cell. In some embodiments, the signal peptide comprises an endoplasmic reticulum export signal (ERES). In some embodiments, the bioengineered immune cell further comprises a hemagglutinin (HA) tag operably linked to the cellodextrin transporter protein or functional fragment thereof, the beta-glucosidase protein or functional fragment thereof, or the cellobiose phosphorylase protein or functional fragment thereof. In some embodiments, the bioengineered immune cell further comprises a 2A ribosomal skipping peptide operably linked to the cellodextrin transporter protein or a functional fragment thereof, the beta-glucosidase protein or a functional fragment thereof, or the cellobiose phosphorylase protein or a functional fragment thereof.

In some embodiments, the vector comprises a retroviral vector, a viral vector, or a plasmid vector.

In some embodiments, the xenobiotic fuel-enabled bioengineered immune cell comprises (a) a vector comprising a nucleic acid sequence encoding the cellodextrin transporter protein or a functional fragment thereof, the nucleic acid sequence at least 90% identical to SEQ ID NO: 28; and/or (b) a vector comprising a nucleic acid sequence encoding the cellodextrin transporter protein or a functional fragment thereof, the nucleic acid sequence at least 90% identical to SEQ ID NO: 29.

In some embodiments, the bioengineered immune cell is a mammalian cell or an avian cell.

In some embodiments, modulating the immune response comprises stimulating the immune response and wherein the bioengineered immune cell comprising a T-cell, a chimeric antigen receptor (CAR)-T cell, a T cell engineered to alter the specificity of the T-cell receptor (TCR), a B-cell, a CAR-B cell, a dendritic cell, a macrophage, an M1 polarized macrophage, a B cell receptor (BCR)-stimulated B cell, a tumor-infiltrating lymphocyte (TIL), or a natural killer cell (NK). In some embodiments, the bioengineered immune cell comprises a T-cell or a CAR-T cell and modulating the immune response comprises increasing proliferation of cytotoxic T cells, increasing proliferation of helper T cells, maintaining the population of helper T cells at the site of said tumor, activating cytotoxic T cells at the site of said solid tumor or infection, or any combination thereof. In other embodiments, the bioengineered immune cell comprises a B-cell or a CAR-B cell and modulating the immune response comprises increasing production of antibodies from the B-cell or CAR-B cell. In still other embodiments, modulating the immune response comprises suppressing the immune response and wherein the bioengineered immune cell comprises a regulatory T cell (Treg), a chimeric antigen receptor (CAR)-Treg, or a T-cell engineered to alter the specificity of the T-cell receptor (TCR). In some embodiments, the bioengineered immune cell comprises a Treg cell or a CAR-Treg cell and modulating the immune response comprises decreasing proliferation of cytotoxic T cells; decreasing proliferation of helper T cells; suppressing cytotoxic T cells at the site of said focus of interest; or any combination thereof.

In some embodiments, the focus of interest comprises a solid tumor. In some embodiments, the solid tumor comprises a cancerous, pre-cancerous, or non-cancerous tumor. In some embodiments, the solid tumor comprises a tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodenroglioma, a schwannoma, a meningioma, a melanoma, a neuroblastoma, or a retinoblastoma.

In some embodiments, the method further comprises reducing the size of the solid tumor, eliminating said solid tumor, slowing the growth of the solid tumor, or prolonging survival of said subject, or any combination thereof.

In other embodiments, the focus of interest comprises: (a) an autoimmune-targeted or symptomatic focus of an autoimmune disease; (b) a reactive focus of an allergic reaction or hypersensitivity reaction; (c) a focus of infection or symptoms of a localized infection or infectious disease; (d) an injury or a site of chronic damage; (e) a surgical site; (f) a site of a transplanted organ, tissue, or cell; or (g) a site of blood clot causing or at risk for causing a myocardial infarction, ischemic stroke, or pulmonary embolism.

In some embodiments, modulating the immune response: (a) reduces or eliminates inflammation or another symptom of said autoimmune-targeted or symptomatic focus of said autoimmune disease, prolongs survival of said subject, or any combination thereof; (b) reduces or eliminates inflammation or another symptom of allergic reaction or hypersensitivity reaction at said reactive focus of said allergic reaction or hypersensitivity reaction, prolongs survival of said subject, or any combination thereof; (c) reduces or eliminates infection or symptoms at said focus of infection or symptoms of said localized infection or infectious disease, prolongs survival of said subject, or any combination thereof; (d) reduces, eliminates, inhibits or prevents structural, organ, tissue, or cell damage, inflammation, infection, or another symptom at said site of injury or said site of chronic damage, improves structural, organ, tissue, or cell function at said site of injury or said site of chronic damage, improves mobility of said subject, prolongs survival of said subject, or any combination thereof; (e) reduces, eliminates, inhibits, or prevents structural, organ, tissue, or cell damage, inflammation, infection, or another symptom at said surgical site, improves structural, organ, tissue, or cell function at said surgical site, improves mobility of said subject, prolongs survival of said subject, or any combination thereof; (f) reduces, eliminates, inhibits or prevents transplanted organ, tissue, or cell damage or rejection, inflammation, infection or another symptom at said transplant site, improves mobility of said subject, prolongs survival of said transplanted organ, tissue, or cell, prolongs survival of said subject, or any combination thereof; or (g) reduces or eliminates said blood clot causing or at risk for causing said myocardial infarction, said ischemic stroke, or said pulmonary embolism in said subject, improves function or survival of a heart, brain, or lung organ, tissue, or cell in said subject, reduces damage to a heart, brain, or lung organ, tissue, or cell in said subject, prolongs survival of a heart, brain, or lung organ, tissue, or cell in said subject, prolongs survival of said subject, or any combination thereof.

In other related aspects, disclosed herein is a method of modulating an immune response at the site of a solid tumor or infection, said method comprising: administering a cellobiose-enabled bioengineered T cell to said subject adjacent to a solid tumor or infection, said cellobiose-enabled bioengineered T cell comprising a vector comprising a nucleic acid encoding a cellodextrin transporter protein or a functional fragment thereof and a vector comprising a nucleic acid encoding a beta-glucosidase protein or a functional fragment thereof; and administering cellobiose to said subject or implanting a scaffold that releases cellobiose adjacent to said solid tumor or infection, said modulating the immune response comprising increasing proliferation of cytotoxic T cells; increasing proliferation of helper T cells; maintaining the population of helper T cells at the site of said tumor; activating cytotoxic T cells at the site of said solid tumor or infection; or any combination thereof.

In still other related aspects, disclosed herein is a method of modulating an immune response at the site of a solid tumor or infection, said method comprising: administering a cellobiose-enabled bioengineered B cell to said subject adjacent to a solid tumor or infection, said cellobiose-enabled bioengineered B cell comprising a vector comprising a nucleic acid encoding a cellodextrin transporter protein or a functional fragment thereof and a vector comprising a nucleic acid encoding a beta-glucosidase protein or a functional fragment thereof; and administering cellobiose to said subject or implanting a scaffold that releases cellobiose adjacent to said solid tumor or infection, said modulating the immune response comprising increasing production of antibodies from the B cell; increasing isotype switching; increasing affinity maturation; or any combination thereof.

In yet other related aspects, disclosed herein is a method of modulating an immune response at a focus of interest of an autoimmune disease, an allergic reaction, a localized infection or an infectious disease, an injury or other damage, a transplant or other surgical site, or a symptom thereof, or a combination thereof, in a subject in need thereof, comprising administering to said subject a bioengineered T regulatory (Treg) cell, adjacent to said focus of interest, said cellobiose-enabled bioengineered Treg cell comprising a vector comprising a nucleic acid encoding a cellodextrin transporter protein or a functional fragment thereof and a vector comprising a nucleic acid encoding a beta-glucosidase protein or a functional fragment thereof; and administering cellobiose to said subject or implanting a scaffold that release said cellobiose adjacent to said focus of interest; wherein said regulating the immune response comprises decreasing proliferation of cytotoxic T cells; decreasing proliferation of helper T cells; suppressing cytotoxic T cells at the site of said focus of interest; or any combination thereof.

In related aspects, disclosed herein is a vector comprising at least one nucleic acid sequence encoding at least one protein for modifying a bioengineered cell to enable metabolism of a xenobiotic fuel in the cell, the xenobiotic fuel not metabolized by a corresponding unmodified cell, the vector comprising: (a) a promoter, the promoter operably linked to (i) a nucleic acid encoding a transporter protein or a functional fragment thereof for transport of the xenobiotic fuel into the bioengineered cell; (ii) a nucleic acid encoding a protein or a functional fragment thereof for enabling the metabolizing of the xenobiotic fuel in the bioengineered cell; or (iii) a combination of (i) and (ii); and (b) a selective marker.

In some embodiments, (a) the transporter protein or functional fragment thereof comprises a cellodextrin transporter protein or a functional fragment thereof; or (b) the protein or functional fragment thereof for enabling the metabolizing of the xenobiotic fuel in the bioengineered cell comprises a beta-glucosidase protein or a functional fragment thereof or a cellobiose phosphorylase protein or a functional fragment thereof.

In some embodiments, the sequence of the nucleic acid encoding the transporter protein or a functional fragment thereof or the sequence of the nucleic acid encoding a protein or a functional fragment thereof for enabling the metabolizing of the xenobiotic fuel in the bioengineered cell is codon-optimized for the bioengineered cell. In some embodiments, (a) the nucleic acid sequence encoding the cellodextrin transporter protein or a functional fragment thereof encodes a protein at least 90% identical to SEQ ID NO: 32 or SEQ ID NO: 3; or (b) the nucleic acid sequence encoding the beta-glucosidase protein or a functional fragment thereof encodes a protein at least 90% identical to SEQ ID NO: 37 or SEQ ID NO: 6.

In some embodiments, (a) the nucleic acid sequence encoding the cellodextrin transporter protein or a functional fragment thereof is at least 90% identical to SEQ ID NO: 1, SEQ ID NO:2, SEQ ID NO: 17, SEQ ID NO: 18, or SEQ ID NO: 19; or (b) the nucleic acid sequence encoding the beta-glucosidase protein or a functional fragment thereof is at least 90% identical to SEQ ID NO: 4 or SEQ ID NO: 5.

In some embodiments, the vector comprises a nucleic acid sequence comprising a Woodchuck hepatitis virus post-transcriptional regulatory element (WPRE) operably linked to the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof. In some embodiments, the WPRE is downstream of the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof.

In some embodiments, the nucleic acid sequence encoding the cellodextrin transporter protein or functional fragment thereof is operably linked to a nucleic acid sequence encoding a signal peptide, the signal peptide translocating the cellodextrin transporter protein or functional fragment thereof to the membrane of the bioengineered cell. In some embodiments, the signal peptide comprising an endoplasmic reticulum export signal (ERES)-encoding sequence. In some embodiments, the ERES-encoding sequence is C-terminal to the cellodextrin transporter protein or functional fragment thereof.

In some embodiments, the vector further comprises a nucleic acid sequence encoding a hemagglutinin (HA) tag operably linked to the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof. In some embodiments, the HA tag is C-terminal to the cellodextrin transporter protein or functional fragment thereof, is C-terminal to the beta-glucosidase protein or functional fragment thereof, or is C-terminal to the cellobiose phosphorylase protein or functional fragment thereof.

In some embodiments, the vector further comprises a nucleic acid sequence encoding a 2A ribosomal skipping peptide operably linked to the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof. In some embodiments, the 2A ribosomal skipping peptide is C-terminal to the cellodextrin transporter protein or functional fragment thereof, is C-terminal to the beta-glucosidase protein or functional fragment thereof, or is C-terminal to the cellobiose phosphorylase protein or functional fragment thereof. In some embodiments, the 2A ribosomal skipping peptide comprises a T2A ribosomal skipping peptide, a P2A ribosomal skipping peptide, a E2A ribosomal skipping peptide, or a F2A ribosomal skipping peptide. In some embodiments, the 2A ribosomal skipping peptide comprises a T2A ribosomal skipping peptide.

In some embodiments, the vector comprises a retroviral vector, a viral vector, or a plasmid vector.

In any embodiments described herein, any nuclease system that takes advantage of homologous recombination, such as but not limited to CRISPR, may be employed in the preparation of the bioengineered cells herein.

In some embodiments, (a) the vector has a nucleic acid sequence at least 90% identical to SEQ ID NO: 28 and comprising a nucleic acid sequence encoding the cellodextrin transporter protein or a functional fragment thereof at least 90% identical to SEQ ID NO: 32; (b) the vector has a nucleic acid sequence at least 90% identical to SEQ ID NO: 29 and comprising a nucleic acid sequence encoding the beta-glucosidase protein or a functional fragment thereof at least 90% identical to SEQ ID NO: 37; (c) the vector has a nucleic acid sequence at least 90% identical to SEQ ID NO: 10 and comprising a nucleic acid sequence encoding the beta-glucosidase protein or a functional fragment thereof at least 90% identical to SEQ ID NO: 5; or (d) the vector has a nucleic acid sequence at least 90% identical to SEQ ID NO: 16, SEQ ID NO: 13, SEQ ID NO: 15, SEQ ID NO: 14, SEQ ID NO: 12, SEQ ID NO: 11 or SEQ ID NO: 9 and comprising a nucleic acid sequence encoding the cellodextrin transporter protein or a functional fragment thereof at least 90% identical to SEQ ID NO: 3. In some embodiments, the xenobiotic-enabled bioengineered cell is a xenobiotic-enabled bioengineered immune cell.

In other related aspects, disclosed herein is a method of making a xenobiotic-enabled bioengineered cell, modified to metabolize a xenobiotic fuel, the xenobiotic fuel not metabolized by a corresponding unmodified cell, the method comprising: (a) selecting a xenobiotic fuel; (b) selecting a transporter protein or functional fragment thereof for transport of the xenobiotic fuel and obtaining a nucleic acid sequence encoding the same; (c) selecting a protein or functional fragment thereof for enabling the metabolizing of the xenobiotic fuel and obtaining a nucleic acid sequence encoding the same; (d) providing (i) a vector comprising a promoter, the promoter operably linked to a nucleic acid encoding a transporter protein or a functional fragment thereof for transport of the xenobiotic fuel into the bioengineered cell, and a selective marker; and (ii) a vector comprising a promoter, the promoter operably linked to a nucleic acid encoding a protein or a functional fragment thereof for metabolizing the xenobiotic fuel in the bioengineered cell, and a selective marker; (e) isolating a cell of interest from a subject; (f) transfecting or transducing the cell of interest with (i) the vector comprising a nucleic acid encoding a transporter protein or a functional fragment thereof for transport of the xenobiotic fuel into the bioengineered cell; and (ii) the vector comprising a nucleic acid encoding a protein or a functional fragment thereof for enabling the metabolizing of the xenobiotic fuel in the bioengineered cell.

In some embodiments, (a) the xenobiotic fuel comprises cellobiose; (b) the transporter protein comprises a cellodextrin transporter protein or a functional fragment thereof; (c) the protein for enabling the metabolizing of the xenobiotic fuel comprises a beta-glucosidase protein or a functional fragment thereof or a cellobiose phosphorylase protein or a functional fragment thereof. In some embodiments, the protein for enabling the metabolizing of the xenobiotic fuel comprises a beta-glucosidase protein.

In some embodiments, the method further comprises codon-optimizing the nucleic acid of step (b) and the nucleic acid of step (c) with reference to codon usage in the bioengineered cell.

In some embodiments, (a) the nucleic acid sequence encoding the cellodextrin transporter protein or a functional fragment thereof encodes a protein at least 90% identical to SEQ ID NO: 32 or SEQ ID NO: 3; or (b) the nucleic acid sequence encoding the beta-glucosidase protein or a functional fragment thereof encodes a protein at least 90% identical to SEQ ID NO: 37 or SEQ ID NO: 6.

In some embodiments, (a) the nucleic acid sequence encoding the cellodextrin transporter protein or a functional fragment thereof is at least 90% identical to SEQ ID NO: 1, SEQ ID NO: 2, SEQ ID NO: 17, SEQ ID NO: 18, or SEQ ID NO: 19; or (b) the nucleic acid sequence encoding the beta-glucosidase protein or a functional fragment thereof is at least 90% identical to SEQ ID NO: 4 or SEQ ID NO: 5.

In some embodiments, the cell of interest comprises an immune cell, and the bioengineered cell comprising a bioengineered immune cell.

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure, the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value.

Similarly, when values are expressed as approximations, by use of the antecedent “about,” it is understood that the particular value forms another embodiment. All ranges are inclusive and combinable. In the context of the present disclosure, by “about” a certain amount it is meant that the amount is within ±20% of the stated amount, or preferably within ±10% of the stated amount, or more preferably within ±5% of the stated amount.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized or customized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The term “higher vertebrates” is used herein and includes avians (birds) and mammals. The compositions described herein can be used to treat any suitable mammal, including primates, such as monkeys and humans, horses, cows, cats, dogs, rabbits, sheep, goats, pigs, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine or porcine. In another embodiment, the subject is mammalian.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

As used herein, the terms “treat”, “treatment”, or “therapy” (as well as different forms thereof) refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. Those in need of treatment include those already with the disease or condition as well as those prone to having the disease or condition or those in which the disease or condition is to be prevented.

As used herein, the terms “component,” “composition,” “formulation”, “composition of compounds,” “compound,” “drug,” “pharmacologically active agent,” “active agent,” “therapeutic,” “therapy,” “treatment,” or “medicament,” are used interchangeably herein, as context dictates, to refer to a compound or compounds or composition of matter which, when administered to a subject (human or animal) induces a desired pharmacological and/or physiologic effect by local and/or systemic action. A personalized composition or method refers to a product or use of the product in a regimen tailored or individualized to meet specific needs identified or contemplated in the subject.

The terms “subject,” “individual,” and “patient” are used interchangeably herein, and refer to an animal, for example a human, to whom treatment with a composition or formulation in accordance with the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g. mouse or rat), guinea pig, goat, pig, cat, rabbits, cows, horses and non-mammals such as reptiles, amphibians, chickens, and turkeys. The term “higher vertebrates” is used herein and includes avians (birds) and mammals. The compositions described herein can be used to treat any suitable mammal, including primates, such as monkeys and humans, horses, cows, cats, dogs, rabbits, sheep, goats, pigs, and rodents such as rats and mice. In one embodiment, the mammal to be treated is human. The human can be any human of any age. In an embodiment, the human is an adult. In another embodiment, the human is a child. The human can be male, female, pregnant, middle-aged, adolescent, or elderly. According to any of the methods of the present invention and in one embodiment, the subject is human. In another embodiment, the subject is a non-human primate. In another embodiment, the subject is murine, which in one embodiment is a mouse, and, in another embodiment is a rat. In another embodiment, the subject is canine, feline, bovine, equine, laprine, or porcine. In another embodiment, the subject is mammalian.

Conditions and disorders in a subject for which a particular drug, compound, composition, formulation (or combination thereof) is said herein to be “indicated” are not restricted to conditions and disorders for which that drug or compound or composition or formulation has been expressly approved by a regulatory authority, but also include other conditions and disorders known or reasonably believed by a physician or other health or nutritional practitioner to be amenable to treatment with that drug or compound or composition or formulation or combination thereof.

In some embodiments, the bioengineered cells and methods herein are used for the treatment of vertebrate organisms. In some embodiments, the bioengineered cells and methods are used for the treatment of homeothermic vertebrate organisms (e.g., mammals and birds). In some embodiments, the bioengineered cells and methods are used for the treatment of human or non-human mammals.

### Xenobiotic Fuel-Enabled Bioengineered Cells and Methods of Making Them

A “xenobiotic” comprises a chemical substance found within an organism that is not naturally produced or expected to be present within the organism. A “xenobiotic fuel” comprises an energy source substance (e.g., a carbohydrate, such as a sugar or a starch) found within an organism that is not naturally produced or metabolized or expected to be present within the organism. In some instances, the organism may not be able to metabolize the energy source substance or may only partially or inefficiently metabolize the energy source substance, e.g., due to the absence of an enzyme capable of recognizing or transporting the energy source substance into the cell, due to the absence of an enzyme capable of metabolizing the energy source substance, or due to the toxicity of the energy source substance due to an improper processing of the energy source.

Cellobiose, a glucose disaccharide found abundantly in plant matter (e.g., wood pulp), has great potential to serve as a carbon and energy source but remains inert to catabolic processes in mammalian systems for two primary reasons. First, metazoan sugar transport is restricted to monosaccharides. Second, the β-1,4-glycosidic bond that joins glucose molecules in cellobiose is inefficiently hydrolyzed by mammalian glycoside hydrolases. These processes, that is the transport and hydrolyzation of cellobiose, are efficiently carried out in cellulolytic microbes, but not in most mammals, including humans. Most mammals have limited ability to digest dietary fiber such as cellulose. The glucoses in cellobiose are joined together by a bond that is not readily breakable in most mammals, including humans.

In some embodiments, the xenobiotic fuel comprises cellobiose. In some embodiments, the subject is a vertebrate. In some embodiments, the subject is a homeothermic vertebrate (e.g., a mammal or a bird). In some embodiments the subject is a human or non-human mammal.

In some embodiments, a protein for transporting a xenobiotic fuel and/or a protein for metabolizing a xenobiotic fuel is selected, and the nucleic acid encoding the protein or proteins is optimized for use in a subject or species in need thereof.

In some embodiments, the xenobiotic fuel comprises cellobiose, the transporter protein comprises cellodextrin transporter protein (CDT-1) or a functional fragment thereof, and the protein for metabolizing the xenobiotic fuel comprises a beta glucosidase protein (GH1-1) or a functional fragment thereof and/or a cellobiose phosphorylase protein or a functional fragment thereof. In some embodiments, the proteins selected comprise CDT-1 or a functional fragment thereof and (i) GH1-1 or a functional fragment thereof and/or (ii) cellobiose phosphorylase protein or a functional fragment thereof. In some embodiments, the proteins selected comprise CDT-1 or a functional fragment thereof and GH1-1 or a functional fragment thereof. In some embodiments, the proteins selected comprise CDT-1 or a functional fragment thereof and cellobiose phosphorylase protein or a functional fragment thereof.

Because a foreign protein may not undergo the appropriate post-translational processing and localization in the host species, the foreign protein in the vector may be operably linked to an appropriate signal peptide.

In some embodiments, the cellodextrin transporter protein or functional fragment thereof operably linked to a signal peptide, the signal peptide translocating the cellodextrin transporter protein or functional fragment thereof to the membrane of the bioengineered cell.

In some embodiments, the signal peptide comprising an endoplasmic reticulum export signal (ERES).

In some embodiments, a hemagglutinin (HA) tag is operably linked to the foreign protein.

In some embodiments, a 2A ribosomal skipping peptide (2A self-cleaving peptide, 2A peptide) is operably linked to the foreign protein. In some embodiments, a 2A ribosomal skipping protein is operably linked C-terminal to the foreign protein. 2A ribosomal skipping peptides include, but are not limited to, P2A, E2A, F2A, and T2A. Adding an optional glycine (Gly) and/or serine (Ser) linkers on the N-terminal of a 2A peptide can improve efficiency.

In some embodiments, a Woodchuck hepatitis virus post-transcriptional regulatory element (WPRE) is operably linked to the foreign protein or to a 2A ribosomal skipping protein operably linked to the foreign protein. In some embodiments, the WPRE is downstream of the foreign protein. The Woodchuck Hepatitis Virus (WHV) Posttranscriptional Regulatory Element (WPRE) is a DNA sequence that, when transcribed, creates a tertiary structure enhancing expression, e.g., in a viral vector.

In some embodiments, a cellodextrin transporter protein or functional fragment thereof, the beta-glucosidase protein or functional fragment thereof, or the cellobiose phosphorylase protein or functional fragment thereof.

Although each codon of DNA or RNA is specific for only one amino acid (or one stop signal), the genetic code is described as degenerate, or redundant, because a single amino acid may be coded for by more than one codon. For a given amino acid, a particular species of organism may preferentially favor a particular codon. Methods of optimizing nucleic acid sequences (codon optimization) of one species to be expressed more efficiently in another species are available. Examples of codon-optimization tools include, but are not limited to, the IDT Codon Optimization Tool (https://www.idtdna.com/pages/tools/codon-optimization-tool), BLUE HERON™ BioTech Codon Optimization Tool (https://www.blueheronbio.com/codon-optimization/?gclid=CjwKCAjw9MuCBhBUEiwAbDZ-7jQJqeOS6NfjW40raaApv_wPSBk6kTzS7V3D1CxiQifvAfUBvJ_6hhoCttEQAvD_BwE) (EUROFINS GENOMICS™), or OPTIMUM GENE™ BioTech Codon Optimization Tool (https://www.genscript.com/codon-opt.html?src=google&gclid=CjwKCAjw9MuCBhBUEiwAbDZ-7sdh1Ve2q8emWgomPW4wxh9pigffndWQJefv7ay19-rB-s919Rbp9BoCt7oQAvD_BwE) (GENSCRIPT®).

In some embodiments, a vector comprising the optimized nucleic acid is constructed having an operably linked promoter and a selectable marker. A vector comprises a DNA or RNA molecule used as a vehicle to artificially carry foreign genetic material into another cell, where it can be replicated and/or expressed (e.g., plasmid, cosmid, Lambda phages). A vector has an origin of replication, a multicloning site, and a selectable marker. A vector containing foreign DNA or RNA is termed recombinant DNA or RNA, respectively. Vectors include, but are not limited to, plasmids, viral vectors, cosmids, and artificial chromosomes. Viral vectors include, but are not limited to, retroviral vectors. Examples of vectors include, but are not limited to, those disclosed herein. Additional examples of vectors include MSCV plasmid (ADDGENE™; Plasmid #24828; https://www.addgene.org/248284/) and MSCV-internal ribosome entry site (IRES)-GFP (ADDGENE™; Plasmid #20672; https://www.addgene.org/20672/). In some embodiments, the vector comprises a MSCV MCS PGK-GFP vector or a MSCV MCS PGK-mCherry vector.

In some embodiments, more than one protein for metabolizing a xenobiotic fuel is selected, and the nucleic acid for each is optimized for use in a subject or species in need thereof. In some embodiments, a vector comprising the optimized nucleic acid is constructed having a selectable marker. In some embodiments, separate vectors (one for each protein) are constructed, each having a discrete selectable marker. In some embodiments, the selectable marker comprises a fluorescent or colorimetric marker. In some embodiments, the selectable marker comprises an antibiotic resistance gene or marker. In some embodiments, the selectable marker comprises a fluorescent marker.

In some embodiments, a cell of interest in the subject is harvested and transfected with the vector to render the cell xenobiotic fuel-enabled, namely, enabling the cell of the subject to, e.g., transport, metabolize, process, or store, the xenobiotic fuel. The xenobiotic fuel-enabled bioengineered or transgenic cell is administered to the subject.

In some embodiments, the method comprises providing a xenobiotic fuel-enabled bioengineered cell. In some embodiments, the method comprises providing a xenobiotic fuel-enabled transgenic cell.

In some embodiments, the cell is administered to the subject at or near the focus of interest, as described herein. In some embodiments, the bioengineered or transgenic cell is surgically implanted, systemically or locally infused, or injected. In some embodiments, a scaffold is provided for delivery of the cell.

The xenobiotic fuel is also administered to the subject, either simultaneously or separately. In some embodiments, the subject is placed on a diet that includes the xenobiotic fuel (e.g., cellobiose) and is low on one or more other fuel sources (e.g., glucose) that form a part of the normal diet for the subject's species or are derived from metabolism of foods part of the normal diet for the subject's species.

In some embodiments, the xenobiotic fuel is administered to the subject at or near the focus of interest, as described herein. In some embodiments, the xenobiotic fuel is injected or provided intravenously, administered as a pill, tablet, or liquid, or other types of administration as described herein. In some embodiments, a scaffold is provided for delivery of the xenobiotic fuel over time.

In some embodiments, the xenobiotic fuel-enabled bioengineered cell comprises a bioengineered immune cell (e.g., a T cell, a B cell, a dendritic cell, a natural killer (NK) cell, or a T regulatory cell (Treg)) bioengineered to express proteins to break down cellobiose to enable the immune cell to be used to treat a disease or abnormal physiological condition, the disease or abnormal physiological condition resulting in a low glucose environment.

### Immune Cells and Regulatory Compounds

In some embodiments the xenobiotic fuel-enabled bioengineered cell comprises a bioengineered or transgenic immune cell comprising a T-cell bioengineered to express proteins to break down cellobiose, a regulatory T-cell (Treg) bioengineered to express proteins to break down cellobiose, a B-cell bioengineered to express proteins to break down cellobiose, a dendritic cell bioengineered to express proteins to break down cellobiose, or a natural killer cell (NK) bioengineered to express proteins to break down cellobiose. In some embodiments, immune cells, for example T cells, bioengineered to express proteins to break down cellobiose, are generated and expanded by the presence of cytokines in vivo. In some embodiments, cytokines that affect generation and maintenance to T-helper cells in vivo comprise IL-2, IL-12, and IL-15. In some embodiments, TGF-β and/or IL-2 play a role in differentiating naïve T cells to become Treg cells.

“Cytokines” are a category of small proteins (˜5-20 kDa) critical to cell signaling. Cytokines are peptides and usually are unable to cross the lipid bilayer of cells to enter the cytoplasm. Among other functions, cytokines may be involved in autocrine, paracrine and endocrine signaling as immunomodulating agents. Cytokines may be proinflammatory or anti-inflammatory. Cytokines include, but are not limited to, chemokines (cytokines with chemotactic activities), interferons, interleukins (ILs; cytokines made by one leukocyte and acting on one or more other leukocytes), lymphokines (produced by lymphocytes), monokines (produced by monocytes), and tumor necrosis factors. Cells producing cytokines include, but are not limited to, immune cells (e.g., macrophages, B lymphocytes, T lymphocytes and mast cells), as well as endothelial cells, fibroblasts, and various stromal cells. A particular cytokine may be produced by more than one cell type.

A skilled artisan would appreciate that the term “cytokine” may encompass cytokines beneficial to enhancing an immune response targeted against a cancer or a pre-cancerous or non-cancerous tumor or lesion. A skilled artisan would also appreciate that the term “cytokine” may encompass cytokines beneficial to enhancing an immune response against a disease or inflammation (e.g., resulting from surgery, an injury, or damage from an autoimmune response) or that the term “cytokine” may encompass cytokines beneficial to reducing an abnormal autoimmune response.

In some embodiments, the cytokine comprises an interleukin (IL). A skilled artisan would appreciate that interleukins comprise a large family of molecules, including, but not limited to, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and IL-36. In some embodiments, the interleukin comprises an IL-2, IL-4, IL-6, IL-7, IL-10, IL-12, or an IL-15, or any combination thereof. In some embodiments, the cytokine comprises an IL-2. In some embodiments, the IL-2 cytokine comprises an IL-2 superkine (super IL-2 cytokine, Super2). IL-2 is a 133 amino acid glycoprotein with one intramolecular disulfide bond and variable glycosylation. “IL-2 superkine” or “Super2” (Fc) is an artificial variant of IL-2 containing mutations at positions L80F/R81D/L85V/I86V/I92F. These mutations are located in the molecule's core that acts to stabilize the structure and to give it a receptor-binding conformation mimicking native IL-2 bound to CD25. These mutations effectively eliminate the functional requirement of IL-2 for CD25 expression and elicit proliferation of T cells. Compared to IL-2, the IL-2 superkine induces superior expansion of cytotoxic T cells, leading to improved antitumor responses in vivo, and elicits proportionally less toxicity by lowering the expansion of T regulatory cells and reducing pulmonary edema.

A “T cell” is characterized and distinguished by the T cell receptor (TCR) on the surface. A T cell is a type of lymphocyte that arises from a precursor cell in the bone marrow before migrating to the thymus, where it differentiates into one of several kinds of T cells. Differentiation continues after a T cell has left the thymus. A “cytotoxic T cell” (CTL) is a CD8+ T cell able to kill, e.g., virus-infected cells or cancer cells. A “T helper cell” is a CD4+ T cell that interacts directly with other immune cells (e.g., regulatory B cells) and indirectly with other cells to recognize foreign cells to be killed. “Regulatory T cells” (T regulatory cells; Treg), also known as “suppressor T cells,” enable tolerance and prevent immune cells from inappropriately mounting an immune response against “self,” but may be co-opted by cancer or other cells. In autoimmune disease, “self-reactive T cells” mount an immune response against “self” that damages healthy, normal cells.

One skilled in the art appreciates the many mechanisms of T cell immunostimulation and/or immunosuppression. Likewise, one skilled in the art appreciates the many mechanisms of Treg induction and/or suppression of Treg induction.

T cell immunostimulatory compounds include, but are not limited to, T cell activators, T cell attractants, or T cell adhesion compounds. T cell immunostimulatory compounds include, but are not limited to, cytokines, chemokine ligands, and anti-CD antibodies or fragments thereof. Non-limiting examples include interleukins (e.g., IL-2, IL-12, or IL-15), chemokine ligands (e.g., CCL ligands, including CCL21), and anti-CD antibodies (e.g., anti-CD3 or anti-CD28) or fragments thereof, or any combination(s) thereof.

T cell immunosuppression compounds include, but are not limited to cytokines, chemokines, antibodies, or enzymes.

Compounds that suppress induction of Tregs include, but are not limited to, inhibitors of transforming growth factor-beta (TGF-β), such as an inhibitor of the TGF-β receptor. Non-limiting examples of TGF-β receptor inhibitors include galinusertib (LY2157299), SB505124, small molecule inhibitors, antibodies, chemokines, apoptosis signals (e.g., cytotoxic T-lymphocyte-associated protein 4/programmed cell death protein 1 (CTLA-4/PD-1); Granzyme; tumor necrosis factor (TNF)-related apoptosis-inducing ligand (TRAIL); Fas/Fas-L, Galectin-9/transmembrane immunoglobulin and mucin domain 3 (TIM-3)). Compounds that induce Tregs include TGF-β and activators thereof (e.g., SB 431542, A 83-01, RepSox, LY 364947, D 4476, SB 525334, GW 788388, SD 208, R 268712, IN 1130, SM 16, A 77-01, AZ 12799734).

As used herein, a “targeting agent,” or “affinity reagent,” is a molecule that binds to an antigen or receptor or other molecule. In some embodiments, a “targeting agent” is a molecule that specifically binds to an antigen or receptor or other molecule. In certain embodiments, some or all of a targeting agent is composed of amino acids (including natural, non-natural, and modified amino acids), nucleic acids, or saccharides. In certain embodiments, a “targeting agent” is a small molecule.

As used herein, the term “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains VL and CL, and each heavy chain comprising immunoglobulin domains VH, C-gamma-1 (Cγ1), C-gamma-2 (Cy2), and C-gamma-3 (Cy3). In each pair, the light and heavy chain variable regions (VL and VH) are together responsible for binding to an antigen, and the constant regions (CL, Cγ1, Cy2, and Cy3, particularly Cy2, and Cy3) are responsible for antibody effector functions. In some mammals, for example in camels and llamas, full-length antibodies may consist of only two heavy chains, each heavy chain comprising immunoglobulin domains VH, Cy2, and Cy3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains including but not limited to VH, Cγ1, Cy2, Cy3, VL, and CL.

Depending on the amino acid sequence of the constant domain of their heavy chains, intact antibodies can be assigned to different “classes”. There are five-major classes (isotypes) of intact antibodies: IgA, IgD, IgE, IgG, and IgM, and several of these may be further divided into “subclasses”, e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art.

As used herein, the term “immunoglobulin G” or “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, IgG3. As used herein, the term “modified immunoglobulin G” refers to a molecule that is derived from an antibody of the “G” class. As used herein, the term “antibody” refers to a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA isotypes or classes, respectively.

The term “antibody” is meant to include full-length antibodies, and may refer to a natural antibody from any organism, an engineered antibody, or an antibody generated recombinantly for experimental, therapeutic, or other purposes as further defined below. Furthermore, full-length antibodies comprise conjugates as described and exemplified herein. As used herein, the term “antibody” comprises monoclonal and polyclonal antibodies. Antibodies can be antagonists, agonists, neutralizing, inhibitory, or stimulatory. Specifically included within the definition of “antibody” are full-length antibodies described and exemplified herein. By “full length antibody” herein is meant the structure that constitutes the natural biological form of an antibody, including variable and constant regions.

The “variable region” of an antibody contains the antigen binding determinants of the molecule, and thus determines the specificity of an antibody for its target antigen. The variable region is so named because it is the most distinct in sequence from other antibodies within the same isotype. The majority of sequence variability occurs in the complementarity determining regions (CDRs). There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens.

Furthermore, antibodies may exist in a variety of other forms including, for example, Fv, Fab, and (Fab′)2, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986)).

The term “epitope” as used herein refers to a region of the antigen that binds to the antibody or antigen-binding fragment. It is the region of an antigen recognized by a first antibody wherein the binding of the first antibody to the region prevents binding of a second antibody or other bivalent molecule to the region. The region encompasses a particular core sequence or sequences selectively recognized by a class of antibodies. In general, epitopes are comprised by local surface structures that can be formed by contiguous or noncontiguous amino acid sequences.

As used herein, the terms “selectively recognizes”, “selectively bind” or “selectively recognized” mean that binding of the antibody, antigen-binding fragment or other bivalent molecule to an epitope is at least 2-fold greater, preferably 2-5 fold greater, and most preferably more than 5-fold greater than the binding of the molecule to an unrelated epitope or than the binding of an antibody, antigen-binding fragment or other bivalent molecule to the epitope, as determined by techniques known in the art and described herein, such as, for example, ELISA or cold displacement assays.

As used herein, the term “Fc domain” encompasses the constant region of an immunoglobulin molecule. The Fc region of an antibody interacts with a number of Fc receptors and ligands, imparting an array of important functional capabilities referred to as effector functions, as described herein. For IgG, the Fc region comprises Ig domains CH2 and CH3. An important family of Fc receptors for the IgG isotype are the Fc gamma receptors (FcγRs). These receptors mediate communication between antibodies and the cellular arm of the immune system.

As used herein, the term “Fab domain” encompasses the region of an antibody that binds to antigens. The Fab region is composed of one constant and one variable domain of each of the heavy and the light chains.

In one embodiment, the term “antibody” or “antigen-binding fragment” respectively refer to intact molecules as well as functional fragments thereof, such as Fab, a scFv-Fc bivalent molecule, F(ab′)2, and Fv that are capable of specifically interacting with a desired target. In some embodiments, the antigen-binding fragments comprise:
(1) Fab, the fragment which contains a monovalent antigen-binding fragment of an antibody molecule, which can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)2 is a dimer of two Fab′ fragments held together by two disulfide bonds;
(4) Fv, a genetically engineered fragment containing the variable region of the light chain and the variable region of the heavy chain expressed as two chains; and
(5) Single chain antibody (“SCA”), a genetically engineered molecule containing the variable region of the light chain and the variable region of the heavy chain, linked by a suitable polypeptide linker as a genetically fused single chain molecule.
(6) scFv-Fc, is produced in one embodiment, by fusing single-chain Fv (scFv) with a hinge region from an immunoglobulin (Ig) such as an IgG, and Fc regions.

In some embodiments, an antibody provided herein is a monoclonal antibody. In some embodiments, the antigen-binding fragment provided herein is a single chain Fv (scFv), a diabody, a tri(a)body, a di- or tri-tandem scFv, a scFv-Fc bivalent molecule, an Fab, Fab′, Fv, F(ab′)2 or an antigen binding scaffold (e.g., affibody, monobody, anticalin, DARPin, Knottin, etc.). “Affibodies” are small proteins engineered to bind to a large number of target proteins or peptides with high affinity, often imitating monoclonal antibodies, and are antibody mimetics.

As used herein, the terms “bivalent molecule” or “BV” refer to a molecule capable of binding to two separate targets at the same time. The bivalent molecule is not limited to having two and only two binding domains and can be a polyvalent molecule or a molecule comprised of linked monovalent molecules. The binding domains of the bivalent molecule can selectively recognize the same epitope or different epitopes located on the same target or located on a target that originates from different species. The binding domains can be linked in any of a number of ways including, but not limited to, disulfide bonds, peptide bridging, amide bonds, and other natural or synthetic linkages known in the art (Spatola et al., “Chemistry and Biochemistry of Amino Acids, Peptides and Proteins,” B. Weinstein, eds., Marcel Dekker, New York, p. 267 (1983); Morley, J. S., “Trends Pharm Sci.” (1980) pp. 463-468; Hudson et al., Int. J. Pept. Prot. Res. (1979) 14, 177-185; Spatola et al., Life Sci. (1986) 38, 1243-1249; Hann, M. M., J. Chem. Soc. Perkin Trans. I (1982) 307-314; Almquist et al., J. Med. Chem. (1980) 23, 1392-1398; Jennings-White et al., Tetrahedron Lett. (1982) 23, 2533; Szelke et al., European Application EP 45665; Chemical Abstracts 97, 39405 (1982); Holladay, et al., Tetrahedron Lett. (1983) 24, 4401-4404; and Hruby, V. J., Life Sci. (1982) 31, 189-199).

As used herein, the terms “binds” or “binding” or grammatical equivalents, refer to compositions having affinity for each other. “Specific binding” is where the binding is selective between two molecules. A particular example of specific binding is that which occurs between an antibody and an antigen. Typically, specific binding can be distinguished from non-specific when the dissociation constant (KD) is less than about 1×10-5 M or less than about 1×10-6 M or 1×10-7 M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In addition to antibody sequences, an antibody may comprise other amino acids, e.g., forming a peptide or polypeptide, such as a folded domain, or to impart to the molecule another functional characteristic in addition to ability to bind antigen. For example, antibodies may carry a detectable label, such as fluorescent or radioactive label, or may be conjugated to a toxin (such as a holotoxin or a hemitoxin) or an enzyme, such as beta-galactosidase or alkaline phosphatase (e.g., via a peptidyl bond or linker).

In one embodiment, an antibody comprises a stabilized hinge region. The term “stabilized hinge region” will be understood to mean a hinge region that has been modified to reduce Fab arm exchange or the propensity to undergo Fab arm exchange or formation of a half-antibody or a propensity to form a half-antibody. “Fab arm exchange” refers to a type of protein modification for human immunoglobulin, in which a human immunoglobulin heavy chain and attached light chain (half-molecule) is swapped for a heavy-light chain pair from another human immunoglobulin molecule. Thus, human immunoglobulin molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half-antibody” forms when a human immunoglobulin antibody dissociates to form two molecules, each containing a single heavy chain and a single light chain. In one embodiment, the stabilized hinge region of human immunoglobulin comprises a substitution in the hinge region.

In one embodiment, the term “hinge region” as used herein refers to a proline-rich portion of an immunoglobulin heavy chain between the Fc and Fab regions that confers mobility on the two Fab arms of the antibody molecule. It is located between the first and second constant domains of the heavy chain. The hinge region includes cysteine residues which are involved in inter-heavy chain disulfide bonds. In one embodiment, the hinge region includes cysteine residues which are involved in inter-heavy chain disulfide bonds.

In one embodiment, the antibody or antigen-binding fragment binds its target with a KD of 0.1 nM-10 mM. In one embodiment, the antibody or antigen-binding fragment binds its target with a KD of 0.1 nM-1 mM. In one embodiment, the antibody or antigen-binding fragment binds its target with a KD within the 0.1 nM range. In one embodiment, the antibody or antigen-binding fragment binds its target with a KD of 0.1-2 nM. In another embodiment, the antibody or antigen-binding fragment binds its target with a KD of 0.1-1 nM. In another embodiment, the antibody or antigen-binding fragment binds its target with a KD of 0.05-1 nM. In another embodiment, the antibody or antigen-binding fragment binds its target with a KD of 0.1-0.5 nM. In another embodiment, the antibody or antigen-binding fragment binds its target with a KD of 0.1-0.2 nM.

In some embodiments, the antibody or antigen-binding fragment thereof provided herein comprises a modification. In another embodiment, the modification minimizes conformational changes during the shift from displayed to secreted forms of the antibody or antigen-binding fragment. It is to be understood by a skilled artisan that the modification can be a modification known in the art to impart a functional property that would not otherwise be present if it were not for the presence of the modification. Encompassed are antibodies which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

In some embodiments, the modification is one as further defined herein below. In some embodiments, the modification is a N-terminus modification. In some embodiments, the modification is a C-terminal modification. In some embodiments, the modification is an N-terminus biotinylation. In some embodiments, the modification is a C-terminus biotinylation. In some embodiments, the secretable form of the antibody or antigen-binding fragment comprises an N-terminal modification that allows binding to an Immunoglobulin (Ig) hinge region. In some embodiments, the Ig hinge region is from but is not limited to, an IgA hinge region. In some embodiments, the secretable form of the antibody or antigen-binding fragment comprises an N-terminal modification that allows binding to an enzymatically biotinylatable site. In some embodiments, the secretable form of the antibody or antigen-binding fragment comprises a C-terminal modification that allows binding to an enzymatically biotinylatable site. In some embodiments, biotinylation of said site functionalizes the site to bind to any surface coated with streptavidin, avidin, avidin-derived moieties, or a secondary reagent.

It will be appreciated that the term “modification” can encompass an amino acid modification such as an amino acid substitution, insertion, and/or deletion in a polypeptide sequence.

In one embodiment, a variety of radioactive isotopes are available for the production of radioconjugate antibodies and other proteins and can be of use in the methods and compositions provided herein. Examples include, but are not limited to, At211, Cu64, 1131, 1125, Y90, Re186, Re188, Sm153, Bi212, P32, Zr89 and radioactive isotopes of Lu. In a further embodiment, the amino acid sequences of the invention may be homologues, variants, isoforms, or fragments of the sequences presented. The term “homolog” as used herein refers to a polypeptide having a sequence homology of a certain amount, namely of at least 70%, e.g. at least 80%, 90%, 95%, 96%, 97%, 98%, 99% of the amino acid sequence it is referred to. Homology refers to the magnitude of identity between two sequences. Homolog sequences have the same or similar characteristics, in particular, have the same or similar property of the sequence as identified. The term ‘variant’ as used herein refers to a polypeptide wherein the amino acid sequence exhibits substantially 70, 80, 95, or 99% homology with the amino acid sequence as set forth in the sequence listing. It should be appreciated that the variant may result from a modification of the native amino acid sequences, or by modifications including insertion, substitution or deletion of one or more amino acids. The term “isoform” as used herein refers to variants of a polypeptide that are encoded by the same gene, but that differ in their isoelectric point (pI) or molecular weight (MW), or both. Such isoforms can differ in their amino acid composition (e.g. as a result of alternative splicing or limited proteolysis) and in addition, or in the alternative, may arise from differential post-translational modification (e.g., glycosylation, acylation, phosphorylation deamidation, or sulphation). As used herein, the term “isoform” also refers to a protein that exists in only a single form, i.e., it is not expressed as several variants. The term “fragment” as used herein refers to any portion of the full-length amino acid sequence of protein of a polypeptide of the invention which has less amino acids than the full-length amino acid sequence of a polypeptide of the invention. The fragment may or may not possess a functional activity of such polypeptides.

In an alternate embodiment, enzymatically active toxin or fragments thereof that can be used in the compositions and methods provided herein include, but are not limited, to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

A chemotherapeutic or other cytotoxic agent may be conjugated to the protein, according to the methods provided herein, as an active drug or as a prodrug. The term “prodrug” refers to a precursor or derivative form of a pharmaceutically active substance that is less cytotoxic to tumor cells compared to the parent drug and is capable of being enzymatically activated or converted into the more active parent form. (See, for example Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985.) The prodrugs that may find use with the compositions and methods as provided herein include but are not limited to phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies and Fc fusions of the compositions and methods as provided herein include but are not limited to any of the aforementioned chemotherapeutic.

Non-limiting examples of antibodies, antibody fragments and antigen-binding proteins include single-chain antibodies such as scFvs. A non-limiting example, a scFv that blocks PD-1 for the treatment of cancer or tumor, including in association with CAR-T therapy, wherein activation of scFv production can be directed at a particular site in the body, in one embodiment, at or near a tumor. Another non-limiting example includes brolucizumab, which targets VEGF-A and is used to treat wet age-related macular degeneration.

In another example, the therapeutic protein is an immune checkpoint inhibitor, such as an antibody fragment, or antigen-binding protein, that inhibits a checkpoint molecule, such as, but not limited to, PD-1, PD-L1, CTLA-4, CTLA-4 receptor, PD1-L2, 4-1BB, OX40, LAG-3, and TIM-3. In one embodiment, a scFv that inhibits a checkpoint protein.

### Cell Adhesion/Attraction Components

Any one or more cell adhesion and/or cell attraction and/or immunostimulatory and/or immunosuppression compounds or components may be included or as part of the treatments described herein. In one embodiment, such components attract or activate cellobiose-enabled bioengineered T cells. Non-limiting examples include CCL21, anti-CD3 antibodies, anti-CD28 antibodies, or any combination thereof. In one embodiment, a combination of anti-CD3 and an anti-CD28 antibodies are used. Any one or more immunostimulatory components may be included. In some embodiments, components such as but not limited to IL-2, IL-4, IL-6, IL-7, IL-10, IL-12 and IL-15 are used, singly or in any combination. In other embodiments, such compounds or components or others (e.g., anti-CD3 or anti-CD28 antibodies) suppress cellobiose-enabled bioengineered T cell attraction or cellobiose-enabled bioengineered T cell activation. Additional embodiments are described elsewhere herein.

### Treg Regulators

In some embodiments, a bioengineered Treg (suppressor T cell) is selectively activated by administration of cellobiose. Such activation may be useful in the suppression of an immune response, such as an autoimmune response and thus for the treatment of an autoimmune disease. Alternately, as described below, for immunotherapy of cancer and infection, regulating or suppressing Tregs is desirable.

In some embodiments, glycolytic metabolism destabilizes Treg function (e.g., by promoting interferon-gamma (IFN-γ) production) to promote inflammation (e.g., destabilizing Tregs in tumor cells).

In some embodiments, any one of various methods of regulating Treg induction and/or suppression of Treg induction may be used. A TGF-β inhibitor (TGF-βi) such as a TGF-β receptor inhibitor may be used concomitantly. Non-limiting examples include galinusertib (LY2157299) or SB505124. In one embodiment, the TGF-βi suppresses the formation of induced Tregs and thus enhances the tumoricidal activity of T cells attracted to, activated, or delivered by the scaffolds described herein. In one embodiment the TGF-β inhibitor or inducer is slowly released from the microparticles. Alternatively, compounds that induce Tregs may be used. Non-limiting examples include TGF-β and activators thereof (e.g., IL-2, IL-4, IL-6, IL-7, IL-10, IL-12, IL-15). Additional embodiments are described elsewhere herein.

### Scaffold for Xenobiotic Fuel Delivery

In some aspects, the bioengineered immune cells are engineered to transport and break down, e.g., cellobiose or another xenobiotic fuel for subsequent metabolism, unlike tumor or cancer cells and many infectious agents. Described herein is an approach to enable the local delivery of cellobiose or another xenobiotic fuel into the tumor or cancer environment for the enhancement of the immune responses during immunotherapy. Also described herein is an approach to enable the local delivery of cellobiose or another xenobiotic fuel into the environment of the infection for the enhancement of the immune responses during immunotherapy. An implantable scaffold is provided comprising means for local delivery of, e.g., cellobiose or alternative xenobiotic fuel (e.g., in PLGA nanoparticles embedded in the scaffold) and also one or more immunostimulatory compounds to attract and activate bioengineered cytotoxic T cells to target the tumor or infection (e.g., IL-2 on silica-heparin microparticles embedded in the scaffold). Systemic effects are avoided by employing local effects of the scaffold, which can induce a potent T cell response to a tumor or remaining tumor after resection, or even treat inoperable tumors, or to sites of infection, and then the scaffold can biodegrade over time.

To facilitate the immune response against solid tumors, provided herein is a multifunctional biomaterial that is placed adjacent to a tumor and which carries or attracts and potentiates bioengineered cytotoxic T cells and suppresses local regulatory T cells. Together these activities allow for the much sought-after materials and methods for overcoming the immunosuppressive effects of the microenvironment of solid tumors, localized infections, and other localized medical conditions. Examples of this scaffold and its related agents, materials, and methods can be found, e.g., in WO 2021/055658 (published 25 Mar. 2021; PCT/US2020/051363, filed 18 Sep. 2020), the disclosure of which is incorporated herein by reference.

Additionally, provided herein is a multifunctional biomaterial placed in a treatment area to deliver compositions treating localized symptoms of, for example, but not limited to, infectious and non-infectious medical conditions, injuries, damage, surgery, and transplant, where most needed in the treatment of localized conditions or symptoms, while avoiding systemic exposure to immunomodulatory agents.

In some embodiments, a porous scaffold is provided comprising at least one compound that regulates T cell immune response; and at least one compound that regulates induction of regulatory T cells (Tregs).

In some embodiments, the implantable scaffold provides means for local delivery of inhibitors of, e.g., tumor or cancer growth, cancer metastasis, infectious agents, or immunostimulatory or other activator compounds. In a non-limiting example, TGF-β is known to be a potent component of the tumor microenvironment, which promotes cancer growth and metastasis and promotes the induction of Tregs from the helper T cells drawn to the tumor. Suppression of TGF-β could allow for a reduction in regulatory T cells and more effective CD8+ T cell killing, resulting in rapid clearance of solid tumors. Described herein is an approach to enable the local delivery of TGF-β inhibitor (TGF-βi) into the tumor environment for the enhancement of the immune responses during immunotherapy. An implantable scaffold is provided comprising means for local delivery of TGF-βi (e.g., in PLGA nanoparticles embedded in the scaffold) and also one or more immunostimulatory compounds to attract and activate bioengineered cytotoxic T cells to target the tumor (e.g., IL-2 on silica-heparin microparticles embedded in the scaffold). Systemic effects are avoided by employing local effects of the scaffold, which can induce a potent T cell response to a tumor or remaining tumor after resection, or even treat inoperable tumors, and then the scaffold can biodegrade over time.

As described herein, the studies described emphasize the local delivery of xenobiotic fuel, inhibitors, and activators based in a biodegradable scaffold. Once administered, the scaffold can provide a localized fuel source to the bioengineered immune cells and/or attract lymphocytes to the site of the tumor and allow simultaneous immune cell stimulation and controlled release of inhibitory compounds. The combined response of the immune system in the tumor microenvironment is then enhanced: in one non-limiting example, T cells are provided with a localized, concentrated source of cellobiose, Treg development is reduced in favor of effector T cell activation, and tumor rejection is achieved by the activated T cells. This method provides complex immunotherapy treatments that are more effective by directly altering the effects of the tumor microenvironment.

Details of each component of the scaffold are provided below. The implantable scaffold can be made of various biocompatible and biodegradable polymers. To further encourage cell trafficking within these structures, cell adhesion peptides such as but not limited to the chemokine CCL21, and immunostimulatory compounds such as IL-2, IL-4, IL-6, IL7, IL-10, IL-12, IL-15, or IL-2 superkine, or antibodies such as anti-CD3 and anti-CD28 are provided. To improve the resemblance of these 3D matrices to natural tissues techniques are used that create microscale pores within these structures that both allows for maximizing the loading capacity for delivering T cells and facilitates their expansion as well. The scaffolds are modified with anti-CD3/anti-CD28 antibodies and further comprise a TGF-βi, as well as IL-2 cytokine to provide activation signal for T cells and prevent formation of regulatory T cells.

The scaffold may comprise a polymer such as but not limited to alginate, hyaluronic acid, or chitosan, or any combination thereof. It comprises one of more the components described below. The scaffold can be fabricated into a shape and size for facile insertion or implantation during a surgical or transdermal procedure. In one embodiment, the scaffold is about the shape and size of a pencil eraser. However, the shape and size can be configured for a particular application, for ease of insertion, and/or for retention at a particular site near a tumor or resected tumor site.

### Scaffold Pores

Pores are created in the scaffold by freeze drying process such as that described in Biopolymer-Based Hydrogels As Scaffolds for Tissue Engineering Applications: A Review Biomacromolecules 2011, 12, 5, 1387-1408; https://pubs.acs.org/doi/abs/10.1021/bm200083n. In one embodiment, the pores are between about 1 and about 7 nm in size.

### Scaffold Microparticles

In some embodiments, the scaffold comprises one or more microparticles. In some embodiments, the microparticles comprise a polymer. In some embodiments, the polymer comprises a biocompatible polymer. In some embodiments, the biocompatible polymer comprises alginate, chitosan, or mesoporous silica. In some embodiments, the microparticles comprise silica microparticles. Silica microparticles such as mesoporous silica may be embedded in the scaffold. In some embodiments, the silica is bound to heparin. In some embodiments, about 2 nmol of heparin is bound per mg of silica. In some embodiments, the microparticle has a size comprising 1-1000 micrometers. In some embodiments, the particles are from about 3 to about 24 μm in diameter. In some embodiments, the microparticles comprise hyaluronic acid. In some embodiments, the microparticles comprise heparin.

In some embodiments, microparticles may be encapsulated by a coating. In some embodiments, coatings provide microparticles with enhanced biological characteristics, including interactions with cells, with compounds that regulate immune response (e.g., T cell immune response), with compounds that regulate induction of immune cells (e.g., compounds that regulate induction of regulatory T cells), and with other biomolecules. In some embodiments, microparticles are encapsulated with a coating comprising heparin. In some embodiments, microparticles are encapsulated with alginate or alginate-heparin. In some embodiments, an alginate-heparin coating may be sulfated.

In some embodiments, microparticles may comprise a “coating” material. In some embodiments, these materials provide microparticles with enhanced biological characteristics, including interactions with cells and biomolecules. In some embodiments, microparticles are formed in the presence of a mix of alginate-heparin. In some embodiments, microparticles are formed in the presence of a mix of alginate. In some embodiments, an alginate may be sulfated.

A skilled artisan would appreciate that a description of a microparticle comprising an alginate or alginate-heparin coating may in certain embodiments, encompass a microparticle prepared in the presence of alginate or alginate and heparin, wherein these molecules and integral components of the microparticle synthesized. Paramagnetic nanoparticles may be included in the microparticles, e.g., for purification or for ease of separation, and are commercially available (e.g., CHEMICELL™ GmbH). In some embodiments, the paramagnetic nanoparticles comprise superparamagnetic iron oxide nanoparticles (SPIONs). In some embodiments, a SPION comprises a particle having a size about 50-200 mm. This addition may in certain embodiments enhance purification of microparticles using methods well known in the art.

In some embodiments, microparticles may be targeted to xenobiotic fuel-enabled bioengineered immune cells (e.g., cellobiose-enabled bioengineered T cells). In some embodiments, a microparticle coat comprises biomolecules that recognize and bind cell surface markers on immune cells. In some embodiments, the microparticle coat comprises biomolecules that recognize and bind cell surface markers on T cells and the cell surface markers on T cells include CD3 and CD28. In some embodiments, a biomolecule that recognized an immune cell surface marker comprises an antibody or a fragment thereof.

### Scaffold Nanoparticles

In some embodiments, the scaffold comprises one or more nanoparticles. In some exemplary, but non-limiting, embodiments, the nanoparticle comprises a poly(lactic-co-glycolic acid) (PLGA, PLG), a copolymer, produced using methods known in the art. In some embodiments, the nanoparticle is sized between 1-100 nm. In some embodiments, the nanoparticle is biocompatible and/or biodegradable. This addition may in certain embodiments enhance purification of microparticles or nanoparticles using methods well known in the art.

In some embodiments, the nanoparticle is bound to at least one compound that regulates induction of regulatory T cells, as described herein.

### Methods of Making Scaffolds

Implantable scaffolds are made of various biocompatible and biodegradable polymers, such as alginate, hyaluronic acid, and chitosan. Microscale pores are created within the structures. To create scaffold with nutrition capability by this artificial niche, mesoporous silica microparticles are embedded in the scaffolds. These microparticles are loaded with at least one xenobiotic fuel capable of being digested by the bioengineered immune cell. In a non-limiting embodiment, the microparticles are loaded with cellobiose, which the bioengineered immune cells (e.g., T cells) have been modified to be capable of transporting and metabolizing. To create scaffolds with stimulatory capability by this artificial niche, mesoporous silica microparticles are embedded in the scaffolds. These microparticles are loaded with at least one compound that regulates T cell immune response (e.g., with cytokines [e.g., IL-2, IL-4, IL-6, IL-7, IL-10, IL-12, IL-15, IL-2 superkine] to improve T cells' proliferation and effector functions).

The implantable scaffold can be made of various biocompatible and biodegradable polymers. To further encourage cell trafficking within these structures, cell adhesion peptides such as but not limited to the chemokine CCL21, and immunostimulatory compounds such as IL-2, IL-4, IL-6, IL-7, IL-10, IL-12 IL-15, or IL-2 superkine, or antibodies such as anti-CD3 and anti-CD28 are provided. To improve the resemblance of these 3D matrices to natural tissues techniques are used that create microscale pores within these structures that both allows for maximizing the loading capacity for delivering T cells and facilitates their expansion as well. The scaffolds are modified with anti-CD3/anti-CD28 antibodies and further comprise a TGF-βi, as well as IL-2 cytokine to provide activation signal for T cells and prevent formation of regulatory T cells.

### Methods of Using Scaffolds

The scaffolds described herein can be fabricated for various applications. In one aspect, the porous scaffold is provided at a site at or near a focus of interest in a subject in need. In one embodiment, one or more scaffolds are inserted surgically at or near the site of a tumor during resection or biopsy. In one embodiment, the scaffold is implanted at or near the site of a tumor or cancer. In one embodiment, the scaffold is implanted at or near the site of an infection. In one embodiment, the scaffold biodegrades. In some embodiments, the mechanical properties of the scaffold, as well as the degradation, time can be modified for a particular use by changing the formulation.

In some embodiments, the scaffold comprises a microparticle not comprising alginate, heparin, or a lipid coating. In some embodiments, the scaffold comprises a microparticle comprising alginate. In some embodiments, the scaffold comprises a microparticle comprising alginate-heparin. In certain embodiments, this scaffold can be administered via a catheter. In certain embodiments, this scaffold can be implanted or injected locally at the site of a tumor, cancer, or infection. Scaffolding comprising microparticles provides in some embodiments, further control over the release of the xenobiotic fuel, the compound regulating T cell immune response, and/or the compound regulating induction of Tregs, and also localizes the effects. In some embodiments, implantation, injection, or other administration of the scaffold provides a stronger cytokine gradient to boost up the therapeutic effects.

In some embodiments, application of the scaffold, or compositions thereof is for local use. This may, in certain embodiments, provide an advantage, wherein the controlled localized release of, e.g., the xenobiotic fuel (e.g., cellobiose), the compound regulating T cell immune response, and/or the compound regulating induction of Tregs may provide a local immune effect thereby avoiding a toxic systemic effect of the cytokine. In one example, controlled release of IL-2 or an IL-2 superkine, may increase proliferation of cytotoxic T cells and or helper T cells in the area adjacent to the cancer or tumor, thereby promoting clearance of the cancer or tumor. In some embodiments, controlled release of IL-2 or an IL-2 superkine, may maintain a helper T cell population in the area adjacent to the tumor. In some embodiments, controlled release of IL-2 or an IL-2 superkine, may activate a cytotoxic T cell population in the area adjacent to the tumor. In some embodiments, controlled release of IL-2 or an IL-2 superkine, may lead to enhanced killing of tumor cells in the localized area at and adjacent to the tumor. In some embodiments, controlled release of IL-2 or an IL-2 superkine, provides enhanced clearance of a tumor. This technique may also be used for the treatment of other diseases, reactions, injuries, transplants, blood clots, and the like, recited herein, particularly in a subject who is on a low-glucose or ketogenic diet.

In some embodiments, a pharmaceutical composition comprises a porous scaffold, as described in detail above. In still another embodiment, a pharmaceutical composition for the treatment of a disease or medical condition, as described herein, comprises an effective amount of the xenobiotic fuel, the compound regulating T cell immune response, and/or the compound regulating induction of Tregs, and a pharmaceutically acceptable excipient. In some embodiments, a composition comprising the porous scaffold comprising the xenobiotic fuel, the compound regulating T cell immune response, and/or the compound regulating induction of Tregs, and a pharmaceutically acceptable excipient is used in methods for regulating an immune response.

### Methods of Using Bioengineered Cells

The immune cells and other aspects and embodiments described above in detail may in certain embodiments be used for therapeutic treatments, for example but not limit to cancer or tumor therapy. Administration thereof provides a regulatory source of cytokines that may in certain embodiments, beneficially regulate an immune response against a cancer. Thus, these immune cells may also be used to regulate the immune response in a subject in need, therapy enhancing therapy, for example but not limited to a cancer or tumor therapy. In a non-limiting example, a bioengineered T cell is administered and stimulated by cellobiose to release toxic cytokines, to increase proliferation of cytotoxic T cells, to increase proliferation of helper T cells, to maintain the population of helper T cells at the site of a focus of interest (e.g., a tumor, infection, etc.), and/or to activate cytotoxic T cells at the site of the focus of interest (e.g., solid tumor, infection, etc.). In a non-limiting example, a bioengineered B cell is administered and stimulated by cellobiose to release and/or increase the production of antibodies, to increase isotype switching, and/or to increase affinity maturation. In a non-limiting example, a bioengineered T regulatory cell is administered and stimulated by cellobiose to suppress a T cell or other immune cells, to suppress an immune response, e.g., by regulating the immune response comprises decreasing proliferation of cytotoxic T cells, decreasing proliferation of helper T cells, and/or suppressing cytotoxic T cells at the site of said focus of interest. In a non-limiting example, a bioengineered macrophage is administered and stimulated by cellobiose to engulf and/or digest (i.e., phagocytosis) an abnormal or foreign cell (e.g., a cancer cell or a microbe), a dead or dying cell, a part of a cell (e.g., cellular debris), or a foreign substance and to activate an immune response. A macrophage comprises, e.g., a phagocyte that detects a cell, a part of a cell, or a foreign substance that does not have on its surface those proteins specific to healthy cell of the organism. In a non-limiting example, a bioengineered M1 polarized macrophage is administered and stimulated by cellobiose to produce proinflammatory cytokines, phagocytize microbes, and/or initiate an immune response (e.g., against a bacterium or a virus). In a non-limiting example, a bioengineered B cell receptor (BCR)-stimulated B cell is administered and stimulated by cellobiose to promote the differentiation of B cells (e.g., into plasma cells).

“Apheresis” or “pheresis” comprises an ex vivo blood purification procedure during which a patient's blood is subjected to a separation apparatus or technique ex vivo to separate out a given constituent prior to the reinfusion of the blood back into the patient (or a different patient). “Leukapheresis” comprises apheretic separation of leukocytes from the blood.

In one embodiment, immune cells may be transfected with vectors expressing proteins (e.g., a cellodextrin transporter protein, a beta-glucosidase protein, and/or a cellobiose phosphorylase protein) during a leukapheresis or other blood cell purification procedure and infused into the patient. Such transfection of leukocytes or other cell types after administration to the body or during a leukapheresis procedure or other ex vivo procedure provides the therapeutic protein in association with a cell type to effect its desired function.

In some embodiments, cells from a cell line may be used to prepare bioengineered immune or other cells for the various purposes described herein including but not limited to adoptive cell therapy. In some embodiments, such cell line may be selected that is HLA matched or compatible for a particular patient population or particular subject to be administered and/or treated by the methods described herein.

In some embodiments, the bioengineered cells and methods herein are used for the treatment of vertebrate organisms. In some embodiments, the bioengineered cells and methods are used for the treatment of homeothermic vertebrate organisms (e.g., mammals and birds). In some embodiments, the bioengineered cells and methods are used for the treatment of human or non-human mammals.

Any of various diseases or medical conditions may be treated by the methods described herein. The methods described herein are of particular use in situations involving treatments of inoperable or inaccessible targets of interest (e.g., an inoperable tumor) or in situations in which it is particularly desirable to target a specific cell population located in multiple, discrete areas of the body.

In some embodiments, “treating” comprises therapeutic treatment including prophylactic or preventive measures, wherein the object is to prevent or lessen the targeted pathologic condition or disorder, for example to treat or prevent cancer. Thus, in some embodiments, treating may include directly affecting or curing, suppressing, inhibiting, preventing, reducing the severity of, delaying the onset of, reducing symptoms associated with cancer or a combination thereof. Thus, in other embodiments, treating may include directly affecting or curing, suppressing, inhibiting, preventing, reducing the severity of, delaying the onset of, reducing symptoms associated with a non-cancerous tumor or a combination thereof. Thus, in some embodiments, “treating,” “ameliorating,” and “alleviating” refer inter alia to delaying progression, expediting remission, inducing remission, augmenting remission, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

A “focus of interest” a “localized environment,” or a “localized site” comprises a site in which the disease, reaction, infection, injury, or other medical condition is specific to one part or area of the body; in which a symptom or condition of the medical condition is specific to one part or area of the body; or in which treatment is desired for one part or area of the body (even if the disease, reaction, infection, injury, or other medical condition affects other parts or areas of the body or the body as a whole).

As used herein, the terms “composition” and “pharmaceutical composition” may in some embodiments, be used interchangeably having all the same qualities and meanings. In some embodiments, disclosed herein is a pharmaceutical composition for the treatment of a cancer or tumor as described herein. In some embodiments, disclosed herein is a pharmaceutical composition for the treatment of cancer or tumor. In some embodiments, disclosed herein is a pharmaceutical composition for the use in methods locally regulating an immune response. In some embodiments, disclosed herein are pharmaceutical compositions for the treatment of an autoimmune disease, an allergic reaction, a hypersensitivity reaction, a localized site of an infection or infectious disease, a localized site of an injury or other damage, a transplant or other surgical site, a blood clot causing or at risk for causing a myocardial infarction, an ischemic stroke, or a pulmonary embolism or a symptom thereof, or a combination thereof.

A “cancer” is one of a group of diseases characterized by uncontrollable growth and having the ability to invade normal tissues and to metastasize to other parts of the body. Cancers have many causes, including, but not limited to, diet, alcohol consumption, tobacco use, environmental toxins, heredity, and viral infections. In most instances, multiple genetic changes are required for the development of a cancer cell. Progression from normal to cancerous cells involves a number of steps to produce typical characteristics of cancer including, e.g., cell growth and division in the absence of normal signals and/or continuous growth and division due to failure to respond to inhibitors thereof; loss of programmed cell death (apoptosis); unlimited numbers of cell divisions (in contrast to a finite number of divisions in normal cells); aberrant promotion of angiogenesis; and invasion of tissue and metastasis.

A “pre-cancerous” condition, lesion, or tumor is a condition, lesion, or tumor comprising abnormal cells associated with a risk of developing cancer. Non-limiting examples of pre-cancerous lesions include colon polyps (which can progress into colon cancer), cervical dysplasia (which can progress into cervical cancer), and monoclonal monopathy (which can progress into multiple myeloma). Premalignant lesions comprise morphologically atypical tissue which appears abnormal when viewed under the microscope, and which are more likely to progress to cancer than normal tissue.

A “non-cancerous tumor” or “benign tumor” is one in which the cells demonstrate normal growth, but are produced, e.g., more rapidly, giving rise to an “aberrant lump” or “compact mass,” which is typically self-contained and does not invade tissues or metastasize to other parts of the body. Nevertheless, a non-cancerous tumor can have devastating effects based upon its location (e.g., a non-cancerous abdominal tumor that prevents pregnancy or causes a ureter, urethral, or bowel blockage, or a benign brain tumor that is inaccessible to normal surgery and yet damages the brain due to unrelieved pressure as it grows).

In one embodiment, the tumor or the cancer for which enhancement of immunity or expansion of CTLS is provided to treat a tumor or cancer. Non-limiting examples include esophageal cancer, pancreatic cancer, metastatic pancreatic cancer, metastatic adenocarcinoma of the pancreas, bladder cancer, stomach cancer, fibrotic cancer, glioma, malignant glioma, diffuse intrinsic pontine glioma, recurrent childhood brain neoplasm renal cell carcinoma, clear-cell metastatic renal cell carcinoma, kidney cancer, prostate cancer, metastatic castration resistant prostate cancer, stage IV prostate cancer, metastatic melanoma, melanoma, malignant melanoma, recurrent melanoma of the skin, melanoma brain metastases, stage IIA skin melanoma; stage IIIB skin melanoma, stage IIIC skin melanoma; stage IV skin melanoma, malignant melanoma of head and neck, lung cancer, non-small cell lung cancer (NSCLC), squamous cell non-small cell lung cancer, breast cancer, recurrent metastatic breast cancer, hepatocellular carcinoma, Hodgkin's lymphoma, follicular lymphoma, non-Hodgkin's lymphoma, advanced B-cell NHL, HL including diffuse large B-cell lymphoma (DLBCL), multiple myeloma, chronic myeloid leukemia, adult acute myeloid leukemia in remission; adult acute myeloid leukemia with Inv(16)(p13.1q22); CBFB-MYH11; adult acute myeloid leukemia with t(16;16)(p13.1;q22); CBFB-MYH11; adult acute myeloid leukemia with t(8;21)(q22;q22); RUNX1-RUNX1T1; adult acute myeloid leukemia with t(9;11)(p22;q23); MLLT3-MLL; adult acute promyelocytic leukemia with t(15;17)(q22;q12); PML-RARA; alkylating agent-related acute myeloid leukemia, chronic lymphocytic leukemia, Richter's syndrome; Waldenstrom's macroglobulinemia, adult glioblastoma; adult gliosarcoma, recurrent glioblastoma, recurrent childhood rhabdomyosarcoma, recurrent Ewing sarcoma/peripheral primitive neuroectodermal tumor, recurrent neuroblastoma; recurrent osteosarcoma, colorectal cancer, MSI positive colorectal cancer; MSI negative colorectal cancer, nasopharyngeal nonkeratinizing carcinoma; recurrent nasopharyngeal undifferentiated carcinoma, cervical adenocarcinoma; cervical adenosquamous carcinoma; cervical squamous cell carcinoma; recurrent cervical carcinoma; stage IVA cervical cancer; stage IVB cervical cancer, anal canal squamous cell carcinoma; metastatic anal canal carcinoma; recurrent anal canal carcinoma, recurrent head and neck cancer; carcinoma, squamous cell of head and neck, head and neck squamous cell carcinoma (HNSCC), ovarian carcinoma, colon cancer, gastric cancer, advanced GI cancer, gastric adenocarcinoma; gastroesophageal junction adenocarcinoma, bone neoplasms, soft tissue sarcoma; bone sarcoma, thymic carcinoma, urothelial carcinoma, recurrent Merkel cell carcinoma; stage III Merkel cell carcinoma; stage IV Merkel cell carcinoma, myelodysplastic syndrome and recurrent mycosis fungoides and Sezary syndrome. In another related aspect, the tumor or cancer comprises a metastasis of a tumor or cancer. In some embodiments, a solid tumor treated using a method described herein, originated as a blood tumor or diffuse tumor.

In some embodiments, the method of using the bioengineered cell comprises treatment of solid tumors, cancers, autoimmune, inflammatory and neuroinflammatory disease.

In some embodiments, the method of using the bioengineered cell comprises treatment of a solid tumor in a glucose-depleted microenvironment. In some embodiments, a “microenvironment” refers to a very small, specific area in an organism (or in a part of an organism, e.g., an organ, a limb), distinguished from its immediate surroundings by, a difference in concentration of a nutrient (e.g., glucose). In some embodiments, a microenvironment comprises a small or relatively small usually distinctly specialized and effectively isolated biophysical environment (e.g., as of a tumor cell).

In some embodiments, the cancer comprises a melanoma, a neuroblastoma, an esophageal cancer, a colorectal cancer, a breast cancer, a T-cell leukemia or a pancreatic cancer.

In some embodiments, methods disclosed herein treat a cancer or a pre-cancerous or non-cancerous tumor. In some embodiments, disclosed herein is a method of treating cancer in a subject in need thereof. In some embodiments, a cancer comprises a solid tumor. In some embodiments, the solid tumor is selected from the group comprising any tumor of cellular or organ origin including a tumor of unknown origin; any peritoneal tumor either primary or metastatic; a tumor of gynecological origin or gastrointestinal origin or pancreatic origin or blood vessel origin, any solid tumor, i.e. adenocarcinoma, hematological solid tumor, melanoma etc. In some embodiments, a solid tumor comprises a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, abasal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodenroglioma, a schwannoma, a meningioma, a melanoma, a neuroblastoma, or a retinoblastoma. In another related aspect, the tumor or cancer comprises a metastasis of a tumor or cancer.

In some embodiments, a solid tumor treated using a method described herein, originated as a blood tumor or diffuse tumor.

In some embodiments, disclosed herein is a method of regulating an immune response at the site of a tumor, said method comprising: administering bioengineered immune cells to said subject, adjacent to a solid tumor; and administering a xenobiotic fuel (e.g., cellobiose) to said subject or implanting a scaffold that releases said xenobiotic fuel (e.g., cellobiose) adjacent to the solid tumor; wherein said regulating the immune response comprises increasing or maintaining the immune response at the site of said tumor.

In some embodiments, disclosed herein is a method of regulating an immune response at the site of a tumor, said method comprising: administering a bioengineered T cells to said subject, adjacent to a solid tumor; and administering cellobiose to said subject or implanting a scaffold that releases cellobiose adjacent to the solid tumor; wherein said regulating the immune response comprises increases proliferation of cytotoxic T cells; increases proliferation of helper T cells; maintains the population of helper T cells at the site of said tumor; activates cytotoxic T cells at the site of said tumor; or any combination thereof.

In some embodiments, disclosed herein is a method of regulating an immune response at the site of a tumor, said method comprising: administering bioengineered B cells to said subject, adjacent to a solid tumor; and administering cellobiose to said subject or implanting a scaffold that releases said cellobiose adjacent to the solid tumor; wherein said regulating the immune response comprises increases release of antibodies at the site of said tumor.

In some embodiments, administration comprises injection and/or infusion directly into a solid tumor. In some embodiments, administration comprises injection and/or infusion adjacent to a solid tumor. Injection may be in the form of a pharmaceutical composition formulated as a sterile injectable solution.

In some embodiments, injection comprises subcutaneous injection. In some embodiments, administration comprises infiltrating a tissue adjacent to a solid tumor with the bioengineered immune cell and/or the scaffold. In some embodiments, the bioengineered immune cell and/or the scaffold is administered via a guided catheter. In some embodiments, the composition is administered, in a non-limiting example, together with angioplasty (e.g., a balloon catheter) or another clot removal treatment.

In some embodiments, “treating” comprises therapeutic treatment including prophylactic or preventive measures, wherein the object is to prevent or lessen the targeted pathologic condition or disorder, for example to treat or prevent an autoimmune disease, an allergic reaction, a localized infection or an infectious disease, an injury or other damage, a transplant or other surgical site, or a symptom thereof, or a combination thereof. Thus, in some embodiments, treating may include directly affecting or curing, suppressing, inhibiting, preventing, reducing the severity of, delaying the onset of, reducing symptoms associated with an autoimmune disease, an allergic reaction, a localized infection or an infectious disease, an injury or other damage, a transplant or other surgical site, or a symptom thereof, or a combination thereof. Thus, in some embodiments, “treating,” “ameliorating,” and “alleviating” refer inter alia to delaying progression, expediting remission, inducing remission, augmenting remission, speeding recovery, increasing efficacy of or decreasing resistance to alternative therapeutics, or a combination thereof. In some embodiments, “preventing” refers, inter alia, to delaying the onset of symptoms, preventing relapse to a disease, decreasing the number or frequency of relapse episodes, increasing latency between symptomatic episodes, or a combination thereof. In some embodiments, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

A “focus of interest,” a “localized environment,” or a “localized site” comprises a site in which the disease, reaction, infection, injury, or other medical condition is specific to one part or area of the body; in which a symptom or condition of the medical condition is specific to one part or area of the body; or in which treatment is desired for one part or area of the body (even if the disease, reaction, infection, injury, or other medical condition affects other parts or areas of the body or the body as a whole).

In some embodiments, methods disclosed herein treat a focus of interest of an autoimmune disease, an allergic reaction, a localized site of an infection or infectious disease, a localized site of an injury or other damage, a transplant or other surgical site, or a symptom thereof, or a combination thereof.

In some embodiments, disclosed herein is a method of treating a focus of interest of an autoimmune disease, an allergic reaction, a localized infection or an infectious disease, an injury or other damage, a transplant or other surgical site, or a symptom thereof, or a combination thereof, in a subject in need thereof, comprising the step of administering to said subject bioengineered T regulatory cells to said subject, adjacent to an autoimmune disease site; and administering cellobiose to said subject or implanting a scaffold that release said cellobiose adjacent to the autoimmune disease site; wherein said regulating the immune response comprises decreases proliferation of cytotoxic T cells; decreases proliferation of helper T cells; suppresses cytotoxic T cells at the site of said autoimmune disease site; or any combination thereof.

In some embodiments, an autoimmune disease, an allergic reaction, a localized infection or an infectious disease, an injury or other damage, a transplant or other surgical site, or a symptom thereof, or a combination thereof, comprises a localized site of an autoimmune disease or allergic reaction, a localized site of an infection or infectious disease, a localized site of injury or damage, a transplant or other surgical site, a blood clot causing or at risk for causing a myocardial infarction, an ischemic stroke, or a pulmonary embolism, or another site comprising one or more localized symptoms thereof, or a combination thereof.

In some embodiments, the autoimmune disease includes, for example, but is not limited to, rheumatoid arthritis, juvenile dermatomyositis, psoriasis, psoriatic arthritis, sarcoidosis, lupus, Crohn's disease, eczema, vasculitis, ulcerative colitis, multiple sclerosis, or type 1 diabetes, achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Baló disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, congenital hear block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis) giant cell myocarditis, glomerulonephritis, Goodpasture's syndrome, granulomatosis with polyangiitis, Grave's disease, Guillain-Barre syndrome, Hashimoto's thyroiditis, hemolytic anemia, Henoch-Schonlein purpura (HSP), Herpes gestationis or pemphigoid gestationis (PG), Hidradenitis suppurativa (HS; acne inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease, lupus, Lyme disease chronic, Menier's disease, microscopic polyangiitis (MPA), mixed connective tissue disease (MCTD), Mooren's ulcer, Mucha-Habermann disease, multifocoal motor neuropathy (MMN, MMNCB), multiple sclerosis, myasthenia gravis, myositis, narcolepsy, neonatallupus, neuromyelitis optica, neutropenia, ocular cicatricial pemphigoid, optic neuritis, palindromic rheumatism (PR), PANDAS, paraneoplasticcerebellar degeneration (PCD), paroxysmal nocturnal hemoglobinuria (PNH), Parry Romberg syndrome, Pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes types I-III, polymyalgia rheumatica, polymyositis, postmyocadial infarction syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy (RSD; complex regional pain syndrome [CRPS]), relapsing polychondritis, restless leg syndrome (RLS), retroperitoneal fibrosis, rheumatic fever, rheumatoid arthritis (RA), sarcoidosis, Schmidt syndrome, scleritis, scleroderma, Sjörgren's syndrome, sperm & testicular autoimmunity, stiff person syndrome (SPS), subacute bacterial endocarditis (SBE), Susac's syndrome, sympathetic ophthalmia (SO), Takayasu arteritis, temporal arteritis/giant cell arteritis, thrombocytopenic purpura (TTP), thyroid eye disease (TED), Tolosa-Hunt syndrome (THS), transverse myelitis, type 1 diabetes, ulcerative colitis (UC), undifferentiated connective tissue disease (UCTD), uveitis, vasculitis, vitiligo, or Vogt-Koyanagi-Harada disease. In some embodiments, the localized site of an autoimmune disease includes, for example, but is not limited to, a joint or other area with inflammation, pain or damage from rheumatoid arthritis; an area affected by juvenile dermatomyositis; psoriatic rash or a joint or other area with psoriatic inflammation; a dermal or other region with symptoms of lupus or eczema; a vascular region damaged by vasculitis; an area of myelin sheath damaged by multiple sclerosis; or a pancreatic islet damaged by type 1 diabetes.

Alternatively, protein production locally for autoimmune diseases targets the pathogenic antibodies in the disease, for example, a protein that breaks down antibodies in the vicinity (an IgG endopeptidase) or a protein that binds antibodies (a decoy of the antibody's autoimmune target).

In some embodiments, the allergic reaction includes, for example, but is not limited to, a localized allergic reaction or hypersensitivity reaction including a skin rash, hives, localized swelling (e.g., from an insect bite), esophageal inflammation from food allergies or eosinophilic esophagitis, other enteric inflammation from food allergies or eosinophilic gastrointestinal disease, localized drug allergies when the drug treatment was local to a part of the body, or allergic conjunctivitis.

In some embodiments, the localized site of an infection or the localized site of an infectious disease includes, for example, but is not limited to, a fungal infection (e.g., aspergillus, coccidioidomycosis), a bacterial infection (e.g., methicillin-resistant Staphylococcus aureus, localized skin infections, abscesses, necrotizing facsciitis, pulmonary bacterial infections [e.g., pneumonia], bacterial meningitis, bacterial sinus infections), a viral infection (e.g., varicella-zoster/herpes zoster [shingles], Herpes simplex I [e.g., cold sores/fever blisters], Herpes simplex II [genital herpes], human papilloma virus [e.g., cervical cancer, throat cancer, esophageal cancer, mouse cancer], Epstein-Barr virus [e.g., nasopharyngeal cancer], encephalitis viruses [e.g., brain inflammation], or hepatitis viruses [e.g., liver disease; hepatitis A, hepatitis B, hepatitis C, hepatitis D, hepatitis E, hepatitis F, hepatitis G]), or a parasitic infection (e.g., an area infected by scabies, Chagas, Hypoderma tarandi, amoebae, roundworm, or Toxoplasma gondii).

In some embodiments, the injury or other damage includes, for example, but is not limited to traumatic injury (e.g., resulting from an accident or violence) or chronic injury (e.g., osteoarthritis). In some embodiments, the localized site of injury comprises a muscular-skeletal injury, a neurological injury, an eye or ear injury, an internal or external wound, a localized abscess, an area of mucosa that is affected (e.g., conjunctiva, sinuses, esophagus), or an area of skin that is affected (e.g., infection, autoimmunity. In some embodiments, the transplant or other surgical site includes, for example, but is not limited to, the site and/or its local environment or surroundings of an organ, corneal, skin, limb, face, or other transplant, or a surgical site and/or its local environment or surroundings, for, e.g., but not limited to, treatment of surgical trauma, treatment of a condition related to the transplant or surgery, or prevention of infection. In some embodiments, the site is at or adjacent to a blood clot causing or at risk for causing a myocardial infarction, an ischemic stroke, or a pulmonary embolism.

In some embodiments, the methods disclosed herein treat one or more symptoms of a disease, reaction, infection, injury, transplant, surgery, or blood clot. In some embodiments, the methods disclosed herein treat a combination thereof.

In some embodiments, disclosed herein is a method of regulating an immune response at the localized site of disease, injury, damage, autoimmune or allergic reaction, or other symptom, including, but not limited to, a localized site of an autoimmune disease, a localized infection or an infectious disease, an injury or other damage, a transplant or other surgical site, said method comprising: administering bioengineered T regulatory cells to said subject, adjacent to a solid tumor; and administering cellobiose to said subject or implanting a scaffold that releases said cellobiose adjacent to the solid tumor; wherein said regulating the immune response comprises decreases proliferation of cytotoxic T cells; decreases proliferation of helper T cells; suppresses cytotoxic T cells at the site of said tumor; or any combination thereof, e.g., at the site of said localized site of an autoimmune disease, a localized infection or an infectious disease, an injury or other damage, a transplant or other surgical site, a blood clot causing or at risk for causing a myocardial infarction, an ischemic stroke, or a pulmonary embolism, or any combination thereof.

In some embodiments, administration comprises injection and/or infusion directly into a localized site of an autoimmune disease, an allergic reaction, a localized infection or an infectious disease, an injury or other damage, a transplant or other surgical site, or a symptom thereof, a blood clot, or a combination thereof. In some embodiments, administration comprises injection and/or infusion adjacent to a localized site of an autoimmune disease, an allergic reaction, a localized infection or an infectious disease, an injury or other damage, a transplant or other surgical site, a blood clot, or a symptom thereof, or a combination thereof. Injection may be in the form of a pharmaceutical composition formulated as a sterile injectable solution.

In some embodiments, injection comprises subcutaneous injection. In some embodiments, administration comprises infiltrating a tissue adjacent to a localized site of an autoimmune disease, an allergic reaction, a localized infection or an infectious disease, an injury or other damage, a transplant or other surgical site, a blood clot, or a symptom thereof, or a combination thereof, with nanoliposomes or microparticles, or compositions thereof.

In some embodiments, injection comprises injecting bioengineered immune cells.

Encapsulation into microparticles provides in some embodiments, further control over the release of the cytokine expressed, and also localizes the effects. In some embodiments, injection nanoliposomes from inside microparticles provides a stronger cytokine gradient to boost up the therapeutic effects. Release of ATP by, for example, UV light controls the transcription and translation of the encoded cytokine, thereby providing a regulatable expression of a beneficial cytokine in a localized region at or adjacent to a tumor.

In some embodiments, application of bioengineered immune cells or compositions thereof is for local use. This may, in certain embodiments, provide an advantage.

### Preparation of an Activated Cytotoxic T Cell Population

Provided herein are methods for the preparation of an activated T cell population (e.g., an activated T cell population of bioengineered T cells) specific for a tumor antigen. Here, the plasmids described above are prepared. The antigen is obtained from a sample from the patient or other subject, as described above, and leukocytes are obtained from whole blood or pheresis, e.g., of blood from a patient or other subject. The leukocytes are transfected in vivo, in vitro, or ex vivo with the plasmid or plasmids. The treated leukocytes are then reinfused into the patient or other subject.

In some embodiments, the bioengineered cell is a modified cell for use in an immunotherapy, e.g., as an approach to cancer treatment, treatment of inflammation (including neuroinflammation), autoimmunity, asthma, or allergy (e.g., food allergy). In some embodiments, a cell bioengineered to metabolize a xenobiotic fuel further comprises a monoclonal antibody (mAb) or a bispecific monoclonal antibody, e.g., as a treatment for immunotherapy, such as directing the bioengineered cell to a specific nutrient-starved area for therapy (e.g., treatment of a cancer or a tumor, treatment of inflammation).

In some embodiments, adoptive cell transfer (ACT) enhances cancer treatment by using the subject's bioengineered immune cells to target and treat their cancer. In some embodiments, ACT approaches include, but are not limited to, bioengineered tumor-infiltrating lymphocytes (TILs), T-cells engineered to alter the specificity of the T-cell receptor (TCR), and chimeric antigen receptor (CAR) T-cells, (CAR) B-cells and (CAR) T regulatory cells (CAR Treg) therapies in which the immune cells bioengineered to metabolize a xenobiotic fuel are further modified, e.g., to comprise a CAR, e.g., as a treatment for immunotherapy, such as directing the bioengineered cell to a specific nutrient-starved area for therapy (e.g., treatment of a cancer or a tumor, treatment of inflammation, treatment of an autoimmune disease).

In some embodiments, CAR T-cell therapy utilizes T regulatory cells (Tregs), a subpopulation of T cells that can regulate ongoing immune reactions and play an important role in the control of autoimmunity, e.g., by secreting inhibitory cytokines, by interfering with the metabolism of T cells or other contacts, or by blocking T cell activation indirectly by interacting with antigen-presenting cells (APCs). In some embodiments, the Tregs may be polyclonal or antigen-specific (e.g., alloantigen-specific). A CAR typically has an ectodomain outside the cell, a transmembrane domain, and an endodomain inside the cell.

In some embodiments, CAR T-cell, CAR B-cell or CAR Treg therapy involves removing blood from the patient in order to obtain the patient's T, B or Treg cells, bioengineering the patient's T, B, or Treg-cells to metabolize a xenobiotic fuel, inserting the chimeric antigen receptor (CAR) gene into the patient's T, B or Treg-cells (before, during, or after the bioengineering of the T, B, or Treg-cells) to produce a bioengineered CAR T-cell, CAR B-cell, or CAR Treg cell (as T, B or Treg cells respectively with a specific chimeric antigen receptor and bioengineered to metabolize a xenobiotic fuel), culturing and propagating the bioengineered CAR T, B or Treg-cells, and infusing the bioengineered CAR T, B or Treg-cells into the patient, where the antigens e.g., bind to cancer cells and kill them or regulate inflammation (as with bioengineered CAR-Treg).

### Immune Response Stimulation and Suppression

In some embodiments, the bioengineered immune cell comprises a T-cell bioengineered to express proteins to break down cellobiose, a B-cell bioengineered to express proteins to break down cellobiose, a dendritic cell bioengineered to express proteins to break down cellobiose, or a natural killer cell (NK) bioengineered to express proteins to break down cellobiose. In some embodiments the bioengineered T-cell, bioengineered B-cell, bioengineered dendritic cell, or bioengineered NK cell is selectively activated by administration of cellobiose, thereby stimulating the immune response.

In some embodiments, the disease or medical condition comprises a tumor or a cancer, and the focus of interest comprising the tumor or the cancer; the disease or medical condition comprises an autoimmune disease, and the focus of interest comprising an autoimmune-targeted or symptomatic focus of said autoimmune disease; the disease or medical condition comprises an allergic reaction or hypersensitivity reaction, and the focus of interest comprising a reactive focus of said allergic reaction or hypersensitivity reaction; the disease or medical condition comprises a localized infection or an infectious disease, and the focus of interest comprising a focus of infection or symptoms; the disease or medical condition comprises an injury or a site of chronic damage, and the focus of interest comprising the injury or the site of chronic damage; the disease or medical condition comprises a surgical site, and the focus of interest comprising the surgical site; the disease or medical condition comprises a transplanted organ, tissue, or cell, and the focus of interest comprising a transplant site; or the disease or medical condition comprises a blood clot causing or at risk for causing a myocardial infarction, an ischemic stroke, or a pulmonary embolism, and the focus of interest comprises the site of the blood clot.

In some embodiments, the autoimmune disease includes, for example, but is not limited to, rheumatoid arthritis, juvenile dermatomyositis, psoriasis, psoriatic arthritis, sarcoidosis, lupus, Crohn's disease, eczema, vasculitis, ulcerative colitis, multiple sclerosis, or type 1 diabetes, achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, autoimmune urticaria, axonal & neuronal neuropathy (AMAN), Bald disease, Behcet's disease, benign mucosal pemphigoid, bullous pemphigoid, Castleman disease (CD), celiac disease, Chagas disease, chronic inflammatory demyelinating polyneuropathy (CIDP), chronic recurrent multifocal osteomyelitis (CRMO), Churg-Strauss syndrome (CSS) or eosinophilic granulomatosis (EGPA), cicatricial pemphigoid, Cogan's syndrome, cold agglutinin disease, congenital hear block, Coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis) giant cell myocarditis, glomerulonephritis, Goodpasture's syndrome, granulomatosis with polyangiitis, Grave's disease, Guillain-Barre syndrome, Hashimoto's thyroiditis, hemolytic anemia, Henoch-Schonlein purpura (HSP), Herpes gestationis or pemphigoid gestationis (PG), Hidradenitis suppurativa (HS; acne inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease, lupus, Lyme disease chronic, Menier's disease, microscopic polyangiitis (MPA), mixed connective tissue disease (MCTD), Mooren's ulcer, Mucha-Habermann disease, multifocoal motor neuropathy (MMN, MMNCB), multiple sclerosis, myasthenia gravis, myositis, narcolepsy, neonatallupus, neuromyelitis optica, neutropenia, ocular cicatricial pemphigoid, optic neuritis, palindromic rheumatism (PR), PANDAS, paraneoplasticcerebellar degeneration (PCD), paroxysmal nocturnal hemoglobinuria (PNH), Parry Romberg syndrome, Pars planitis (peripheral uveitis), Parsonage-Turner syndrome, pemphigus, peripheral neuropathy, perivenous encephalomyelitis, pernicious anemia (PA), POEMS syndrome, polyarteritis nodosa, polyglandular syndromes types I-III, polymyalgia rheumatica, polymyositis, postmyocadial infarction syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy (RSD; complex regional pain syndrome [CRPS]), relapsing polychondritis, restless leg syndrome (RLS), retroperitoneal fibrosis, rheumatic fever, rheumatoid arthritis (RA), sarcoidosis, Schmidt syndrome, scleritis, scleroderma, Sjörgren's syndrome, sperm & testicular autoimmunity, stiff person syndrome (SPS), subacute bacterial endocarditis (SBE), Susac's syndrome, sympathetic ophthalmia (SO), Takayasu arteritis, temporal arteritis/giant cell arteritis, thrombocytopenic purpura (TTP), thyroid eye disease (TED), Tolosa-Hunt syndrome (THS), transverse myelitis, type 1 diabetes, ulcerative colitis (UC), undifferentiated connective tissue disease (UCTD), uveitis, vasculitis, vitiligo, or Vogt-Koyanagi-Harada disease. In some embodiments, the localized site of an autoimmune disease includes, for example, but is not limited to, a joint or other area with inflammation, pain or damage from rheumatoid arthritis; an area affected by juvenile dermatomyositis; psoriatic rash or a joint or other area with psoriatic inflammation; a dermal or other region with symptoms of lupus or eczema; a vascular region damaged by vasculitis; an area of myelin sheath damaged by multiple sclerosis; or a pancreatic islet damaged by type 1 diabetes.

In some embodiments, the allergic reaction includes, for example, but is not limited to, a localized allergic reaction or hypersensitivity reaction including a skin rash, hives, localized swelling (e.g., from an insect bite), or esophageal inflammation from food allergies or eosinophilic esophagitis, other enteric inflammation from food allergies or eosinophilic gastrointestinal disease, localized drug allergies when the drug treatment was local to a part of the body, or allergic conjunctivitis.

In some embodiments, the localized site of an infection or the localized site of an infectious disease includes, for example, but is not limited to, a fungal infection (e.g., aspergillus, coccidioidomycosis, tinea pedis (foot), tinea corporis (body), tinea cruris (groin), tinea capitis (scalp), and tinea unguium (nail)), a bacterial infection (e.g., methicillin-resistant Staphylococcus aureus [MRSA], localized skin infections, abscesses, necrotizing facsciitis, pulmonary bacterial infections [e.g., pneumonia], bacterial meningitis, bacterial sinus infections, bacterial cellulitis, such as due to Staphylococcus aureus (MRSA), bacterial vaginosis, gonorrhea, chlamydia, syphilis, Clostridium difficile (C. diff), tuberculosis, cholera, botulism, tetanus, anthrax, pneumococcal pneumonia, bacterial meningitis, Lyme disease), a viral infection (e.g., varicella-zoster/herpes zoster [shingles], Herpes simplex I [e.g., cold sores/fever blisters], Herpes simplex II [genital herpes], or human papilloma virus [e.g., cervical cancer, throat cancer, esophageal cancer, mouse cancer], Epstein-Barr virus [e.g., nasopharyngeal cancer], encephalitis viruses [e.g., brain inflammation], or hepatitis viruses [e.g., liver disease; hepatitis A, hepatitis B, hepatitis C, hepatitis D, hepatitis E, hepatitis F, hepatitis G] or COVID-19), aparasitic infection (e.g., an area infected by scabies, Chagas, Hypoderma tarandi, amoebae, roundworm, Toxoplasma gondii). In some embodiments, the injury or other damage includes, for example, but is not limited to traumatic injury (e.g., resulting from an accident or violence) or chronic injury (e.g., osteoarthritis). In some embodiments, the localized site of injury comprises a muscular-skeletal injury, a neurological injury, an eye or ear injury, an internal or external wound, or a localized abscess, an area of mucosa that is affected (e.g., conjunctiva, sinuses, esophagus), or an area of skin that is affected (e.g., infection, autoimmunity). In some embodiments, the transplant or other surgical site includes, for example, but is not limited to, the site and/or its local environment or surroundings of an organ, corneal, skin, limb, face, or other transplant, or a surgical site and/or its local environment or surroundings, for, e.g., but not limited to, treatment of surgical trauma, treatment of a condition related to the transplant or surgery, or prevention of infection. In some embodiments, the site is at or adjacent to a blood clot causing or at risk for causing a myocardial infarction, an ischemic stroke, or a pulmonary embolism. In some embodiments, the methods disclosed herein treat one or more symptoms of a disease, reaction, infection, injury, transplant, surgery, or blood clot. In some embodiments, the methods disclosed herein treat a combination thereof.

In some embodiments, methods of treating described herein for promoting clearance of or alleviating localized symptoms of the autoimmune disease, allergic reaction, hypersensitivity reaction, infection or infectious disease; for facilitating healing and/or preventing or inhibiting infection or rejection of a localized site of an injury or other damage, a transplant or other surgical site; for reducing or eliminating a blood clot causing or at risk for causing a myocardial infarction, an ischemic stroke, or a pulmonary embolism; or for alleviating localized symptoms thereof; or for a combination thereof.

In some embodiments, the method further comprises a step of administering activated T cells, such as bioengineered activated T cells, to said subject. Methods of preparing T cells are known in the art. In some embodiments, these cells may be administered prior to or after administering the treated leukocytes. In some embodiments, T cells are administered by intravenous (i.v., IV) injection.

Treatment of the subject the methods herein may also be used in conjunction with other known treatments. In a non-limiting example, when the disease or medical condition comprises a blood clot causing or at risk for causing a myocardial infarction, an ischemic stroke, or a pulmonary embolism, and the treatment may also include angioplasty or another clot removal treatment. Other examples of treatment include various other immunotherapies.

In some embodiments, regulating the immune response increases proliferation of cytotoxic T cells; increases proliferation of helper T cells; maintains the population of helper T cells at the site of said localized site of an autoimmune disease, a localized infection or an infectious disease, an injury or other damage, a transplant or other surgical site; activated cytotoxic T cells at the site of said localized site of an autoimmune disease, a localized infection or an infectious disease, an injury or other damage, a transplant or other surgical site, a blood clot causing or at risk for causing a myocardial infarction, an ischemic stroke, or a pulmonary embolism, or any combination thereof.

In some embodiments, methods of treating described herein reduce the size of the tumor, eliminate said tumor, slow the growth or regrowth of the tumor, or prolong survival of said subject, or any combination thereof. In some embodiments, treating reduces or eliminates inflammation or another symptom of the autoimmune-targeted or symptomatic focus of an autoimmune disease, prolongs survival of the subject, or any combination thereof; reduces or eliminates inflammation or another symptom of allergic reaction or hypersensitivity reaction at the reactive focus of an allergic reaction or hypersensitivity reaction, prolongs survival of the subject, or any combination thereof; reduces or eliminates infection or symptoms at the focus of infection or symptoms of a localized infection or infectious disease, prolongs survival of the subject, or any combination thereof; reduces, eliminates, inhibits or prevents structural, organ, tissue, or cell damage, inflammation, infection, or another symptom at a site of injury or a site of chronic damage, improves structural, organ, tissue, or cell function at a site of injury or a site of chronic damage, improves mobility of the subject, prolongs survival of the subject, or any combination thereof; reduces, eliminates, inhibits, or prevents structural, organ, tissue, or cell damage, inflammation, infection, or another symptom at a surgical site, improves structural, organ, tissue, or cell function at a surgical site, improves mobility of the subject, prolongs survival of the subject, or any combination thereof; reduces, eliminates, inhibits or prevents transplanted organ, tissue, or cell damage or rejection, inflammation, infection or another symptom at a transplant site, improves mobility of the subject, prolongs survival of a transplanted organ, tissue, or cell, prolongs survival of the subject, or any combination thereof; or reduces or eliminates a blood clot causing or at risk for causing a myocardial infarction, an ischemic stroke, or a pulmonary embolism in the subject, improves function or survival of a heart, brain, or lung organ, tissue, or cell in the subject, reduces damage to a heart, brain, or lung organ, tissue, or cell in the subject, prolongs survival of a heart, brain, or lung organ, tissue, or cell in the subject, prolongs survival of the subject, or any combination thereof.

### Preparation of an Activated Cytotoxic T Cell Population

Provided herein are methods for the preparation of an activated T cell population (e.g., an activated T cell population of bioengineered T cells) specific for a tumor antigen. Here, the plasmids described above are prepared. The antigen is obtained from a sample from the patient or other subject, as described above, and leukocytes are obtained from whole blood or pheresis, e.g., of blood from a patient or other subject. The leukocytes are transfected in vivo, in vitro, or ex vivo with the plasmid or plasmids. The treated leukocytes are then reinfused into the patient or other subject.

In some embodiments, the bioengineered cell is a modified cell for use in an immunotherapy, e.g., as an approach to cancer treatment, treatment of inflammation (including neuroinflammation), autoimmune disease treatment, asthma treatment, or allergy treatment (e.g., a food allergy treatment). In some embodiments, a cell bioengineered to metabolize a xenobiotic fuel further comprises a monoclonal antibody (mAb) or a bispecific monoclonal antibody, e.g., as a treatment for immunotherapy, such as directing the bioengineered cell to a specific nutrient-starved area for therapy (e.g., treatment of a cancer or a tumor, treatment of inflammation).

In some embodiments, adoptive cell transfer (ACT) enhances cancer treatment by using the subject's bioengineered immune cells to target and treat their cancer. In some embodiments, ACT approaches include, but are not limited to, bioengineered tumor-infiltrating lymphocytes (TILs), T-cells engineered to alter the specificity of the T-cell receptor (TCR), and chimeric antigen receptor (CAR) T-cells, (CAR) B-cells and (CAR) T regulatory cells (CAR Treg) therapies in which the immune cells bioengineered to metabolize a xenobiotic fuel are further modified, e.g., to comprise a CAR, e.g., as a treatment for immunotherapy, such as directing the bioengineered cell to a specific nutrient-starved area for therapy (e.g., treatment of a cancer or a tumor, treatment of inflammation, treatment of an autoimmune disease).

In some embodiments, CAR T-cell therapy utilizes T regulatory cells (Tregs), a subpopulation of T cells that can regulate ongoing immune reactions and play an important role in the control of autoimmunity, e.g., by secreting inhibitory cytokines, by interfering with the metabolism of T cells or other contacts, or by blocking T cell activation indirectly by interacting with antigen-presenting cells (APCs). In some embodiments, the Tregs may be polyclonal or antigen-specific (e.g., alloantigen-specific). A CAR typically has an ectodomain outside the cell, a transmembrane domain, and an endodomain inside the cell.

In some embodiments, CAR T-cell, CAR B-cell or CAR Treg therapy involves removing blood from the patient in order to obtain the patient's T, B or Treg cells, bioengineering the patient's T, B, or Treg-cells to metabolize a xenobiotic fuel, inserting the chimeric antigen receptor (CAR) gene into the patient's T, B or Treg-cells (before, during, or after the bioengineering of the T, B, or Treg-cells) to produce a bioengineered CAR T-cell, CAR B-cell, or CAR Treg cell (as T, B or Treg cells respectively with a specific chimeric antigen receptor and bioengineered to metabolize a xenobiotic fuel), culturing and propagating the bioengineered CAR T, B or Treg-cells, and infusing the bioengineered CAR T, B or Treg-cells into the patient, where the antigens e.g., bind to cancer cells and kill them or regulate inflammation (as with bioengineered CAR-Treg).

### Suppressing Tregs

T regulatory cells (Tregs or regulatory T cells) are suppressive of adaptive immune responses through a variety of mechanisms. Overall, Tregs are a major bane of therapy against cancers. To address this problem, the method may include the ability to suppress the development of induced Tregs. In one embodiment, particles comprise and secrete an inhibitor of TGF-β, and the effect is that Tregs are suppressed while “conventional” effector T cells against the tumor antigens are promoted.

Thus, in one embodiment, an inhibitor or blocker of TGF-β is included. A TGF-β inhibitor (TGF-βi) such as a TGF-β receptor inhibitor may be used. Non-limiting examples include galinusertib (LY2157299) or SB505124.

### Inducing Tregs for Treating Autoimmunity

In another embodiment, rather than activating T cells to respond to tumor antigens and kill tumor cells, another embodiment induces cellobiose-enabled bioengineered T regulatory cells (Tregs). This approach can elicit regulatory T cells in response to cellobiose administration. Regulatory T cells can be delivered to mitigate autoimmune diseases. It is not often (ever) known what the self-antigen is for autoimmune diseases, as there are thousands of unique proteins that may be specific for a particular tissue that is under autoimmune attack. Thus, it has been difficult to develop a strategy for tolerizing T cells to the right autoantigen.

In this embodiment, Tregs are bioengineered to metabolize cellobiose, and the subject is given a low glucose diet and cellobiose is administered, which together promote regulatory T cell development (so called induced regulatory T cells).

The Tregs are bioengineered in the same manner as described above.

In some embodiments, the methods are used for the treatment of vertebrate organisms. In some embodiments, the methods are used for the treatment of homeothermic vertebrate organisms (e.g., mammals and birds). In some embodiments, the methods are used for the treatment of human or non-human mammals.

### Pharmaceutical Compositions

In As used herein, the terms “composition” and “pharmaceutical composition” may in some embodiments, be used interchangeably having all the same qualities and meanings. In some embodiments, disclosed herein is a pharmaceutical composition for the treatment of a cancer or tumor as described herein. In some embodiments, disclosed herein is a pharmaceutical composition for the treatment of cancer or tumor. In some embodiments, disclosed herein is a pharmaceutical composition for the use in methods locally regulating an immune response. In some embodiments, disclosed herein are pharmaceutical compositions for the treatment of an autoimmune disease, an allergic reaction, a localized site of an infection or infectious disease, a localized site of an injury or other damage, a transplant or other surgical site, a blood clot causing or at risk for causing a myocardial infarction, an ischemic stroke, or a pulmonary embolism or a symptom thereof, or a combination thereof.

In some embodiments, a pharmaceutical composition comprises a xenobiotic fuel, as described in detail above. In some embodiments, a pharmaceutical composition comprises an effective amount of a xenobiotic fuel and a pharmaceutically acceptable carrier or excipient. In some embodiments, the pharmaceutical composition is for the treatment of cancer or tumor, as described herein. In some embodiments, the pharmaceutical composition is used in methods for regulating an immune response. In some embodiments, the pharmaceutical composition is used in methods to reduce the size of a tumor. In some embodiments, the pharmaceutical composition is used in methods to eliminate the tumor. In some embodiments, the pharmaceutical composition is used in methods to slow the growth of a tumor. In some embodiments, the pharmaceutical composition is used in methods to prolong the survival of the subject. In some embodiments, methods of treating described herein reduce the size of the tumor, eliminate said tumor, slow the growth of the tumor, or prolong survival of said subject, or any combination thereof.

In some embodiments, a pharmaceutical composition comprises cellobiose as described in detail above. In some embodiments, a pharmaceutical composition comprises an effective amount of cellobiose and a pharmaceutically acceptable carrier or excipient.

In some embodiments, the pharmaceutically acceptable carrier comprises a saline solution, a gel, or a polar solvent.

In still another embodiment, a pharmaceutical composition for the treatment of an autoimmune disease, an allergic reaction, a localized site of an infection or infectious disease, a localized site of an injury or other damage, a transplant or other surgical site, a blood clot, or a symptom thereof of any one of these, or a combination thereof, as described herein, comprises an effective amount of xenobiotic fuel and a pharmaceutically acceptable carrier or excipient. In some embodiments, the pharmaceutical composition comprises cellobiose and a pharmaceutically acceptable carrier or excipient. In some embodiments, the pharmaceutically acceptable carrier comprises a saline solution, a gel, or a polar solvent. In some embodiments, the pharmaceutical composition is used in methods for regulating an immune response. In some embodiments, the pharmaceutical composition is used in methods for promoting clearance of or alleviating localized symptoms of the autoimmune disease, allergic reaction, infection or infectious disease. In some embodiments, the pharmaceutical composition is used in methods for facilitating healing and/or preventing or inhibiting infection or rejection of a localized site of an injury or other damage, a transplant or other surgical site. In some embodiments, the pharmaceutical composition is used in methods for alleviating localized symptoms relating to an autoimmune disease, an allergic reaction, a localized site of an infection or infectious disease, a localized site of an injury or other damage, a transplant or other surgical site, or a symptom thereof, or a combination thereof. In some embodiments, the pharmaceutical composition is used in methods to prolong the survival of the subject. In some embodiments, methods of treating described herein for promoting clearance of or alleviating localized symptoms of the autoimmune disease, allergic reaction, infection or infectious disease; for facilitating healing and/or preventing or inhibiting infection or rejection of a localized site of an injury or other damage, a transplant or other surgical site; for reducing or eliminating a blood clot causing or at risk for causing a myocardial infarction, an ischemic stroke, or a pulmonary embolism; or for alleviating localized symptoms thereof; or for a combination thereof.

In some embodiments, a method of use of the bioengineered or transgenic cell further comprises a step of administering activated T cells to said subject. Methods of preparing T cells are known in the art and are described herein. In other embodiments comprising a UV caged or IR caged ATP is administering activated T cells is prior to or after exposing the site to UV or IR light, respectively. In some embodiments, T cells are administered by intravenous (i.v.) injection. In some embodiments, administration of T cells enhances the therapeutic effect provided by the regulated, local expression of a cytokine from administered nanoliposomes or microparticles.

In some embodiments, the methods are used for the treatment of vertebrate organisms. In some embodiments, the methods are used for the treatment of homeothermic vertebrate organisms (e.g., mammals and birds). In some embodiments, the methods are used for the treatment of human or non-human mammals.

Unless otherwise indicated, all numbers expressing quantities, ratios, and numerical properties of ingredients, reaction conditions, and so forth used in the specification and claims are to be understood as being modified in all instances by the term “about”. All parts, percentages, ratios, etc. herein are by weight unless indicated otherwise.

As used herein, the singular forms “a” or “an” or “the” are used interchangeably and intended to include the plural forms as well and fall within each meaning, unless expressly stated otherwise or unless the context clearly dictates otherwise. For example, the term “a compound” or “at least one compound” may include a plurality of compounds, including mixtures thereof.

Also as used herein, “at least one” is intended to mean “one or more” of the listed elements. Singular word forms are intended to include plural word forms and are likewise used herein interchangeably where appropriate and fall within each meaning, unless expressly stated otherwise. Except where noted otherwise, capitalized and non-capitalized forms of all terms fall within each meaning.

“Consisting of” shall thus mean excluding more than traces of other elements. The skilled artisan would appreciate that while, in some embodiments the term “comprising” is used, such a term may be replaced by the term “consisting of”, wherein such a replacement would narrow the scope of inclusion of elements not specifically recited. The terms “comprises”, “comprising”, “includes”, “including”, “having” and their conjugates encompass “including but not limited to”.

The term “about” or “approximately” means within an acceptable error range for the particular value as determined by one of ordinary skill in the art, which will depend in part on how the value is measured or determined. In some embodiments, the term “about” refers to a deviance of between 0.0001-5% from the indicated number or range of numbers. In some embodiments, the term “about” refers to a deviance of between 1-10% from the indicated number or range of numbers. In some embodiments, the term “about” refers to a deviance of up to 25% from the indicated number or range of numbers. In some embodiments, the term “about” refers to ±10%.

Throughout this application, various embodiments may be presented in a range format. It should be understood that the description in range format is merely for convenience and brevity and should not be construed as an inflexible limitation on the scope of certain embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Any patent, patent application publication, or scientific publication, cited herein, is incorporated by reference herein in its entirety.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### Examples

### Example 1: Production of Bioengineered Immune Cells and Methods of Use

A subject is in need of treatment for a disease or medical condition of interest, or for alleviation of localized symptoms, or combinations thereof, in which the disease, medical condition, or symptoms result in a low glucose environment or in which cells or infectious agents responsible for causing or maintaining the disease, medical condition, or symptoms or the progression thereof utilize an increased level of glucose compared with the corresponding wild-type or uninfected cells of the subject.

One or more immune cells are transfected with one or more vectors expressing one or more proteins capable of transporting or metabolizing a xenobiotic fuel.

The immune cell is an immunotherapeutic immune cell selected for the treatment of the disease or medical condition of interest, or for the alleviation of the localized symptoms, or combinations thereof, in the subject. Alternatively, the immune cell is a diagnostic immune cell selected for detecting the presence of a disease or medical condition of interest, or a component or indicator thereof, in a subject.

The immune cell is administered to the subject in need thereof, optionally at the focus of interest on or within the subject. The subject is placed on a low glucose diet comprising the xenobiotic fuel. Optionally, a scaffold comprising the xenobiotic fuel is implanted subject, e.g., at the focus of interest on or within the subject. Optionally a scaffold comprising the bioengineered immune cell is implanted in the subject, e.g., at the focus of interest on or within the subject.

### Example 2: Production of Bioengineered Immune Cells and Methods of Use

A subject is in need of treatment for a disease or medical condition of interest, or for alleviation of localized symptoms, or combinations thereof, in which the disease, medical condition, or symptoms result in a low glucose environment or in which cells or infectious agents responsible for causing or maintaining the disease, medical condition, or symptoms or the progression thereof utilize an increased level of glucose compared with the corresponding wild-type or uninfected cells of the subject.

One or more immune cells are transfected with one or more vectors expressing (a) a cellodextrin transporter protein and (b) a beta-glucosidase protein and/or a cellobiose phosphorylase protein.

The immune cell is an immunotherapeutic immune cell selected for the treatment of the disease or medical condition of interest, or for the alleviation of the localized symptoms, or combinations thereof, in the subject. Alternatively, the immune cell is a diagnostic immune cell selected for detecting the presence of a disease or medical condition of interest, or a component or indicator thereof, in a subject.

The immune cell is administered to the subject in need thereof, optionally at the focus of interest on or within the subject. The subject is placed on a low glucose diet comprising cellobiose. Optionally, a scaffold comprising cellobiose is implanted subject, e.g., at the focus of interest on or within the subject. Optionally a scaffold comprising the bioengineered immune cell is implanted in the subject, e.g., at the focus of interest on or within the subject.

### Example 3: Production of Bioengineered Immune Cells and Methods of Use

A subject is in need of treatment for a disease or medical condition of interest, or for alleviation of localized symptoms, or combinations thereof, in which the disease, medical condition, or symptoms result in a low glucose environment or in which cells or infectious agents responsible for causing or maintaining the disease, medical condition, or symptoms or the progression thereof utilize an increased level of glucose compared with the corresponding wild-type or uninfected cells of the subject.

One or more immune cells are transfected with one or more vectors expressing (a) a cellodextrin transporter protein and (b) a beta-glucosidase protein.

The immune cell is an immunotherapeutic immune cell selected for the treatment of the disease or medical condition of interest, or for the alleviation of the localized symptoms, or combinations thereof, in the subject. Alternatively, the immune cell is a diagnostic immune cell selected for detecting the presence of a disease or medical condition of interest, or a component or indicator thereof, in a subject.

The immune cell is administered to the subject in need thereof, optionally at the focus of interest on or within the subject. The subject is placed on a low glucose diet comprising cellobiose. Optionally, a scaffold comprising cellobiose is implanted subject, e.g., at the focus of interest on or within the subject. Optionally a scaffold comprising the bioengineered immune cell is implanted in the subject.

### Example 4: Production of Bioengineered Immune Cells and Methods of Use

A subject is in need of treatment for a disease or medical condition of interest, or for alleviation of localized symptoms, or combinations thereof, in which the disease, medical condition, or symptoms result in a low glucose environment or in which cells or infectious agents responsible for causing or maintaining the disease, medical condition, or symptoms or the progression thereof utilize an increased level of glucose compared with the corresponding wild-type or uninfected cells of the subject.

One or more immune cells are transfected with one or more vectors expressing (a) a cellodextrin transporter protein and (b) a cellobiose phosphorylase protein.

The immune cell is an immunotherapeutic immune cell selected for the treatment of the disease or medical condition of interest, or for the alleviation of the localized symptoms, or combinations thereof, in the subject. Alternatively, the immune cell is a diagnostic immune cell selected for detecting the presence of a disease or medical condition of interest, or a component or indicator thereof, in a subject.

The immune cell is administered to the subject in need thereof, optionally at the focus of interest on or within the subject. The subject is placed on a low glucose diet comprising cellobiose. Optionally, a scaffold comprising cellobiose is implanted subject, e.g., at the focus of interest on or within the subject. Optionally a scaffold comprising the bioengineered immune cell is implanted in the subject, e.g., at the focus of interest on or within the subject.

### Example 5: Production of Bioengineered T Cells and Methods of Use

A subject is in need of treatment for a disease or medical condition of interest, or for alleviation of localized symptoms, or combinations thereof, in which the disease, medical condition, or symptoms result in a low glucose environment or in which cells or infectious agents responsible for causing or maintaining the disease, medical condition, or symptoms or the progression thereof utilize an increased level of glucose compared with the corresponding wild-type or uninfected cells of the subject.

One or more T cells are transfected with one or more vectors expressing (a) a cellodextrin transporter protein and (b) a beta-glucosidase protein and/or a cellobiose phosphorylase protein.

The T cell is an immunogenic T cell selected for the treatment of the disease or medical condition of interest, or for the alleviation of the localized symptoms, or combinations thereof, in the subject. Alternatively, the T cell is a diagnostic T cell selected for detecting the presence of a disease or medical condition of interest, or a component or indicator thereof, in a subject.

Optionally, the bioengineered T cell is bioengineered further to comprise a bioengineered T cell receptor (TCR) specific to the target cell of interest or the bioengineered T cell is a CAR-T cell.

The T cell is administered to the subject in need thereof, optionally at the focus of interest on or within the subject. The subject is placed on a low glucose diet comprising cellobiose. Optionally, a scaffold comprising cellobiose is implanted subject, e.g., at the focus of interest on or within the subject. Optionally a scaffold comprising the bioengineered T cell is implanted in the subject, e.g., at the focus of interest on or within the subject.

### Example 6: Production of Bioengineered T Cells and Methods of Use

A subject is in need of treatment for a disease or medical condition of interest, or for alleviation of localized symptoms, or combinations thereof, in which the disease, medical condition, or symptoms result in a low glucose environment or in which cells or infectious agents responsible for causing or maintaining the disease, medical condition, or symptoms or the progression thereof utilize an increased level of glucose compared with the corresponding wild-type or uninfected cells of the subject.

One or more T cells are transfected with one or more vectors expressing (a) a cellodextrin transporter protein and (b) a beta-glucosidase protein.

The T cell is an immunotherapeutic T cell selected for the treatment of the disease or medical condition of interest, or for the alleviation of the localized symptoms, or combinations thereof, in the subject. Alternatively, the T cell is a diagnostic T cell selected for detecting the presence of a disease or medical condition of interest, or a component or indicator thereof, in a subject.

Optionally, the bioengineered T cell is bioengineered further to comprise a bioengineered T cell receptor (TCR) specific to the target cell of interest or the bioengineered T cell is a CAR-T cell.

The T cell is administered to the subject in need thereof, optionally at the focus of interest on or within the subject. The subject is placed on a low glucose diet comprising cellobiose. Optionally, a scaffold comprising cellobiose is implanted subject, e.g., at the focus of interest on or within the subject. Optionally a scaffold comprising the bioengineered T cell is implanted in the subject, e.g., at the focus of interest on or within the subject.

### Example 7: Production of Bioengineered T Cells and Methods of Use

A subject is in need of treatment for a disease or medical condition of interest, or for alleviation of localized symptoms, or combinations thereof, in which the disease, medical condition, or symptoms result in a low glucose environment or in which cells or infectious agents responsible for causing or maintaining the disease, medical condition, or symptoms or the progression thereof utilize an increased level of glucose compared with the corresponding wild-type or uninfected cells of the subject.

One or more T cells are transfected with one or more vectors expressing (a) a cellodextrin transporter protein and (b) a cellobiose phosphorylase protein.

The T cell is an immunotherapeutic T cell selected for the treatment of the disease or medical condition of interest, or for the alleviation of the localized symptoms, or combinations thereof, in the subject. Alternatively, the T cell is a diagnostic T cell selected for detecting the presence of a disease or medical condition of interest, or a component or indicator thereof, in a subject.

Optionally, the bioengineered T cell is bioengineered further to comprise a bioengineered T cell receptor (TCR) specific to the target cell of interest or the bioengineered T cell is a CAR-T cell.

The T cell is administered to the subject in need thereof, optionally at the focus of interest on or within the subject. The subject is placed on a low glucose diet comprising cellobiose. Optionally, a scaffold comprising cellobiose is implanted subject, e.g., at the focus of interest on or within the subject. Optionally a scaffold comprising the bioengineered T cell is implanted in the subject, e.g., at the focus of interest on or within the subject.

### Example 8: Production of Bioengineered B Cells and Methods of Use

A subject is in need of treatment for a disease or medical condition of interest, or for alleviation of localized symptoms, or combinations thereof, in which the disease, medical condition, or symptoms result in a low glucose environment or in which cells or infectious agents responsible for causing or maintaining the disease, medical condition, or symptoms or the progression thereof utilize an increased level of glucose compared with the corresponding wild-type or uninfected cells of the subject.

One or more B cells are transfected with one or more vectors expressing (a) a cellodextrin transporter protein and (b) a beta-glucosidase protein and/or a cellobiose phosphorylase protein.

The B cell is an immunogenic B cell selected for production of antibodies in the treatment of the disease or medical condition of interest, or for the production of antibodies for the alleviation of the localized symptoms, or combinations thereof, in the subject.

Optionally, the bioengineered B cell is bioengineered further to comprise a bioengineered antibody specific to the target cell of interest or the bioengineered B cell is a CAR-B cell.

The B cell is administered to the subject in need thereof, optionally at the focus of interest on or within the subject. The subject is placed on a low glucose diet comprising cellobiose. Optionally, a scaffold comprising cellobiose is implanted subject, e.g., at the focus of interest on or within the subject. Optionally a scaffold comprising the bioengineered B cell is implanted in the subject, e.g., at the focus of interest on or within the subject.

### Example 9: Production of Bioengineered B Cells and Methods of Use

A subject is in need of treatment for a disease or medical condition of interest, or for alleviation of localized symptoms, or combinations thereof, in which the disease, medical condition, or symptoms result in a low glucose environment or in which cells or infectious agents responsible for causing or maintaining the disease, medical condition, or symptoms or the progression thereof utilize an increased level of glucose compared with the corresponding wild-type or uninfected cells of the subject.

One or more B cells are transfected with one or more vectors expressing (a) a cellodextrin transporter protein and (b) a beta-glucosidase protein.

The B cell is an immunogenic B cell selected for production of antibodies in the treatment of the disease or medical condition of interest, or for the production of antibodies for the alleviation of the localized symptoms, or combinations thereof, in the subject.

Optionally, the bioengineered B cell is bioengineered further to comprise a bioengineered antibody specific to the target cell of interest or the bioengineered B cell is a CAR-B cell.

The B cell is administered to the subject in need thereof, optionally at the focus of interest on or within the subject. The subject is placed on a low glucose diet comprising cellobiose. Optionally, a scaffold comprising cellobiose is implanted subject, e.g., at the focus of interest on or within the subject. Optionally a scaffold comprising the bioengineered B cell is implanted in the subject, e.g., at the focus of interest on or within the subject.

### Example 10: Production of Bioengineered B Cells and Methods of Use

A subject is in need of treatment for a disease or medical condition of interest, or for alleviation of localized symptoms, or combinations thereof, in which the disease, medical condition, or symptoms result in a low glucose environment or in which cells or infectious agents responsible for causing or maintaining the disease, medical condition, or symptoms or the progression thereof utilize an increased level of glucose compared with the corresponding wild-type or uninfected cells of the subject.

One or more B cells are transfected with one or more vectors expressing (a) a cellodextrin transporter protein and (b) a cellobiose phosphorylase protein.

The B cell is an immunogenic B cell selected for production of antibodies in the treatment of the disease or medical condition of interest, or for the production of antibodies for the alleviation of the localized symptoms, or combinations thereof, in the subject.

Optionally, the bioengineered B cell is bioengineered further to comprise a bioengineered antibody specific to the target cell of interest or the bioengineered B cell is a CAR-B cell.

The B cell is administered to the subject in need thereof, optionally at the focus of interest on or within the subject. The subject is placed on a low glucose diet comprising cellobiose. Optionally, a scaffold comprising cellobiose is implanted subject, e.g., at the focus of interest on or within the subject. Optionally a scaffold comprising the bioengineered B cell is implanted in the subject, e.g., at the focus of interest on or within the subject.

### Example 11: Production of Bioengineered Treg Cells and Methods of Use

A subject is in need of treatment for an autoimmune disease or medical condition of interest, or for alleviation of localized symptoms, or combinations thereof, in which the autoimmune disease, inflammation or other medical condition or symptoms result in a low glucose environment or in which T cells responsible for causing or maintaining the autoimmune disease, inflammation or other medical condition or symptoms or the progression thereof utilize an increased level of glucose compared with the corresponding wild-type cells of the subject.

One or more Treg cells are transfected with one or more vectors expressing (a) a cellodextrin transporter protein and (b) a beta-glucosidase protein and/or a cellobiose phosphorylase protein.

The Treg cell is Treg cell selected for the treatment of the autoimmune disease, inflammation, or other medical condition of interest, or for the alleviation of the localized symptoms, or combinations thereof, in the subject, e.g., by suppressing the activity of the T cells responsible for causing or maintaining the autoimmune disease, inflammation or other medical condition or symptoms or the progression thereof.

Optionally, the bioengineered Treg cell is bioengineered further to comprise a bioengineered CAR-Treg cell.

The Treg cell is administered to the subject in need thereof, optionally at the focus of interest on or within the subject. The subject is placed on a low glucose diet comprising cellobiose to selectively activate the Treg cell. Optionally, a scaffold comprising cellobiose is implanted subject, e.g., at the focus of interest on or within the subject. Optionally a scaffold comprising the bioengineered Treg cell is implanted in the subject, e.g., at the focus of interest on or within the subject.

### Example 12: Production of Bioengineered Treg Cells and Methods of Use

A subject is in need of treatment for an autoimmune disease or medical condition of interest, or for alleviation of localized symptoms, or combinations thereof, in which the autoimmune disease, inflammation or other medical condition or symptoms result in a low glucose environment or in which T cells responsible for causing or maintaining the autoimmune disease, inflammation or other medical condition or symptoms or the progression thereof utilize an increased level of glucose compared with the corresponding wild-type cells of the subject.

One or more Treg cells are transfected with one or more vectors expressing (a) a cellodextrin transporter protein and (b) a beta-glucosidase protein.

The Treg cell is Treg cell selected for the treatment of the autoimmune disease, inflammation, or other medical condition of interest, or for the alleviation of the localized symptoms, or combinations thereof, in the subject, e.g., by suppressing the activity of the T cells responsible for causing or maintaining the autoimmune disease, inflammation or other medical condition or symptoms or the progression thereof.

Optionally, the bioengineered Treg cell is bioengineered further to comprise a bioengineered CAR-Treg cell.

The Treg cell is administered to the subject in need thereof, optionally at the focus of interest on or within the subject. The subject is placed on a low glucose diet comprising cellobiose to selectively activate the Treg cell. Optionally, a scaffold comprising cellobiose is implanted subject, e.g., at the focus of interest on or within the subject. Optionally a scaffold comprising the bioengineered Treg cell is implanted in the subject, e.g., at the focus of interest on or within the subject.

### Example 13: Production of Bioengineered Treg Cells and Methods of Use

A subject is in need of treatment for an autoimmune disease or medical condition of interest, or for alleviation of localized symptoms, or combinations thereof, in which the autoimmune disease, inflammation or other medical condition or symptoms result in a low glucose environment or in which T cells responsible for causing or maintaining the autoimmune disease, inflammation or other medical condition or symptoms or the progression thereof utilize an increased level of glucose compared with the corresponding wild-type cells of the subject.

One or more Treg cells are transfected with one or more vectors expressing (a) a cellodextrin transporter protein and (b) a cellobiose phosphorylase protein.

The Treg cell is Treg cell selected for the treatment of the autoimmune disease, inflammation, or other medical condition of interest, or for the alleviation of the localized symptoms, or combinations thereof, in the subject, e.g., by suppressing the activity of the T cells responsible for causing or maintaining the autoimmune disease, inflammation or other medical condition or symptoms or the progression thereof.

Optionally, the bioengineered Treg cell is bioengineered further to comprise a bioengineered CAR-Treg cell.

The Treg cell is administered to the subject in need thereof, optionally at the focus of interest on or within the subject. The subject is placed on a low glucose diet comprising cellobiose to selectively activate the Treg cell. Optionally, a scaffold comprising cellobiose is implanted subject, e.g., at the focus of interest on or within the subject. Optionally a scaffold comprising the bioengineered Treg cell is implanted in the subject, e.g., at the focus of interest on or within the subject.

### Materials and Methods for Examples 14-25

### Construction of Expression Plasmids

As shown in Table 1, the amino acid sequences for cellodextrin transport-1 (cdt-1; https://www.genome.jp/dbget-bin/www_bget?ncr:NCU00801; SEQ ID NO: 3) from Neurospora crassa and glycosylhydrolase family 1-1 (gh1-1; https://www.genome.jp/dbget-bin/www_bget?ncr:NCU:)130; SEQ ID NO: 6) from Neurospora crassa were codon-optimized using the IDT Codon Optimization Tool (https://www.idtdna.com/pages/tools/codon-optimization-tool), BLUE HERON™ BioTech Codon Optimization Tool (https://www.blueheronbio.com/codon-optimization?gclid=CjwKCAjw9MuCBhBUEiwAbDZ-7jQJqeOS6NfjW40raaApv_wPSBk6kTzS7V3D1CxiQifvAfUJBvJ_6hhoCttEQAvD_BwE) (EUROFINS GENOMICS™), or OPTIMUM GENE™ BioTech Codon Optimization Tool (https://www.genscript.com/codon-opt.html?src=google&gclid=CjwKCAjw9MuCBhBUEiwAbDZ-7sdhlVe2q8emWgomPW4wxh9piqffndWQJefv7ay 19-rB-s919Rbp9BoCt7oQAvD_BwE) (GENSCRIPT®). Results are shown in Table 1.

TABLE 1
Optimized Sequences for Construction of Expression Plasmids.

| Construct | |
|---|---|
| (Nucleic Acid | Sequence |
| Type) | (SEQ ID NO:) |
| cdt-1 | ATGTCGTCTCACGGCTCCCATGACGGGGCCAGCACCGAG |
| (DNA prior to | AAGCATCTTGCTACTCATGACATTGCGCCCACCCACGAC |
| optimization) | GCCATCAAGATAGTGCCCAAGGGCCATGGCCAGACAGCC |
| | ACAAAGCCCGGTGCCCAAGAGAAGGAGGTCCGCAACGC |
| | CGCCCTATTTGCGGCCATCAAGGAGTCCAATATCAAGCC |
| | CTGGAGCAAGGAGTCCATCCACCTCTATTTCGCCATCTTC |
| | GTCGCCTTTTGTTGTGCATGCGCCAACGGTTACGATGGTT |
| | CACTCATGACCGGAATCATCGCTATGGACAAGTTCCAGA |
| | ACCAATTCCACACTGGTGACACTGGTCCTAAAGTCTCTGT |
| | CATCTTTTCTCTCTATACCGTTGGTGCCATGGTTGGAGCT |
| | CCCTTCGCTGCTATCCTCTCTGATCGTTTTGGCCGTAAGA |
| | AGGGCATGTTCATCGGTGGTATCTTTATCATTGTCGGCTC |
| | CATTATTGTTGCTAGCTCCTCCAAGCTCGCTCAGTTTGTC |
| | GTTGGCCGCTTCGTTCTTGGCCTCGGTATCGCCATCATGA |
| | CCGTTGCTGCCCCGGCCTACTCCATCGAAATCGCCCCTCC |
| | TCACTGGCGCGGCCGCTGCACTGGCTTCTACAACTGCGGT |
| | TGGTTCGGAGGTTCGATTCCTGCCGCCTGCATCACCTATG |
| | GCTGCTACTTCATTAAGAGCAACTGGTCATGGCGTATCCC |
| | CTTGATCCTTCAGGCTTTCACGTGCCTTATCGTCATGTCCT |
| | CCGTCTTCTTCCTCCCAGAATCCCCTCGCTTCCTATTTGCC |
| | AACGGCCGCGACGCTGAGGCTGTTGCCTTTCTTGTCAAGT |
| | ATCACGGCAACGGCGATCCCAATTCCAAGCTGGTGTTGC |
| | TCGAGACTGAGGAGATGAGGGACGGTATCAGGACCGAC |
| | GGTGTCGACAAGGTCTGGTGGGATTACCGCCCGCTCTTC |
| | ATGACCCACAGCGGCCGCTGGCGCATGGCCCAGGTGCTC |
| | ATGATCTCCATCTTTGGCCAGTTCTCCGGCAACGGTCTCG |
| | GTTACTTCAATACCGTCATCTTCAAGAACATTGGTGTCAC |
| | CAGCACCTCCCAACAGCTCGCCTACAACATCCTCAACTCC |
| | GTCATCTCCGCTATCGGTGCCTTGACCGCCGTCTCCATGA |
| | CTGATCGTATGCCCCGCCGCGCGGTGCTCATTATCGGTAC |
| | CTTCATGTGCGCCGCTGCTCTTGCCACCAACTCGGGTCTT |
| | TCGGCTACTCTCGACAAGCAGACTCAAAGAGGCACGCAA |
| | ATCAACCTGAACCAGGGTATGAACGAGCAGGATGCCAAG |
| | GACAACGCCTACCTCCACGTCGACAGCAACTACGCCAAG |
| | GGTGCCCTGGCCGCTTACTTCCTCTTCAACGTCATCTTCT |
| | CCTTCACCTACACTCCCCTCCAGGGTGTTATTCCCACCGA |
| | GGCTCTCGAGACCACCATCCGTGGCAAGGGTCTTGCCCTT |
| | TCCGGCTTCATTGTCAACGCCATGGGCTTCATCAACCAGT |
| | TCGCTGGCCCCATCGCTCTCCACAACATTGGCTACAAGTA |
| | CATCTTTGTCTTTGTCGGCTGGGATCTTATCGAGACCGTC |
| | GCTTGGTACTTCTTTGGTGTCGAATCCCAAGGCCGTACCC |
| | TCGAGCAGCTCGAATGGGTCTACGACCAGCCCAACCCCG |
| | TCAAGGCCTCCCTAAAAGTCGAAAAGGTCGTCGTCCAGG |
| | CCGACGGCCATGTGTCCGAAGCTATCGTTGCTTAG |
| | (SEQ ID NO: 1) |
| cdt-1 | ATGTCTTCCCACGGTTCACACGACGGAGCTAGTACTGAA |
| (optimized DNA, | AAGCATCTGGCCACCCACGACATAGCCCCCACACATGAT |
| IDT™, version 1; | GCAATCAAGATAGTCCCAAAAGGGCATGGACAGACTGCA |
| IDT v1) | ACTAAACCCGGTGCACAAGAGAAGGAAGTCAGAAATGC |
| | AGCCCTGTTTGCTGCAATAAAAGAGTCCAATATAAAACC |
| | TTGGTCAAAGGAGTCCATTCACTTGTATTTCGCCATCTTT |
| | GTAGCCTTCTGTTGTGCCTGCGCTAATGGGTATGACGGAA |
| | GTCTTATGACAGGGATAATTGCAATGGACAAGTTCCAGA |
| | ACCAGTTCCACACTGGAGACACAGGTCCCAAAGTCAGCG |
| | TTATTTTTTCACTCTACACCGTAGGTGCTATGGTAGGGGC |
| | TCCATTTGCAGCAATCCTCAGTGATCGATTCGGACGAAA |
| | AAAAGGCATGTTCATAGGCGGGATCTTTATCATAGTGGG |
| | CTCCATCATTGTAGCCTCTTCCTCAAAATTGGCACAATTT |
| | GTGGTCGGTCGCTTCGTTCTCGGGCTGGGTATAGCCATCA |
| | TGACCGTCGCAGCTCCAGCATATTCAATAGAGATCGCCC |
| | CACCCCATTGGCGGGGTCGCTGCACCGGCTTCTACAACT |
| | GCGGGTGGTTCGGCGGGTCAATCCCAGCCGCTTGTATAA |
| | CTTATGGGTGCTATTTTATTAAATCAAATTGGTCATGGCG |
| | AATCCCACTCATACTGCAAGCTTTTACCTGTCTTATTGTC |
| | ATGAGCTCAGTCTTCTTCTTGCCAGAATCTCCTCGGTTTTT |
| | GTTCGCCAATGGAAGGGATGCTGAAGCTGTCGCCTTCCT |
| | GGTCAAGTATCACGGAAACGGAGACCCAAACTCTAAATT |
| | GGTTCTGTTGGAGACCGAGGAAATGCGAGACGGAATCCG |
| | GACAGATGGGGTTGACAAGGTATGGTGGGATTATAGGCC |
| | ACTGTTCATGACTCACTCCGGGCGCTGGCGCATGGCCCA |
| | GGTATTGATGATTTCAATTTTCGGGCAATTTAGTGGCAAT |
| | GGACTTGGATACTTCAATACTGTCATCTTCAAAAACATCG |
| | GCGTCACTAGCACCTCACAGCAGCTCGCCTACAATATAC |
| | TCAACAGCGTTATATCTGCTATTGGTGCACTCACCGCTGT |
| | GTCTATGACAGACAGAATGCCCAGGCGCGCAGTTCTCAT |
| | AATAGGCACTTTTATGTGCGCTGCTGCTCTGGCAACTAAC |
| | AGTGGGCTCAGTGCTACTCTTGATAAACAAACTCAGAGA |
| | GGGACCCAGATTAACCTTAACCAAGGGATGAATGAGCAG |
| | GATGCAAAAGATAACGCATACCTTCACGTGGATTCAAAC |
| | TATGCTAAGGGCGCTCTGGCTGCATATTTCCTCTTTAATG |
| | TAATTTTTAGCTTCACATATACCCCTCTTCAAGGTGTCAT |
| | CCCCACCGAGGCCCTGGAAACCACCATTCGGGGGAAGGG |
| | TCTCGCTCTGTCAGGATTCATTGTAAATGCCATGGGCTTT |
| | ATCAATCAATTTGCCGGCCCAATAGCCTTGCACAATATCG |
| | GATATAAATATATCTTTGTATTTGTCGGTTGGGATTTGAT |
| | AGAAACAGTTGCATGGTACTTTTTCGGAGTTGAATCCCA |
| | GGGCAGAACCTTGGAACAACTGGAATGGGTGTACGACCA |
| | ACCTAATCCAGTGAAAGCAAGTCTCAAGGTCGAGAAAGT |
| | CGTAGTTCAAGCCGACGGCCATGTGAGTGAAGCCATCGT |
| | GGCC |
| | (SEQ ID NO: 2) |
| cdt-1 | TCTTCCCACGGTTCACACGACGGAGCTAGTACTGAAAAG |
| (optimized DNA, | CATCTGGCCACCCACGACATAGCCCCCACACATGATGCA |
| IDT™, version 2; | ATCAAGATAGTCCCAAAAGGGCACGGACAGACTGCAACT |
| IDT v2) | AAACCCGGTGCACAAGAGAAGGAAGTCAGAAATGCAGC |
| | CCTGTTTGCTGCAATAAAAGAGTCCAATATAAAACCTTG |
| | GTCAAAGGAGTCCATTCACTTGTATTTCGCCATCTTTGTA |
| | GCCTTCTGTTGTGCCTGCGCTAATGGGTATGACGGATCTT |
| | TGATGACAGGGATAATTGCTATGGACAAGTTCCAGAACC |
| | AGTTCCACACTGGAGACACAGGTCCCAAAGTCAGCGTTA |
| | TTTTTTCACTCTACACCGTAGGTGCTATGGTAGGGGCTCC |
| | ATTTGCAGCAATCCTCAGTGATCGATTCGGACGAAAAAA |
| | AGGTATGTTTATAGGCGGGATCTTTATCATAGTGGGCTCC |
| | ATCATTGTAGCCTCTTCCTCAAAATTGGCACAATTTGTGG |
| | TCGGTCGCTTCGTTCTCGGGCTGGGTATAGCTATTATGAC |
| | AGTCGCAGCTCCAGCATATTCAATAGAGATCGCCCCACC |
| | CCATTGGCGGGGTCGCTGCACCGGCTTCTACAACTGCGG |
| | GTGGTTCGGCGGGTCAATCCCAGCCGCTTGTATAACTTAT |
| | GGGTGCTATTTTATTAAATCAAATTGGTCCTGGCGAATCC |
| | CACTCATACTGCAAGCTTTTACCTGTCTTATTGTTATGTCA |
| | TCAGTCTTCTTCTTGCCAGAATCTCCTCGGTTTTTGTTCGC |
| | CAATGGAAGGGATGCTGAAGCTGTCGCCTTCCTGGTCAA |
| | GTATCACGGAAACGGAGACCCAAACTCTAAATTGGTTCT |
| | GTTGGAGACCGAAGAAATGCGTGACGGAATCCGGACAG |
| | ATGGGGTTGACAAGGTCTGGTGGGATTATAGGCCACTGT |
| | TTATGACTCACTCCGGGCGCTGGCGAATGGCACAGGTAT |
| | TGATGATTTCAATTTTCGGGCAATTTAGTGGCAACGGACT |
| | TGGATACTTCAATACTGTCATCTTCAAAAACATCGGCGTC |
| | ACTAGCACCTCACAGCAGCTCGCCTACAATATACTCAAC |
| | AGCGTTATATCTGCTATTGGTGCACTCACCGCTGTGTCAA |
| | TGACAGATCGAATGCCCAGGCGCGCAGTTCTCATAATAG |
| | GCACTTTTATGTGCGCTGCTGCTCTGGCAACTAACAGTGG |
| | GCTCAGTGCTACTCTTGATAAACAAACTCAGAGAGGGAC |
| | CCAGATTAACCTTAACCAAGGTATGAATGAGCAGGATGC |
| | AAAAGATAACGCATACCTTCACGTGGATTCAAACTATGC |
| | TAAGGGCGCTCTGGCTGCATATTTCCTCTTTAATGTAATT |
| | TTTAGCTTCACATATACCCCTCTTCAAGGTGTCATCCCCA |
| | CCGAGGCCCTGGAAACCACCATTCGGGGGAAGGGTCTCG |
| | CTCTGTCAGGATTCATTGTAAATGCTATGGGATTTATCAA |
| | TCAATTTGCCGGCCCAATAGCCTTGCACAATATCGGATAT |
| | AAATATATCTTTGTATTTGTCGGTTGGGATTTGATAGAAA |
| | CAGTTGCATGGTACTTTTTCGGAGTTGAATCCCAGGGCAG |
| | AACCTTGGAACAACTGGAGTGGGTGTACGACCAACCTAA |
| | TCCAGTGAAAGCAAGTCTCAAGGTCGAGAAAGTCGTAGT |
| | TCAAGCCGACGGCCATGTGAGTGAAGCCATCGTGGCC |
| | (SEQ ID NO: 17) |
| cdt-1 | TCTAGTCACGGAAGTCACGACGGCGCTAGCACCGAAAAG |
| (optimized DNA, | CACCTGGCCACTCACGATATTGCCCCTACCCACGACGCTA |
| BLUE | TCAAGATCGTACCCAAAGGTCACGGGCAGACTGCTACTA |
| HERON™, | AGCCCGGAGCGCAGGAAAAAGAGGTGCGCAACGCTGCC |
| BlueHeron) | CTTTTCGCAGCTATCAAGGAAAGTAATATTAAACCGTGG |
| | AGTAAGGAGAGTATCCATCTCTATTTCGCTATCTTCGTAG |
| | CTTTCTGCTGTGCGTGCGCCAACGGGTATGACGGATCTTT |
| | GATGACAGGAATCATTGCTATGGACAAATTCCAGAATCA |
| | GTTCCATACAGGAGACACAGGTCCCAAGGTCAGTGTTAT |
| | ATTTTCTCTGTACACAGTCGGTGCTATGGTAGGTGCCCCC |
| | TTCGCTGCTATTCTGTCCGACCGCTTCGGACGGAAAAAA |
| | GGTATGTTTATCGGGGGAATTTTTATCATTGTGGGCAGCA |
| | TTATCGTGGCAAGTTCAAGCAAACTGGCTCAATTCGTTGT |
| | TGGCAGGTTCGTCCTGGGACTGGGTATCGCTATTATGACA |
| | GTCGCAGCTCCCGCTTATTCTATCGAAATCGCACCACCGC |
| | ACTGGAGAGGACGCTGCACTGGTTTTTATAACTGCGGCT |
| | GGTTTGGCGGCAGCATCCCGGCGGCATGCATCACCTATG |
| | GCTGCTATTTTATCAAGTCCAACTGGAGCTGGCGAATCCC |
| | CTTGATCCTCCAGGCCTTCACTTGTCTCATTGTTATGTCAT |
| | CTGTTTTTTTTCTCCCTGAGTCCCCTAGATTTCTTTTCGCC |
| | AACGGTAGAGACGCTGAGGCTGTTGCCTTCCTGGTAAAG |
| | TACCACGGCAACGGCGACCCCAACTCCAAACTCGTGCTG |
| | CTGGAGACTGAAGAAATGCGTGACGGGATTCGGACCGAC |
| | GGGGTCGACAAGGTCTGGTGGGACTATCGCCCTCTTTTTA |
| | TGACCCATAGTGGGCGGTGGCGAATGGCACAGGTATTGA |
| | TGATCTCTATCTTTGGGCAATTCTCTGGGAACGGACTTGG |
| | TTACTTTAACACCGTTATCTTTAAAAACATCGGGGTCACT |
| | TCAACCTCTCAGCAATTGGCGTATAACATTCTGAACTCCG |
| | TCATCAGCGCAATCGGGGCACTGACAGCGGTCTCAATGA |
| | CTGATCGAATGCCTCGCAGAGCGGTGCTTATCATCGGAA |
| | CTTTTATGTGCGCTGCTGCCTTGGCCACTAACAGCGGCCT |
| | TTCCGCGACTTTGGATAAACAAACACAGCGGGGTACGCA |
| | GATTAACCTCAATCAGGGTATGAACGAACAAGATGCTAA |
| | AGACAATGCGTATTTGCACGTCGATAGCAATTACGCTAA |
| | GGGTGCTTTGGCCGCCTATTTCCTGTTCAACGTGATTTTT |
| | AGCTTCACGTACACTCCTCTGCAGGGTGTTATTCCAACCG |
| | AGGCACTCGAAACCACGATCCGAGGCAAGGGACTGGCA |
| | CTCAGCGGCTTTATCGTGAACGCTATGGGATTCATTAATC |
| | AGTTTGCTGGCCCTATTGCTCTGCACAACATTGGGTACAA |
| | GTACATCTTCGTTTTCGTGGGCTGGGACCTCATCGAAACT |
| | GTGGCGTGGTATTTCTTCGGAGTGGAGAGTCAGGGGCGA |
| | ACGCTGGAACAGCTCGAATGGGTGTATGATCAACCCAAT |
| | CCTGTAAAAGCAAGTCTGAAGGTGGAGAAAGTTGTGGTG |
| | CAGGCTGATGGACACGTGTCTGAAGCCATCGTGGCG |
| | (SEQ ID NO: 18) |
| cdt-1 | TCATCTCACGGTTCTCACGACGGGGCCTCCACCGAGAAA |
| (optimized DNA, | CATCTCGCTACTCATGACATCGCTCCAACACATGATGCCA |
| GENSCRIPT™; | TAAAGATCGTGCCCAAGGGTCACGGACAGACAGCCACAA |
| GenScript) | AGCCTGGGGCTCAGGAAAAGGAAGTTAGAAATGCAGCC |
| | CTGTTCGCTGCTATTAAAGAAAGTAACATCAAACCGTGG |
| | AGTAAGGAAAGCATCCACCTGTATTTCGCAATATTTGTG |
| | GCTTTCTGCTGCGCCTGTGCCAATGGCTATGACGGATCTT |
| | TGATGACAGGAATAATTGCTATGGACAAGTTCCAGAACC |
| | AGTTCCACACTGGGGACACCGGCCCCAAAGTCTCCGTGA |
| | TCTTTTCTTTATACACCGTTGGTGCTATGGTAGGTGCCCC |
| | CTTTGCTGCGATACTGAGTGACAGATTTGGTAGGAAGAA |
| | AGGTATGTTTATTGGGGGCATTTTTATCATAGTCGGGTCT |
| | ATTATTGTGGCATCCTCCAGCAAACTGGCTCAATTTGTCG |
| | TGGGGCGGTTCGTATTGGGCCTGGGGATTGCTATTATGAC |
| | AGTTGCAGCACCTGCATACAGCATTGAGATCGCTCCGCC |
| | ACACTGGGGGGACGATGTACAGGATTCTACAACTGTGG |
| | GTGGTTTGGAGGCTCCATCCCAGCCGCCTGCATCACCTAT |
| | GGCTGCTACTTCATCAAGAGCAACTGGAGCTGGCGCATC |
| | CCCCTCATCCTCCAAGCCTTCACCTGCCTGATTGTTATGT |
| | CAAGCGTCTTCTTTCTCCCTGAGTCACCACGCTTCCTGTTT |
| | GCCAACGGGCGTGATGCAGAGGCCGTAGCCTTTCTGGTG |
| | AAATACCACGGGAACGGAGACCCAAATTCAAAACTTGTG |
| | CTGCTCGAGACAGAAGAAATGCGTGACGGCATCAGGACA |
| | GATGGTGTTGATAAAGTGTGGTGGGACTACCGGCCTCTTT |
| | TTATGACGCACTCCGGACGCTGGCGAATGGCACAGGTAT |
| | TGATGATCTCCATTTTCGGGCAATTCTCTGGAAACGGACT |
| | AGGATATTTTAACACAGTCATCTTTAAGAATATTGGAGTC |
| | ACATCAACCAGTCAGCAGTTGGCGTATAACATTCTGAAC |
| | AGCGTTATTTCAGCGATCGGCGCTTTAACGGCTGTTTCAA |
| | TGACAGATCGAATGCCCAGGAGAGCTGTGCTTATCATCG |
| | GGACTTTTATGTGTGCTGCTGCGCTGGCCACGAATAGTGG |
| | CCTGTCAGCCACTTTGGATAAGCAGACCCAGCGTGGTAC |
| | TCAGATCAACCTCAACCAGGGTATGAATGAGCAGGACGC |
| | CAAGGACAACGCCTATCTGCACGTGGACAGCAACTATGC |
| | TAAAGGCGCGTTGGCAGCCTACTTTCTCTTCAATGTCATC |
| | TTCAGCTTTACCTACACACCTCTGCAGGGCGTGATTCCTA |
| | CAGAAGCTTTAGAAACCACCATCCGAGGCAAAGGACTCG |
| | CTTTGTCTGGTTTCATAGTGAATGCTATGGGATTTATCAA |
| | TCAGTTTGCAGGGCCCATTGCACTTCACAACATCGGCTAC |
| | AAGTACATCTTCGTCTTTGTTGGCTGGGATCTTATTGAAA |
| | CTGTGGCCTGGTACTTCTTCGGAGTGGAGTCTCAAGGTCG |
| | GACTCTAGAACAGCTGGAGTGGGTGTATGACCAGCCAAA |
| | CCCAGTGAAGGCATCGCTGAAAGTAGAGAAGGTGGTGGT |
| | ACAAGCGGACGGTCATGTCAGTGAAGCAATAGTCGCA |
| | (SEQ ID NO: 19) |
| CDT-1 | MSSHGSHDGASTEKHLATHDIAPTHDAIKIVPKGHGQTATK |
| (protein | PGAQEKEVRNAALFAAIKESNIKPWSKESIHLYFAIFVAFCC |
| expressed from | ACANGYDGSLMTGIIAMDKFQNQFHTGDTGPKVSVIFSLYT |
| SEQ ID NO: 2) | VGAMVGAPFAAILSDRFGRKKGMFIGGIFIIVGSIIVASSSKL |
| | AQFVVGRFVLGLGIAIMTVAAPAYSIEIAPPHWRGRCTGFY |
| | NCGWFGGSIPAACITYGCYFIKSNWSWRIPLILQAFTCLIVM |
| | SSVFFLPESPRFLFANGRDAEAVAFLVKYHGNGDPNSKLVL |
| | LETEEMRDGIRTDGVDKVWWDYRPLFMTHSGRWRMAQV |
| | LMISIFGQFSGNGLGYFNTVIFKNIGVTSTSQQLAYNILNSVIS |
| | AIGALTAVSMTDRMPRRAVLIIGTFMCAAALATNSGLSATL |
| | DKQTQRGTQINLNQGMNEQDAKDNAYLHVDSNYAKGALA |
| | AYFLFNVIFSFTYTPLQGVIPTEALET |
| | TIRGKGLALSGFIVNAMGFINQFAGPIALHNIGYKYIFVFVG |
| | WDLIETVAWYFFGVESQGRTLEQLEWVYDQPNPVKASLKV |
| | EKVVVQADGHVSEAIVA |
| | (SEQ ID NO: 3) |
| gh1-1 | ATGTCTCTTCCTAAGGATTTCCTCTGGGGCTTCGCTACTG |
| (DNA prior to | CGGCCTATCAGATTGAGGGTGCTATCCACGCCGACGGCC |
| optimization) | GTGGCCCCTCTATCTGGGATACTTTCTGCAACATTCCCGG |
| | TAAAATCGCCGACGGCAGCTCTGGTGCCGTCGCCTGCGA |
| | CTCTTACAACCGCACCAAGGAGGACATTGACCTCCTCAA |
| | GTCTCTCGGCGCCACCGCCTACCGCTTCTCCATCTCCTGG |
| | TCTCGCATCATCCCCGTTGGTGGTCGCAACGACCCCATCA |
| | ACCAGAAGGGCATCGACCACTATGTCAAGTTTGTCGATG |
| | ACCTGCTCGAGGCTGGTATTACCCCCTTTATCACCCTCTT |
| | CCACTGGGATCTTCCCGATGGTCTCGACAAGCGCTACGG |
| | CGGTCTTCTGAACCGTGAAGAGTTCCCCCTCGACTTTGAG |
| | CACTACGCCCGCACTATGTTCAAGGCCATTCCCAAGTGC |
| | AAGCACTGGATCACCTTCAACGAGCCCTGGTGCAGCTCC |
| | ATCCTCGGCTACAACTCGGGCTACTTTGCCCCTGGCCACA |
| | CCTCCGACCGTACCAAGTCACCCGTTGGTGACAGCGCTC |
| | GCGAGCCCTGGATCGTCGGCCATAACCTGCTCATCGCTC |
| | ACGGGCGTGCCGTCAAGGTGTACCGAGAAGACTTCAAGC |
| | CCACGCAGGGCGGCGAGATCGGTATCACCTTGAACGGCG |
| | ACGCCACTCTTCCCTGGGATCCAGAGGACCCCTTGGACG |
| | TCGAGGCGTGCGACCGCAAGATTGAGTTCGCCATCAGCT |
| | GGTTCGCAGACCCCATCTACTTTGGAAAGTACCCCGACTC |
| | GATGCGCAAACAGCTCGGTGACCGGCTGCCCGAGTTTAC |
| | GCCCGAGGAGGTGGCGCTTGTCAAGGGTTCCAACGACTT |
| | CTACGGCATGAACCACTACACAGCCAACTACATCAAGCA |
| | CAAGAAGGGCGTCCCTCCCGAGGACGACTTCCTCGGCAA |
| | CCTCGAGACGCTCTTCTACAACAAGAAGGGTAACTGCAT |
| | CGGGCCCGAGACCCAGTCGTTCTGGCTCCGGCCGCACGC |
| | CCAGGGCTTCCGCGACCTGCTCAACTGGCTCAGCAAGCG |
| | CTACGGATACCCCAAGATCTACGTGACCGAGAACGGGAC |
| | CAGTCTCAAGGGCGAGAACGCCATGCCGCTCAAGCAAAT |
| | TGTCGAGGACGACTTCCGCGTCAAGTACTTCAACGACTA |
| | CGTCAACGCCATGGCCAAGGCGCATAGCGAGGACGGCGT |
| | CAACGTCAAGGGATATCTTGCCTGGAGCTTGATGGACAA |
| | CTTTGAGTGGGCCGAGGGCTATGAGACGCGGTTCGGCGT |
| | TACCTATGTCGACTATGAGAACGACCAGAAGAGGTACCC |
| | CAAGAAGAGCGCCAAGAGCTTGAAGCCGCTCTTTGACTC |
| | TTTGATCAAGAAGGACTAA |
| | (SEQ ID NO: 4) |
| gh1-1 | TCCTTGCCCAAGGATTTTCTGTGGGGGTTTGCCACAGCT |
| (optimized DNA) | GCCTATCAAATTGAGGGCGCTATTCACGCAGATGGAAG |
| | AGGACCATCCATTTGGGACACATTTTGCAACATCCCTGG |
| | CAAGATAGCAGACGGATCTAGCGGTGCCGTGGCTTGCG |
| | ACTCATACAACAGAACTAAAGAGGATATTGACCTCCTG |
| | AAGAGCTTGGGCGCAACAGCATACAGGTTTAGTATTTC |
| | ATGGAGCAGAATCATCCCAGTAGGAGGCAGAAACGACC |
| | CTATTAACCAGAAGGGTATAGATCACTACGTTAAGTTTG |
| | TGGATGATCTGCTTGAGGCAGGTATCACCCCATTTATTA |
| | CCCTCTTTCATTGGGATTTGCCTGATGGTCTCGATAAGC |
| | GCTATGGCGGGCTCTTGAATCGGGAGGAGTTCCCTCTGG |
| | ACTTCGAGCATTACGCTAGGACTATGTTCAAGGCTATAC |
| | CAAAATGTAAGCATTGGATCACTTTCAACGAACCCTGGT |
| | GCTCCTCAATCCTCGGATACAACTCAGGATATTTTGCTC |
| | CAGGACACACTTCTGACAGAACAAAAAGTCCAGTAGGC |
| | GATAGCGCCCGCGAGCCCTGGATAGTTGGCCATAATCT |
| | GTTGATCGCACATGGGCGAGCTGTCAAAGTTTATCGGG |
| | AAGATTTCAAGCCTACACAGGGAGGCGAAATTGGCATC |
| | ACCCTGAACGGGGACGCCACCCTGCCCTGGGACCCAGA |
| | GGACCCTCTCGATGTCGAGGCCTGCGATCGCAAGATAG |
| | AGTTTGCAATTTCATGGTTTGCTGATCCCATTTATTTTGG |
| | AAAGTACCCTGACTCCATGAGAAAGCAGCTGGGTGACA |
| | GGCTTCCAGAGTTCACACCTGAAGAAGTTGCTCTTGTCA |
| | AGGGATCCAACGATTTCTACGGTATGAATCATTATACAG |
| | CTAACTATATCAAACATAAAAAAGGTGTTCCACCCGAG |
| | GACGATTTTTTGGGTAATCTCGAAACCTTGTTTTATAAC |
| | AAAAAGGGAAACTGTATAGGCCCAGAGACCCAGAGTTT |
| | CTGGCTCCGACCCCATGCTCAAGGGTTCCGCGACCTCCT |
| | GAATTGGTTGTCCAAGCGATACGGCTATCCTAAGATTTA |
| | TGTGACAGAGAACGGTACTTCATTGAAGGGCGAGAATG |
| | CAATGCCTTTGAAGCAAATTGTAGAAGATGATTTCCGCG |
| | TTAAGTACTTTAATGACTATGTAAATGCTATGGCTAAGG |
| | CACACTCCGAAGATGGAGTTAATGTCAAAGGATACCTC |
| | GCTTGGTCTCTTATGGATAATTTCGAGTGGGCAGAAGGC |
| | TATGAGACTAGATTCGGTGTGACATATGTGGATTACGAG |
| | AACGATCAGAAGCGCTATCCCAAGAAATCAGCCAAATC |
| | CCTCAAACCATTGTTTGATTCATTGATTAAGAAAGAC |
| | (SEQ ID NO: 5) |
| GH1-1 | MSLPKDFLWGFATAAYQIEGAIHADGRGPSIWDTFCNIPGKI |
| (protein | ADGSSGAVACDSYNRTKEDIDLLKSLGATAYRESISWSRIIP |
| expressed from | VGGRNDPINQKGIDHYVKFVDDLLEAGITPFITLFHWDLPDG |
| SEQ ID NO: 5) | LDKRYGGLLNREEFPLDFEHYARTMFKAIPKCKHWITFNEP |
| | WCSSILGYNSGYFAPGHTSDRTKSPVGDSAREPWIVGHNLLI |
| | AHGRAVKVYREDFKPTQGGEIGITLNGDATLPWDPEDPLDV |
| | EACDRKIEFAISWFADPIYFGKYPDSMRKQLGDRLPEFTPEE |
| | VALVKGSNDFYGMNHYTANYIKHKKGVPPEDDFLGNLETL |
| | FYNKKGNCIGPETQSFWLRPHAQGFRDLLNWLSKRYGYPKI |
| | YVTENGTSLKGENAMPLKQIVEDDFRVKYFNDYVNAMAK |
| | AHSEDGVNVKGYLAWSLMDNFEWAEGYETRFGVTYVDYE |
| | NDQKRYPKKSAKSLKPLFDSLIKKD |
| | (SEQ ID NO: 6) |

Each of the resulting sequences had an HA-tag (TACCCATACGATGTTCCAGATTACGCT (SEQ ID NO: 20)) addended to the N-terminus and 25 base pair overlaps (5′-ACTCCTTCTCTAGGCGCCGGAATTA (SEQ ID NO: 21); 3′-AATTCTACCGGGTAGGTGAGGCGCT (SEQ ID NO: 22)) for integration into the expression plasmid at the N- and C-terminus were also added. The ATG start codon is upstream of the HA-tag, which is N-terminal on this protein. The cdt-1 or gh1-1 sequences were then synthesized as GBLOCKS™ Gene Fragments by INTEGRATED DNA TECHNOLOGIES™ (IDT). Each of the resultant double-stranded DNA fragments was cloned into either an MSCV MCS PGK-GFP vector or MSCV MCS PGK-mCherry vector, which were restriction digested using BglII and EcoRI restriction endonucleases (NEW ENGLAND BIOLABS®). The MSCV MCS PGK-mCherry vector was cloned by removing the green fluorescent protein (GFP) sequence and replacing it with mCherry. The linearized plasmid was then combined with the cdt-1 or gh1-1 gBlock (GBLOCKS™ Gene Fragment, INTEGRATED DNA TECHNOLOGIES™) and NEBUILDER® HiFi DNA Assembly Master Mix (NEW ENGLAND BIOLABS®, #E2621S) before transformation into NEB® 5-alpha Competent E. coli (High Efficiency) (NEW ENGLAND BIOLABS®, #C2987H). Later iterations of the cdt-1 plasmid followed the same protocol, but with the HA-tag (see above (SEQ ID NO: 20)) and ERES signal (DNA sequence: TTTTGCTATGAAAATGAA (SEQ ID NO: 23); or DNA sequence: TTCTGCTACGAGAATGAA (SEQ ID NO: SEQ ID NO: 24); amino acid sequence: FCYENE (SEQ ID NO: 25); https://www.sciencedirect.com/science/article/pii/S009286741000190X) addended to the C-terminus.

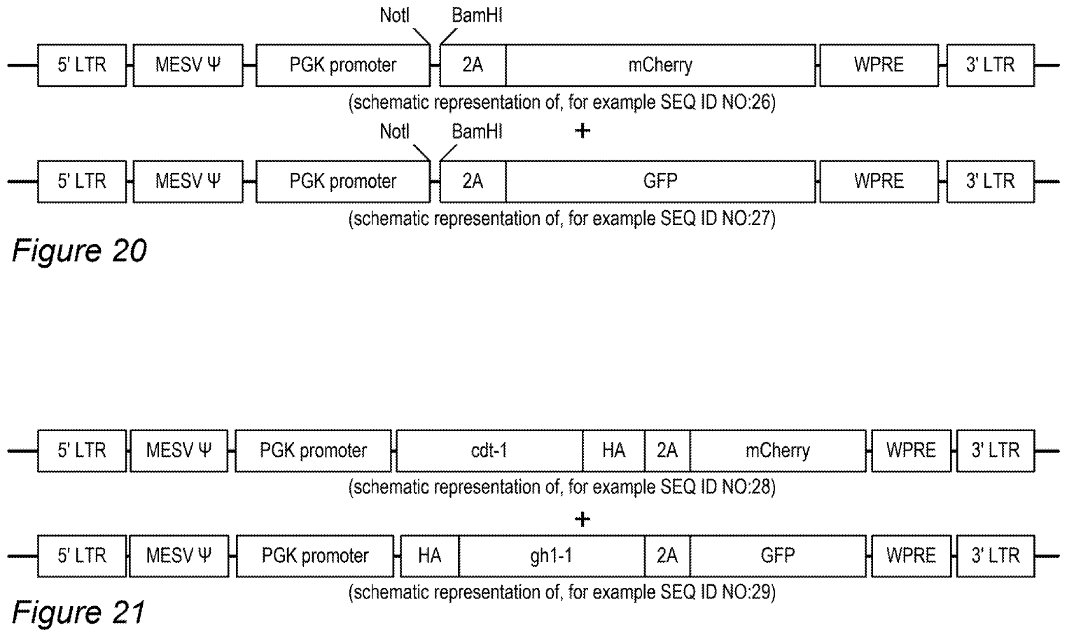

Further iterations of the cdt-1 and gh1-1 plasmids utilized different elements in redesigned viral delivery vectors (FIG. 20, top and bottom). The transgenes, cdt-1 and gh1-1, were located under the control of the strong, constitutive promoter PGK (FIG. 21, top and bottom). The 3′ ends of the genes were modified to contain an optional linker to a 2A ribosomal skipping sequence (here, T2A ribosomal skipping sequence) that was then followed in-frame by either the mCherry or GFP coding sequence. This design allowed for (1) enhanced expression of the transgene and (2) coupling of the fluorescent protein markers to cells actively expressing the transgene. Additionally, the Woodchuck hepatitis virus post-transcriptional regulatory element (WPRE) was added downstream from the transgene. WPRE has been shown to increase transcript stability and leads to enhanced protein expression on transcripts where it is present.

The sequences of the resulting vector constructs are shown in Table 2.

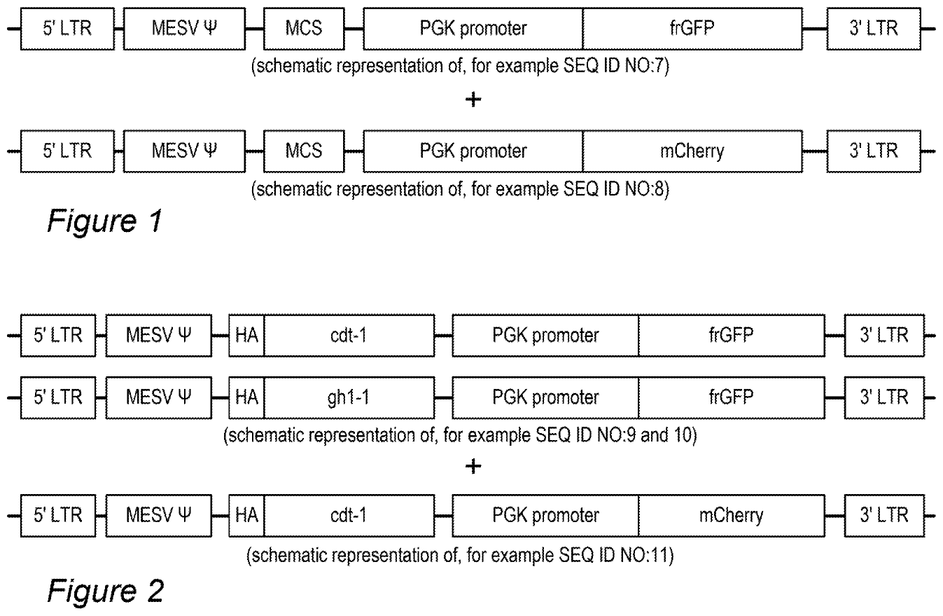

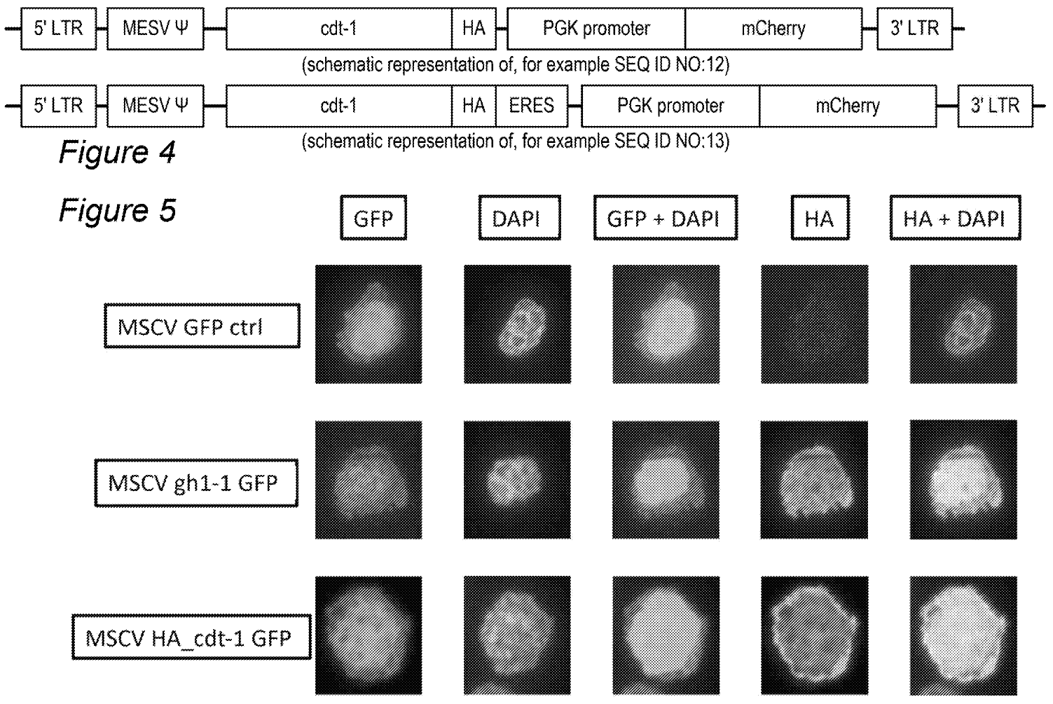

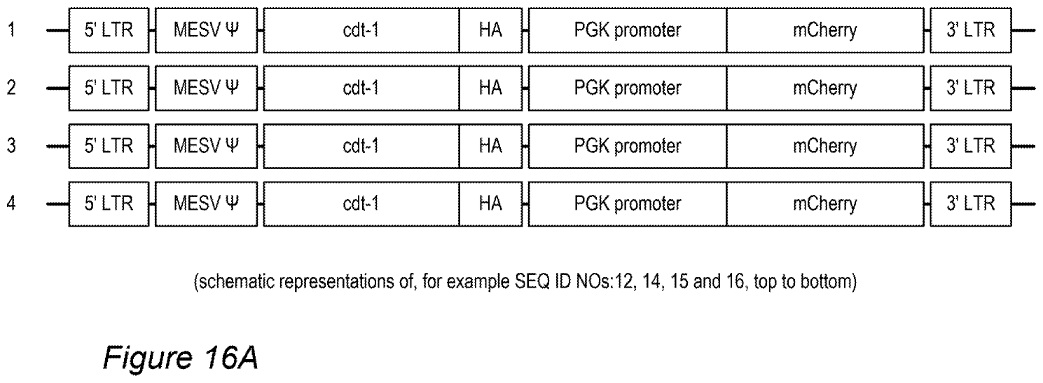

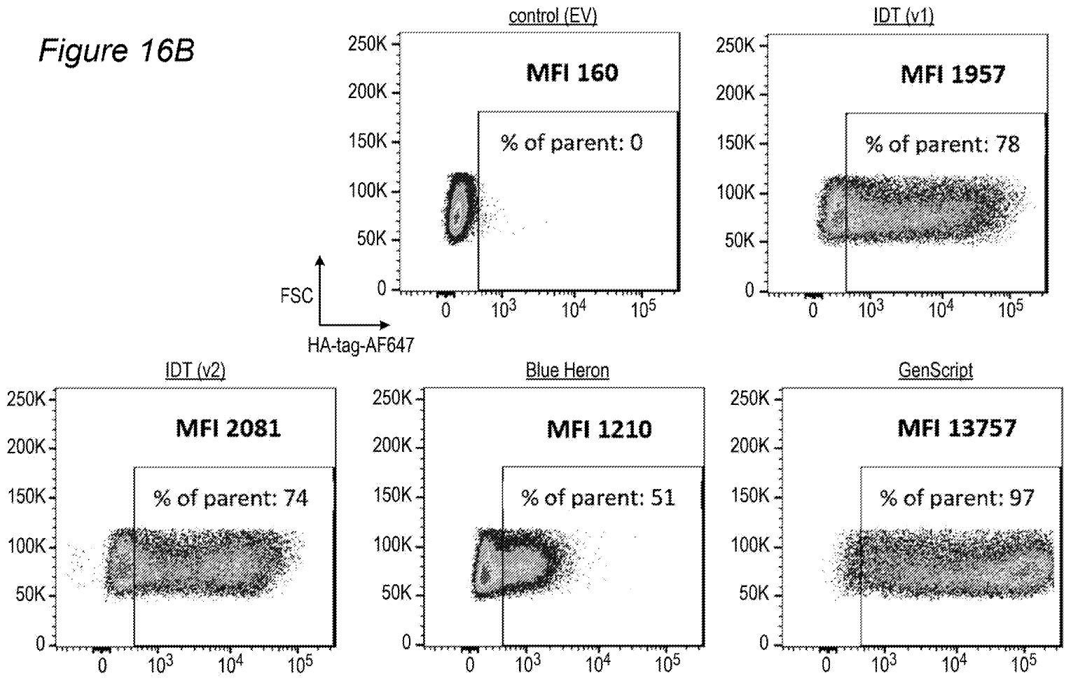

TABLE 2
Vector Constructs.

| Construct | |
|---|---|
| (Corresponding | Sequence |
| FIG.) | (SEQ ID NO:) |
| MSCV-MCS- | TGAAAGACCC CACCTGTAGG TTTGGCAAGC TAGCTTAAGT |
| PGK-GFP | AACGCCATTT TGCAAGGCAT GGAAAATACA TAACTGAGAA |
| (FIG. 1, top) | TAGAGAAGTT CAGATCAAGG TTAGGAACAG AGAGACAGCA |
| | GAATATGGGC CAAACAGGAT ATCTGTGGTA AGCAGTTCCT |
| | GCCCCGGCTC AGGGCCAAGA ACAGATGGTC CCCAGATGCG |
| | GTCCCGCCCT CAGCAGTTTC TAGAGAACCA TCAGATGTTT |
| | CCAGGGTGCC CCAAGGACCT GAAATGACCC TGTGCCTTAT |
| | TTGAACTAAC CAATCAGTTC GCTTCTCGCT TCTGTTCGCG |
| | CGCTTCTGCT CCCCGAGCTC AATAAAAGAG CCCACAACCC |
| | CTCACTCGGC GCGCCAGTCC TCCGATAGAC TGCGTCGCCC |
| | GGGTACCCGT ATTCCCAATA AAGCCTCTTG CTGTTTGCAT |
| | CCGAATCGTG GACTCGCTGA TCCTTGGGAG GGTCTCCTCA |
| | GATTGATTGA CTGCCCACCT CGGGGGTCTT TCATTTGGAG |
| | GTTCCACCGA GATTTGGAGA CCCCTGCCCA GGGACCACCG |
| | ACCCCCCCGC CGGGAGGTAA GCTGGCCAGC GGTCGTTTCG |
| | TGTCTGTCTC TGTCTTTGTG CGTGTTTGTG CCGGCATCTA |
| | ATGTTTGCGC CTGCGTCTGT ACTAGTTAGC TAACTAGCTC |
| | TGTATCTGGC GGACCCGTGG TGGAACTGAC GAGTTCTGAA |
| | CACCCGGCCG CAACCCTGGG AGACGTCCCA GGGACTTTGG |
| | GGGCCGTTTT TGTGGCCCGA CCTGAGGAAG GGAGTCGATG |
| | TGGAATCCGA CCCCGTCAGG ATATGTGGTT CTGGTAGGAG |
| | ACGAGAACCT AAAACAGTTC CCGCCTCCGT CTGAATTTTT |
| | GCTTTCGGTT TGGAACCGAA GCCGCGCGTC TTGTCTGCTG |
| | CAGCGCTGCA GCATCGTTCT GTGTTGTCTC TGTCTGACTG |
| | TGTTTCTGTA TTTGTCTGAA AATTAGGGCC AGACTGTTAC |
| | CACTCCCTTA AGTTTGACCT TAGGTCACTG GAAAGATGTC |
| | GAGCGGATCG CTCACAACCA GTCGGTAGAT GTCAAGAAGA |
| | GACGTTGGGT TACCTTCTGC TCTGCAGAAT GGCCAACCTT |
| | TAACGTCGGA TGGCCGCGAG ACGGCACCTT TAACCGAGAC |
| | CTCATCACCC AGGTTAAGAT CAAGGTCTTT TCACCTGGCC |
| | CGCATGGACA CCCAGACCAG GTCCCCTACA TCGTGACCTG |
| | GGAAGCCTTG GCTTTTGACC CCCCTCCCTG GGTCAAGCCC |
| | TTTGTACACC CTAAGCCTCC GCCTCCTCTT CCTCCATCCG |
| | CCCCGTCTCT CCCCCTTGAA CCTCCTCGTT CGACCCCGCC |
| | TCGATCCTCC CTTTATCCAG CCCTCACTCC TTCTCTAGGC |
| | GCCGGAATTA GATCTCTCGA GGTTATCACA AGTTTGTACA |
| | AAAAAGCAGG CTTCGAAGGA GATAGAACCA ATTCTCTAAG |
| | GAAATACTTA ACCATGGTCG ACTGGATCCG GTACCGAATT |
| | CGCGGCCGCA CTCGAGATAT CTAGACCCAG CTTTCTTGTA |
| | CAAAGTGGTG ATAACGAATT CTACCGGGTA GGTGAGGCGC |
| | TTTTCCCAAG GCAGTCTGGA GCATGCGCTT TAGCAGCCCC |
| | GCTGGGCACT TGGCGCTACA CAAGTGGCCT CTGGCCTCGC |
| | ACACATTCCA CATCCACCGG TAGGCGCCAA CCGGCTCCGT |
| | TCTTTGGTGG CCCCTTCGCG CCACCTTCTA CTCCTCCCCT |
| | AGTCAGGAAG TTCCCCCCCG CCCCGCAGCT CGCGTCGTGC |
| | AGGACGTGAC AAATGGAAGT AGCACGTCTC ACTAGTCTCG |
| | TGCAGATGGA CAGCACCGCT GAGCAATGGA AGCGGGTAGG |
| | CCTTTGGGGC AGCGGCCAAT AGCAGCTTTG CTCCTTCGCT |
| | TTCTGGGCTC AGAGGCTGGG AAGGGGTGGG TCCGGGGGCG |
| | GGCTCAGGGG CGGGCTCAGG GGCGGGGCGG GCGCCCGAAG |
| | GTCCTCCGGA GGCCCGGCAT TCTGCACGCT TCAAAAGCGC |
| | ACGTCTGCCG CGCTGTTCTC CTCTTCCTCA TCTCCGGGCC |
| | TTTCGACCTG CAGCCCAAGC TAGGACCATG GTGAGCAAGG |
| | GCGAGGAGCT GTTCACCGGG GTGGTGCCCA TCCTGGTCGA |
| | GCTGGACGGC GACGTAAACG GCCACAAGTT CAGCGTGTCC |
| | GGCGAGGGCG AGGGCGATGC CACCTACGGC AAGCTGACCC |
| | TGAAGTTCAT CTGCACCACC GGCAAGCTGC CCGTGCCCTG |
| | GCCCACCCTC GTGACCACCT TCACCTACGG CGTGCAGTGC |
| | TTCAGCCGCT ACCCCGACCA CATGAAGCAG CACGACTTCT |
| | TCAAGTCCGC CATGCCCGAA GGCTACGTCC AGGAGCGCAC |
| | CATCTCTTTC AAGGACGACG GCAACTACAA GACCCGCGCC |
| | GAGGTGAAGT TCGAGGGCGA CACCCTGGTG AACCGCATCG |
| | AGCTGAAGGG CATCGACTTC AAGGAGGACG GCAACATCCT |
| | GGGGCACAAG CTGGAGTACA ACTACAACAG CCACAACGTC |
| | TATATCACGG CCGACAAGCA GAAGAACGGC ATCAAGGCTA |
| | ACTTCAAGAT CCGCCACAAC ATCGAGGACG GCAGCGTGCA |
| | GCTCGCCGAC CACTACCAGC AGAACACCCC CATCGGCGAC |
| | GGCCCCGTGC TGCTGCCCGA CAACCACTAC CTGAGCACCC |
| | AGTCCGCCCT GAGCAAAGAC CCCAACGAGA AGCGCGATCA |
| | CATGGTCCTG CTGGAGTTCG TGACCGCCGC CGGGATCACT |
| | CTCGGCATGG ACGAGCTGTA CAAGTGAATG CATCGATAAA |
| | ATAAAAGATT TTATTTAGTC TCCAGAAAAA GGGGGGAATG |
| | AAAGACCCCA CCTGTAGGTT TGGCAAGCTA GCTTAAGTAA |
| | CGCCATTTTG CAAGGCATGG AAAATACATA ACTGAGAATA |
| | GAGAAGTTCA GATCAAGGTT AGGAACAGAG AGACAGCAGA |
| | ATATGGGCCA AACAGGATAT CTGTGGTAAG CAGTTCCTGC |
| | CCCGGCTCAG GGCCAAGAAC AGATGGTCCC CAGATGCGGT |
| | CCCGCCCTCA GCAGTTTCTA GAGAACCATC AGATGTTTCC |
| | AGGGTGCCCC AAGGACCTGA AATGACCCTG TGCCTTATTT |
| | GAACTAACCA ATCAGTTCGC TTCTCGCTTC TGTTCGCGCG |
| | CTTCTGCTCC CCGAGCTCAA TAAAAGAGCC CACAACCCCT |
| | CACTCGGCGC GCCAGTCCTC CGATAGACTG CGTCGCCCGG |
| | GTACCCGTGT ATCCAATAAA CCCTCTTGCA GTTGCATCCG |
| | ACTTGTGGTC TCGCTGTTCC TTGGGAGGGT CTCCTCTGAG |
| | TGATTGACTA CCCGTCAGCG GGGGTCTTTC ATGGGTAACA |
| | GTTTCTTGAA GTTGGAGAAC AACATTCTGA GGGTAGGAGT |
| | CGAATATTAA GTAATCCTGA CTCAATTAGC CACTGTTTTG |
| | AATCCACATA CTCCAATACT CCTGAAATAG TTCATTATGG |
| | ACAGCGCAGA AGAGCTGGGG AGAATTGTGA AATTGTTATC |
| | CGCTCACAAT TCCACACAAC ATACGAGCCG GAAGCATAAA |
| | GTGTAAAGCC TGGGGTGCCT AATGAGTGAG CTAACTCACA |
| | TTAATTGCGT TGCGCTCACT GCCCGCTTTC CAGTCGGGAA |
| | ACCTGTCGTG CCAGCTGCAT TAATGAATCG GCCAACGCGC |
| | GGGGAGAGGC GGTTTGCGTA TTGGGCGCTC TTCCGCTTCC |
| | TCGCTCACTG ACTCGCTGCG CTCGGTCGTT CGGCTGCGGC |
| | GAGCGGTATC AGCTCACTCA AAGGCGGTAA TACGGTTATC |
| | CACAGAATCA GGGGATAACG CAGGAAAGAA CATGTGAGCA |
| | AAAGGCCAGC AAAAGGCCAG GAACCGTAAA AAGGCCGCGT |
| | TGCTGGCGTT TTTCCATAGG CTCCGCCCCC CTGACGAGCA |
| | TCACAAAAAT CGACGCTCAA GTCAGAGGTG GCGAAACCCG |
| | ACAGGACTAT AAAGATACCA GGCGTTTCCC CCTGGAAGCT |
| | CCCTCGTGCG CTCTCCTGTT CCGACCCTGC CGCTTACCGG |
| | ATACCTGTCC GCCTTTCTCC CTTCGGGAAG CGTGGCGCTT |
| | TCTCATAGCT CACGCTGTAG GTATCTCAGT TCGGTGTAGG |
| | TCGTTCGCTC CAAGCTGGGC TGTGTGCACG AACCCCCCGT |
| | TCAGCCCGAC CGCTGCGCCT TATCCGGTAA CTATCGTCTT |
| | GAGTCCAACC CGGTAAGACA CGACTTATCG CCACTGGCAG |
| | CAGCCACTGG TAACAGGATT AGCAGAGCGA GGTATGTAGG |
| | CGGTGCTACA GAGTTCTTGA AGTGGTGGCC TAACTACGGC |
| | TACACTAGAA GGACAGTATT TGGTATCTGC GCTCTGCTGA |
| | AGCCAGTTAC CTTCGGAAAA AGAGTTGGTA GCTCTTGATC |
| | CGGCAAACAA ACCACCGCTG GTAGCGGTGG TTTTTTTGTT |
| | TGCAAGCAGC AGATTACGCG CAGAAAAAAA GGATCTCAAG |
| | AAGATCCTTT GATCTTTTCT ACGGGGTCTG ACGCTCAGTG |
| | GAACGAAAAC TCACGTTAAG GGATTTTGGT CATGAGATTA |
| | TCAAAAAGGA TCTTCACCTA GATCCTTTTA AATTAAAAAT |
| | GAAGTTTTAA ATCAATCTAA AGTATATATG AGTAAACTTG |
| | GTCTGACAGT TACCAATGCT TAATCAGTGA GGCACCTATC |
| | TCAGCGATCT GTCTATTTCG TTCATCCATA GTTGCCTGAC |
| | TCCCCGTCGT GTAGATAACT ACGATACGGG AGGGCTTACC |
| | ATCTGGCCCC AGTGCTGCAA TGATACCGCG AGACCCACGC |
| | TCACCGGCTC CAGATTTATC AGCAATAAAC CAGCCAGCCG |
| | GAAGGGCCGA GCGCAGAAGT GGTCCTGCAA CTTTATCCGC |
| | CTCCATCCAG TCTATTAATT GTTGCCGGGA AGCTAGAGTA |
| | AGTAGTTCGC CAGTTAATAG TTTGCGCAAC GTTGTTGCCA |
| | TTGCTACAGG CATCGTGGTG TCACGCTCGT CGTTTGGTAT |
| | GGCTTCATTC AGCTCCGGTT CCCAACGATC AAGGCGAGTT |
| | ACATGATCCC CCATGTTGTG CAAAAAAGCG GTTAGCTCCT |
| | TCGGTCCTCC GATCGTTGTC AGAAGTAAGT TGGCCGCAGT |
| | GTTATCACTC ATGGTTATGG CAGCACTGCA TAATTCTCTT |
| | ACTGTCATGC CATCCGTAAG ATGCTTTTCT GTGACTGGTG |
| | AGTACTCAAC CAAGTCATTC TGAGAATAGT GTATGCGGCG |
| | ACCGAGTTGC TCTTGCCCGG CGTCAATACG GGATAATACC |
| | GCGCCACATA GCAGAACTTT AAAAGTGCTC ATCATTGGAA |
| | AACGTTCTTC GGGGCGAAAA CTCTCAAGGA TCTTACCGCT |
| | GTTGAGATCC AGTTCGATGT AACCCACTCG TGCACCCAAC |
| | TGATCTTCAG CATCTTTTAC TTTCACCAGC GTTTCTGGGT |
| | GAGCAAAAAC AGGAAGGCAA AATGCCGCAA |
| | AAAAGGGAAT AAGGGCGACA CGGAAATGTT GAATACTCAT |
| | ACTCTTCCTT TTTCAATATT ATTGAAGCAT TTATCAGGGT |
| | TATTGTCTCA TGAGCGGATA CATATTTGAA TGTATTTAGA |
| | AAAATAAACA AATAGGGGTT CCGCGCACAT TTCCCCGAAA |
| | AGTGCCACCT GACGTCTAAG AAACCATTAT TATCATGACA |
| | TTAACCTATA AAAATAGGCG TATCACGAGG CCCTTTCGTC |
| | TCGCGCGTTT CGGTGATGAC GGTGAAAACC TCTGACACAT |
| | GCAGCTCCCG GAGACGGTCA CAGCTTGTCT GTAAGCGGAT |
| | GCCGGGAGCA GACAAGCCCG TCAGGGCGCG TCAGCGGGTG |
| | TTGGCGGGTG TCGGGGCTGG CTTAACTATG CGGCATCAGA |
| | GCAGATTGTA CTGAGAGTGC ACCATATGCG GTGTGAAATA |
| | CCGCACAGAT GCGTAAGGAG AAAATACCGC ATCAGGCGCC |
| | ATTCGCCATT CAGGCTGCGC AACTGTTGGG AAGGGCGATC |
| | GGTGCGGGCC TCTTCGCTAT TACGCCAGCT GGCGAAAGGG |
| | GGATGTGCTG CAAGGCGATT AAGTTGGGTA ACGCCAGGGT |
| | TTTCCCAGTC ACGACGTTGT AAAACGACGG CGCAAGGAAT |
| | GGTGCATGCA AGGAGATGGC GCCCAACAGT CCCCCGGCCA |
| | CGGGGCCTGC CACCATACCC ACGCCGAAAC AAGCGCTCAT |
| | GAGCCCGAAG TGGCGAGCCC GATCTTCCCC ATCGGTGATG |
| | TCGGCGATAT AGGCGCCAGC AACCGCACCT GTGGCGCCGG |
| | TGATGCCGGC CACGATGCGT CCGGCGTAGA GGCGATTAGT |
| | CCAATTTGTT AAAGACAGGA TATCAGTGGT CCAGGCTCTA |
| | GTTTTGACTC AACAATATCA CCAGCTGAAG CCTATAGAGT |
| | ACGAGCCATA GATAAAATAA AAGATTTTAT TTAGTCTCCA |
| | GAAAAAGGGG GGAA |
| | (SEQ ID NO: 7) |
| MSCV-MCS- | TGAAAGACCC CACCTGTAGG TTTGGCAAGC TAGCTTAAGT |
| PGK-mCherry | AACGCCATTT TGCAAGGCAT GGAAAATACA TAACTGAGAA |
| (FIG. 1, | TAGAGAAGTT CAGATCAAGG TTAGGAACAG AGAGACAGCA |
| bottom) | GAATATGGGC CAAACAGGAT ATCTGTGGTA AGCAGTTCCT |
| | GCCCCGGCTC AGGGCCAAGA ACAGATGGTC CCCAGATGCG |
| | GTCCCGCCCT CAGCAGTTTC TAGAGAACCA TCAGATGTTT |
| | CCAGGGTGCC CCAAGGACCT GAAATGACCC TGTGCCTTAT |
| | TTGAACTAAC CAATCAGTTC GCTTCTCGCT TCTGTTCGCG |
| | CGCTTCTGCT CCCCGAGCTC AATAAAAGAG CCCACAACCC |
| | CTCACTCGGC GCGCCAGTCC TCCGATAGAC TGCGTCGCCC |
| | GGGTACCCGT ATTCCCAATA AAGCCTCTTG CTGTTTGCAT |
| | CCGAATCGTG GACTCGCTGA TCCTTGGGAG GGTCTCCTCA |
| | GATTGATTGA CTGCCCACCT CGGGGGTCTT TCATTTGGAG |
| | GTTCCACCGA GATTTGGAGA CCCCTGCCCA GGGACCACCG |
| | ACCCCCCCGC CGGGAGGTAA GCTGGCCAGC GGTCGTTTCG |
| | TGTCTGTCTC TGTCTTTGTG CGTGTTTGTG CCGGCATCTA |
| | ATGTTTGCGC CTGCGTCTGT ACTAGTTAGC TAACTAGCTC |
| | TGTATCTGGC GGACCCGTGG TGGAACTGAC GAGTTCTGAA |
| | CACCCGGCCG CAACCCTGGG AGACGTCCCA GGGACTTTGG |
| | GGGCCGTTTT TGTGGCCCGA CCTGAGGAAG GGAGTCGATG |
| | TGGAATCCGA CCCCGTCAGG ATATGTGGTT CTGGTAGGAG |
| | ACGAGAACCT AAAACAGTTC CCGCCTCCGT CTGAATTTTT |
| | GCTTTCGGTT TGGAACCGAA GCCGCGCGTC TTGTCTGCTG |
| | CAGCGCTGCA GCATCGTTCT GTGTTGTCTC TGTCTGACTG |
| | TGTTTCTGTA TTTGTCTGAA AATTAGGGCC AGACTGTTAC |
| | CACTCCCTTA AGTTTGACCT TAGGTCACTG GAAAGATGTC |
| | GAGCGGATCG CTCACAACCA GTCGGTAGAT GTCAAGAAGA |
| | GACGTTGGGT TACCTTCTGC TCTGCAGAAT GGCCAACCTT |
| | TAACGTCGGA TGGCCGCGAG ACGGCACCTT TAACCGAGAC |
| | CTCATCACCC AGGTTAAGAT CAAGGTCTTT TCACCTGGCC |
| | CGCATGGACA CCCAGACCAG GTCCCCTACA TCGTGACCTG |
| | GGAAGCCTTG GCTTTTGACC CCCCTCCCTG GGTCAAGCCC |
| | TTTGTACACC CTAAGCCTCC GCCTCCTCTT CCTCCATCCG |
| | CCCCGTCTCT CCCCCTTGAA CCTCCTCGTT CGACCCCGCC |
| | TCGATCCTCC CTTTATCCAG CCCTCACTCC TTCTCTAGGC |
| | GCCGGAATTA GATCTCTCGA GGTTATCACA AGTTTGTACA |
| | AAAAAGCAGG CTTCGAAGGA GATAGAACCA ATTCTCTAAG |
| | GAAATACTTA ACCATGGTCG ACTGGATCCG GTACCGAATT |
| | CGCGGCCGCA CTCGAGATAT CTAGACCCAG CTTTCTTGTA |
| | CAAAGTGGTG ATAACGAATT CTACCGGGTA GGTGAGGCGC |
| | TTTTCCCAAG GCAGTCTGGA GCATGCGCTT TAGCAGCCCC |
| | GCTGGGCACT TGGCGCTACA CAAGTGGCCT CTGGCCTCGC |
| | ACACATTCCA CATCCACCGG TAGGCGCCAA CCGGCTCCGT |
| | TCTTTGGTGG CCCCTTCGCG CCACCTTCTA CTCCTCCCCT |
| | AGTCAGGAAG TTCCCCCCCG CCCCGCAGCT CGCGTCGTGC |
| | AGGACGTGAC AAATGGAAGT AGCACGTCTC ACTAGTCTCG |
| | TGCAGATGGA CAGCACCGCT GAGCAATGGA AGCGGGTAGG |
| | CCTTTGGGGC AGCGGCCAAT AGCAGCTTTG CTCCTTCGCT |
| | TTCTGGGCTC AGAGGCTGGG AAGGGGTGGG TCCGGGGGCG |
| | GGCTCAGGGG CGGGCTCAGG GGCGGGGCGG GCGCCCGAAG |
| | GTCCTCCGGA GGCCCGGCAT TCTGCACGCT TCAAAAGCGC |
| | ACGTCTGCCG CGCTGTTCTC CTCTTCCTCA TCTCCGGGCC |
| | TTTCGACCTG CAGCCCAAGC TAGGACCATG GTGAGCAAGG |
| | GCGAGGAGGA TAACATGGCC ATCATCAAGG AGTTCATGCG |
| | CTTCAAGGTG CACATGGAGG GCTCCGTGAA CGGCCACGAG |
| | TTCGAGATCG AGGGCGAGGG CGAGGGCCGC CCCTACGAGG |
| | GCACCCAGAC CGCCAAGCTG AAGGTGACCA AGGGTGGCCC |
| | CCTGCCCTTC GCCTGGGACA TCCTGTCCCC TCAGTTCATG |
| | TACGGCTCCA AGGCCTACGT GAAGCACCCC GCCGACATCC |
| | CCGACTACTT GAAGCTGTCC TTCCCCGAGG GCTTCAAGTG |
| | GGAGCGCGTG ATGAACTTCG AGGACGGCGG CGTGGTGACC |
| | GTGACCCAGG ACTCCTCCCT GCAGGACGGC GAGTTCATCT |
| | ACAAGGTGAA GCTGCGCGGC ACCAACTTCC CCTCCGACGG |
| | CCCCGTAATG CAGAAGAAGA CCATGGGCTG GGAGGCCTCC |
| | TCCGAGCGGA TGTACCCCGA GGACGGCGCC CTGAAGGGCG |
| | AGATCAAGCA GAGGCTGAAG CTGAAGGACG GCGGCCACTA |
| | CGACGCTGAG GTCAAGACCA CCTACAAGGC CAAGAAGCCC |
| | GTGCAGCTGC CCGGCGCCTA CAACGTCAAC ATCAAGTTGG |
| | ACATCACCTC CCACAACGAG GACTACACCA TCGTGGAACA |
| | GTACGAACGC GCCGAGGGCC GCCACTCCAC CGGCGGCATG |
| | GACGAGCTGT ACAAGTGAAT GCATCGATAA AATAAAAGAT |
| | TTTATTTAGT CTCCAGAAAA AGGGGGGAAT GAAAGACCCC |
| | ACCTGTAGGT TTGGCAAGCT AGCTTAAGTA ACGCCATTTT |
| | GCAAGGCATG GAAAATACAT AACTGAGAAT AGAGAAGTTC |
| | AGATCAAGGT TAGGAACAGA GAGACAGCAG AATATGGGCC |
| | AAACAGGATA TCTGTGGTAA GCAGTTCCTG CCCCGGCTCA |
| | GGGCCAAGAA CAGATGGTCC CCAGATGCGG TCCCGCCCTC |
| | AGCAGTTTCT AGAGAACCAT CAGATGTTTC CAGGGTGCCC |
| | CAAGGACCTG AAATGACCCT GTGCCTTATT TGAACTAACC |
| | AATCAGTTCG CTTCTCGCTT CTGTTCGCGC GCTTCTGCTC |
| | CCCGAGCTCA ATAAAAGAGC CCACAACCCC TCACTCGGCG |
| | CGCCAGTCCT CCGATAGACT GCGTCGCCCG GGTACCCGTG |
| | TATCCAATAA ACCCTCTTGC AGTTGCATCC GACTTGTGGT |
| | CTCGCTGTTC CTTGGGAGGG TCTCCTCTGA GTGATTGACT |
| | ACCCGTCAGC GGGGGTCTTT CATGGGTAAC AGTTTCTTGA |
| | AGTTGGAGAA CAACATTCTG AGGGTAGGAG TCGAATATTA |
| | AGTAATCCTG ACTCAATTAG CCACTGTTTT GAATCCACAT |
| | ACTCCAATAC TCCTGAAATA GTTCATTATG GACAGCGCAG |
| | AAGAGCTGGG GAGAATTGTG AAATTGTTAT CCGCTCACAA |
| | TTCCACACAA CATACGAGCC GGAAGCATAA AGTGTAAAGC |
| | CTGGGGTGCC TAATGAGTGA GCTAACTCAC ATTAATTGCG |
| | TTGCGCTCAC TGCCCGCTTT CCAGTCGGGA AACCTGTCGT |
| | GCCAGCTGCA TTAATGAATC GGCCAACGCG CGGGGAGAGG |
| | CGGTTTGCGT ATTGGGCGCT CTTCCGCTTC CTCGCTCACT |
| | GACTCGCTGC GCTCGGTCGT TCGGCTGCGG CGAGCGGTAT |
| | CAGCTCACTC AAAGGCGGTA ATACGGTTAT CCACAGAATC |
| | AGGGGATAAC GCAGGAAAGA ACATGTGAGC AAAAGGCCAG |
| | CAAAAGGCCA GGAACCGTAA AAAGGCCGCG TTGCTGGCGT |
| | TTTTCCATAG GCTCCGCCCC CCTGACGAGC ATCACAAAAA |
| | TCGACGCTCA AGTCAGAGGT GGCGAAACCC GACAGGACTA |
| | TAAAGATACC AGGCGTTTCC CCCTGGAAGC TCCCTCGTGC |
| | GCTCTCCTGT TCCGACCCTG CCGCTTACCG GATACCTGTC |
| | CGCCTTTCTC CCTTCGGGAA GCGTGGCGCT TTCTCATAGC |
| | TCACGCTGTA GGTATCTCAG TTCGGTGTAG GTCGTTCGCT |
| | CCAAGCTGGG CTGTGTGCAC GAACCCCCCG TTCAGCCCGA |
| | CCGCTGCGCC TTATCCGGTA ACTATCGTCT TGAGTCCAAC |
| | CCGGTAAGAC ACGACTTATC GCCACTGGCA GCAGCCACTG |
| | GTAACAGGAT TAGCAGAGCG AGGTATGTAG GCGGTGCTAC |
| | AGAGTTCTTG AAGTGGTGGC CTAACTACGG CTACACTAGA |
| | AGGACAGTAT TTGGTATCTG CGCTCTGCTG AAGCCAGTTA |
| | CCTTCGGAAA AAGAGTTGGT AGCTCTTGAT CCGGCAAACA |
| | AACCACCGCT GGTAGCGGTG GTTTTTTTGT TTGCAAGCAG |
| | CAGATTACGC GCAGAAAAAA AGGATCTCAA GAAGATCCTT |
| | TGATCTTTTC TACGGGGTCT GACGCTCAGT GGAACGAAAA |
| | CTCACGTTAA GGGATTTTGG TCATGAGATT ATCAAAAAGG |
| | ATCTTCACCT AGATCCTTTT AAATTAAAAA TGAAGTTTTA |
| | AATCAATCTA AAGTATATAT GAGTAAACTT GGTCTGACAG |
| | TTACCAATGC TTAATCAGTG AGGCACCTAT CTCAGCGATC |
| | TGTCTATTTC GTTCATCCAT AGTTGCCTGA CTCCCCGTCG |
| | TGTAGATAAC TACGATACGG GAGGGCTTAC CATCTGGCCC |
| | CAGTGCTGCA ATGATACCGC GAGACCCACG CTCACCGGCT |
| | CCAGATTTAT CAGCAATAAA CCAGCCAGCC GGAAGGGCCG |
| | AGCGCAGAAG TGGTCCTGCA ACTTTATCCG CCTCCATCCA |
| | GTCTATTAAT TGTTGCCGGG AAGCTAGAGT AAGTAGTTCG |
| | CCAGTTAATA GTTTGCGCAA CGTTGTTGCC ATTGCTACAG |
| | GCATCGTGGT GTCACGCTCG TCGTTTGGTA TGGCTTCATT |
| | CAGCTCCGGT TCCCAACGAT CAAGGCGAGT TACATGATCC |
| | CCCATGTTGT GCAAAAAAGC GGTTAGCTCC TTCGGTCCTC |
| | CGATCGTTGT CAGAAGTAAG TTGGCCGCAG TGTTATCACT |
| | CATGGTTATG GCAGCACTGC ATAATTCTCT TACTGTCATG |
| | CCATCCGTAA GATGCTTTTC TGTGACTGGT GAGTACTCAA |
| | CCAAGTCATT CTGAGAATAG TGTATGCGGC GACCGAGTTG |
| | CTCTTGCCCG GCGTCAATAC GGGATAATAC CGCGCCACAT |
| | AGCAGAACTT TAAAAGTGCT CATCATTGGA AAACGTTCTT |
| | CGGGGCGAAA ACTCTCAAGG ATCTTACCGC TGTTGAGATC |
| | CAGTTCGATG TAACCCACTC GTGCACCCAA CTGATCTTCA |
| | GCATCTTTTA CTTTCACCAG CGTTTCTGGG TGAGCAAAAA |
| | CAGGAAGGCA AAATGCCGCA AAAAAGGGAA TAAGGGCGAC |
| | ACGGAAATGT TGAATACTCA TACTCTTCCT TTTTCAATAT |
| | TATTGAAGCA TTTATCAGGG TTATTGTCTC ATGAGCGGAT |
| | ACATATTTGA ATGTATTTAG AAAAATAAAC AAATAGGGGT |
| | TCCGCGCACA TTTCCCCGAA AAGTGCCACC TGACGTCTAA |
| | GAAACCATTA TTATCATGAC ATTAACCTAT AAAAATAGGC |
| | GTATCACGAG GCCCTTTCGT CTCGCGCGTT TCGGTGATGA |
| | CGGTGAAAAC CTCTGACACA TGCAGCTCCC GGAGACGGTC |
| | ACAGCTTGTC TGTAAGCGGA TGCCGGGAGC AGACAAGCCC |
| | GTCAGGGCGC GTCAGCGGGT GTTGGCGGGT GTCGGGGCTG |
| | GCTTAACTAT GCGGCATCAG AGCAGATTGT ACTGAGAGTG |
| | CACCATATGC GGTGTGAAAT ACCGCACAGA TGCGTAAGGA |
| | GAAAATACCG CATCAGGCGC CATTCGCCAT TCAGGCTGCG |
| | CAACTGTTGG GAAGGGCGAT CGGTGCGGGC CTCTTCGCTA |
| | TTACGCCAGC TGGCGAAAGG GGGATGTGCT GCAAGGCGAT |
| | TAAGTTGGGT AACGCCAGGG TTTTCCCAGT CACGACGTTG |
| | TAAAACGACG GCGCAAGGAA TGGTGCATGC AAGGAGATGG |
| | CGCCCAACAG TCCCCCGGCC ACGGGGCCTG CCACCATACC |
| | CACGCCGAAA CAAGCGCTCA TGAGCCCGAA GTGGCGAGCC |
| | CGATCTTCCC CATCGGTGAT GTCGGCGATA TAGGCGCCAG |
| | CAACCGCACC TGTGGCGCCG GTGATGCCGG CCACGATGCG |
| | TCCGGCGTAG AGGCGATTAG TCCAATTTGT TAAAGACAGG |
| | ATATCAGTGG TCCAGGCTCT AGTTTTGACT CAACAATATC |
| | ACCAGCTGAA GCCTATAGAG TACGAGCCAT AGATAAAATA |
| | AAAGATTTTA TTTAGTCTCC AGAAAAAGGG GGGAA |
| | (SEQ ID NO: 8) |
| MSCV-HA-CDT- | TGAAAGACCC CACCTGTAGG TTTGGCAAGC TAGCTTAAGT |
| 1-PGK-GFP | AACGCCATTT TGCAAGGCAT GGAAAATACA TAACTGAGAA |
| (FIG. 2, | TAGAGAAGTT CAGATCAAGG TTAGGAACAG AGAGACAGCA |
| above top) | GAATATGGGC CAAACAGGAT ATCTGTGGTA AGCAGTTCCT |
| | GCCCCGGCTC AGGGCCAAGA ACAGATGGTC CCCAGATGCG |
| | GTCCCGCCCT CAGCAGTTTC TAGAGAACCA TCAGATGTTT |
| | CCAGGGTGCC CCAAGGACCT GAAATGACCC TGTGCCTTAT |
| | TTGAACTAAC CAATCAGTTC GCTTCTCGCT TCTGTTCGCG |
| | CGCTTCTGCT CCCCGAGCTC AATAAAAGAG CCCACAACCC |
| | CTCACTCGGC GCGCCAGTCC TCCGATAGAC TGCGTCGCCC |
| | GGGTACCCGT ATTCCCAATA AAGCCTCTTG CTGTTTGCAT |
| | CCGAATCGTG GACTCGCTGA TCCTTGGGAG GGTCTCCTCA |
| | GATTGATTGA CTGCCCACCT CGGGGGTCTT TCATTTGGAG |
| | GTTCCACCGA GATTTGGAGA CCCCTGCCCA GGGACCACCG |
| | ACCCCCCCGC CGGGAGGTAA GCTGGCCAGC GGTCGTTTCG |
| | TGTCTGTCTC TGTCTTTGTG CGTGTTTGTG CCGGCATCTA |
| | ATGTTTGCGC CTGCGTCTGT ACTAGTTAGC TAACTAGCTC |
| | TGTATCTGGC GGACCCGTGG TGGAACTGAC GAGTTCTGAA |
| | CACCCGGCCG CAACCCTGGG AGACGTCCCA GGGACTTTGG |
| | GGGCCGTTTT TGTGGCCCGA CCTGAGGAAG GGAGTCGATG |
| | TGGAATCCGA CCCCGTCAGG ATATGTGGTT CTGGTAGGAG |
| | ACGAGAACCT AAAACAGTTC CCGCCTCCGT CTGAATTTTT |
| | GCTTTCGGTT TGGAACCGAA GCCGCGCGTC TTGTCTGCTG |
| | CAGCGCTGCA GCATCGTTCT GTGTTGTCTC TGTCTGACTG |
| | TGTTTCTGTA TTTGTCTGAA AATTAGGGCC AGACTGTTAC |
| | CACTCCCTTA AGTTTGACCT TAGGTCACTG GAAAGATGTC |
| | GAGCGGATCG CTCACAACCA GTCGGTAGAT GTCAAGAAGA |
| | GACGTTGGGT TACCTTCTGC TCTGCAGAAT GGCCAACCTT |
| | TAACGTCGGA TGGCCGCGAG ACGGCACCTT TAACCGAGAC |
| | CTCATCACCC AGGTTAAGAT CAAGGTCTTT TCACCTGGCC |
| | CGCATGGACA CCCAGACCAG GTCCCCTACA TCGTGACCTG |
| | GGAAGCCTTG GCTTTTGACC CCCCTCCCTG GGTCAAGCCC |
| | TTTGTACACC CTAAGCCTCC GCCTCCTCTT CCTCCATCCG |
| | CCCCGTCTCT CCCCCTTGAA CCTCCTCGTT CGACCCCGCC |
| | TCGATCCTCC CTTTATCCAG CCCTCACTCC TTCTCTAGGC |
| | GCCGGAATTA GCCGCCACCA TGGCGTACCC ATACGATGTT |
| | CCAGATTACG CTTCTTCCCA CGGTTCACAC GACGGAGCTA |
| | GTACTGAAAA GCATCTGGCC ACCCACGACA TAGCCCCCAC |
| | ACATGATGCA ATCAAGATAG TCCCAAAAGG GCATGGACAG |
| | ACTGCAACTA AACCCGGTGC ACAAGAGAAG GAAGTCAGAA |
| | ATGCAGCCCT GTTTGCTGCA ATAAAAGAGT CCAATATAAA |
| | ACCTTGGTCA AAGGAGTCCA TTCACTTGTA TTTCGCCATC |
| | TTTGTAGCCT TCTGTTGTGC CTGCGCTAAT GGGTATGACG |
| | GAAGTCTTAT GACAGGGATA ATTGCAATGG ACAAGTTCCA |
| | GAACCAGTTC CACACTGGAG ACACAGGTCC CAAAGTCAGC |
| | GTTATTTTTT CACTCTACAC CGTAGGTGCT ATGGTAGGGG |
| | CTCCATTTGC AGCAATCCTC AGTGATCGAT TCGGACGAAA |
| | AAAAGGCATG TTCATAGGCG GGATCTTTAT CATAGTGGGC |
| | TCCATCATTG TAGCCTCTTC CTCAAAATTG GCACAATTTG |
| | TGGTCGGTCG CTTCGTTCTC GGGCTGGGTA TAGCCATCAT |
| | GACCGTCGCA GCTCCAGCAT ATTCAATAGA GATCGCCCCA |
| | CCCCATTGGC GGGGTCGCTG CACCGGCTTC TACAACTGCG |
| | GGTGGTTCGG CGGGTCAATC CCAGCCGCTT GTATAACTTA |
| | TGGGTGCTAT TTTATTAAAT CAAATTGGTC ATGGCGAATC |
| | CCACTCATAC TGCAAGCTTT TACCTGTCTT ATTGTCATGA |
| | GCTCAGTCTT CTTCTTGCCA GAATCTCCTC GGTTTTTGTT |
| | CGCCAATGGA AGGGATGCTG AAGCTGTCGC CTTCCTGGTC |
| | AAGTATCACG GAAACGGAGA CCCAAACTCT AAATTGGTTC |
| | TGTTGGAGAC CGAGGAAATG CGAGACGGAA TCCGGACAGA |
| | TGGGGTTGAC AAGGTATGGT GGGATTATAG GCCACTGTTC |
| | ATGACTCACT CCGGGCGCTG GCGCATGGCC CAGGTATTGA |
| | TGATTTCAAT TTTCGGGCAA TTTAGTGGCA ATGGACTTGG |
| | ATACTTCAAT ACTGTCATCT TCAAAAACAT CGGCGTCACT |
| | AGCACCTCAC AGCAGCTCGC CTACAATATA CTCAACAGCG |
| | TTATATCTGC TATTGGTGCA CTCACCGCTG TGTCTATGAC |
| | AGACAGAATG CCCAGGCGCG CAGTTCTCAT AATAGGCACT |
| | TTTATGTGCG CTGCTGCTCT GGCAACTAAC AGTGGGCTCA |
| | GTGCTACTCT TGATAAACAA ACTCAGAGAG GGACCCAGAT |
| | TAACCTTAAC CAAGGGATGA ATGAGCAGGA TGCAAAAGAT |
| | AACGCATACC TTCACGTGGA TTCAAACTAT GCTAAGGGCG |
| | CTCTGGCTGC ATATTTCCTC TTTAATGTAA TTTTTAGCTT |
| | CACATATACC CCTCTTCAAG GTGTCATCCC CACCGAGGCC |
| | CTGGAAACCA CCATTCGGGG GAAGGGTCTC GCTCTGTCAG |
| | GATTCATTGT AAATGCCATG GGCTTTATCA ATCAATTTGC |
| | CGGCCCAATA GCCTTGCACA ATATCGGATA TAAATATATC |
| | TTTGTATTTG TCGGTTGGGA TTTGATAGAA ACAGTTGCAT |
| | GGTACTTTTT CGGAGTTGAA TCCCAGGGCA GAACCTTGGA |
| | ACAACTGGAA TGGGTGTACG ACCAACCTAA TCCAGTGAAA |
| | GCAAGTCTCA AGGTCGAGAA AGTCGTAGTT CAAGCCGACG |
| | GCCATGTGAG TGAAGCCATC GTGGCCTGAT AAGATCTGAA |
| | TTCTACCGGG TAGGGGAGGC GCTTTTCCCA AGGCAGTCTG |
| | GAGCATGCGC TTTAGCAGCC CCGCTGGGCA CTTGGCGCTA |
| | CACAAGTGGC CTCTGGCCTC GCACACATTC CACATCCACC |
| | GGTAGGCGCC AACCGGCTCC GTTCTTTGGT GGCCCCTTCG |
| | CGCCACCTTC TACTCCTCCC CTAGTCAGGA AGTTCCCCCC |
| | CGCCCCGCAG CTCGCGTCGT GCAGGACGTG ACAAATGGAA |
| | GTAGCACGTC TCACTAGTCT CGTGCAGATG GACAGCACCG |
| | CTGAGCAATG GAAGCGGGTA GGCCTTTGGG GCAGCGGCCA |
| | ATAGCAGCTT TGCTCCTTCG CTTTCTGGGC TCAGAGGCTG |
| | GGAAGGGGTG GGTCCGGGGG CGGGCTCAGG GGCGGGCTCA |
| | GGGGCGGGGC GGGCGCCCGA AGGTCCTCCG GAGGCCCGGC |
| | ATTCTGCACG CTTCAAAAGC GCACGTCTGC CGCGCTGTTC |
| | TCCTCTTCCT CATCTCCGGG CCTTTCGACC TGCAGCCCAA |
| | GCTAGGACCA TGGTGAGCAA GGGCGAGGAG CTGTTCACCG |
| | GGGTGGTGCC CATCCTGGTC GAGCTGGACG GCGACGTAAA |
| | CGGCCACAAG TTCAGCGTGT CCGGCGAGGG CGAGGGCGAT |
| | GCCACCTACG GCAAGCTGAC CCTGAAGTTC ATCTGCACCA |
| | CCGGCAAGCT GCCCGTGCCC TGGCCCACCC TCGTGACCAC |
| | CTTCACCTAC GGCGTGCAGT GCTTCAGCCG CTACCCCGAC |
| | CACATGAAGC AGCACGACTT CTTCAAGTCC GCCATGCCCG |
| | AAGGCTACGT CCAGGAGCGC ACCATCTCTT TCAAGGACGA |
| | CGGCAACTAC AAGACCCGCG CCGAGGTGAA GTTCGAGGGC |
| | GACACCCTGG TGAACCGCAT CGAGCTGAAG GGCATCGACT |
| | TCAAGGAGGA CGGCAACATC CTGGGGCACA AGCTGGAGTA |
| | CAACTACAAC AGCCACAACG TCTATATCAC GGCCGACAAG |
| | CAGAAGAACG GCATCAAGGC TAACTTCAAG ATCCGCCACA |
| | ACATCGAGGA CGGCAGCGTG CAGCTCGCCG ACCACTACCA |
| | GCAGAACACC CCCATCGGCG ACGGCCCCGT GCTGCTGCCC |
| | GACAACCACT ACCTGAGCAC CCAGTCCGCC CTGAGCAAAG |
| | ACCCCAACGA GAAGCGCGAT CACATGGTCC TGCTGGAGTT |
| | CGTGACCGCC GCCGGGATCA CTCTCGGCAT GGACGAGCTG |
| | TACAAGTGAA TGCATCGATA AAATAAAAGA TTTTATTTAG |
| | TCTCCAGAAA AAGGGGGGAA TGAAAGACCC CACCTGTAGG |
| | TTTGGCAAGC TAGCTTAAGT AACGCCATTT TGCAAGGCAT |
| | GGAAAATACA TAACTGAGAA TAGAGAAGTT CAGATCAAGG |
| | TTAGGAACAG AGAGACAGCA GAATATGGGC CAAACAGGAT |
| | ATCTGTGGTA AGCAGTTCCT GCCCCGGCTC AGGGCCAAGA |
| | ACAGATGGTC CCCAGATGCG GTCCCGCCCT CAGCAGTTTC |
| | TAGAGAACCA TCAGATGTTT CCAGGGTGCC CCAAGGACCT |
| | GAAATGACCC TGTGCCTTAT TTGAACTAAC CAATCAGTTC |
| | GCTTCTCGCT TCTGTTCGCG CGCTTCTGCT CCCCGAGCTC |
| | AATAAAAGAG CCCACAACCC CTCACTCGGC GCGCCAGTCC |
| | TCCGATAGAC TGCGTCGCCC GGGTACCCGT GTATCCAATA |
| | AACCCTCTTG CAGTTGCATC CGACTTGTGG TCTCGCTGTT |
| | CCTTGGGAGG GTCTCCTCTG AGTGATTGAC TACCCGTCAG |
| | CGGGGGTCTT TCATGGGTAA CAGTTTCTTG AAGTTGGAGA |
| | ACAACATTCT GAGGGTAGGA GTCGAATATT AAGTAATCCT |
| | GACTCAATTA GCCACTGTTT TGAATCCACA TACTCCAATA |
| | CTCCTGAAAT AGTTCATTAT GGACAGCGCA GAAGAGCTGG |
| | GGAGAATTGT GAAATTGTTA TCCGCTCACA ATTCCACACA |
| | ACATACGAGC CGGAAGCATA AAGTGTAAAG CCTGGGGTGC |
| | CTAATGAGTG AGCTAACTCA CATTAATTGC GTTGCGCTCA |
| | CTGCCCGCTT TCCAGTCGGG AAACCTGTCG TGCCAGCTGC |
| | ATTAATGAAT CGGCCAACGC GCGGGGAGAG GCGGTTTGCG |
| | TATTGGGCGC TCTTCCGCTT CCTCGCTCAC TGACTCGCTG |
| | CGCTCGGTCG TTCGGCTGCG GCGAGCGGTA TCAGCTCACT |
| | CAAAGGCGGT AATACGGTTA TCCACAGAAT CAGGGGATAA |
| | CGCAGGAAAG AACATGTGAG CAAAAGGCCA GCAAAAGGCC |
| | AGGAACCGTA AAAAGGCCGC GTTGCTGGCG TTTTTCCATA |
| | GGCTCCGCCC CCCTGACGAG CATCACAAAA ATCGACGCTC |
| | AAGTCAGAGG TGGCGAAACC CGACAGGACT ATAAAGATAC |
| | CAGGCGTTTC CCCCTGGAAG CTCCCTCGTG CGCTCTCCTG |
| | TTCCGACCCT GCCGCTTACC GGATACCTGT CCGCCTTTCT |
| | CCCTTCGGGA AGCGTGGCGC TTTCTCATAG CTCACGCTGT |
| | AGGTATCTCA GTTCGGTGTA GGTCGTTCGC TCCAAGCTGG |
| | GCTGTGTGCA CGAACCCCCC GTTCAGCCCG ACCGCTGCGC |
| | CTTATCCGGT AACTATCGTC TTGAGTCCAA CCCGGTAAGA |
| | CACGACTTAT CGCCACTGGC AGCAGCCACT GGTAACAGGA |
| | TTAGCAGAGC GAGGTATGTA GGCGGTGCTA CAGAGTTCTT |
| | GAAGTGGTGG CCTAACTACG GCTACACTAG AAGGACAGTA |
| | TTTGGTATCT GCGCTCTGCT GAAGCCAGTT ACCTTCGGAA |
| | AAAGAGTTGG TAGCTCTTGA TCCGGCAAAC AAACCACCGC |
| | TGGTAGCGGT GGTTTTTTTG TTTGCAAGCA GCAGATTACG |
| | CGCAGAAAAA AAGGATCTCA AGAAGATCCT TTGATCTTTT |
| | CTACGGGGTC TGACGCTCAG TGGAACGAAA ACTCACGTTA |
| | AGGGATTTTG GTCATGAGAT TATCAAAAAG GATCTTCACC |
| | TAGATCCTTT TAAATTAAAA ATGAAGTTTT AAATCAATCT |
| | AAAGTATATA TGAGTAAACT TGGTCTGACA GTTACCAATG |
| | CTTAATCAGT GAGGCACCTA TCTCAGCGAT CTGTCTATTT |
| | CGTTCATCCA TAGTTGCCTG ACTCCCCGTC GTGTAGATAA |
| | CTACGATACG GGAGGGCTTA CCATCTGGCC CCAGTGCTGC |
| | AATGATACCG CGAGACCCAC GCTCACCGGC TCCAGATTTA |
| | TCAGCAATAA ACCAGCCAGC CGGAAGGGCC GAGCGCAGAA |
| | GTGGTCCTGC AACTTTATCC GCCTCCATCC AGTCTATTAA |
| | TTGTTGCCGG GAAGCTAGAG TAAGTAGTTC GCCAGTTAAT |
| | AGTTTGCGCA ACGTTGTTGC CATTGCTACA GGCATCGTGG |
| | TGTCACGCTC GTCGTTTGGT ATGGCTTCAT TCAGCTCCGG |
| | TTCCCAACGA TCAAGGCGAG TTACATGATC CCCCATGTTG |
| | TGCAAAAAAG CGGTTAGCTC CTTCGGTCCT CCGATCGTTG |
| | TCAGAAGTAA GTTGGCCGCA GTGTTATCAC TCATGGTTAT |
| | GGCAGCACTG CATAATTCTC TTACTGTCAT GCCATCCGTA |
| | AGATGCTTTT CTGTGACTGG TGAGTACTCA ACCAAGTCAT |
| | TCTGAGAATA GTGTATGCGG CGACCGAGTT GCTCTTGCCC |
| | GGCGTCAATA CGGGATAATA CCGCGCCACA TAGCAGAACT |
| | TTAAAAGTGC TCATCATTGG AAAACGTTCT TCGGGGCGAA |
| | AACTCTCAAG GATCTTACCG CTGTTGAGAT CCAGTTCGAT |
| | GTAACCCACT CGTGCACCCA ACTGATCTTC AGCATCTTTT |
| | ACTTTCACCA GCGTTTCTGG GTGAGCAAAA ACAGGAAGGC |
| | AAAATGCCGC AAAAAAGGGA ATAAGGGCGA CACGGAAATG |
| | TTGAATACTC ATACTCTTCC TTTTTCAATA TTATTGAAGC |
| | ATTTATCAGG GTTATTGTCT CATGAGCGGA TACATATTTG |
| | AATGTATTTA GAAAAATAAA |
| | CAAATAGGGG TTCCGCGCAC ATTTCCCCGA AAAGTGCCAC |
| | CTGACGTCTA AGAAACCATT ATTATCATGA CATTAACCTA |
| | TAAAAATAGG CGTATCACGA GGCCCTTTCG TCTCGCGCGT |
| | TTCGGTGATG ACGGTGAAAA CCTCTGACAC ATGCAGCTCC |
| | CGGAGACGGT CACAGCTTGT CTGTAAGCGG ATGCCGGGAG |
| | CAGACAAGCC CGTCAGGGCG CGTCAGCGGG TGTTGGCGGG |
| | TGTCGGGGCT GGCTTAACTA TGCGGCATCA GAGCAGATTG |
| | TACTGAGAGT GCACCATATG CGGTGTGAAA TACCGCACAG |
| | ATGCGTAAGG AGAAAATACC GCATCAGGCG CCATTCGCCA |
| | TTCAGGCTGC GCAACTGTTG GGAAGGGCGA TCGGTGCGGG |
| | CCTCTTCGCT ATTACGCCAG CTGGCGAAAG GGGGATGTGC |
| | TGCAAGGCGA TTAAGTTGGG TAACGCCAGG GTTTTCCCAG |
| | TCACGACGTT GTAAAACGAC GGCGCAAGGA ATGGTGCATG |
| | CAAGGAGATG GCGCCCAACA GTCCCCCGGC CACGGGGCCT |
| | GCCACCATAC CCACGCCGAA ACAAGCGCTC ATGAGCCCGA |
| | AGTGGCGAGC CCGATCTTCC CCATCGGTGA TGTCGGCGAT |
| | ATAGGCGCCA GCAACCGCAC CTGTGGCGCC GGTGATGCCG |
| | GCCACGATGC GTCCGGCGTA GAGGCGATTA GTCCAATTTG |
| | TTAAAGACAG GATATCAGTG GTCCAGGCTC TAGTTTTGAC |
| | TCAACAATAT CACCAGCTGA AGCCTATAGA GTACGAGCCA |
| | TAGATAAAAT AAAAGATTTT ATTTAGTCTC CAGAAAAAGG |
| | GGGGAA |
| | (SEQ ID NO: 9) |
| MSCV-HA-GH1- | TGAAAGACCC CACCTGTAGG TTTGGCAAGC TAGCTTAAGT |
| 1-PGK-GFP | AACGCCATTT TGCAAGGCAT GGAAAATACA TAACTGAGAA |
| (FIG. 2, | TAGAGAAGTT CAGATCAAGG TTAGGAACAG AGAGACAGCA |
| below top) | GAATATGGGC CAAACAGGAT ATCTGTGGTA AGCAGTTCCT |
| | GCCCCGGCTC AGGGCCAAGA ACAGATGGTC CCCAGATGCG |
| | GTCCCGCCCT CAGCAGTTTC TAGAGAACCA TCAGATGTTT |
| | CCAGGGTGCC CCAAGGACCT GAAATGACCC TGTGCCTTAT |
| | TTGAACTAAC CAATCAGTTC GCTTCTCGCT TCTGTTCGCG |
| | CGCTTCTGCT CCCCGAGCTC AATAAAAGAG CCCACAACCC |
| | CTCACTCGGC GCGCCAGTCC TCCGATAGAC TGCGTCGCCC |
| | GGGTACCCGT ATTCCCAATA AAGCCTCTTG CTGTTTGCAT |
| | CCGAATCGTG GACTCGCTGA TCCTTGGGAG GGTCTCCTCA |
| | GATTGATTGA CTGCCCACCT CGGGGGTCTT TCATTTGGAG |
| | GTTCCACCGA GATTTGGAGA CCCCTGCCCA GGGACCACCG |
| | ACCCCCCCGC CGGGAGGTAA GCTGGCCAGC GGTCGTTTCG |
| | TGTCTGTCTC TGTCTTTGTG CGTGTTTGTG CCGGCATCTA |
| | ATGTTTGCGC CTGCGTCTGT ACTAGTTAGC TAACTAGCTC |
| | TGTATCTGGC GGACCCGTGG TGGAACTGAC GAGTTCTGAA |
| | CACCCGGCCG CAACCCTGGG AGACGTCCCA GGGACTTTGG |
| | GGGCCGTTTT TGTGGCCCGA CCTGAGGAAG GGAGTCGATG |
| | TGGAATCCGA CCCCGTCAGG ATATGTGGTT CTGGTAGGAG |
| | ACGAGAACCT AAAACAGTTC CCGCCTCCGT CTGAATTTTT |
| | GCTTTCGGTT TGGAACCGAA GCCGCGCGTC TTGTCTGCTG |
| | CAGCGCTGCA GCATCGTTCT GTGTTGTCTC TGTCTGACTG |
| | TGTTTCTGTA TTTGTCTGAA AATTAGGGCC AGACTGTTAC |
| | CACTCCCTTA AGTTTGACCT TAGGTCACTG GAAAGATGTC |
| | GAGCGGATCG CTCACAACCA GTCGGTAGAT GTCAAGAAGA |
| | GACGTTGGGT TACCTTCTGC TCTGCAGAAT GGCCAACCTT |
| | TAACGTCGGA TGGCCGCGAG ACGGCACCTT TAACCGAGAC |
| | CTCATCACCC AGGTTAAGAT CAAGGTCTTT TCACCTGGCC |
| | CGCATGGACA CCCAGACCAG GTCCCCTACA TCGTGACCTG |
| | GGAAGCCTTG GCTTTTGACC CCCCTCCCTG GGTCAAGCCC |
| | TTTGTACACC CTAAGCCTCC GCCTCCTCTT CCTCCATCCG |
| | CCCCGTCTCT CCCCCTTGAA CCTCCTCGTT CGACCCCGCC |
| | TCGATCCTCC CTTTATCCAG CCCTCACTCC TTCTCTAGGC |
| | GCCGGAATTA GCCGCCACCA TGGCGTACCC ATACGATGTT |
| | CCAGATTACG CTTCCTTGCC CAAGGATTTT CTGTGGGGGT |
| | TTGCCACAGC TGCCTATCAA ATTGAGGGCG CTATTCACGC |
| | AGATGGAAGA GGACCATCCA TTTGGGACAC ATTTTGCAAC |
| | ATCCCTGGCA AGATAGCAGA CGGATCTAGC GGTGCCGTGG |
| | CTTGCGACTC ATACAACAGA ACTAAAGAGG ATATTGACCT |
| | CCTGAAGAGC TTGGGCGCAA CAGCATACAG GTTTAGTATT |
| | TCATGGAGCA GAATCATCCC AGTAGGAGGC AGAAACGACC |
| | CTATTAACCA GAAGGGTATA GATCACTACG TTAAGTTTGT |
| | GGATGATCTG CTTGAGGCAG GTATCACCCC ATTTATTACC |
| | CTCTTTCATT GGGATTTGCC TGATGGTCTC GATAAGCGCT |
| | ATGGCGGGCT CTTGAATCGG GAGGAGTTCC CTCTGGACTT |
| | CGAGCATTAC GCTAGGACTA TGTTCAAGGC TATACCAAAA |
| | TGTAAGCATT GGATCACTTT CAACGAACCC TGGTGCTCCT |
| | CAATCCTCGG ATACAACTCA GGATATTTTG CTCCAGGACA |
| | CACTTCTGAC AGAACAAAAA GTCCAGTAGG CGATAGCGCC |
| | CGCGAGCCCT GGATAGTTGG CCATAATCTG TTGATCGCAC |
| | ATGGGCGAGC TGTCAAAGTT TATCGGGAAG ATTTCAAGCC |
| | TACACAGGGA GGCGAAATTG GCATCACCCT GAACGGGGAC |
| | GCCACCCTGC CCTGGGACCC AGAGGACCCT CTCGATGTCG |
| | AGGCCTGCGA TCGCAAGATA GAGTTTGCAA TTTCATGGTT |
| | TGCTGATCCC ATTTATTTTG GAAAGTACCC TGACTCCATG |
| | AGAAAGCAGC TGGGTGACAG GCTTCCAGAG TTCACACCTG |
| | AAGAAGTTGC TCTTGTCAAG GGATCCAACG ATTTCTACGG |
| | TATGAATCAT TATACAGCTA ACTATATCAA ACATAAAAAA |
| | GGTGTTCCAC CCGAGGACGA TTTTTTGGGT AATCTCGAAA |
| | CCTTGTTTTA TAACAAAAAG GGAAACTGTA TAGGCCCAGA |
| | GACCCAGAGT TTCTGGCTCC GACCCCATGC TCAAGGGTTC |
| | CGCGACCTCC TGAATTGGTT GTCCAAGCGA TACGGCTATC |
| | CTAAGATTTA TGTGACAGAG AACGGTACTT CATTGAAGGG |
| | CGAGAATGCA ATGCCTTTGA AGCAAATTGT AGAAGATGAT |
| | TTCCGCGTTA AGTACTTTAA TGACTATGTA AATGCTATGG |
| | CTAAGGCACA CTCCGAAGAT GGAGTTAATG TCAAAGGATA |
| | CCTCGCTTGG TCTCTTATGG ATAATTTCGA GTGGGCAGAA |
| | GGCTATGAGA CTAGATTCGG TGTGACATAT GTGGATTACG |
| | AGAACGATCA GAAGCGCTAT CCCAAGAAAT CAGCCAAATC |
| | CCTCAAACCA TTGTTTGATT CATTGATTAA GAAAGACTGA |
| | TAAGATCTGA ATTCTACCGG GTAGGTGAGG CGCTTTTCCC |
| | AAGGCAGTCT GGAGCATGCG CTTTAGCAGC CCCGCTGGGC |
| | ACTTGGCGCT ACACAAGTGG CCTCTGGCCT CGCACACATT |
| | CCACATCCAC CGGTAGGCGC CAACCGGCTC CGTTCTTTGG |
| | TGGCCCCTTC GCGCCACCTT CTACTCCTCC CCTAGTCAGG |
| | AAGTTCCCCC CCGCCCCGCA GCTCGCGTCG TGCAGGACGT |
| | GACAAATGGA AGTAGCACGT CTCACTAGTC TCGTGCAGAT |
| | GGACAGCACC GCTGAGCAAT GGAAGCGGGT AGGCCTTTGG |
| | GGCAGCGGCC AATAGCAGCT TTGCTCCTTC GCTTTCTGGG |
| | CTCAGAGGCT GGGAAGGGGT GGGTCCGGGG GCGGGCTCAG |
| | GGGCGGGCTC AGGGGCGGGG CGGGCGCCCG AAGGTCCTCC |
| | GGAGGCCCGG CATTCTGCAC GCTTCAAAAG CGCACGTCTG |
| | CCGCGCTGTT CTCCTCTTCC TCATCTCCGG GCCTTTCGAC |
| | CTGCAGCCCA AGCTAGGACC ATGGTGAGCA AGGGCGAGGA |
| | GCTGTTCACC GGGGTGGTGC CCATCCTGGT CGAGCTGGAC |
| | GGCGACGTAA ACGGCCACAA GTTCAGCGTG TCCGGCGAGG |
| | GCGAGGGCGA TGCCACCTAC GGCAAGCTGA CCCTGAAGTT |
| | CATCTGCACC ACCGGCAAGC TGCCCGTGCC CTGGCCCACC |
| | CTCGTGACCA CCTTCACCTA CGGCGTGCAG TGCTTCAGCC |
| | GCTACCCCGA CCACATGAAG CAGCACGACT TCTTCAAGTC |
| | CGCCATGCCC GAAGGCTACG TCCAGGAGCG CACCATCTCT |
| | TTCAAGGACG ACGGCAACTA CAAGACCCGC GCCGAGGTGA |
| | AGTTCGAGGG CGACACCCTG GTGAACCGCA TCGAGCTGAA |
| | GGGCATCGAC TTCAAGGAGG ACGGCAACAT CCTGGGGCAC |
| | AAGCTGGAGT ACAACTACAA CAGCCACAAC GTCTATATCA |
| | CGGCCGACAA GCAGAAGAAC GGCATCAAGG CTAACTTCAA |
| | GATCCGCCAC AACATCGAGG ACGGCAGCGT GCAGCTCGCC |
| | GACCACTACC AGCAGAACAC CCCCATCGGC GACGGCCCCG |
| | TGCTGCTGCC CGACAACCAC TACCTGAGCA CCCAGTCCGC |
| | CCTGAGCAAA GACCCCAACG AGAAGCGCGA TCACATGGTC |
| | CTGCTGGAGT TCGTGACCGC CGCCGGGATC ACTCTCGGCA |
| | TGGACGAGCT GTACAAGTGA ATGCATCGAT AAAATAAAAG |
| | ATTTTATTTA GTCTCCAGAA AAAGGGGGGA ATGAAAGACC |
| | CCACCTGTAG GTTTGGCAAG CTAGCTTAAG TAACGCCATT |
| | TTGCAAGGCA TGGAAAATAC ATAACTGAGA ATAGAGAAGT |
| | TCAGATCAAG GTTAGGAACA GAGAGACAGC AGAATATGGG |
| | CCAAACAGGA TATCTGTGGT AAGCAGTTCC TGCCCCGGCT |
| | CAGGGCCAAG AACAGATGGT CCCCAGATGC GGTCCCGCCC |
| | TCAGCAGTTT CTAGAGAACC ATCAGATGTT TCCAGGGTGC |
| | CCCAAGGACC TGAAATGACC CTGTGCCTTA TTTGAACTAA |
| | CCAATCAGTT CGCTTCTCGC TTCTGTTCGC GCGCTTCTGC |
| | TCCCCGAGCT CAATAAAAGA GCCCACAACC CCTCACTCGG |
| | CGCGCCAGTC CTCCGATAGA CTGCGTCGCC CGGGTACCCG |
| | TGTATCCAAT AAACCCTCTT GCAGTTGCAT CCGACTTGTG |
| | GTCTCGCTGT TCCTTGGGAG GGTCTCCTCT GAGTGATTGA |
| | CTACCCGTCA GCGGGGGTCT TTCATGGGTA ACAGTTTCTT |
| | GAAGTTGGAG AACAACATTC TGAGGGTAGG AGTCGAATAT |
| | TAAGTAATCC TGACTCAATT AGCCACTGTT TTGAATCCAC |
| | ATACTCCAAT ACTCCTGAAA TAGTTCATTA TGGACAGCGC |
| | AGAAGAGCTG GGGAGAATTG TGAAATTGTT ATCCGCTCAC |
| | AATTCCACAC AACATACGAG CCGGAAGCAT AAAGTGTAAA |
| | GCCTGGGGTG CCTAATGAGT GAGCTAACTC ACATTAATTG |
| | CGTTGCGCTC ACTGCCCGCT TTCCAGTCGG GAAACCTGTC |
| | GTGCCAGCTG CATTAATGAA TCGGCCAACG CGCGGGGAGA |
| | GGCGGTTTGC GTATTGGGCG CTCTTCCGCT TCCTCGCTCA |
| | CTGACTCGCT GCGCTCGGTC GTTCGGCTGC GGCGAGCGGT |
| | ATCAGCTCAC TCAAAGGCGG TAATACGGTT ATCCACAGAA |
| | TCAGGGGATA ACGCAGGAAA GAACATGTGA GCAAAAGGCC |
| | AGCAAAAGGC CAGGAACCGT AAAAAGGCCG CGTTGCTGGC |
| | GTTTTTCCAT AGGCTCCGCC CCCCTGACGA GCATCACAAA |
| | AATCGACGCT CAAGTCAGAG GTGGCGAAAC CCGACAGGAC |
| | TATAAAGATA CCAGGCGTTT CCCCCTGGAA GCTCCCTCGT |
| | GCGCTCTCCT GTTCCGACCC TGCCGCTTAC CGGATACCTG |
| | TCCGCCTTTC TCCCTTCGGG AAGCGTGGCG CTTTCTCATA |
| | GCTCACGCTG TAGGTATCTC AGTTCGGTGT AGGTCGTTCG |
| | CTCCAAGCTG GGCTGTGTGC ACGAACCCCC CGTTCAGCCC |
| | GACCGCTGCG CCTTATCCGG TAACTATCGT CTTGAGTCCA |
| | ACCCGGTAAG ACACGACTTA TCGCCACTGG CAGCAGCCAC |
| | TGGTAACAGG ATTAGCAGAG CGAGGTATGT AGGCGGTGCT |
| | ACAGAGTTCT TGAAGTGGTG GCCTAACTAC GGCTACACTA |
| | GAAGGACAGT ATTTGGTATC TGCGCTCTGC TGAAGCCAGT |
| | TACCTTCGGA AAAAGAGTTG GTAGCTCTTG ATCCGGCAAA |
| | CAAACCACCG CTGGTAGCGG TGGTTTTTTT GTTTGCAAGC |
| | AGCAGATTAC GCGCAGAAAA AAAGGATCTC AAGAAGATCC |
| | TTTGATCTTT TCTACGGGGT CTGACGCTCA GTGGAACGAA |
| | AACTCACGTT AAGGGATTTT GGTCATGAGA TTATCAAAAA |
| | GGATCTTCAC CTAGATCCTT TTAAATTAAA AATGAAGTTT |
| | TAAATCAATC TAAAGTATAT ATGAGTAAAC TTGGTCTGAC |
| | AGTTACCAAT GCTTAATCAG TGAGGCACCT ATCTCAGCGA |
| | TCTGTCTATT TCGTTCATCC ATAGTTGCCT GACTCCCCGT |
| | CGTGTAGATA ACTACGATAC GGGAGGGCTT ACCATCTGGC |
| | CCCAGTGCTG CAATGATACC GCGAGACCCA CGCTCACCGG |
| | CTCCAGATTT ATCAGCAATA AACCAGCCAG CCGGAAGGGC |
| | CGAGCGCAGA AGTGGTCCTG CAACTTTATC CGCCTCCATC |
| | CAGTCTATTA ATTGTTGCCG GGAAGCTAGA GTAAGTAGTT |
| | CGCCAGTTAA TAGTTTGCGC AACGTTGTTG CCATTGCTAC |
| | AGGCATCGTG GTGTCACGCT CGTCGTTTGG TATGGCTTCA |
| | TTCAGCTCCG GTTCCCAACG ATCAAGGCGA GTTACATGAT |
| | CCCCCATGTT GTGCAAAAAA GCGGTTAGCT CCTTCGGTCC |
| | TCCGATCGTT GTCAGAAGTA AGTTGGCCGC AGTGTTATCA |
| | CTCATGGTTA TGGCAGCACT GCATAATTCT CTTACTGTCA |
| | TGCCATCCGT AAGATGCTTT TCTGTGACTG GTGAGTACTC |
| | AACCAAGTCA TTCTGAGAAT AGTGTATGCG GCGACCGAGT |
| | TGCTCTTGCC CGGCGTCAAT ACGGGATAAT ACCGCGCCAC |
| | ATAGCAGAAC TTTAAAAGTG CTCATCATTG GAAAACGTTC |
| | TTCGGGGCGA AAACTCTCAA GGATCTTACC GCTGTTGAGA |
| | TCCAGTTCGA TGTAACCCAC TCGTGCACCC AACTGATCTT |
| | CAGCATCTTT TACTTTCACC AGCGTTTCTG GGTGAGCAAA |
| | AACAGGAAGG CAAAATGCCG CAAAAAAGGG |
| | AATAAGGGCG ACACGGAAAT GTTGAATACT CATACTCTTC |
| | CTTTTTCAAT ATTATTGAAG CATTTATCAG GGTTATTGTC |
| | TCATGAGCGG ATACATATTT GAATGTATTT AGAAAAATAA |
| | ACAAATAGGG GTTCCGCGCA CATTTCCCCG AAAAGTGCCA |
| | CCTGACGTCT AAGAAACCAT TATTATCATG ACATTAACCT |
| | ATAAAAATAG GCGTATCACG AGGCCCTTTC GTCTCGCGCG |
| | TTTCGGTGAT GACGGTGAAA ACCTCTGACA CATGCAGCTC |
| | CCGGAGACGG TCACAGCTTG TCTGTAAGCG GATGCCGGGA |
| | GCAGACAAGC CCGTCAGGGC GCGTCAGCGG GTGTTGGCGG |
| | GTGTCGGGGC TGGCTTAACT ATGCGGCATC AGAGCAGATT |
| | GTACTGAGAG TGCACCATAT GCGGTGTGAA ATACCGCACA |
| | GATGCGTAAG GAGAAAATAC CGCATCAGGC GCCATTCGCC |
| | ATTCAGGCTG CGCAACTGTT GGGAAGGGCG ATCGGTGCGG |
| | GCCTCTTCGC TATTACGCCA GCTGGCGAAA GGGGGATGTG |
| | CTGCAAGGCG ATTAAGTTGG GTAACGCCAG GGTTTTCCCA |
| | GTCACGACGT TGTAAAACGA CGGCGCAAGG AATGGTGCAT |
| | GCAAGGAGAT GGCGCCCAAC AGTCCCCCGG CCACGGGGCC |
| | TGCCACCATA CCCACGCCGA AACAAGCGCT CATGAGCCCG |
| | AAGTGGCGAG CCCGATCTTC CCCATCGGTG ATGTCGGCGA |
| | TATAGGCGCC AGCAACCGCA CCTGTGGCGC CGGTGATGCC |
| | GGCCACGATG CGTCCGGCGT AGAGGCGATT AGTCCAATTT |
| | GTTAAAGACA GGATATCAGT GGTCCAGGCT CTAGTTTTGA |
| | CTCAACAATA TCACCAGCTG AAGCCTATAG AGTACGAGCC |
| | ATAGATAAAA TAAAAGATTT TATTTAGTCT CCAGAAAAAG |
| | GGGGGAA |
| | (SEQ ID NO: 10) |
| MSCV-HA-CDT- | TGAAAGACCC CACCTGTAGG TTTGGCAAGC TAGCTTAAGT |
| 1-PGK-mCherry | AACGCCATTT TGCAAGGCAT GGAAAATACA TAACTGAGAA |
| (FIG. 2, | TAGAGAAGTT CAGATCAAGG TTAGGAACAG AGAGACAGCA |
| bottom) | GAATATGGGC CAAACAGGAT ATCTGTGGTA AGCAGTTCCT |
| | GCCCCGGCTC AGGGCCAAGA ACAGATGGTC CCCAGATGCG |
| | GTCCCGCCCT CAGCAGTTTC TAGAGAACCA TCAGATGTTT |
| | CCAGGGTGCC CCAAGGACCT GAAATGACCC TGTGCCTTAT |
| | TTGAACTAAC CAATCAGTTC GCTTCTCGCT TCTGTTCGCG |
| | CGCTTCTGCT CCCCGAGCTC AATAAAAGAG CCCACAACCC |
| | CTCACTCGGC GCGCCAGTCC TCCGATAGAC TGCGTCGCCC |
| | GGGTACCCGT ATTCCCAATA AAGCCTCTTG CTGTTTGCAT |
| | CCGAATCGTG GACTCGCTGA TCCTTGGGAG GGTCTCCTCA |
| | GATTGATTGA CTGCCCACCT CGGGGGTCTT TCATTTGGAG |
| | GTTCCACCGA GATTTGGAGA CCCCTGCCCA GGGACCACCG |
| | ACCCCCCCGC CGGGAGGTAA GCTGGCCAGC GGTCGTTTCG |
| | TGTCTGTCTC TGTCTTTGTG CGTGTTTGTG CCGGCATCTA |
| | ATGTTTGCGC CTGCGTCTGT ACTAGTTAGC TAACTAGCTC |
| | TGTATCTGGC GGACCCGTGG TGGAACTGAC GAGTTCTGAA |
| | CACCCGGCCG CAACCCTGGG AGACGTCCCA GGGACTTTGG |
| | GGGCCGTTTT TGTGGCCCGA CCTGAGGAAG GGAGTCGATG |
| | TGGAATCCGA CCCCGTCAGG ATATGTGGTT CTGGTAGGAG |
| | ACGAGAACCT AAAACAGTTC CCGCCTCCGT CTGAATTTTT |
| | GCTTTCGGTT TGGAACCGAA GCCGCGCGTC TTGTCTGCTG |
| | CAGCGCTGCA GCATCGTTCT GTGTTGTCTC TGTCTGACTG |
| | TGTTTCTGTA TTTGTCTGAA AATTAGGGCC AGACTGTTAC |
| | CACTCCCTTA AGTTTGACCT TAGGTCACTG GAAAGATGTC |
| | GAGCGGATCG CTCACAACCA GTCGGTAGAT GTCAAGAAGA |
| | GACGTTGGGT TACCTTCTGC TCTGCAGAAT GGCCAACCTT |
| | TAACGTCGGA TGGCCGCGAG ACGGCACCTT TAACCGAGAC |
| | CTCATCACCC AGGTTAAGAT CAAGGTCTTT TCACCTGGCC |
| | CGCATGGACA CCCAGACCAG GTCCCCTACA TCGTGACCTG |
| | GGAAGCCTTG GCTTTTGACC CCCCTCCCTG GGTCAAGCCC |
| | TTTGTACACC CTAAGCCTCC GCCTCCTCTT CCTCCATCCG |
| | CCCCGTCTCT CCCCCTTGAA CCTCCTCGTT CGACCCCGCC |
| | TCGATCCTCC CTTTATCCAG CCCTCACTCC TTCTCTAGGC |
| | GCCGGAATTA GCCGCCACCA TGGCGTACCC ATACGATGTT |
| | CCAGATTACG CTTCTTCCCA CGGTTCACAC GACGGAGCTA |
| | GTACTGAAAA GCATCTGGCC ACCCACGACA TAGCCCCCAC |
| | ACATGATGCA ATCAAGATAG TCCCAAAAGG GCATGGACAG |
| | ACTGCAACTA AACCCGGTGC ACAAGAGAAG GAAGTCAGAA |
| | ATGCAGCCCT GTTTGCTGCA ATAAAAGAGT CCAATATAAA |
| | ACCTTGGTCA AAGGAGTCCA TTCACTTGTA TTTCGCCATC |
| | TTTGTAGCCT TCTGTTGTGC CTGCGCTAAT GGGTATGACG |
| | GAAGTCTTAT GACAGGGATA ATTGCAATGG ACAAGTTCCA |
| | GAACCAGTTC CACACTGGAG ACACAGGTCC CAAAGTCAGC |
| | GTTATTTTTT CACTCTACAC CGTAGGTGCT ATGGTAGGGG |
| | CTCCATTTGC AGCAATCCTC AGTGATCGAT TCGGACGAAA |
| | AAAAGGCATG TTCATAGGCG GGATCTTTAT CATAGTGGGC |
| | TCCATCATTG TAGCCTCTTC CTCAAAATTG GCACAATTTG |
| | TGGTCGGTCG CTTCGTTCTC GGGCTGGGTA TAGCCATCAT |
| | GACCGTCGCA GCTCCAGCAT ATTCAATAGA GATCGCCCCA |
| | CCCCATTGGC GGGGTCGCTG CACCGGCTTC TACAACTGCG |
| | GGTGGTTCGG CGGGTCAATC CCAGCCGCTT GTATAACTTA |
| | TGGGTGCTAT TTTATTAAAT CAAATTGGTC ATGGCGAATC |
| | CCACTCATAC TGCAAGCTTT TACCTGTCTT ATTGTCATGA |
| | GCTCAGTCTT CTTCTTGCCA GAATCTCCTC GGTTTTTGTT |
| | CGCCAATGGA AGGGATGCTG AAGCTGTCGC CTTCCTGGTC |
| | AAGTATCACG GAAACGGAGA CCCAAACTCT AAATTGGTTC |
| | TGTTGGAGAC CGAGGAAATG CGAGACGGAA TCCGGACAGA |
| | TGGGGTTGAC AAGGTATGGT GGGATTATAG GCCACTGTTC |
| | ATGACTCACT CCGGGCGCTG GCGCATGGCC CAGGTATTGA |
| | TGATTTCAAT TTTCGGGCAA TTTAGTGGCA ATGGACTTGG |
| | ATACTTCAAT ACTGTCATCT TCAAAAACAT CGGCGTCACT |
| | AGCACCTCAC AGCAGCTCGC CTACAATATA CTCAACAGCG |
| | TTATATCTGC TATTGGTGCA CTCACCGCTG TGTCTATGAC |
| | AGACAGAATG CCCAGGCGCG CAGTTCTCAT AATAGGCACT |
| | TTTATGTGCG CTGCTGCTCT GGCAACTAAC AGTGGGCTCA |
| | GTGCTACTCT TGATAAACAA ACTCAGAGAG GGACCCAGAT |
| | TAACCTTAAC CAAGGGATGA ATGAGCAGGA TGCAAAAGAT |
| | AACGCATACC TTCACGTGGA TTCAAACTAT GCTAAGGGCG |
| | CTCTGGCTGC ATATTTCCTC TTTAATGTAA TTTTTAGCTT |
| | CACATATACC CCTCTTCAAG GTGTCATCCC CACCGAGGCC |
| | CTGGAAACCA CCATTCGGGG GAAGGGTCTC GCTCTGTCAG |
| | GATTCATTGT AAATGCCATG GGCTTTATCA ATCAATTTGC |
| | CGGCCCAATA GCCTTGCACA ATATCGGATA TAAATATATC |
| | TTTGTATTTG TCGGTTGGGA TTTGATAGAA ACAGTTGCAT |
| | GGTACTTTTT CGGAGTTGAA TCCCAGGGCA GAACCTTGGA |
| | ACAACTGGAA TGGGTGTACG ACCAACCTAA TCCAGTGAAA |
| | GCAAGTCTCA AGGTCGAGAA AGTCGTAGTT CAAGCCGACG |
| | GCCATGTGAG TGAAGCCATC GTGGCCTGAT AAGATCTGAA |
| | TTCTACCGGG TAGGGGAGGC GCTTTTCCCA AGGCAGTCTG |
| | GAGCATGCGC TTTAGCAGCC CCGCTGGGCA CTTGGCGCTA |
| | CACAAGTGGC CTCTGGCCTC GCACACATTC CACATCCACC |
| | GGTAGGCGCC AACCGGCTCC GTTCTTTGGT GGCCCCTTCG |
| | CGCCACCTTC TACTCCTCCC CTAGTCAGGA AGTTCCCCCC |
| | CGCCCCGCAG CTCGCGTCGT GCAGGACGTG ACAAATGGAA |
| | GTAGCACGTC TCACTAGTCT CGTGCAGATG GACAGCACCG |
| | CTGAGCAATG GAAGCGGGTA GGCCTTTGGG GCAGCGGCCA |
| | ATAGCAGCTT TGCTCCTTCG CTTTCTGGGC TCAGAGGCTG |
| | GGAAGGGGTG GGTCCGGGGG CGGGCTCAGG GGCGGGCTCA |
| | GGGGCGGGGC GGGCGCCCGA AGGTCCTCCG GAGGCCCGGC |
| | ATTCTGCACG CTTCAAAAGC GCACGTCTGC CGCGCTGTTC |
| | TCCTCTTCCT CATCTCCGGG CCTTTCGACC TGCAGCCCAA |
| | GCTAGGACCA TGGTGAGCAA GGGCGAGGAG GATAACATGG |
| | CCATCATCAA GGAGTTCATG CGCTTCAAGG TGCACATGGA |
| | GGGCTCCGTG AACGGCCACG AGTTCGAGAT CGAGGGCGAG |
| | GGCGAGGGCC GCCCCTACGA GGGCACCCAG ACCGCCAAGC |
| | TGAAGGTGAC CAAGGGTGGC CCCCTGCCCT TCGCCTGGGA |
| | CATCCTGTCC CCTCAGTTCA TGTACGGCTC CAAGGCCTAC |
| | GTGAAGCACC CCGCCGACAT CCCCGACTAC TTGAAGCTGT |
| | CCTTCCCCGA GGGCTTCAAG TGGGAGCGCG TGATGAACTT |
| | CGAGGACGGC GGCGTGGTGA CCGTGACCCA GGACTCCTCC |
| | CTGCAGGACG GCGAGTTCAT CTACAAGGTG AAGCTGCGCG |
| | GCACCAACTT CCCCTCCGAC GGCCCCGTAA TGCAGAAGAA |
| | GACCATGGGC TGGGAGGCCT CCTCCGAGCG GATGTACCCC |
| | GAGGACGGCG CCCTGAAGGG CGAGATCAAG CAGAGGCTGA |
| | AGCTGAAGGA CGGCGGCCAC TACGACGCTG AGGTCAAGAC |
| | CACCTACAAG GCCAAGAAGC CCGTGCAGCT GCCCGGCGCC |
| | TACAACGTCA ACATCAAGTT GGACATCACC TCCCACAACG |
| | AGGACTACAC CATCGTGGAA CAGTACGAAC GCGCCGAGGG |
| | CCGCCACTCC ACCGGCGGCA TGGACGAGCT GTACAAGTGA |
| | ATGCATCGAT AAAATAAAAG ATTTTATTTA GTCTCCAGAA |
| | AAAGGGGGGA ATGAAAGACC CCACCTGTAG GTTTGGCAAG |
| | CTAGCTTAAG TAACGCCATT TTGCAAGGCA TGGAAAATAC |
| | ATAACTGAGA ATAGAGAAGT TCAGATCAAG GTTAGGAACA |
| | GAGAGACAGC AGAATATGGG CCAAACAGGA TATCTGTGGT |
| | AAGCAGTTCC TGCCCCGGCT CAGGGCCAAG AACAGATGGT |
| | CCCCAGATGC GGTCCCGCCC TCAGCAGTTT CTAGAGAACC |
| | ATCAGATGTT TCCAGGGTGC CCCAAGGACC TGAAATGACC |
| | CTGTGCCTTA TTTGAACTAA CCAATCAGTT CGCTTCTCGC |
| | TTCTGTTCGC GCGCTTCTGC TCCCCGAGCT CAATAAAAGA |
| | GCCCACAACC CCTCACTCGG CGCGCCAGTC CTCCGATAGA |
| | CTGCGTCGCC CGGGTACCCG TGTATCCAAT AAACCCTCTT |
| | GCAGTTGCAT CCGACTTGTG GTCTCGCTGT TCCTTGGGAG |
| | GGTCTCCTCT GAGTGATTGA CTACCCGTCA GCGGGGGTCT |
| | TTCATGGGTA ACAGTTTCTT GAAGTTGGAG AACAACATTC |
| | TGAGGGTAGG AGTCGAATAT TAAGTAATCC TGACTCAATT |
| | AGCCACTGTT TTGAATCCAC ATACTCCAAT ACTCCTGAAA |
| | TAGTTCATTA TGGACAGCGC AGAAGAGCTG GGGAGAATTG |
| | TGAAATTGTT ATCCGCTCAC AATTCCACAC AACATACGAG |
| | CCGGAAGCAT AAAGTGTAAA GCCTGGGGTG CCTAATGAGT |
| | GAGCTAACTC ACATTAATTG CGTTGCGCTC ACTGCCCGCT |
| | TTCCAGTCGG GAAACCTGTC GTGCCAGCTG CATTAATGAA |
| | TCGGCCAACG CGCGGGGAGA GGCGGTTTGC GTATTGGGCG |
| | CTCTTCCGCT TCCTCGCTCA CTGACTCGCT GCGCTCGGTC |
| | GTTCGGCTGC GGCGAGCGGT ATCAGCTCAC TCAAAGGCGG |
| | TAATACGGTT ATCCACAGAA TCAGGGGATA ACGCAGGAAA |
| | GAACATGTGA GCAAAAGGCC AGCAAAAGGC CAGGAACCGT |
| | AAAAAGGCCG CGTTGCTGGC GTTTTTCCAT AGGCTCCGCC |
| | CCCCTGACGA GCATCACAAA AATCGACGCT CAAGTCAGAG |
| | GTGGCGAAAC CCGACAGGAC TATAAAGATA CCAGGCGTTT |
| | CCCCCTGGAA GCTCCCTCGT GCGCTCTCCT GTTCCGACCC |
| | TGCCGCTTAC CGGATACCTG TCCGCCTTTC TCCCTTCGGG |
| | AAGCGTGGCG CTTTCTCATA GCTCACGCTG TAGGTATCTC |
| | AGTTCGGTGT AGGTCGTTCG CTCCAAGCTG GGCTGTGTGC |
| | ACGAACCCCC CGTTCAGCCC GACCGCTGCG CCTTATCCGG |
| | TAACTATCGT CTTGAGTCCA ACCCGGTAAG ACACGACTTA |
| | TCGCCACTGG CAGCAGCCAC TGGTAACAGG ATTAGCAGAG |
| | CGAGGTATGT AGGCGGTGCT ACAGAGTTCT TGAAGTGGTG |
| | GCCTAACTAC GGCTACACTA GAAGGACAGT ATTTGGTATC |
| | TGCGCTCTGC TGAAGCCAGT TACCTTCGGA AAAAGAGTTG |
| | GTAGCTCTTG ATCCGGCAAA CAAACCACCG CTGGTAGCGG |
| | TGGTTTTTTT GTTTGCAAGC AGCAGATTAC GCGCAGAAAA |
| | AAAGGATCTC AAGAAGATCC TTTGATCTTT TCTACGGGGT |
| | CTGACGCTCA GTGGAACGAA AACTCACGTT AAGGGATTTT |
| | GGTCATGAGA TTATCAAAAA GGATCTTCAC CTAGATCCTT |
| | TTAAATTAAA AATGAAGTTT TAAATCAATC TAAAGTATAT |
| | ATGAGTAAAC TTGGTCTGAC AGTTACCAAT GCTTAATCAG |
| | TGAGGCACCT ATCTCAGCGA TCTGTCTATT TCGTTCATCC |
| | ATAGTTGCCT GACTCCCCGT CGTGTAGATA ACTACGATAC |
| | GGGAGGGCTT ACCATCTGGC CCCAGTGCTG CAATGATACC |
| | GCGAGACCCA CGCTCACCGG CTCCAGATTT ATCAGCAATA |
| | AACCAGCCAG CCGGAAGGGC CGAGCGCAGA AGTGGTCCTG |
| | CAACTTTATC CGCCTCCATC CAGTCTATTA ATTGTTGCCG |
| | GGAAGCTAGA GTAAGTAGTT CGCCAGTTAA TAGTTTGCGC |
| | AACGTTGTTG CCATTGCTAC AGGCATCGTG GTGTCACGCT |
| | CGTCGTTTGG TATGGCTTCA TTCAGCTCCG GTTCCCAACG |
| | ATCAAGGCGA GTTACATGAT CCCCCATGTT GTGCAAAAAA |
| | GCGGTTAGCT CCTTCGGTCC TCCGATCGTT GTCAGAAGTA |
| | AGTTGGCCGC AGTGTTATCA CTCATGGTTA TGGCAGCACT |
| | GCATAATTCT CTTACTGTCA TGCCATCCGT AAGATGCTTT |
| | TCTGTGACTG GTGAGTACTC AACCAAGTCA TTCTGAGAAT |
| | AGTGTATGCG GCGACCGAGT TGCTCTTGCC CGGCGTCAAT |
| | ACGGGATAAT ACCGCGCCAC ATAGCAGAAC TTTAAAAGTG |
| | CTCATCATTG GAAAACGTTC TTCGGGGCGA AAACTCTCAA |
| | GGATCTTACC GCTGTTGAGA TCCAGTTCGA TGTAACCCAC |
| | TCGTGCACCC AACTGATCTT CAGCATCTTT TACTTTCACC |
| | AGCGTTTCTG GGTGAGCAAA AACAGGAAGG CAAAATGCCG |
| | CAAAAAAGGG AATAAGGGCG ACACGGAAAT GTTGAATACT |
| | CATACTCTTC CTTTTTCAAT ATTATTGAAG CATTTATCAG |
| | GGTTATTGTC TCATGAGCGG ATACATATTT GAATGTATTT |
| | AGAAAAATAA ACAAATAGGG GTTCCGCGCA CATTTCCCCG |
| | AAAAGTGCCA CCTGACGTCT AAGAAACCAT TATTATCATG |
| | ACATTAACCT ATAAAAATAG GCGTATCACG AGGCCCTTTC |
| | GTCTCGCGCG TTTCGGTGAT GACGGTGAAA ACCTCTGACA |
| | CATGCAGCTC CCGGAGACGG TCACAGCTTG TCTGTAAGCG |
| | GATGCCGGGA GCAGACAAGC CCGTCAGGGC GCGTCAGCGG |
| | GTGTTGGCGG GTGTCGGGGC TGGCTTAACT ATGCGGCATC |
| | AGAGCAGATT GTACTGAGAG TGCACCATAT GCGGTGTGAA |
| | ATACCGCACA GATGCGTAAG GAGAAAATAC CGCATCAGGC |
| | GCCATTCGCC ATTCAGGCTG CGCAACTGTT GGGAAGGGCG |
| | ATCGGTGCGG GCCTCTTCGC TATTACGCCA GCTGGCGAAA |
| | GGGGGATGTG CTGCAAGGCG ATTAAGTTGG GTAACGCCAG |
| | GGTTTTCCCA GTCACGACGT TGTAAAACGA CGGCGCAAGG |
| | AATGGTGCAT GCAAGGAGAT GGCGCCCAAC AGTCCCCCGG |
| | CCACGGGGCC TGCCACCATA CCCACGCCGA AACAAGCGCT |
| | CATGAGCCCG AAGTGGCGAG CCCGATCTTC CCCATCGGTG |
| | ATGTCGGCGA TATAGGCGCC AGCAACCGCA CCTGTGGCGC |
| | CGGTGATGCC GGCCACGATG CGTCCGGCGT AGAGGCGATT |
| | AGTCCAATTT GTTAAAGACA GGATATCAGT GGTCCAGGCT |
| | CTAGTTTTGA CTCAACAATA TCACCAGCTG AAGCCTATAG |
| | AGTACGAGCC ATAGATAAAA TAAAAGATTT TATTTAGTCT |
| | CCAGAAAAAG GGGGGAA |
| | (SEQ ID NO: 11) |
| MSCV-CDT-1- | TGAAAGACCC CACCTGTAGG TTTGGCAAGC TAGCTTAAGT |
| HA-IDT™-v1- | AACGCCATTT TGCAAGGCAT GGAAAATACA TAACTGAGAA |
| PGK-mCherry | TAGAGAAGTT CAGATCAAGG TTAGGAACAG AGAGACAGCA |
| (FIG. 4, top & | GAATATGGGC CAAACAGGAT ATCTGTGGTA AGCAGTTCCT |
| FIG. 16A, | GCCCCGGCTC AGGGCCAAGA ACAGATGGTC CCCAGATGCG |
| vector 1) | GTCCCGCCCT CAGCAGTTTC TAGAGAACCA TCAGATGTTT |
| | CCAGGGTGCC CCAAGGACCT GAAATGACCC TGTGCCTTAT |
| | TTGAACTAAC CAATCAGTTC GCTTCTCGCT TCTGTTCGCG |
| | CGCTTCTGCT CCCCGAGCTC AATAAAAGAG CCCACAACCC |
| | CTCACTCGGC GCGCCAGTCC TCCGATAGAC TGCGTCGCCC |
| | GGGTACCCGT ATTCCCAATA AAGCCTCTTG CTGTTTGCAT |
| | CCGAATCGTG GACTCGCTGA TCCTTGGGAG GGTCTCCTCA |
| | GATTGATTGA CTGCCCACCT CGGGGGTCTT TCATTTGGAG |
| | GTTCCACCGA GATTTGGAGA CCCCTGCCCA GGGACCACCG |
| | ACCCCCCCGC CGGGAGGTAA GCTGGCCAGC GGTCGTTTCG |
| | TGTCTGTCTC TGTCTTTGTG CGTGTTTGTG CCGGCATCTA |
| | ATGTTTGCGC CTGCGTCTGT ACTAGTTAGC TAACTAGCTC |
| | TGTATCTGGC GGACCCGTGG TGGAACTGAC GAGTTCTGAA |
| | CACCCGGCCG CAACCCTGGG AGACGTCCCA GGGACTTTGG |
| | GGGCCGTTTT TGTGGCCCGA CCTGAGGAAG GGAGTCGATG |
| | TGGAATCCGA CCCCGTCAGG ATATGTGGTT CTGGTAGGAG |
| | ACGAGAACCT AAAACAGTTC CCGCCTCCGT CTGAATTTTT |
| | GCTTTCGGTT TGGAACCGAA GCCGCGCGTC TTGTCTGCTG |
| | CAGCGCTGCA GCATCGTTCT GTGTTGTCTC TGTCTGACTG |
| | TGTTTCTGTA TTTGTCTGAA AATTAGGGCC AGACTGTTAC |
| | CACTCCCTTA AGTTTGACCT TAGGTCACTG GAAAGATGTC |
| | GAGCGGATCG CTCACAACCA GTCGGTAGAT GTCAAGAAGA |
| | GACGTTGGGT TACCTTCTGC TCTGCAGAAT GGCCAACCTT |
| | TAACGTCGGA TGGCCGCGAG ACGGCACCTT TAACCGAGAC |
| | CTCATCACCC AGGTTAAGAT CAAGGTCTTT TCACCTGGCC |
| | CGCATGGACA CCCAGACCAG GTCCCCTACA TCGTGACCTG |
| | GGAAGCCTTG GCTTTTGACC CCCCTCCCTG GGTCAAGCCC |
| | TTTGTACACC CTAAGCCTCC GCCTCCTCTT CCTCCATCCG |
| | CCCCGTCTCT CCCCCTTGAA CCTCCTCGTT CGACCCCGCC |
| | TCGATCCTCC CTTTATCCAG CCCTCACTCC TTCTCTAGGC |
| | GCCGGAATTA GCCGCCACCA TGTCTTCCCA CGGTTCACAC |
| | GACGGAGCTA GTACTGAAAA GCATCTGGCC ACCCACGACA |
| | TAGCCCCCAC ACATGATGCA ATCAAGATAG TCCCAAAAGG |
| | GCATGGACAG ACTGCAACTA AACCCGGTGC ACAAGAGAAG |
| | GAAGTCAGAA ATGCAGCCCT GTTTGCTGCA ATAAAAGAGT |
| | CCAATATAAA ACCTTGGTCA AAGGAGTCCA TTCACTTGTA |
| | TTTCGCCATC TTTGTAGCCT TCTGTTGTGC CTGCGCTAAT |
| | GGGTATGACG GAAGTCTTAT GACAGGGATA ATTGCAATGG |
| | ACAAGTTCCA GAACCAGTTC CACACTGGAG ACACAGGTCC |
| | CAAAGTCAGC GTTATTTTTT CACTCTACAC CGTAGGTGCT |
| | ATGGTAGGGG CTCCATTTGC AGCAATCCTC AGTGATCGAT |
| | TCGGACGAAA AAAAGGCATG TTCATAGGCG GGATCTTTAT |
| | CATAGTGGGC TCCATCATTG TAGCCTCTTC CTCAAAATTG |
| | GCACAATTTG TGGTCGGTCG CTTCGTTCTC GGGCTGGGTA |
| | TAGCCATCAT GACCGTCGCA GCTCCAGCAT ATTCAATAGA |
| | GATCGCCCCA CCCCATTGGC GGGGTCGCTG CACCGGCTTC |
| | TACAACTGCG GGTGGTTCGG CGGGTCAATC CCAGCCGCTT |
| | GTATAACTTA TGGGTGCTAT TTTATTAAAT CAAATTGGTC |
| | ATGGCGAATC CCACTCATAC TGCAAGCTTT TACCTGTCTT |
| | ATTGTCATGA GCTCAGTCTT CTTCTTGCCA GAATCTCCTC |
| | GGTTTTTGTT CGCCAATGGA AGGGATGCTG AAGCTGTCGC |
| | CTTCCTGGTC AAGTATCACG GAAACGGAGA CCCAAACTCT |
| | AAATTGGTTC TGTTGGAGAC CGAGGAAATG CGAGACGGAA |
| | TCCGGACAGA TGGGGTTGAC AAGGTATGGT GGGATTATAG |
| | GCCACTGTTC ATGACTCACT CCGGGCGCTG GCGCATGGCC |
| | CAGGTATTGA TGATTTCAAT TTTCGGGCAA TTTAGTGGCA |
| | ATGGACTTGG ATACTTCAAT ACTGTCATCT TCAAAAACAT |
| | CGGCGTCACT AGCACCTCAC AGCAGCTCGC CTACAATATA |
| | CTCAACAGCG TTATATCTGC TATTGGTGCA CTCACCGCTG |
| | TGTCTATGAC AGACAGAATG CCCAGGCGCG CAGTTCTCAT |
| | AATAGGCACT TTTATGTGCG CTGCTGCTCT GGCAACTAAC |
| | AGTGGGCTCA GTGCTACTCT TGATAAACAA ACTCAGAGAG |
| | GGACCCAGAT TAACCTTAAC CAAGGGATGA ATGAGCAGGA |
| | TGCAAAAGAT AACGCATACC TTCACGTGGA TTCAAACTAT |
| | GCTAAGGGCG CTCTGGCTGC ATATTTCCTC TTTAATGTAA |
| | TTTTTAGCTT CACATATACC CCTCTTCAAG GTGTCATCCC |
| | CACCGAGGCC CTGGAAACCA CCATTCGGGG GAAGGGTCTC |
| | GCTCTGTCAG GATTCATTGT AAATGCCATG GGCTTTATCA |
| | ATCAATTTGC CGGCCCAATA GCCTTGCACA ATATCGGATA |
| | TAAATATATC TTTGTATTTG TCGGTTGGGA TTTGATAGAA |
| | ACAGTTGCAT GGTACTTTTT CGGAGTTGAA TCCCAGGGCA |
| | GAACCTTGGA ACAACTGGAA TGGGTGTACG ACCAACCTAA |
| | TCCAGTGAAA GCAAGTCTCA AGGTCGAGAA AGTCGTAGTT |
| | CAAGCCGACG GCCATGTGAG TGAAGCCATC GTGGCCTACC |
| | CATACGATGT TCCAGATTAC GCTTGAAATT CTACCGGGTA |
| | GGTGAGGCGC TTTTCCCAAG GCAGTCTGGA GCATGCGCTT |
| | TAGCAGCCCC GCTGGGCACT TGGCGCTACA CAAGTGGCCT |
| | CTGGCCTCGC ACACATTCCA CATCCACCGG TAGGCGCCAA |
| | CCGGCTCCGT TCTTTGGTGG CCCCTTCGCG CCACCTTCTA |
| | CTCCTCCCCT AGTCAGGAAG TTCCCCCCCG CCCCGCAGCT |
| | CGCGTCGTGC AGGACGTGAC AAATGGAAGT AGCACGTCTC |
| | ACTAGTCTCG TGCAGATGGA CAGCACCGCT GAGCAATGGA |
| | AGCGGGTAGG CCTTTGGGGC AGCGGCCAAT AGCAGCTTTG |
| | CTCCTTCGCT TTCTGGGCTC AGAGGCTGGG AAGGGGTGGG |
| | TCCGGGGGCG GGCTCAGGGG CGGGCTCAGG GGCGGGGCGG |
| | GCGCCCGAAG GTCCTCCGGA GGCCCGGCAT TCTGCACGCT |
| | TCAAAAGCGC ACGTCTGCCG CGCTGTTCTC CTCTTCCTCA |
| | TCTCCGGGCC TTTCGACCTG CAGCCCAAGC TAGGACCATG |
| | GTGAGCAAGG GCGAGGAGGA TAACATGGCC ATCATCAAGG |
| | AGTTCATGCG CTTCAAGGTG CACATGGAGG GCTCCGTGAA |
| | CGGCCACGAG TTCGAGATCG AGGGCGAGGG CGAGGGCCGC |
| | CCCTACGAGG GCACCCAGAC CGCCAAGCTG AAGGTGACCA |
| | AGGGTGGCCC CCTGCCCTTC GCCTGGGACA TCCTGTCCCC |
| | TCAGTTCATG TACGGCTCCA AGGCCTACGT GAAGCACCCC |
| | GCCGACATCC CCGACTACTT GAAGCTGTCC TTCCCCGAGG |
| | GCTTCAAGTG GGAGCGCGTG ATGAACTTCG AGGACGGCGG |
| | CGTGGTGACC GTGACCCAGG ACTCCTCCCT GCAGGACGGC |
| | GAGTTCATCT ACAAGGTGAA GCTGCGCGGC ACCAACTTCC |
| | CCTCCGACGG CCCCGTAATG CAGAAGAAGA CCATGGGCTG |
| | GGAGGCCTCC TCCGAGCGGA TGTACCCCGA GGACGGCGCC |
| | CTGAAGGGCG AGATCAAGCA GAGGCTGAAG CTGAAGGACG |
| | GCGGCCACTA CGACGCTGAG GTCAAGACCA CCTACAAGGC |
| | CAAGAAGCCC GTGCAGCTGC CCGGCGCCTA CAACGTCAAC |
| | ATCAAGTTGG ACATCACCTC CCACAACGAG GACTACACCA |
| | TCGTGGAACA GTACGAACGC GCCGAGGGCC GCCACTCCAC |
| | CGGCGGCATG GACGAGCTGT ACAAGTGAAT GCATCGATAA |
| | AATAAAAGAT TTTATTTAGT CTCCAGAAAA AGGGGGGAAT |
| | GAAAGACCCC ACCTGTAGGT TTGGCAAGCT AGCTTAAGTA |
| | ACGCCATTTT GCAAGGCATG GAAAATACAT AACTGAGAAT |
| | AGAGAAGTTC AGATCAAGGT TAGGAACAGA GAGACAGCAG |
| | AATATGGGCC AAACAGGATA TCTGTGGTAA GCAGTTCCTG |
| | CCCCGGCTCA GGGCCAAGAA CAGATGGTCC CCAGATGCGG |
| | TCCCGCCCTC AGCAGTTTCT AGAGAACCAT CAGATGTTTC |
| | CAGGGTGCCC CAAGGACCTG AAATGACCCT GTGCCTTATT |
| | TGAACTAACC AATCAGTTCG CTTCTCGCTT CTGTTCGCGC |
| | GCTTCTGCTC CCCGAGCTCA ATAAAAGAGC CCACAACCCC |
| | TCACTCGGCG CGCCAGTCCT CCGATAGACT GCGTCGCCCG |
| | GGTACCCGTG TATCCAATAA ACCCTCTTGC AGTTGCATCC |
| | GACTTGTGGT CTCGCTGTTC CTTGGGAGGG TCTCCTCTGA |
| | GTGATTGACT ACCCGTCAGC GGGGGTCTTT CATGGGTAAC |
| | AGTTTCTTGA AGTTGGAGAA CAACATTCTG AGGGTAGGAG |
| | TCGAATATTA AGTAATCCTG ACTCAATTAG CCACTGTTTT |
| | GAATCCACAT ACTCCAATAC TCCTGAAATA GTTCATTATG |
| | GACAGCGCAG AAGAGCTGGG GAGAATTGTG AAATTGTTAT |
| | CCGCTCACAA TTCCACACAA CATACGAGCC GGAAGCATAA |
| | AGTGTAAAGC CTGGGGTGCC TAATGAGTGA GCTAACTCAC |
| | ATTAATTGCG TTGCGCTCAC TGCCCGCTTT CCAGTCGGGA |
| | AACCTGTCGT GCCAGCTGCA TTAATGAATC GGCCAACGCG |
| | CGGGGAGAGG CGGTTTGCGT ATTGGGCGCT CTTCCGCTTC |
| | CTCGCTCACT GACTCGCTGC GCTCGGTCGT TCGGCTGCGG |
| | CGAGCGGTAT CAGCTCACTC AAAGGCGGTA ATACGGTTAT |
| | CCACAGAATC AGGGGATAAC GCAGGAAAGA ACATGTGAGC |
| | AAAAGGCCAG CAAAAGGCCA GGAACCGTAA AAAGGCCGCG |
| | TTGCTGGCGT TTTTCCATAG GCTCCGCCCC CCTGACGAGC |
| | ATCACAAAAA TCGACGCTCA AGTCAGAGGT GGCGAAACCC |
| | GACAGGACTA TAAAGATACC AGGCGTTTCC CCCTGGAAGC |
| | TCCCTCGTGC GCTCTCCTGT TCCGACCCTG CCGCTTACCG |
| | GATACCTGTC CGCCTTTCTC CCTTCGGGAA GCGTGGCGCT |
| | TTCTCATAGC TCACGCTGTA GGTATCTCAG TTCGGTGTAG |
| | GTCGTTCGCT CCAAGCTGGG CTGTGTGCAC GAACCCCCCG |
| | TTCAGCCCGA CCGCTGCGCC TTATCCGGTA ACTATCGTCT |
| | TGAGTCCAAC CCGGTAAGAC ACGACTTATC GCCACTGGCA |
| | GCAGCCACTG GTAACAGGAT TAGCAGAGCG AGGTATGTAG |
| | GCGGTGCTAC AGAGTTCTTG AAGTGGTGGC CTAACTACGG |
| | CTACACTAGA AGGACAGTAT TTGGTATCTG CGCTCTGCTG |
| | AAGCCAGTTA CCTTCGGAAA AAGAGTTGGT AGCTCTTGAT |
| | CCGGCAAACA AACCACCGCT GGTAGCGGTG GTTTTTTTGT |
| | TTGCAAGCAG CAGATTACGC GCAGAAAAAA AGGATCTCAA |
| | GAAGATCCTT TGATCTTTTC TACGGGGTCT GACGCTCAGT |
| | GGAACGAAAA CTCACGTTAA GGGATTTTGG TCATGAGATT |
| | ATCAAAAAGG ATCTTCACCT AGATCCTTTT AAATTAAAAA |
| | TGAAGTTTTA AATCAATCTA AAGTATATAT GAGTAAACTT |
| | GGTCTGACAG TTACCAATGC TTAATCAGTG AGGCACCTAT |
| | CTCAGCGATC TGTCTATTTC GTTCATCCAT AGTTGCCTGA |
| | CTCCCCGTCG TGTAGATAAC TACGATACGG GAGGGCTTAC |
| | CATCTGGCCC CAGTGCTGCA ATGATACCGC GAGACCCACG |
| | CTCACCGGCT CCAGATTTAT CAGCAATAAA CCAGCCAGCC |
| | GGAAGGGCCG AGCGCAGAAG TGGTCCTGCA ACTTTATCCG |
| | CCTCCATCCA GTCTATTAAT TGTTGCCGGG AAGCTAGAGT |
| | AAGTAGTTCG CCAGTTAATA GTTTGCGCAA CGTTGTTGCC |
| | ATTGCTACAG GCATCGTGGT GTCACGCTCG TCGTTTGGTA |
| | TGGCTTCATT CAGCTCCGGT TCCCAACGAT CAAGGCGAGT |
| | TACATGATCC CCCATGTTGT GCAAAAAAGC GGTTAGCTCC |
| | TTCGGTCCTC CGATCGTTGT CAGAAGTAAG TTGGCCGCAG |
| | TGTTATCACT CATGGTTATG GCAGCACTGC ATAATTCTCT |
| | TACTGTCATG CCATCCGTAA GATGCTTTTC TGTGACTGGT |
| | GAGTACTCAA CCAAGTCATT CTGAGAATAG TGTATGCGGC |
| | GACCGAGTTG CTCTTGCCCG GCGTCAATAC GGGATAATAC |
| | CGCGCCACAT AGCAGAACTT TAAAAGTGCT CATCATTGGA |
| | AAACGTTCTT CGGGGCGAAA ACTCTCAAGG ATCTTACCGC |
| | TGTTGAGATC CAGTTCGATG TAACCCACTC GTGCACCCAA |
| | CTGATCTTCA GCATCTTTTA CTTTCACCAG CGTTTCTGGG |
| | TGAGCAAAAA CAGGAAGGCA AAATGCCGCA |
| | AAAAAGGGAA TAAGGGCGAC ACGGAAATGT TGAATACTCA |
| | TACTCTTCCT TTTTCAATAT TATTGAAGCA TTTATCAGGG |
| | TTATTGTCTC ATGAGCGGAT ACATATTTGA ATGTATTTAG |
| | AAAAATAAAC AAATAGGGGT TCCGCGCACA TTTCCCCGAA |
| | AAGTGCCACC TGACGTCTAA GAAACCATTA TTATCATGAC |
| | ATTAACCTAT AAAAATAGGC GTATCACGAG GCCCTTTCGT |
| | CTCGCGCGTT TCGGTGATGA CGGTGAAAAC CTCTGACACA |
| | TGCAGCTCCC GGAGACGGTC ACAGCTTGTC TGTAAGCGGA |
| | TGCCGGGAGC AGACAAGCCC GTCAGGGCGC GTCAGCGGGT |
| | GTTGGCGGGT GTCGGGGCTG GCTTAACTAT GCGGCATCAG |
| | AGCAGATTGT ACTGAGAGTG CACCATATGC GGTGTGAAAT |
| | ACCGCACAGA TGCGTAAGGA GAAAATACCG CATCAGGCGC |
| | CATTCGCCAT TCAGGCTGCG CAACTGTTGG GAAGGGCGAT |
| | CGGTGCGGGC CTCTTCGCTA TTACGCCAGC TGGCGAAAGG |
| | GGGATGTGCT GCAAGGCGAT TAAGTTGGGT AACGCCAGGG |
| | TTTTCCCAGT CACGACGTTG TAAAACGACG GCGCAAGGAA |
| | TGGTGCATGC AAGGAGATGG CGCCCAACAG TCCCCCGGCC |
| | ACGGGGCCTG CCACCATACC CACGCCGAAA CAAGCGCTCA |
| | TGAGCCCGAA GTGGCGAGCC CGATCTTCCC CATCGGTGAT |
| | GTCGGCGATA TAGGCGCCAG CAACCGCACC TGTGGCGCCG |
| | GTGATGCCGG CCACGATGCG TCCGGCGTAG AGGCGATTAG |
| | TCCAATTTGT TAAAGACAGG ATATCAGTGG TCCAGGCTCT |
| | AGTTTTGACT CAACAATATC ACCAGCTGAA GCCTATAGAG |
| | TACGAGCCAT AGATAAAATA AAAGATTTTA TTTAGTCTCC |
| | AGAAAAAGGG GGGAA |
| | (SEQ ID NO: 12) |
| MSCV-CDT-1- | TGAAAGACCC CACCTGTAGG TTTGGCAAGC TAGCTTAAGT |
| HA-ERES-PGK- | AACGCCATTT TGCAAGGCAT GGAAAATACA TAACTGAGAA |
| mCherry | TAGAGAAGTT CAGATCAAGG TTAGGAACAG AGAGACAGCA |
| (FIG. 4, | GAATATGGGC CAAACAGGAT ATCTGTGGTA AGCAGTTCCT |
| bottom) | GCCCCGGCTC AGGGCCAAGA ACAGATGGTC CCCAGATGCG |
| | GTCCCGCCCT CAGCAGTTTC TAGAGAACCA TCAGATGTTT |
| | CCAGGGTGCC CCAAGGACCT GAAATGACCC TGTGCCTTAT |
| | TTGAACTAAC CAATCAGTTC GCTTCTCGCT TCTGTTCGCG |
| | CGCTTCTGCT CCCCGAGCTC AATAAAAGAG CCCACAACCC |
| | CTCACTCGGC GCGCCAGTCC TCCGATAGAC TGCGTCGCCC |
| | GGGTACCCGT ATTCCCAATA AAGCCTCTTG CTGTTTGCAT |
| | CCGAATCGTG GACTCGCTGA TCCTTGGGAG GGTCTCCTCA |
| | GATTGATTGA CTGCCCACCT CGGGGGTCTT TCATTTGGAG |
| | GTTCCACCGA GATTTGGAGA CCCCTGCCCA GGGACCACCG |
| | ACCCCCCCGC CGGGAGGTAA GCTGGCCAGC GGTCGTTTCG |
| | TGTCTGTCTC TGTCTTTGTG CGTGTTTGTG CCGGCATCTA |
| | ATGTTTGCGC CTGCGTCTGT ACTAGTTAGC TAACTAGCTC |
| | TGTATCTGGC GGACCCGTGG TGGAACTGAC GAGTTCTGAA |
| | CACCCGGCCG CAACCCTGGG AGACGTCCCA GGGACTTTGG |
| | GGGCCGTTTT TGTGGCCCGA CCTGAGGAAG GGAGTCGATG |
| | TGGAATCCGA CCCCGTCAGG ATATGTGGTT CTGGTAGGAG |
| | ACGAGAACCT AAAACAGTTC CCGCCTCCGT CTGAATTTTT |
| | GCTTTCGGTT TGGAACCGAA GCCGCGCGTC TTGTCTGCTG |
| | CAGCGCTGCA GCATCGTTCT GTGTTGTCTC TGTCTGACTG |
| | TGTTTCTGTA TTTGTCTGAA AATTAGGGCC AGACTGTTAC |
| | CACTCCCTTA AGTTTGACCT TAGGTCACTG GAAAGATGTC |
| | GAGCGGATCG CTCACAACCA GTCGGTAGAT GTCAAGAAGA |
| | GACGTTGGGT TACCTTCTGC TCTGCAGAAT GGCCAACCTT |
| | TAACGTCGGA TGGCCGCGAG ACGGCACCTT TAACCGAGAC |
| | CTCATCACCC AGGTTAAGAT CAAGGTCTTT TCACCTGGCC |
| | CGCATGGACA CCCAGACCAG GTCCCCTACA TCGTGACCTG |
| | GGAAGCCTTG GCTTTTGACC CCCCTCCCTG GGTCAAGCCC |
| | TTTGTACACC CTAAGCCTCC GCCTCCTCTT CCTCCATCCG |
| | CCCCGTCTCT CCCCCTTGAA CCTCCTCGTT CGACCCCGCC |
| | TCGATCCTCC CTTTATCCAG CCCTCACTCC TTCTCTAGGC |
| | GCCGGAATTA GCCGCCACCA TGTCTTCCCA CGGTTCACAC |
| | GACGGAGCTA GTACTGAAAA GCATCTGGCC ACCCACGACA |
| | TAGCCCCCAC ACATGATGCA ATCAAGATAG TCCCAAAAGG |
| | GCATGGACAG ACTGCAACTA AACCCGGTGC ACAAGAGAAG |
| | GAAGTCAGAA ATGCAGCCCT GTTTGCTGCA ATAAAAGAGT |
| | CCAATATAAA ACCTTGGTCA AAGGAGTCCA TTCACTTGTA |
| | TTTCGCCATC TTTGTAGCCT TCTGTTGTGC CTGCGCTAAT |
| | GGGTATGACG GAAGTCTTAT GACAGGGATA ATTGCAATGG |
| | ACAAGTTCCA GAACCAGTTC CACACTGGAG ACACAGGTCC |
| | CAAAGTCAGC GTTATTTTTT CACTCTACAC CGTAGGTGCT |
| | ATGGTAGGGG CTCCATTTGC AGCAATCCTC AGTGATCGAT |
| | TCGGACGAAA AAAAGGCATG TTCATAGGCG GGATCTTTAT |
| | CATAGTGGGC TCCATCATTG TAGCCTCTTC CTCAAAATTG |
| | GCACAATTTG TGGTCGGTCG CTTCGTTCTC GGGCTGGGTA |
| | TAGCCATCAT GACCGTCGCA GCTCCAGCAT ATTCAATAGA |
| | GATCGCCCCA CCCCATTGGC GGGGTCGCTG CACCGGCTTC |
| | TACAACTGCG GGTGGTTCGG CGGGTCAATC CCAGCCGCTT |
| | GTATAACTTA TGGGTGCTAT TTTATTAAAT CAAATTGGTC |
| | ATGGCGAATC CCACTCATAC TGCAAGCTTT TACCTGTCTT |
| | ATTGTCATGA GCTCAGTCTT CTTCTTGCCA GAATCTCCTC |
| | GGTTTTTGTT CGCCAATGGA AGGGATGCTG AAGCTGTCGC |
| | CTTCCTGGTC AAGTATCACG GAAACGGAGA CCCAAACTCT |
| | AAATTGGTTC TGTTGGAGAC CGAGGAAATG CGAGACGGAA |
| | TCCGGACAGA TGGGGTTGAC AAGGTATGGT GGGATTATAG |
| | GCCACTGTTC ATGACTCACT CCGGGCGCTG GCGCATGGCC |
| | CAGGTATTGA TGATTTCAAT TTTCGGGCAA TTTAGTGGCA |
| | ATGGACTTGG ATACTTCAAT ACTGTCATCT TCAAAAACAT |
| | CGGCGTCACT AGCACCTCAC AGCAGCTCGC CTACAATATA |
| | CTCAACAGCG TTATATCTGC TATTGGTGCA CTCACCGCTG |
| | TGTCTATGAC AGACAGAATG CCCAGGCGCG CAGTTCTCAT |
| | AATAGGCACT TTTATGTGCG CTGCTGCTCT GGCAACTAAC |
| | AGTGGGCTCA GTGCTACTCT TGATAAACAA ACTCAGAGAG |
| | GGACCCAGAT TAACCTTAAC CAAGGGATGA ATGAGCAGGA |
| | TGCAAAAGAT AACGCATACC TTCACGTGGA TTCAAACTAT |
| | GCTAAGGGCG CTCTGGCTGC ATATTTCCTC TTTAATGTAA |
| | TTTTTAGCTT CACATATACC CCTCTTCAAG GTGTCATCCC |
| | CACCGAGGCC CTGGAAACCA CCATTCGGGG GAAGGGTCTC |
| | GCTCTGTCAG GATTCATTGT AAATGCCATG GGCTTTATCA |
| | ATCAATTTGC CGGCCCAATA GCCTTGCACA ATATCGGATA |
| | TAAATATATC TTTGTATTTG TCGGTTGGGA TTTGATAGAA |
| | ACAGTTGCAT GGTACTTTTT CGGAGTTGAA TCCCAGGGCA |
| | GAACCTTGGA ACAACTGGAA TGGGTGTACG ACCAACCTAA |
| | TCCAGTGAAA GCAAGTCTCA AGGTCGAGAA AGTCGTAGTT |
| | CAAGCCGACG GCCATGTGAG TGAAGCCATC GTGGCCTACC |
| | CATACGATGT TCCAGATTAC GCTTTTTGCT ATGAAAATGA |
| | ATGAAATTCT ACCGGGTAGG TGAGGCGCTT TTCCCAAGGC |
| | AGTCTGGAGC ATGCGCTTTA GCAGCCCCGC TGGGCACTTG |
| | GCGCTACACA AGTGGCCTCT GGCCTCGCAC ACATTCCACA |
| | TCCACCGGTA GGCGCCAACC GGCTCCGTTC TTTGGTGGCC |
| | CCTTCGCGCC ACCTTCTACT CCTCCCCTAG TCAGGAAGTT |
| | CCCCCCCGCC CCGCAGCTCG CGTCGTGCAG GACGTGACAA |
| | ATGGAAGTAG CACGTCTCAC TAGTCTCGTG CAGATGGACA |
| | GCACCGCTGA GCAATGGAAG CGGGTAGGCC TTTGGGGCAG |
| | CGGCCAATAG CAGCTTTGCT CCTTCGCTTT CTGGGCTCAG |
| | AGGCTGGGAA GGGGTGGGTC CGGGGGCGGG CTCAGGGGCG |
| | GGCTCAGGGG CGGGGGGGGC GCCCGAAGGT CCTCCGGAGG |
| | CCCGGCATTC TGCACGCTTC AAAAGCGCAC GTCTGCCGCG |
| | CTGTTCTCCT CTTCCTCATC TCCGGGCCTT TCGACCTGCA |
| | GCCCAAGCTA GGACCATGGT GAGCAAGGGC GAGGAGGATA |
| | ACATGGCCAT CATCAAGGAG TTCATGCGCT TCAAGGTGCA |
| | CATGGAGGGC TCCGTGAACG GCCACGAGTT CGAGATCGAG |
| | GGCGAGGGCG AGGGCCGCCC CTACGAGGGC ACCCAGACCG |
| | CCAAGCTGAA GGTGACCAAG GGTGGCCCCC TGCCCTTCGC |
| | CTGGGACATC CTGTCCCCTC AGTTCATGTA CGGCTCCAAG |
| | GCCTACGTGA AGCACCCCGC CGACATCCCC GACTACTTGA |
| | AGCTGTCCTT CCCCGAGGGC TTCAAGTGGG AGCGCGTGAT |
| | GAACTTCGAG GACGGCGGCG TGGTGACCGT GACCCAGGAC |
| | TCCTCCCTGC AGGACGGCGA GTTCATCTAC AAGGTGAAGC |
| | TGCGCGGCAC CAACTTCCCC TCCGACGGCC CCGTAATGCA |
| | GAAGAAGACC ATGGGCTGGG AGGCCTCCTC CGAGCGGATG |
| | TACCCCGAGG ACGGCGCCCT GAAGGGCGAG ATCAAGCAGA |
| | GGCTGAAGCT GAAGGACGGC GGCCACTACG ACGCTGAGGT |
| | CAAGACCACC TACAAGGCCA AGAAGCCCGT GCAGCTGCCC |
| | GGCGCCTACA ACGTCAACAT CAAGTTGGAC ATCACCTCCC |
| | ACAACGAGGA CTACACCATC GTGGAACAGT ACGAACGCGC |
| | CGAGGGCCGC CACTCCACCG GCGGCATGGA CGAGCTGTAC |
| | AAGTGAATGC ATCGATAAAA TAAAAGATTT TATTTAGTCT |
| | CCAGAAAAAG GGGGGAATGA AAGACCCCAC CTGTAGGTTT |
| | GGCAAGCTAG CTTAAGTAAC GCCATTTTGC AAGGCATGGA |
| | AAATACATAA CTGAGAATAG AGAAGTTCAG ATCAAGGTTA |
| | GGAACAGAGA GACAGCAGAA TATGGGCCAA ACAGGATATC |
| | TGTGGTAAGC AGTTCCTGCC CCGGCTCAGG GCCAAGAACA |
| | GATGGTCCCC AGATGCGGTC CCGCCCTCAG CAGTTTCTAG |
| | AGAACCATCA GATGTTTCCA GGGTGCCCCA AGGACCTGAA |
| | ATGACCCTGT GCCTTATTTG AACTAACCAA TCAGTTCGCT |
| | TCTCGCTTCT GTTCGCGCGC TTCTGCTCCC CGAGCTCAAT |
| | AAAAGAGCCC ACAACCCCTC ACTCGGCGCG CCAGTCCTCC |
| | GATAGACTGC GTCGCCCGGG TACCCGTGTA TCCAATAAAC |
| | CCTCTTGCAG TTGCATCCGA CTTGTGGTCT CGCTGTTCCT |
| | TGGGAGGGTC TCCTCTGAGT GATTGACTAC CCGTCAGCGG |
| | GGGTCTTTCA TGGGTAACAG TTTCTTGAAG TTGGAGAACA |
| | ACATTCTGAG GGTAGGAGTC GAATATTAAG TAATCCTGAC |
| | TCAATTAGCC ACTGTTTTGA ATCCACATAC TCCAATACTC |
| | CTGAAATAGT TCATTATGGA CAGCGCAGAA GAGCTGGGGA |
| | GAATTGTGAA ATTGTTATCC GCTCACAATT CCACACAACA |
| | TACGAGCCGG AAGCATAAAG TGTAAAGCCT GGGGTGCCTA |
| | ATGAGTGAGC TAACTCACAT TAATTGCGTT GCGCTCACTG |
| | CCCGCTTTCC AGTCGGGAAA CCTGTCGTGC CAGCTGCATT |
| | AATGAATCGG CCAACGCGCG GGGAGAGGCG GTTTGCGTAT |
| | TGGGCGCTCT TCCGCTTCCT CGCTCACTGA CTCGCTGCGC |
| | TCGGTCGTTC GGCTGCGGCG AGCGGTATCA GCTCACTCAA |
| | AGGCGGTAAT ACGGTTATCC ACAGAATCAG GGGATAACGC |
| | AGGAAAGAAC ATGTGAGCAA AAGGCCAGCA AAAGGCCAGG |
| | AACCGTAAAA AGGCCGCGTT GCTGGCGTTT TTCCATAGGC |
| | TCCGCCCCCC TGACGAGCAT CACAAAAATC GACGCTCAAG |
| | TCAGAGGTGG CGAAACCCGA CAGGACTATA AAGATACCAG |
| | GCGTTTCCCC CTGGAAGCTC CCTCGTGCGC TCTCCTGTTC |
| | CGACCCTGCC GCTTACCGGA TACCTGTCCG CCTTTCTCCC |
| | TTCGGGAAGC GTGGCGCTTT CTCATAGCTC ACGCTGTAGG |
| | TATCTCAGTT CGGTGTAGGT CGTTCGCTCC AAGCTGGGCT |
| | GTGTGCACGA ACCCCCCGTT CAGCCCGACC GCTGCGCCTT |
| | ATCCGGTAAC TATCGTCTTG AGTCCAACCC GGTAAGACAC |
| | GACTTATCGC CACTGGCAGC AGCCACTGGT AACAGGATTA |
| | GCAGAGCGAG GTATGTAGGC GGTGCTACAG AGTTCTTGAA |
| | GTGGTGGCCT AACTACGGCT ACACTAGAAG GACAGTATTT |
| | GGTATCTGCG CTCTGCTGAA GCCAGTTACC TTCGGAAAAA |
| | GAGTTGGTAG CTCTTGATCC GGCAAACAAA CCACCGCTGG |
| | TAGCGGTGGT TTTTTTGTTT GCAAGCAGCA GATTACGCGC |
| | AGAAAAAAAG GATCTCAAGA AGATCCTTTG ATCTTTTCTA |
| | CGGGGTCTGA CGCTCAGTGG AACGAAAACT CACGTTAAGG |
| | GATTTTGGTC ATGAGATTAT CAAAAAGGAT CTTCACCTAG |
| | ATCCTTTTAA ATTAAAAATG AAGTTTTAAA TCAATCTAAA |
| | GTATATATGA GTAAACTTGG TCTGACAGTT ACCAATGCTT |
| | AATCAGTGAG GCACCTATCT CAGCGATCTG TCTATTTCGT |
| | TCATCCATAG TTGCCTGACT CCCCGTCGTG TAGATAACTA |
| | CGATACGGGA GGGCTTACCA TCTGGCCCCA GTGCTGCAAT |
| | GATACCGCGA GACCCACGCT CACCGGCTCC AGATTTATCA |
| | GCAATAAACC AGCCAGCCGG AAGGGCCGAG CGCAGAAGTG |
| | GTCCTGCAAC TTTATCCGCC TCCATCCAGT CTATTAATTG |
| | TTGCCGGGAA GCTAGAGTAA GTAGTTCGCC AGTTAATAGT |
| | TTGCGCAACG TTGTTGCCAT TGCTACAGGC ATCGTGGTGT |
| | CACGCTCGTC GTTTGGTATG GCTTCATTCA GCTCCGGTTC |
| | CCAACGATCA AGGCGAGTTA CATGATCCCC CATGTTGTGC |
| | AAAAAAGCGG TTAGCTCCTT CGGTCCTCCG ATCGTTGTCA |
| | GAAGTAAGTT GGCCGCAGTG TTATCACTCA TGGTTATGGC |
| | AGCACTGCAT AATTCTCTTA CTGTCATGCC ATCCGTAAGA |
| | TGCTTTTCTG TGACTGGTGA GTACTCAACC AAGTCATTCT |
| | GAGAATAGTG TATGCGGCGA CCGAGTTGCT CTTGCCCGGC |
| | GTCAATACGG GATAATACCG CGCCACATAG CAGAACTTTA |
| | AAAGTGCTCA TCATTGGAAA ACGTTCTTCG GGGCGAAAAC |
| | TCTCAAGGAT CTTACCGCTG TTGAGATCCA GTTCGATGTA |
| | ACCCACTCGT GCACCCAACT GATCTTCAGC ATCTTTTACT |
| | TTCACCAGCG TTTCTGGGTG AGCAAAAACA GGAAGGCAAA |
| | ATGCCGCAAA AAAGGGAATA AGGGCGACAC GGAAATGTTG |
| | AATACTCATA CTCTTCCTTT TTCAATATTA TTGAAGCATT |
| | TATCAGGGTT ATTGTCTCAT GAGCGGATAC ATATTTGAAT |
| | GTATTTAGAA AAATAAACAA ATAGGGGTTC CGCGCACATT |
| | TCCCCGAAAA GTGCCACCTG ACGTCTAAGA AACCATTATT |
| | ATCATGACAT TAACCTATAA AAATAGGCGT ATCACGAGGC |
| | CCTTTCGTCT CGCGCGTTTC GGTGATGACG GTGAAAACCT |
| | CTGACACATG CAGCTCCCGG AGACGGTCAC AGCTTGTCTG |
| | TAAGCGGATG CCGGGAGCAG ACAAGCCCGT CAGGGCGCGT |
| | CAGCGGGTGT TGGCGGGTGT CGGGGCTGGC TTAACTATGC |
| | GGCATCAGAG CAGATTGTAC TGAGAGTGCA CCATATGCGG |
| | TGTGAAATAC CGCACAGATG CGTAAGGAGA AAATACCGCA |
| | TCAGGCGCCA TTCGCCATTC AGGCTGCGCA ACTGTTGGGA |
| | AGGGCGATCG GTGCGGGCCT CTTCGCTATT ACGCCAGCTG |
| | GCGAAAGGGG GATGTGCTGC AAGGCGATTA AGTTGGGTAA |
| | CGCCAGGGTT TTCCCAGTCA CGACGTTGTA AAACGACGGC |
| | GCAAGGAATG GTGCATGCAA GGAGATGGCG CCCAACAGTC |
| | CCCCGGCCAC GGGGCCTGCC ACCATACCCA CGCCGAAACA |
| | AGCGCTCATG AGCCCGAAGT GGCGAGCCCG ATCTTCCCCA |
| | TCGGTGATGT CGGCGATATA GGCGCCAGCA ACCGCACCTG |
| | TGGCGCCGGT GATGCCGGCC ACGATGCGTC CGGCGTAGAG |
| | GCGATTAGTC CAATTTGTTA AAGACAGGAT ATCAGTGGTC |
| | CAGGCTCTAG TTTTGACTCA ACAATATCAC CAGCTGAAGC |
| | CTATAGAGTA CGAGCCATAG ATAAAATAAA AGATTTTATT |
| | TAGTCTCCAG AAAAAGGGGG GAA |
| | (SEQ ID NO: 13) |
| MSCV-CDT-1- | TGAAAGACCC CACCTGTAGG TTTGGCAAGC TAGCTTAAGT |
| HA-IDT™-v2- | AACGCCATTT TGCAAGGCAT GGAAAATACA TAACTGAGAA |
| PGK-mCherry | TAGAGAAGTT CAGATCAAGG TTAGGAACAG AGAGACAGCA |
| (FIG. 16A, | GAATATGGGC CAAACAGGAT ATCTGTGGTA AGCAGTTCCT |
| vector 2) | GCCCCGGCTC AGGGCCAAGA ACAGATGGTC CCCAGATGCG |
| | GTCCCGCCCT CAGCAGTTTC TAGAGAACCA TCAGATGTTT |
| | CCAGGGTGCC CCAAGGACCT GAAATGACCC TGTGCCTTAT |
| | TTGAACTAAC CAATCAGTTC GCTTCTCGCT TCTGTTCGCG |
| | CGCTTCTGCT CCCCGAGCTC AATAAAAGAG CCCACAACCC |
| | CTCACTCGGC GCGCCAGTCC TCCGATAGAC TGCGTCGCCC |
| | GGGTACCCGT ATTCCCAATA AAGCCTCTTG CTGTTTGCAT |
| | CCGAATCGTG GACTCGCTGA TCCTTGGGAG GGTCTCCTCA |
| | GATTGATTGA CTGCCCACCT CGGGGGTCTT TCATTTGGAG |
| | GTTCCACCGA GATTTGGAGA CCCCTGCCCA GGGACCACCG |
| | ACCCCCCCGC CGGGAGGTAA GCTGGCCAGC GGTCGTTTCG |
| | TGTCTGTCTC TGTCTTTGTG CGTGTTTGTG CCGGCATCTA |
| | ATGTTTGCGC CTGCGTCTGT ACTAGTTAGC TAACTAGCTC |
| | TGTATCTGGC GGACCCGTGG TGGAACTGAC GAGTTCTGAA |
| | CACCCGGCCG CAACCCTGGG AGACGTCCCA GGGACTTTGG |
| | GGGCCGTTTT TGTGGCCCGA CCTGAGGAAG GGAGTCGATG |
| | TGGAATCCGA CCCCGTCAGG ATATGTGGTT CTGGTAGGAG |
| | ACGAGAACCT AAAACAGTTC CCGCCTCCGT CTGAATTTTT |
| | GCTTTCGGTT TGGAACCGAA GCCGCGCGTC TTGTCTGCTG |
| | CAGCGCTGCA GCATCGTTCT GTGTTGTCTC TGTCTGACTG |
| | TGTTTCTGTA TTTGTCTGAA AATTAGGGCC AGACTGTTAC |
| | CACTCCCTTA AGTTTGACCT TAGGTCACTG GAAAGATGTC |
| | GAGCGGATCG CTCACAACCA GTCGGTAGAT GTCAAGAAGA |
| | GACGTTGGGT TACCTTCTGC TCTGCAGAAT GGCCAACCTT |
| | TAACGTCGGA TGGCCGCGAG ACGGCACCTT TAACCGAGAC |
| | CTCATCACCC AGGTTAAGAT CAAGGTCTTT TCACCTGGCC |
| | CGCATGGACA CCCAGACCAG GTCCCCTACA TCGTGACCTG |
| | GGAAGCCTTG GCTTTTGACC CCCCTCCCTG GGTCAAGCCC |
| | TTTGTACACC CTAAGCCTCC GCCTCCTCTT CCTCCATCCG |
| | CCCCGTCTCT CCCCCTTGAA CCTCCTCGTT CGACCCCGCC |
| | TCGATCCTCC CTTTATCCAG CCCTCACTCC TTCTCTAGGC |
| | GCCGGAATTA GCCGCCACCA TGGCGTCTTC CCACGGTTCA |
| | CACGACGGAG CTAGTACTGA AAAGCATCTG GCCACCCACG |
| | ACATAGCCCC CACACATGAT GCAATCAAGA TAGTCCCAAA |
| | AGGGCACGGA CAGACTGCAA CTAAACCCGG TGCACAAGAG |
| | AAGGAAGTCA GAAATGCAGC CCTGTTTGCT GCAATAAAAG |
| | AGTCCAATAT AAAACCTTGG TCAAAGGAGT CCATTCACTT |
| | GTATTTCGCC ATCTTTGTAG CCTTCTGTTG TGCCTGCGCT |
| | AATGGGTATG ACGGATCTTT GATGACAGGG ATAATTGCTA |
| | TGGACAAGTT CCAGAACCAG TTCCACACTG GAGACACAGG |
| | TCCCAAAGTC AGCGTTATTT TTTCACTCTA CACCGTAGGT |
| | GCTATGGTAG GGGCTCCATT TGCAGCAATC CTCAGTGATC |
| | GATTCGGACG AAAAAAAGGT ATGTTTATAG GCGGGATCTT |
| | TATCATAGTG GGCTCCATCA TTGTAGCCTC TTCCTCAAAA |
| | TTGGCACAAT TTGTGGTCGG TCGCTTCGTT CTCGGGCTGG |
| | GTATAGCTAT TATGACAGTC GCAGCTCCAG CATATTCAAT |
| | AGAGATCGCC CCACCCCATT GGCGGGGTCG CTGCACCGGC |
| | TTCTACAACT GCGGGTGGTT CGGCGGGTCA ATCCCAGCCG |
| | CTTGTATAAC TTATGGGTGC TATTTTATTA AATCAAATTG |
| | GTCCTGGCGA ATCCCACTCA TACTGCAAGC TTTTACCTGT |
| | CTTATTGTTA TGTCATCAGT CTTCTTCTTG CCAGAATCTC |
| | CTCGGTTTTT GTTCGCCAAT GGAAGGGATG CTGAAGCTGT |
| | CGCCTTCCTG GTCAAGTATC ACGGAAACGG AGACCCAAAC |
| | TCTAAATTGG TTCTGTTGGA GACCGAAGAA ATGCGTGACG |
| | GAATCCGGAC AGATGGGGTT GACAAGGTCT GGTGGGATTA |
| | TAGGCCACTG TTTATGACTC ACTCCGGGCG CTGGCGAATG |
| | GCACAGGTAT TGATGATTTC AATTTTCGGG CAATTTAGTG |
| | GCAACGGACT TGGATACTTC AATACTGTCA TCTTCAAAAA |
| | CATCGGCGTC ACTAGCACCT CACAGCAGCT CGCCTACAAT |
| | ATACTCAACA GCGTTATATC TGCTATTGGT GCACTCACCG |
| | CTGTGTCAAT GACAGATCGA ATGCCCAGGC GCGCAGTTCT |
| | CATAATAGGC ACTTTTATGT GCGCTGCTGC TCTGGCAACT |
| | AACAGTGGGC TCAGTGCTAC TCTTGATAAA CAAACTCAGA |
| | GAGGGACCCA GATTAACCTT AACCAAGGTA TGAATGAGCA |
| | GGATGCAAAA GATAACGCAT ACCTTCACGT GGATTCAAAC |
| | TATGCTAAGG GCGCTCTGGC TGCATATTTC CTCTTTAATG |
| | TAATTTTTAG CTTCACATAT ACCCCTCTTC AAGGTGTCAT |
| | CCCCACCGAG GCCCTGGAAA CCACCATTCG GGGGAAGGGT |
| | CTCGCTCTGT CAGGATTCAT TGTAAATGCT ATGGGATTTA |
| | TCAATCAATT TGCCGGCCCA ATAGCCTTGC ACAATATCGG |
| | ATATAAATAT ATCTTTGTAT TTGTCGGTTG GGATTTGATA |
| | GAAACAGTTG CATGGTACTT TTTCGGAGTT GAATCCCAGG |
| | GCAGAACCTT GGAACAACTG GAGTGGGTGT ACGACCAACC |
| | TAATCCAGTG AAAGCAAGTC TCAAGGTCGA GAAAGTCGTA |
| | GTTCAAGCCG ACGGCCATGT GAGTGAAGCC ATCGTGGCCT |
| | ACCCATACGA TGTTCCAGAT TACGCTTGAT AAGATCTGAA |
| | TTCTACCGGG TAGGTGAGGC GCTTTTCCCA AGGCAGTCTG |
| | GAGCATGCGC TTTAGCAGCC CCGCTGGGCA CTTGGCGCTA |
| | CACAAGTGGC CTCTGGCCTC GCACACATTC CACATCCACC |
| | GGTAGGCGCC AACCGGCTCC GTTCTTTGGT GGCCCCTTCG |
| | CGCCACCTTC TACTCCTCCC CTAGTCAGGA AGTTCCCCCC |
| | CGCCCCGCAG CTCGCGTCGT GCAGGACGTG ACAAATGGAA |
| | GTAGCACGTC TCACTAGTCT CGTGCAGATG GACAGCACCG |
| | CTGAGCAATG GAAGCGGGTA GGCCTTTGGG GCAGCGGCCA |
| | ATAGCAGCTT TGCTCCTTCG CTTTCTGGGC TCAGAGGCTG |
| | GGAAGGGGTG GGTCCGGGGG CGGGCTCAGG GGCGGGCTCA |
| | GGGGCGGGGC GGGCGCCCGA AGGTCCTCCG GAGGCCCGGC |
| | ATTCTGCACG CTTCAAAAGC GCACGTCTGC CGCGCTGTTC |
| | TCCTCTTCCT CATCTCCGGG CCTTTCGACC TGCAGCCCAA |
| | GCTAGGACCA TGGTGAGCAA GGGCGAGGAG GATAACATGG |
| | CCATCATCAA GGAGTTCATG CGCTTCAAGG TGCACATGGA |
| | GGGCTCCGTG AACGGCCACG AGTTCGAGAT CGAGGGCGAG |
| | GGCGAGGGCC GCCCCTACGA GGGCACCCAG ACCGCCAAGC |
| | TGAAGGTGAC CAAGGGTGGC CCCCTGCCCT TCGCCTGGGA |
| | CATCCTGTCC CCTCAGTTCA TGTACGGCTC CAAGGCCTAC |
| | GTGAAGCACC CCGCCGACAT CCCCGACTAC TTGAAGCTGT |
| | CCTTCCCCGA GGGCTTCAAG TGGGAGCGCG TGATGAACTT |
| | CGAGGACGGC GGCGTGGTGA CCGTGACCCA GGACTCCTCC |
| | CTGCAGGACG GCGAGTTCAT CTACAAGGTG AAGCTGCGCG |
| | GCACCAACTT CCCCTCCGAC GGCCCCGTAA TGCAGAAGAA |
| | GACCATGGGC TGGGAGGCCT CCTCCGAGCG GATGTACCCC |
| | GAGGACGGCG CCCTGAAGGG CGAGATCAAG CAGAGGCTGA |
| | AGCTGAAGGA CGGCGGCCAC TACGACGCTG AGGTCAAGAC |
| | CACCTACAAG GCCAAGAAGC CCGTGCAGCT GCCCGGCGCC |
| | TACAACGTCA ACATCAAGTT GGACATCACC TCCCACAACG |
| | AGGACTACAC CATCGTGGAA CAGTACGAAC GCGCCGAGGG |
| | CCGCCACTCC ACCGGCGGCA TGGACGAGCT GTACAAGTGA |
| | ATGCATCGAT AAAATAAAAG ATTTTATTTA GTCTCCAGAA |
| | AAAGGGGGGA ATGAAAGACC CCACCTGTAG GTTTGGCAAG |
| | CTAGCTTAAG TAACGCCATT TTGCAAGGCA TGGAAAATAC |
| | ATAACTGAGA ATAGAGAAGT TCAGATCAAG GTTAGGAACA |
| | GAGAGACAGC AGAATATGGG CCAAACAGGA TATCTGTGGT |
| | AAGCAGTTCC TGCCCCGGCT CAGGGCCAAG AACAGATGGT |
| | CCCCAGATGC GGTCCCGCCC TCAGCAGTTT CTAGAGAACC |
| | ATCAGATGTT TCCAGGGTGC CCCAAGGACC TGAAATGACC |
| | CTGTGCCTTA TTTGAACTAA CCAATCAGTT CGCTTCTCGC |
| | TTCTGTTCGC GCGCTTCTGC TCCCCGAGCT CAATAAAAGA |
| | GCCCACAACC CCTCACTCGG CGCGCCAGTC CTCCGATAGA |
| | CTGCGTCGCC CGGGTACCCG TGTATCCAAT AAACCCTCTT |
| | GCAGTTGCAT CCGACTTGTG GTCTCGCTGT TCCTTGGGAG |
| | GGTCTCCTCT GAGTGATTGA CTACCCGTCA GCGGGGGTCT |
| | TTCATGGGTA ACAGTTTCTT GAAGTTGGAG AACAACATTC |
| | TGAGGGTAGG AGTCGAATAT TAAGTAATCC TGACTCAATT |
| | AGCCACTGTT TTGAATCCAC ATACTCCAAT ACTCCTGAAA |
| | TAGTTCATTA TGGACAGCGC AGAAGAGCTG GGGAGAATTG |
| | TGAAATTGTT ATCCGCTCAC AATTCCACAC AACATACGAG |
| | CCGGAAGCAT AAAGTGTAAA GCCTGGGGTG CCTAATGAGT |
| | GAGCTAACTC ACATTAATTG CGTTGCGCTC ACTGCCCGCT |
| | TTCCAGTCGG GAAACCTGTC GTGCCAGCTG CATTAATGAA |
| | TCGGCCAACG CGCGGGGAGA GGCGGTTTGC GTATTGGGCG |
| | CTCTTCCGCT TCCTCGCTCA CTGACTCGCT GCGCTCGGTC |
| | GTTCGGCTGC GGCGAGCGGT ATCAGCTCAC TCAAAGGCGG |
| | TAATACGGTT ATCCACAGAA TCAGGGGATA ACGCAGGAAA |
| | GAACATGTGA GCAAAAGGCC AGCAAAAGGC CAGGAACCGT |
| | AAAAAGGCCG CGTTGCTGGC GTTTTTCCAT AGGCTCCGCC |
| | CCCCTGACGA GCATCACAAA AATCGACGCT CAAGTCAGAG |
| | GTGGCGAAAC CCGACAGGAC TATAAAGATA CCAGGCGTTT |
| | CCCCCTGGAA GCTCCCTCGT GCGCTCTCCT GTTCCGACCC |
| | TGCCGCTTAC CGGATACCTG TCCGCCTTTC TCCCTTCGGG |
| | AAGCGTGGCG CTTTCTCATA GCTCACGCTG TAGGTATCTC |
| | AGTTCGGTGT AGGTCGTTCG CTCCAAGCTG GGCTGTGTGC |
| | ACGAACCCCC CGTTCAGCCC GACCGCTGCG CCTTATCCGG |
| | TAACTATCGT CTTGAGTCCA ACCCGGTAAG ACACGACTTA |
| | TCGCCACTGG CAGCAGCCAC TGGTAACAGG ATTAGCAGAG |
| | CGAGGTATGT AGGCGGTGCT ACAGAGTTCT TGAAGTGGTG |
| | GCCTAACTAC GGCTACACTA GAAGGACAGT ATTTGGTATC |
| | TGCGCTCTGC TGAAGCCAGT TACCTTCGGA AAAAGAGTTG |
| | GTAGCTCTTG ATCCGGCAAA CAAACCACCG CTGGTAGCGG |
| | TGGTTTTTTT GTTTGCAAGC AGCAGATTAC GCGCAGAAAA |
| | AAAGGATCTC AAGAAGATCC TTTGATCTTT TCTACGGGGT |
| | CTGACGCTCA GTGGAACGAA AACTCACGTT AAGGGATTTT |
| | GGTCATGAGA TTATCAAAAA GGATCTTCAC CTAGATCCTT |
| | TTAAATTAAA AATGAAGTTT TAAATCAATC TAAAGTATAT |
| | ATGAGTAAAC TTGGTCTGAC AGTTACCAAT GCTTAATCAG |
| | TGAGGCACCT ATCTCAGCGA TCTGTCTATT TCGTTCATCC |
| | ATAGTTGCCT GACTCCCCGT CGTGTAGATA ACTACGATAC |
| | GGGAGGGCTT ACCATCTGGC CCCAGTGCTG CAATGATACC |
| | GCGAGACCCA CGCTCACCGG CTCCAGATTT ATCAGCAATA |
| | AACCAGCCAG CCGGAAGGGC CGAGCGCAGA AGTGGTCCTG |
| | CAACTTTATC CGCCTCCATC CAGTCTATTA ATTGTTGCCG |
| | GGAAGCTAGA GTAAGTAGTT CGCCAGTTAA TAGTTTGCGC |
| | AACGTTGTTG CCATTGCTAC AGGCATCGTG GTGTCACGCT |
| | CGTCGTTTGG TATGGCTTCA TTCAGCTCCG GTTCCCAACG |
| | ATCAAGGCGA GTTACATGAT CCCCCATGTT GTGCAAAAAA |
| | GCGGTTAGCT CCTTCGGTCC TCCGATCGTT GTCAGAAGTA |
| | AGTTGGCCGC AGTGTTATCA CTCATGGTTA TGGCAGCACT |
| | GCATAATTCT CTTACTGTCA TGCCATCCGT AAGATGCTTT |
| | TCTGTGACTG GTGAGTACTC AACCAAGTCA TTCTGAGAAT |
| | AGTGTATGCG GCGACCGAGT TGCTCTTGCC CGGCGTCAAT |
| | ACGGGATAAT ACCGCGCCAC ATAGCAGAAC TTTAAAAGTG |
| | CTCATCATTG GAAAACGTTC TTCGGGGCGA AAACTCTCAA |
| | GGATCTTACC GCTGTTGAGA TCCAGTTCGA TGTAACCCAC |
| | TCGTGCACCC AACTGATCTT CAGCATCTTT TACTTTCACC |
| | AGCGTTTCTG GGTGAGCAAA AACAGGAAGG CAAAATGCCG |
| | CAAAAAAGGG AATAAGGGCG ACACGGAAAT GTTGAATACT |
| | CATACTCTTC CTTTTTCAAT ATTATTGAAG CATTTATCAG |
| | GGTTATTGTC TCATGAGCGG ATACATATTT GAATGTATTT |
| | AGAAAAATAA ACAAATAGGG GTTCCGCGCA CATTTCCCCG |
| | AAAAGTGCCA CCTGACGTCT AAGAAACCAT TATTATCATG |
| | ACATTAACCT ATAAAAATAG GCGTATCACG AGGCCCTTTC |
| | GTCTCGCGCG TTTCGGTGAT GACGGTGAAA ACCTCTGACA |
| | CATGCAGCTC CCGGAGACGG TCACAGCTTG TCTGTAAGCG |
| | GATGCCGGGA GCAGACAAGC CCGTCAGGGC GCGTCAGCGG |
| | GTGTTGGCGG GTGTCGGGGC TGGCTTAACT ATGCGGCATC |
| | AGAGCAGATT GTACTGAGAG TGCACCATAT GCGGTGTGAA |
| | ATACCGCACA GATGCGTAAG GAGAAAATAC CGCATCAGGC |
| | GCCATTCGCC ATTCAGGCTG CGCAACTGTT GGGAAGGGCG |
| | ATCGGTGCGG GCCTCTTCGC TATTACGCCA GCTGGCGAAA |
| | GGGGGATGTG CTGCAAGGCG ATTAAGTTGG GTAACGCCAG |
| | GGTTTTCCCA GTCACGACGT TGTAAAACGA CGGCGCAAGG |
| | AATGGTGCAT GCAAGGAGAT GGCGCCCAAC AGTCCCCCGG |
| | CCACGGGGCC TGCCACCATA CCCACGCCGA AACAAGCGCT |
| | CATGAGCCCG AAGTGGCGAG CCCGATCTTC CCCATCGGTG |
| | ATGTCGGCGA TATAGGCGCC AGCAACCGCA CCTGTGGCGC |
| | CGGTGATGCC GGCCACGATG CGTCCGGCGT AGAGGCGATT |
| | AGTCCAATTT GTTAAAGACA GGATATCAGT GGTCCAGGCT |
| | CTAGTTTTGA CTCAACAATA TCACCAGCTG AAGCCTATAG |
| | AGTACGAGCC ATAGATAAAA TAAAAGATTT TATTTAGTCT |
| | CCAGAAAAAG GGGGGAA |
| | (SEQ ID NO: 14) |
| MSCV-CDT-1- | TGAAAGACCC CACCTGTAGG TTTGGCAAGC TAGCTTAAGT |
| HA- | AACGCCATTT TGCAAGGCAT GGAAAATACA TAACTGAGAA |
| BLUEHERON™- | TAGAGAAGTT CAGATCAAGG TTAGGAACAG AGAGACAGCA |
| PGK-mCherry | GAATATGGGC CAAACAGGAT ATCTGTGGTA AGCAGTTCCT |
| (FIG. 16A, | GCCCCGGCTC AGGGCCAAGA ACAGATGGTC CCCAGATGCG |
| vector 3) | GTCCCGCCCT CAGCAGTTTC TAGAGAACCA TCAGATGTTT |
| | CCAGGGTGCC CCAAGGACCT GAAATGACCC TGTGCCTTAT |
| | TTGAACTAAC CAATCAGTTC GCTTCTCGCT TCTGTTCGCG |
| | CGCTTCTGCT CCCCGAGCTC AATAAAAGAG CCCACAACCC |
| | CTCACTCGGC GCGCCAGTCC TCCGATAGAC TGCGTCGCCC |
| | GGGTACCCGT ATTCCCAATA AAGCCTCTTG CTGTTTGCAT |
| | CCGAATCGTG GACTCGCTGA TCCTTGGGAG GGTCTCCTCA |
| | GATTGATTGA CTGCCCACCT CGGGGGTCTT TCATTTGGAG |
| | GTTCCACCGA GATTTGGAGA CCCCTGCCCA GGGACCACCG |
| | ACCCCCCCGC CGGGAGGTAA GCTGGCCAGC GGTCGTTTCG |
| | TGTCTGTCTC TGTCTTTGTG CGTGTTTGTG CCGGCATCTA |
| | ATGTTTGCGC CTGCGTCTGT ACTAGTTAGC TAACTAGCTC |
| | TGTATCTGGC GGACCCGTGG TGGAACTGAC GAGTTCTGAA |
| | CACCCGGCCG CAACCCTGGG AGACGTCCCA GGGACTTTGG |
| | GGGCCGTTTT TGTGGCCCGA CCTGAGGAAG GGAGTCGATG |
| | TGGAATCCGA CCCCGTCAGG ATATGTGGTT CTGGTAGGAG |
| | ACGAGAACCT AAAACAGTTC CCGCCTCCGT CTGAATTTTT |
| | GCTTTCGGTT TGGAACCGAA GCCGCGCGTC TTGTCTGCTG |
| | CAGCGCTGCA GCATCGTTCT GTGTTGTCTC TGTCTGACTG |
| | TGTTTCTGTA TTTGTCTGAA AATTAGGGCC AGACTGTTAC |
| | CACTCCCTTA AGTTTGACCT TAGGTCACTG GAAAGATGTC |
| | GAGCGGATCG CTCACAACCA GTCGGTAGAT GTCAAGAAGA |
| | GACGTTGGGT TACCTTCTGC TCTGCAGAAT GGCCAACCTT |
| | TAACGTCGGA TGGCCGCGAG ACGGCACCTT TAACCGAGAC |
| | CTCATCACCC AGGTTAAGAT CAAGGTCTTT TCACCTGGCC |
| | CGCATGGACA CCCAGACCAG GTCCCCTACA TCGTGACCTG |
| | GGAAGCCTTG GCTTTTGACC CCCCTCCCTG GGTCAAGCCC |
| | TTTGTACACC CTAAGCCTCC GCCTCCTCTT CCTCCATCCG |
| | CCCCGTCTCT CCCCCTTGAA CCTCCTCGTT CGACCCCGCC |
| | TCGATCCTCC CTTTATCCAG CCCTCACTCC TTCTCTAGGC |
| | GCCGGAATTA GCCGCCACCA TGGCGTCTAG TCACGGAAGT |
| | CACGACGGCG CTAGCACCGA AAAGCACCTG GCCACTCACG |
| | ATATTGCCCC TACCCACGAC GCTATCAAGA TCGTACCCAA |
| | AGGTCACGGG CAGACTGCTA CTAAGCCCGG AGCGCAGGAA |
| | AAAGAGGTGC GCAACGCTGC CCTTTTCGCA GCTATCAAGG |
| | AAAGTAATAT TAAACCGTGG AGTAAGGAGA GTATCCATCT |
| | CTATTTCGCT ATCTTCGTAG CTTTCTGCTG TGCGTGCGCC |
| | AACGGGTATG ACGGATCTTT GATGACAGGA ATCATTGCTA |
| | TGGACAAATT CCAGAATCAG TTCCATACAG GAGACACAGG |
| | TCCCAAGGTC AGTGTTATAT TTTCTCTGTA CACAGTCGGT |
| | GCTATGGTAG GTGCCCCCTT CGCTGCTATT CTGTCCGACC |
| | GCTTCGGACG GAAAAAAGGT ATGTTTATCG GGGGAATTTT |
| | TATCATTGTG GGCAGCATTA TCGTGGCAAG TTCAAGCAAA |
| | CTGGCTCAAT TCGTTGTTGG CAGGTTCGTC CTGGGACTGG |
| | GTATCGCTAT TATGACAGTC GCAGCTCCCG CTTATTCTAT |
| | CGAAATCGCA CCACCGCACT GGAGAGGACG CTGCACTGGT |
| | TTTTATAACT GCGGCTGGTT TGGCGGCAGC ATCCCGGCGG |
| | CATGCATCAC CTATGGCTGC TATTTTATCA AGTCCAACTG |
| | GAGCTGGCGA ATCCCCTTGA TCCTCCAGGC CTTCACTTGT |
| | CTCATTGTTA TGTCATCTGT TTTTTTTCTC CCTGAGTCCC |
| | CTAGATTTCT TTTCGCCAAC GGTAGAGACG CTGAGGCTGT |
| | TGCCTTCCTG GTAAAGTACC ACGGCAACGG CGACCCCAAC |
| | TCCAAACTCG TGCTGCTGGA GACTGAAGAA ATGCGTGACG |
| | GGATTCGGAC CGACGGGGTC GACAAGGTCT GGTGGGACTA |
| | TCGCCCTCTT TTTATGACCC ATAGTGGGCG GTGGCGAATG |
| | GCACAGGTAT TGATGATCTC TATCTTTGGG CAATTCTCTG |
| | GGAACGGACT TGGTTACTTT AACACCGTTA TCTTTAAAAA |
| | CATCGGGGTC ACTTCAACCT CTCAGCAATT GGCGTATAAC |
| | ATTCTGAACT CCGTCATCAG CGCAATCGGG GCACTGACAG |
| | CGGTCTCAAT GACTGATCGA ATGCCTCGCA GAGCGGTGCT |
| | TATCATCGGA ACTTTTATGT GCGCTGCTGC CTTGGCCACT |
| | AACAGCGGCC TTTCCGCGAC TTTGGATAAA CAAACACAGC |
| | GGGGTACGCA GATTAACCTC AATCAGGGTA TGAACGAACA |
| | AGATGCTAAA GACAATGCGT ATTTGCACGT CGATAGCAAT |
| | TACGCTAAGG GTGCTTTGGC CGCCTATTTC CTGTTCAACG |
| | TGATTTTTAG CTTCACGTAC ACTCCTCTGC AGGGTGTTAT |
| | TCCAACCGAG GCACTCGAAA CCACGATCCG AGGCAAGGGA |
| | CTGGCACTCA GCGGCTTTAT CGTGAACGCT ATGGGATTCA |
| | TTAATCAGTT TGCTGGCCCT ATTGCTCTGC ACAACATTGG |
| | GTACAAGTAC ATCTTCGTTT TCGTGGGCTG GGACCTCATC |
| | GAAACTGTGG CGTGGTATTT CTTCGGAGTG GAGAGTCAGG |
| | GGCGAACGCT GGAACAGCTC GAATGGGTGT ATGATCAACC |
| | CAATCCTGTA AAAGCAAGTC TGAAGGTGGA GAAAGTTGTG |
| | GTGCAGGCTG ATGGACACGT GTCTGAAGCC ATCGTGGCGT |
| | ACCCATACGA TGTTCCAGAT TACGCTTGAT AAGATCTGAA |
| | TTCTACCGGG TAGGTGAGGC GCTTTTCCCA AGGCAGTCTG |
| | GAGCATGCGC TTTAGCAGCC CCGCTGGGCA CTTGGCGCTA |
| | CACAAGTGGC CTCTGGCCTC GCACACATTC CACATCCACC |
| | GGTAGGCGCC AACCGGCTCC GTTCTTTGGT GGCCCCTTCG |
| | CGCCACCTTC TACTCCTCCC CTAGTCAGGA AGTTCCCCCC |
| | CGCCCCGCAG CTCGCGTCGT GCAGGACGTG ACAAATGGAA |
| | GTAGCACGTC TCACTAGTCT CGTGCAGATG GACAGCACCG |
| | CTGAGCAATG GAAGCGGGTA GGCCTTTGGG GCAGCGGCCA |
| | ATAGCAGCTT TGCTCCTTCG CTTTCTGGGC TCAGAGGCTG |
| | GGAAGGGGTG GGTCCGGGGG CGGGCTCAGG GGCGGGCTCA |
| | GGGGCGGGGC GGGCGCCCGA AGGTCCTCCG GAGGCCCGGC |
| | ATTCTGCACG CTTCAAAAGC GCACGTCTGC CGCGCTGTTC |
| | TCCTCTTCCT CATCTCCGGG CCTTTCGACC TGCAGCCCAA |
| | GCTAGGACCA TGGTGAGCAA GGGCGAGGAG GATAACATGG |
| | CCATCATCAA GGAGTTCATGCGCTTCAAGG TGCACATGGA |
| | GGGCTCCGTG AACGGCCACG AGTTCGAGAT CGAGGGCGAG |
| | GGCGAGGGCC GCCCCTACGA GGGCACCCAG ACCGCCAAGC |
| | TGAAGGTGAC CAAGGGTGGC CCCCTGCCCT TCGCCTGGGA |
| | CATCCTGTCC CCTCAGTTCA TGTACGGCTC CAAGGCCTAC |
| | GTGAAGCACC CCGCCGACAT CCCCGACTAC TTGAAGCTGT |
| | CCTTCCCCGA GGGCTTCAAG TGGGAGCGCG TGATGAACTT |
| | CGAGGACGGC GGCGTGGTGA CCGTGACCCA GGACTCCTCC |
| | CTGCAGGACG GCGAGTTCAT CTACAAGGTG AAGCTGCGCG |
| | GCACCAACTT CCCCTCCGAC GGCCCCGTAA TGCAGAAGAA |
| | GACCATGGGC TGGGAGGCCT CCTCCGAGCG GATGTACCCC |
| | GAGGACGGCG CCCTGAAGGG CGAGATCAAG CAGAGGCTGA |
| | AGCTGAAGGA CGGCGGCCAC TACGACGCTG AGGTCAAGAC |
| | CACCTACAAG GCCAAGAAGC CCGTGCAGCT GCCCGGCGCC |
| | TACAACGTCA ACATCAAGTT GGACATCACC TCCCACAACG |
| | AGGACTACAC CATCGTGGAA CAGTACGAAC GCGCCGAGGG |
| | CCGCCACTCC ACCGGCGGCA TGGACGAGCT GTACAAGTGA |
| | ATGCATCGAT AAAATAAAAG ATTTTATTTA GTCTCCAGAA |
| | AAAGGGGGGA ATGAAAGACC CCACCTGTAG GTTTGGCAAG |
| | CTAGCTTAAG TAACGCCATT TTGCAAGGCA TGGAAAATAC |
| | ATAACTGAGA ATAGAGAAGT TCAGATCAAG GTTAGGAACA |
| | GAGAGACAGC AGAATATGGG CCAAACAGGA TATCTGTGGT |
| | AAGCAGTTCC TGCCCCGGCT CAGGGCCAAG AACAGATGGT |
| | CCCCAGATGC GGTCCCGCCC TCAGCAGTTT CTAGAGAACC |
| | ATCAGATGTT TCCAGGGTGC CCCAAGGACC TGAAATGACC |
| | CTGTGCCTTA TTTGAACTAA CCAATCAGTT CGCTTCTCGC |
| | TTCTGTTCGC GCGCTTCTGC TCCCCGAGCT CAATAAAAGA |
| | GCCCACAACC CCTCACTCGG CGCGCCAGTC CTCCGATAGA |
| | CTGCGTCGCC CGGGTACCCG TGTATCCAAT AAACCCTCTT |
| | GCAGTTGCAT CCGACTTGTG GTCTCGCTGT TCCTTGGGAG |
| | GGTCTCCTCT GAGTGATTGA CTACCCGTCA GCGGGGGTCT |
| | TTCATGGGTA ACAGTTTCTT GAAGTTGGAG AACAACATTC |
| | TGAGGGTAGG AGTCGAATAT TAAGTAATCC TGACTCAATT |
| | AGCCACTGTT TTGAATCCAC ATACTCCAAT ACTCCTGAAA |
| | TAGTTCATTA TGGACAGCGC AGAAGAGCTG GGGAGAATTG |
| | TGAAATTGTT ATCCGCTCAC AATTCCACAC AACATACGAG |
| | CCGGAAGCAT AAAGTGTAAA GCCTGGGGTG CCTAATGAGT |
| | GAGCTAACTC ACATTAATTG CGTTGCGCTC ACTGCCCGCT |
| | TTCCAGTCGG GAAACCTGTC GTGCCAGCTG CATTAATGAA |
| | TCGGCCAACG CGCGGGGAGA GGCGGTTTGC GTATTGGGCG |
| | CTCTTCCGCT TCCTCGCTCA CTGACTCGCT GCGCTCGGTC |
| | GTTCGGCTGC GGCGAGCGGT ATCAGCTCAC TCAAAGGCGG |
| | TAATACGGTT ATCCACAGAA TCAGGGGATA ACGCAGGAAA |
| | GAACATGTGA GCAAAAGGCC AGCAAAAGGC CAGGAACCGT |
| | AAAAAGGCCG CGTTGCTGGC GTTTTTCCAT AGGCTCCGCC |
| | CCCCTGACGA GCATCACAAA AATCGACGCT CAAGTCAGAG |
| | GTGGCGAAAC CCGACAGGAC TATAAAGATA CCAGGCGTTT |
| | CCCCCTGGAA GCTCCCTCGT GCGCTCTCCT GTTCCGACCC |
| | TGCCGCTTAC CGGATACCTG TCCGCCTTTC TCCCTTCGGG |
| | AAGCGTGGCG CTTTCTCATA GCTCACGCTG TAGGTATCTC |
| | AGTTCGGTGT AGGTCGTTCG CTCCAAGCTG GGCTGTGTGC |
| | ACGAACCCCC CGTTCAGCCC GACCGCTGCG CCTTATCCGG |
| | TAACTATCGT CTTGAGTCCA ACCCGGTAAG ACACGACTTA |
| | TCGCCACTGG CAGCAGCCAC TGGTAACAGG ATTAGCAGAG |
| | CGAGGTATGT AGGCGGTGCT ACAGAGTTCT TGAAGTGGTG |
| | GCCTAACTAC GGCTACACTA GAAGGACAGT ATTTGGTATC |
| | TGCGCTCTGC TGAAGCCAGT TACCTTCGGA AAAAGAGTTG |
| | GTAGCTCTTG ATCCGGCAAA CAAACCACCG CTGGTAGCGG |
| | TGGTTTTTTT GTTTGCAAGC AGCAGATTAC GCGCAGAAAA |
| | AAAGGATCTC AAGAAGATCC TTTGATCTTT TCTACGGGGT |
| | CTGACGCTCA GTGGAACGAA AACTCACGTT AAGGGATTTT |
| | GGTCATGAGA TTATCAAAAA GGATCTTCAC CTAGATCCTT |
| | TTAAATTAAA AATGAAGTTT TAAATCAATC TAAAGTATAT |
| | ATGAGTAAAC TTGGTCTGAC AGTTACCAAT GCTTAATCAG |
| | TGAGGCACCT ATCTCAGCGA TCTGTCTATT TCGTTCATCC |
| | ATAGTTGCCT GACTCCCCGT CGTGTAGATA ACTACGATAC |
| | GGGAGGGCTT ACCATCTGGC CCCAGTGCTG CAATGATACC |
| | GCGAGACCCA CGCTCACCGG CTCCAGATTT ATCAGCAATA |
| | AACCAGCCAG CCGGAAGGGC CGAGCGCAGA AGTGGTCCTG |
| | CAACTTTATC CGCCTCCATC CAGTCTATTA ATTGTTGCCG |
| | GGAAGCTAGA GTAAGTAGTT CGCCAGTTAA TAGTTTGCGC |
| | AACGTTGTTG CCATTGCTAC AGGCATCGTG GTGTCACGCT |
| | CGTCGTTTGG TATGGCTTCA TTCAGCTCCG GTTCCCAACG |
| | ATCAAGGCGA GTTACATGAT CCCCCATGTT GTGCAAAAAA |
| | GCGGTTAGCT CCTTCGGTCC TCCGATCGTT GTCAGAAGTA |
| | AGTTGGCCGC AGTGTTATCA CTCATGGTTA TGGCAGCACT |
| | GCATAATTCT CTTACTGTCA TGCCATCCGT AAGATGCTTT |
| | TCTGTGACTG GTGAGTACTC AACCAAGTCA TTCTGAGAAT |
| | AGTGTATGCG GCGACCGAGT TGCTCTTGCC CGGCGTCAAT |
| | ACGGGATAAT ACCGCGCCAC ATAGCAGAAC TTTAAAAGTG |
| | CTCATCATTG GAAAACGTTC TTCGGGGCGA AAACTCTCAA |
| | GGATCTTACC GCTGTTGAGA TCCAGTTCGA TGTAACCCAC |
| | TCGTGCACCC AACTGATCTT CAGCATCTTT TACTTTCACC |
| | AGCGTTTCTG GGTGAGCAAA AACAGGAAGG CAAAATGCCG |
| | CAAAAAAGGG AATAAGGGCG ACACGGAAAT GTTGAATACT |
| | CATACTCTTC CTTTTTCAAT ATTATTGAAG CATTTATCAG |
| | GGTTATTGTC TCATGAGCGG ATACATATTT GAATGTATTT |
| | AGAAAAATAA ACAAATAGGG GTTCCGCGCA CATTTCCCCG |
| | AAAAGTGCCA CCTGACGTCT AAGAAACCAT TATTATCATG |
| | ACATTAACCT ATAAAAATAG GCGTATCACG AGGCCCTTTC |
| | GTCTCGCGCG TTTCGGTGAT GACGGTGAAA ACCTCTGACA |
| | CATGCAGCTC CCGGAGACGG TCACAGCTTG TCTGTAAGCG |
| | GATGCCGGGA GCAGACAAGC CCGTCAGGGC GCGTCAGCGG |
| | GTGTTGGCGG GTGTCGGGGC TGGCTTAACT ATGCGGCATC |
| | AGAGCAGATT GTACTGAGAG TGCACCATAT GCGGTGTGAA |
| | ATACCGCACA GATGCGTAAG GAGAAAATAC CGCATCAGGC |
| | GCCATTCGCC ATTCAGGCTG CGCAACTGTT GGGAAGGGCG |
| | ATCGGTGCGG GCCTCTTCGC TATTACGCCA GCTGGCGAAA |
| | GGGGGATGTG CTGCAAGGCG ATTAAGTTGG GTAACGCCAG |
| | GGTTTTCCCA GTCACGACGT TGTAAAACGA CGGCGCAAGG |
| | AATGGTGCAT GCAAGGAGAT GGCGCCCAAC AGTCCCCCGG |
| | CCACGGGGCC TGCCACCATA CCCACGCCGA AACAAGCGCT |
| | CATGAGCCCG AAGTGGCGAG CCCGATCTTC CCCATCGGTG |
| | ATGTCGGCGA TATAGGCGCC AGCAACCGCA CCTGTGGCGC |
| | CGGTGATGCC GGCCACGATG CGTCCGGCGT AGAGGCGATT |
| | AGTCCAATTT GTTAAAGACA GGATATCAGT GGTCCAGGCT |
| | CTAGTTTTGA CTCAACAATA TCACCAGCTG AAGCCTATAG |
| | AGTACGAGCC ATAGATAAAA TAAAAGATTT TATTTAGTCT |
| | CCAGAAAAAG GGGGGAA |
| | (SEQ ID NO: 15) |
| MSCV-CDT-1- | TGAAAGACCC CACCTGTAGG TTTGGCAAGC TAGCTTAAGT |
| HA- | AACGCCATTT TGCAAGGCAT GGAAAATACA TAACTGAGAA |
| GENSCRIPT™- | TAGAGAAGTT CAGATCAAGG TTAGGAACAG AGAGACAGCA |
| mCherry | GAATATGGGC CAAACAGGAT ATCTGTGGTA AGCAGTTCCT |
| (FIG. 16A, | GCCCCGGCTC AGGGCCAAGA ACAGATGGTC CCCAGATGCG |
| vector 4) | GTCCCGCCCT CAGCAGTTTC TAGAGAACCA TCAGATGTTT |
| | CCAGGGTGCC CCAAGGACCT GAAATGACCC TGTGCCTTAT |
| | TTGAACTAAC CAATCAGTTC GCTTCTCGCT TCTGTTCGCG |
| | CGCTTCTGCT CCCCGAGCTC AATAAAAGAG CCCACAACCC |
| | CTCACTCGGC GCGCCAGTCC TCCGATAGAC TGCGTCGCCC |
| | GGGTACCCGT ATTCCCAATA AAGCCTCTTG CTGTTTGCAT |
| | CCGAATCGTG GACTCGCTGA TCCTTGGGAG GGTCTCCTCA |
| | GATTGATTGA CTGCCCACCT CGGGGGTCTT TCATTTGGAG |
| | GTTCCACCGA GATTTGGAGA CCCCTGCCCA GGGACCACCG |
| | ACCCCCCCGC CGGGAGGTAA GCTGGCCAGC GGTCGTTTCG |
| | TGTCTGTCTC TGTCTTTGTG CGTGTTTGTG CCGGCATCTA |
| | ATGTTTGCGC CTGCGTCTGT ACTAGTTAGC TAACTAGCTC |
| | TGTATCTGGC GGACCCGTGG TGGAACTGAC GAGTTCTGAA |
| | CACCCGGCCG CAACCCTGGG AGACGTCCCA GGGACTTTGG |
| | GGGCCGTTTT TGTGGCCCGA CCTGAGGAAG GGAGTCGATG |
| | TGGAATCCGA CCCCGTCAGG ATATGTGGTT CTGGTAGGAG |
| | ACGAGAACCT AAAACAGTTC CCGCCTCCGT CTGAATTTTT |
| | GCTTTCGGTT TGGAACCGAA GCCGCGCGTC TTGTCTGCTG |
| | CAGCGCTGCA GCATCGTTCT GTGTTGTCTC TGTCTGACTG |
| | TGTTTCTGTA TTTGTCTGAA AATTAGGGCC AGACTGTTAC |
| | CACTCCCTTA AGTTTGACCT TAGGTCACTG GAAAGATGTC |
| | GAGCGGATCG CTCACAACCA GTCGGTAGAT GTCAAGAAGA |
| | GACGTTGGGT TACCTTCTGC TCTGCAGAAT GGCCAACCTT |
| | TAACGTCGGA TGGCCGCGAG ACGGCACCTT TAACCGAGAC |
| | CTCATCACCC AGGTTAAGAT CAAGGTCTTT TCACCTGGCC |
| | CGCATGGACA CCCAGACCAG GTCCCCTACA TCGTGACCTG |
| | GGAAGCCTTG GCTTTTGACC CCCCTCCCTG GGTCAAGCCC |
| | TTTGTACACC CTAAGCCTCC GCCTCCTCTT CCTCCATCCG |
| | CCCCGTCTCT CCCCCTTGAA CCTCCTCGTT CGACCCCGCC |
| | TCGATCCTCC CTTTATCCAG CCCTCACTCC TTCTCTAGGC |
| | GCCGGAATTA GCCGCCACCA TGGCGTCATC TCACGGTTCT |
| | CACGACGGGG CCTCCACCGA GAAACATCTC GCTACTCATG |
| | ACATCGCTCC AACACATGAT GCCATAAAGA TCGTGCCCAA |
| | GGGTCACGGA CAGACAGCCA CAAAGCCTGG GGCTCAGGAA |
| | AAGGAAGTTA GAAATGCAGC CCTGTTCGCT GCTATTAAAG |
| | AAAGTAACAT CAAACCGTGG AGTAAGGAAA GCATCCACCT |
| | GTATTTCGCA ATATTTGTGG CTTTCTGCTG CGCCTGTGCC |
| | AATGGCTATG ACGGATCTTT GATGACAGGA ATAATTGCTA |
| | TGGACAAGTT CCAGAACCAG TTCCACACTG GGGACACCGG |
| | CCCCAAAGTC TCCGTGATCT TTTCTTTATA CACCGTTGGT |
| | GCTATGGTAG GTGCCCCCTT TGCTGCGATA CTGAGTGACA |
| | GATTTGGTAG GAAGAAAGGT ATGTTTATTG GGGGCATTTT |
| | TATCATAGTC GGGTCTATTA TTGTGGCATC CTCCAGCAAA |
| | CTGGCTCAAT TTGTCGTGGG GCGGTTCGTA TTGGGCCTGG |
| | GGATTGCTAT TATGACAGTT GCAGCACCTG CATACAGCAT |
| | TGAGATCGCT CCGCCACACT GGCGGGGACG ATGTACAGGA |
| | TTCTACAACT GTGGGTGGTT TGGAGGCTCC ATCCCAGCCG |
| | CCTGCATCAC CTATGGCTGC TACTTCATCA AGAGCAACTG |
| | GAGCTGGCGC ATCCCCCTCA TCCTCCAAGC CTTCACCTGC |
| | CTGATTGTTA TGTCAAGCGT CTTCTTTCTC CCTGAGTCAC |
| | CACGCTTCCT GTTTGCCAAC GGGCGTGATG CAGAGGCCGT |
| | AGCCTTTCTG GTGAAATACC ACGGGAACGG AGACCCAAAT |
| | TCAAAACTTG TGCTGCTCGA GACAGAAGAA ATGCGTGACG |
| | GCATCAGGAC AGATGGTGTT GATAAAGTGT GGTGGGACTA |
| | CCGGCCTCTT TTTATGACGC ACTCCGGACG CTGGCGAATG |
| | GCACAGGTAT TGATGATCTC CATTTTCGGG CAATTCTCTG |
| | GAAACGGACT AGGATATTTT AACACAGTCA TCTTTAAGAA |
| | TATTGGAGTC ACATCAACCA GTCAGCAGTT GGCGTATAAC |
| | ATTCTGAACA GCGTTATTTC AGCGATCGGC GCTTTAACGG |
| | CTGTTTCAAT GACAGATCGA ATGCCCAGGA GAGCTGTGCT |
| | TATCATCGGG ACTTTTATGT GTGCTGCTGC GCTGGCCACG |
| | AATAGTGGCC TGTCAGCCAC TTTGGATAAG CAGACCCAGC |
| | GTGGTACTCA GATCAACCTC AACCAGGGTA TGAATGAGCA |
| | GGACGCCAAG GACAACGCCT ATCTGCACGT GGACAGCAAC |
| | TATGCTAAAG GCGCGTTGGC AGCCTACTTT CTCTTCAATG |
| | TCATCTTCAG CTTTACCTAC ACACCTCTGC AGGGCGTGAT |
| | TCCTACAGAA GCTTTAGAAA CCACCATCCG AGGCAAAGGA |
| | CTCGCTTTGT CTGGTTTCAT AGTGAATGCT ATGGGATTTA |
| | TCAATCAGTT TGCAGGGCCC ATTGCACTTC ACAACATCGG |
| | CTACAAGTAC ATCTTCGTCT TTGTTGGCTG GGATCTTATT |
| | GAAACTGTGG CCTGGTACTT CTTCGGAGTG GAGTCTCAAG |
| | GTCGGACTCT AGAACAGCTG GAGTGGGTGT ATGACCAGCC |
| | AAACCCAGTG AAGGCATCGC TGAAAGTAGA GAAGGTGGTG |
| | GTACAAGCGG ACGGTCATGT CAGTGAAGCA ATAGTCGCAT |
| | ACCCATACGA TGTTCCAGAT TACGCTTGAT AAGATCTGAA |
| | TTCTACCGGG TAGGTGAGGC GCTTTTCCCA AGGCAGTCTG |
| | GAGCATGCGC TTTAGCAGCC CCGCTGGGCA CTTGGCGCTA |
| | CACAAGTGGC CTCTGGCCTC GCACACATTC CACATCCACC |
| | GGTAGGCGCC AACCGGCTCC GTTCTTTGGT GGCCCCTTCG |
| | CGCCACCTTC TACTCCTCCC CTAGTCAGGA AGTTCCCCCC |
| | CGCCCCGCAG CTCGCGTCGT GCAGGACGTG ACAAATGGAA |
| | GTAGCACGTC TCACTAGTCT CGTGCAGATG GACAGCACCG |
| | CTGAGCAATG GAAGCGGGTA GGCCTTTGGG GCAGCGGCCA |
| | ATAGCAGCTT TGCTCCTTCG CTTTCTGGGC TCAGAGGCTG |
| | GGAAGGGGTG GGTCCGGGGG CGGGCTCAGG GGCGGGCTCA |
| | GGGGCGGGGC GGGCGCCCGA AGGTCCTCCG GAGGCCCGGC |
| | ATTCTGCACG CTTCAAAAGC GCACGTCTGC CGCGCTGTTC |
| | TCCTCTTCCT CATCTCCGGG CCTTTCGACC TGCAGCCCAA |
| | GCTAGGACCA TGGTGAGCAA GGGCGAGGAG GATAACATGG |
| | CCATCATCAA GGAGTTCATG CGCTTCAAGG TGCACATGGA |
| | GGGCTCCGTG AACGGCCACG AGTTCGAGAT CGAGGGCGAG |
| | GGCGAGGGCC GCCCCTACGA GGGCACCCAG ACCGCCAAGC |
| | TGAAGGTGAC CAAGGGTGGC CCCCTGCCCT TCGCCTGGGA |
| | CATCCTGTCC CCTCAGTTCA TGTACGGCTC CAAGGCCTAC |
| | GTGAAGCACC CCGCCGACAT CCCCGACTAC TTGAAGCTGT |
| | CCTTCCCCGA GGGCTTCAAG TGGGAGCGCG TGATGAACTT |
| | CGAGGACGGC GGCGTGGTGA CCGTGACCCA GGACTCCTCC |
| | CTGCAGGACG GCGAGTTCAT CTACAAGGTG AAGCTGCGCG |
| | GCACCAACTT CCCCTCCGAC GGCCCCGTAA TGCAGAAGAA |
| | GACCATGGGC TGGGAGGCCT CCTCCGAGCG GATGTACCCC |
| | GAGGACGGCG CCCTGAAGGG CGAGATCAAG CAGAGGCTGA |
| | AGCTGAAGGA CGGCGGCCAC TACGACGCTG AGGTCAAGAC |
| | CACCTACAAG GCCAAGAAGC CCGTGCAGCT GCCCGGCGCC |
| | TACAACGTCA ACATCAAGTT GGACATCACC TCCCACAACG |
| | AGGACTACAC CATCGTGGAA CAGTACGAAC GCGCCGAGGG |
| | CCGCCACTCC ACCGGCGGCA TGGACGAGCT GTACAAGTGA |
| | ATGCATCGAT AAAATAAAAG ATTTTATTTA GTCTCCAGAA |
| | AAAGGGGGGA ATGAAAGACC CCACCTGTAG GTTTGGCAAG |
| | CTAGCTTAAG TAACGCCATT TTGCAAGGCA TGGAAAATAC |
| | ATAACTGAGA ATAGAGAAGT TCAGATCAAG GTTAGGAACA |
| | GAGAGACAGC AGAATATGGG CCAAACAGGA TATCTGTGGT |
| | AAGCAGTTCC TGCCCCGGCT CAGGGCCAAG AACAGATGGT |
| | CCCCAGATGC GGTCCCGCCC TCAGCAGTTT CTAGAGAACC |
| | ATCAGATGTT TCCAGGGTGC CCCAAGGACC TGAAATGACC |
| | CTGTGCCTTA TTTGAACTAA CCAATCAGTT CGCTTCTCGC |
| | TTCTGTTCGC GCGCTTCTGC TCCCCGAGCT CAATAAAAGA |
| | GCCCACAACC CCTCACTCGG CGCGCCAGTC CTCCGATAGA |
| | CTGCGTCGCC CGGGTACCCG TGTATCCAAT AAACCCTCTT |
| | GCAGTTGCAT CCGACTTGTG GTCTCGCTGT TCCTTGGGAG |
| | GGTCTCCTCT GAGTGATTGA CTACCCGTCA GCGGGGGTCT |
| | TTCATGGGTA ACAGTTTCTT GAAGTTGGAG AACAACATTC |
| | TGAGGGTAGG AGTCGAATAT TAAGTAATCC TGACTCAATT |
| | AGCCACTGTT TTGAATCCAC ATACTCCAAT ACTCCTGAAA |
| | TAGTTCATTA TGGACAGCGC AGAAGAGCTG GGGAGAATTG |
| | TGAAATTGTT ATCCGCTCAC AATTCCACAC AACATACGAG |
| | CCGGAAGCAT AAAGTGTAAA GCCTGGGGTG CCTAATGAGT |
| | GAGCTAACTC ACATTAATTG CGTTGCGCTC ACTGCCCGCT |
| | TTCCAGTCGG GAAACCTGTC GTGCCAGCTG CATTAATGAA |
| | TCGGCCAACG CGCGGGGAGA GGCGGTTTGC GTATTGGGCG |
| | CTCTTCCGCT TCCTCGCTCA CTGACTCGCT GCGCTCGGTC |
| | GTTCGGCTGC GGCGAGCGGT ATCAGCTCAC TCAAAGGCGG |
| | TAATACGGTT ATCCACAGAA TCAGGGGATA ACGCAGGAAA |
| | GAACATGTGA GCAAAAGGCC AGCAAAAGGC CAGGAACCGT |
| | AAAAAGGCCG CGTTGCTGGC GTTTTTCCAT AGGCTCCGCC |
| | CCCCTGACGA GCATCACAAA AATCGACGCT CAAGTCAGAG |
| | GTGGCGAAAC CCGACAGGAC TATAAAGATA CCAGGCGTTT |
| | CCCCCTGGAA GCTCCCTCGT GCGCTCTCCT GTTCCGACCC |
| | TGCCGCTTAC CGGATACCTG TCCGCCTTTC TCCCTTCGGG |
| | AAGCGTGGCG CTTTCTCATA GCTCACGCTG TAGGTATCTC |
| | AGTTCGGTGT AGGTCGTTCG CTCCAAGCTG GGCTGTGTGC |
| | ACGAACCCCC CGTTCAGCCC GACCGCTGCG CCTTATCCGG |
| | TAACTATCGT CTTGAGTCCA ACCCGGTAAG ACACGACTTA |
| | TCGCCACTGG CAGCAGCCAC TGGTAACAGG ATTAGCAGAG |
| | CGAGGTATGT AGGCGGTGCT ACAGAGTTCT TGAAGTGGTG |
| | GCCTAACTAC GGCTACACTA GAAGGACAGT ATTTGGTATC |
| | TGCGCTCTGC TGAAGCCAGT TACCTTCGGA AAAAGAGTTG |
| | GTAGCTCTTG ATCCGGCAAA CAAACCACCG CTGGTAGCGG |
| | TGGTTTTTTT GTTTGCAAGC AGCAGATTAC GCGCAGAAAA |
| | AAAGGATCTC AAGAAGATCC TTTGATCTTT TCTACGGGGT |
| | CTGACGCTCA GTGGAACGAA AACTCACGTT AAGGGATTTT |
| | GGTCATGAGA TTATCAAAAA GGATCTTCAC CTAGATCCTT |
| | TTAAATTAAA AATGAAGTTT TAAATCAATC TAAAGTATAT |
| | ATGAGTAAAC TTGGTCTGAC AGTTACCAAT GCTTAATCAG |
| | TGAGGCACCT ATCTCAGCGA TCTGTCTATT TCGTTCATCC |
| | ATAGTTGCCT GACTCCCCGT CGTGTAGATA ACTACGATAC |
| | GGGAGGGCTT ACCATCTGGC CCCAGTGCTG CAATGATACC |
| | GCGAGACCCA CGCTCACCGG CTCCAGATTT ATCAGCAATA |
| | AACCAGCCAG CCGGAAGGGC CGAGCGCAGA AGTGGTCCTG |
| | CAACTTTATC CGCCTCCATC CAGTCTATTA ATTGTTGCCG |
| | GGAAGCTAGA GTAAGTAGTT CGCCAGTTAA TAGTTTGCGC |
| | AACGTTGTTG CCATTGCTAC AGGCATCGTG GTGTCACGCT |
| | CGTCGTTTGG TATGGCTTCA TTCAGCTCCG GTTCCCAACG |
| | ATCAAGGCGA GTTACATGAT CCCCCATGTT GTGCAAAAAA |
| | GCGGTTAGCT CCTTCGGTCC TCCGATCGTT GTCAGAAGTA |
| | AGTTGGCCGC AGTGTTATCA CTCATGGTTA TGGCAGCACT |
| | GCATAATTCT CTTACTGTCA TGCCATCCGT AAGATGCTTT |
| | TCTGTGACTG GTGAGTACTC AACCAAGTCA TTCTGAGAAT |
| | AGTGTATGCG GCGACCGAGT TGCTCTTGCC CGGCGTCAAT |
| | ACGGGATAAT ACCGCGCCAC ATAGCAGAAC TTTAAAAGTG |
| | CTCATCATTG GAAAACGTTC TTCGGGGCGA AAACTCTCAA |
| | GGATCTTACC GCTGTTGAGA TCCAGTTCGA TGTAACCCAC |
| | TCGTGCACCC AACTGATCTT CAGCATCTTT TACTTTCACC |
| | AGCGTTTCTG GGTGAGCAAA AACAGGAAGG CAAAATGCCG |
| | CAAAAAAGGG AATAAGGGCG ACACGGAAAT GTTGAATACT |
| | CATACTCTTC CTTTTTCAAT ATTATTGAAG CATTTATCAG |
| | GGTTATTGTC TCATGAGCGG ATACATATTT GAATGTATTT |
| | AGAAAAATAA ACAAATAGGG GTTCCGCGCA CATTTCCCCG |
| | AAAAGTGCCA CCTGACGTCT AAGAAACCAT TATTATCATG |
| | ACATTAACCT ATAAAAATAG GCGTATCACG AGGCCCTTTC |
| | GTCTCGCGCG TTTCGGTGAT GACGGTGAAA ACCTCTGACA |
| | CATGCAGCTC CCGGAGACGG TCACAGCTTG TCTGTAAGCG |
| | GATGCCGGGA GCAGACAAGC CCGTCAGGGC GCGTCAGCGG |
| | GTGTTGGCGG GTGTCGGGGC TGGCTTAACT ATGCGGCATC |
| | AGAGCAGATT GTACTGAGAG TGCACCATAT GCGGTGTGAA |
| | ATACCGCACA GATGCGTAAG GAGAAAATAC CGCATCAGGC |
| | GCCATTCGCC ATTCAGGCTG CGCAACTGTT GGGAAGGGCG |
| | ATCGGTGCGG GCCTCTTCGC TATTACGCCA GCTGGCGAAA |
| | GGGGGATGTG CTGCAAGGCG ATTAAGTTGG GTAACGCCAG |
| | GGTTTTCCCA GTCACGACGT TGTAAAACGA CGGCGCAAGG |
| | AATGGTGCAT GCAAGGAGAT GGCGCCCAAC AGTCCCCCGG |
| | CCACGGGGCC TGCCACCATA CCCACGCCGA AACAAGCGCT |
| | CATGAGCCCG AAGTGGCGAG CCCGATCTTC CCCATCGGTG |
| | ATGTCGGCGA TATAGGCGCC AGCAACCGCA CCTGTGGCGC |
| | CGGTGATGCC GGCCACGATG CGTCCGGCGT AGAGGCGATT |
| | AGTCCAATTT GTTAAAGACA GGATATCAGT GGTCCAGGCT |
| | CTAGTTTTGA CTCAACAATA TCACCAGCTG AAGCCTATAG |
| | AGTACGAGCC ATAGATAAAA TAAAAGATTT TATTTAGTCT |
| | CCAGAAAAAG GGGGGAA |
| | (SEQ ID NO: 16) |
| MSCV_PGK-2A- | AATGAAAGAC CCCACCTGTA GGTTTGGCAA GCTAGCTTAA |
| mCherry vector | GTAACGCCAT TTTGCAAGGC ATGGAAAATA CATAACTGAG |
| (FIG. 20, top) | AATAGAGAAG TTCAGATCAA GGTTAGGAAC AGAGAGACAG |
| | CAGAATATGG GCCAAACAGG ATATCTGTGG TAAGCAGTTC |
| | CTGCCCCGGC TCAGGGCCAA GAACAGATGG TCCCCAGATG |
| | CGGTCCCGCC CTCAGCAGTT TCTAGAGAAC CATCAGATGT |
| | TTCCAGGGTG CCCCAAGGAC CTGAAATGAC CCTGTGCCTT |
| | ATTTGAACTA ACCAATCAGT TCGCTTCTCG CTTCTGTTCG |
| | CGCGCTTCTG CTCCCCGAGC TCAATAAAAG AGCCCACAAC |
| | CCCTCACTCG GCGCGCCAGT CCTCCGATAG ACTGCGTCGC |
| | CCGGGTACCC GTATTCCCAA TAAAGCCTCT TGCTGTTTGC |
| | ATCCGAATCG TGGACTCGCT GATCCTTGGG AGGGTCTCCT |
| | CAGATTGATT GACTGCCCAC CTCGGGGGTC TTTCATTTGG |
| | AGGTTCCACC GAGATTTGGA GACCCCTGCC CAGGGACCAC |
| | CGACCCCCCC GCCGGGAGGT AAGCTGGCCA GCGGTCGTTT |
| | CGTGTCTGTC TCTGTCTTTG TGCGTGTTTG TGCCGGCATC |
| | TAATGTTTGC GCCTGCGTCT GTACTAGTTA GCTAACTAGC |
| | TCTGTATCTG GCGGACCCGT GGTGGAACTG ACGAGTTCTG |
| | AACACCCGGC CGCAACCCTG GGAGACGTCC CAGGGACTTT |
| | GGGGGCCGTT TTTGTGGCCC GACCTGAGGA AGGGAGTCGA |
| | TGTGGAATCC GACCCCGTCA GGATATGTGG TTCTGGTAGG |
| | AGACGAGAAC CTAAAACAGT TCCCGCCTCC GTCTGAATTT |
| | TTGCTTTCGG TTTGGAACCG AAGCCGCGCG TCTTGTCTGC |
| | TGCAGCGCTG CAGCATCGTT CTGTGTTGTC TCTGTCTGAC |
| | TGTGTTTCTG TATTTGTCTG AAAATTAGGG CCAGACTGTT |
| | ACCACTCCCT TAAGTTTGAC CTTAGGTCAC TGGAAAGATG |
| | TCGAGCGGAT CGCTCACAAC CAGTCGGTAG ATGTCAAGAA |
| | GAGACGTTGG GTTACCTTCT GCTCTGCAGA ATGGCCAACC |
| | TTTAACGTCG GATGGCCGCG AGACGGCACC TTTAACCGAG |
| | ACCTCATCAC CCAGGTTAAG ATCAAGGTCT TTTCACCTGG |
| | CCCGCATGGA CACCCAGACC AGGTCCCCTA CATCGTGACC |
| | TGGGAAGCCT TGGCTTTTGA CCCCCCTCCC TGGGTCAAGC |
| | CCTTTGTACA CCCTAAGCCT CCGCCTCCTC TTCCTCCATC |
| | CGCCCCGTCT CTCCCCCTTG AACCTCCTCG TTCGACCCCG |
| | CCTCGATCCT CCCTTTATCC AGCCCTCACT CCTTCTCTAG |
| | GCGCCGGAAT TAGATCTGGT GATAACGAAT TCTACCGGGT |
| | AGGTGAGGCG CTTTTCCCAA GGCAGTCTGG AGCATGCGCT |
| | TTAGCAGCCC CGCTGGGCAC TTGGCGCTAC ACAAGTGGCC |
| | TCTGGCCTCG CACACATTCC ACATCCACCG GTAGGCGCCA |
| | ACCGGCTCCG TTCTTTGGTG GCCCCTTCGC GCCACCTTCT |
| | ACTCCTCCCC TAGTCAGGAA GTTCCCCCCC GCCCCGCAGC |
| | TCGCGTCGTG CAGGACGTGA CAAATGGAAG TAGCACGTCT |
| | CACTAGTCTC GTGCAGATGG ACAGCACCGC TGAGCAATGG |
| | AAGCGGGTAG GCCTTTGGGG CAGCGGCCAA TAGCAGCTTT |
| | GCTCCTTCGC TTTCTGGGCT CAGAGGCTGG GAAGGGGTGG |
| | GTCCGGGGGC GGGCTCAGGG GCGGGCTCAG GGGCGGGGCG |
| | GGCGCCCGAA GGTCCTCCGG AGGCCCGGCA TTCTGCACGC |
| | TTCAAAAGCG CACGTCTGCC GCGCTGTTCT CCTCTTCCTC |
| | ATCTCCGGGC CTTTCGACCT GCAGCCCAAG CTAGGACCGC |
| | GGCCGCACTG GCCGCCACCA TGGGATCCGG CTCCGGAGAG |
| | GGCCGCGGTA GCCTCCTGAC CTGCGGGGAC GTGGAGGAGA |
| | ACCCCGGCCC TATGGTGAGC AAGGGCGAGG AGGATAACAT |
| | GGCCATCATC AAGGAGTTCA TGCGCTTCAA GGTGCACATG |
| | GAGGGCTCCG TGAACGGCCA CGAGTTCGAG ATCGAGGGCG |
| | AGGGCGAGGG CCGCCCCTAC GAGGGCACCC AGACCGCCAA |
| | GCTGAAGGTG ACCAAGGGTG GCCCCCTGCC CTTCGCCTGG |
| | GACATCCTGT CCCCTCAGTT CATGTACGGC TCCAAGGCCT |
| | ACGTGAAGCA CCCCGCCGAC ATCCCCGACT ACTTGAAGCT |
| | GTCCTTCCCC GAGGGCTTCA AGTGGGAGCG CGTGATGAAC |
| | TTCGAGGACG GCGGCGTGGT GACCGTGACC CAGGACTCCT |
| | CCCTGCAGGA CGGCGAGTTC ATCTACAAGG TGAAGCTGCG |
| | CGGCACCAAC TTCCCCTCCG ACGGCCCCGT AATGCAGAAG |
| | AAGACCATGG GCTGGGAGGC CTCCTCCGAG CGGATGTACC |
| | CCGAGGACGG CGCCCTGAAG GGCGAGATCA AGCAGAGGCT |
| | GAAGCTGAAG GACGGCGGCC ACTACGACGC TGAGGTCAAG |
| | ACCACCTACA AGGCCAAGAA GCCCGTGCAG CTGCCCGGCG |
| | CCTACAACGT CAACATCAAG TTGGACATCA CCTCCCACAA |
| | CGAGGACTAC ACCATCGTGG AACAGTACGA ACGCGCCGAG |
| | GGCCGCCACT CCACCGGCGG CATGGACGAG CTGTACAAGT |
| | GACGCCCGCC CCACGACCCG CAGCGCCCGA CCGAAAGGAG |
| | CGCACGACCC CATGCATATA ATTCGATAAT CAACCTCTGG |
| | ATTACAAAAT TTGTGAAAGA TTGACTGGTA TTCTTAACTA |
| | TGTTGCTCCT TTTACGCTAT GTGGATACGC TGCTTTAATG |
| | CCTTTGTATC ATGCTATTGC TTCCCGTATG GCTTTCATTT |
| | TCTCCTCCTT GTATAAATCC TGGTTGCTGT CTCTTTATGA |
| | GGAGTTGTGG CCCGTTGTCA GGCAACGTGG CGTGGTGTGC |
| | ACTGTGTTTG CTGACGCAAC CCCCACTGGT TGGGGCATTG |
| | CCACCACCTG TCAGCTCCTT TCCGGGACTT TCGCTTTCCC |
| | CCTCCCTATT GCCACGGCGG AACTCATCGC CGCCTGCCTT |
| | GCCCGCTGCT GGACAGGGGC TCGGCTGTTG GGCACTGACA |
| | ATTCCGTGGT GTTGTCGGGG AAATCATCGT CCTTTCCTTG |
| | GCTGCTCGCC TGTGTTGCCA CCTGGATTCT GCGCGGGACG |
| | TCCTTCTGCT ACGTCCCTTC GGCCCTCAAT CCAGCGGACC |
| | TTCCTTCCCG CGGCCTGCTG CCGGCTCTGC GGCCTCTTCC |
| | GCGTCTTCGC CTTCGCCCTC AGACGAGTCG GATCTCCCTT |
| | TGGGCCGCCT CCCCGCATCG GGAATTATCG ATAAAATAAA |
| | AGATTTTATT TAGTCTCCAG AAAAAGGGGG GAATGAAAGA |
| | CCCCACCTGT AGGTTTGGCA AGCTAGCTTA AGTAACGCCA |
| | TTTTGCAAGG CATGGAAAAT ACATAACTGA GAATAGAGAA |
| | GTTCAGATCA AGGTTAGGAA CAGAGAGACA GCAGAATATG |
| | GGCCAAACAG GATATCTGTG GTAAGCAGTT CCTGCCCCGG |
| | CTCAGGGCCA AGAACAGATG GTCCCCAGAT GCGGTCCCGC |
| | CCTCAGCAGT TTCTAGAGAA CCATCAGATG TTTCCAGGGT |
| | GCCCCAAGGA CCTGAAATGA CCCTGTGCCT TATTTGAACT |
| | AACCAATCAG TTCGCTTCTC GCTTCTGTTC GCGCGCTTCT |
| | GCTCCCCGAG CTCAATAAAA GAGCCCACAA CCCCTCACTC |
| | GGCGCGCCAG TCCTCCGATA GACTGCGTCG CCCGGGTACC |
| | CGTGTATCCA ATAAACCCTC TTGCAGTTGC ATCCGACTTG |
| | TGGTCTCGCT GTTCCTTGGG AGGGTCTCCT CTGAGTGATT |
| | GACTACCCGT CAGCGGGGGT CTTTCATGGG TAACAGTTTC |
| | TTGAAGTTGG AGAACAACAT TCTGAGGGTA GGAGTCGAAT |
| | ATTAAGTAAT CCTGACTCAA TTAGCCACTG TTTTGAATCC |
| | ACATACTCCA ATACTCCTGA AATAGTTCAT TATGGACAGC |
| | GCAGAAGAGC TGGGGAGAAT TGTGAAATTG TTATCCGCTC |
| | ACAATTCCAC ACAACATACG AGCCGGAAGC ATAAAGTGTA |
| | AAGCCTGGGG TGCCTAATGA GTGAGCTAAC TCACATTAAT |
| | TGCGTTGCGC TCACTGCCCG CTTTCCAGTC GGGAAACCTG |
| | TCGTGCCAGC TGCATTAATG AATCGGCCAA CGCGCGGGGA |
| | GAGGCGGTTT GCGTATTGGG CGCTCTTCCG CTTCCTCGCT |
| | CACTGACTCG CTGCGCTCGG TCGTTCGGCT GCGGCGAGCG |
| | GTATCAGCTC ACTCAAAGGC GGTAATACGG TTATCCACAG |
| | AATCAGGGGA TAACGCAGGA AAGAACATGT GAGCAAAAGG |
| | CCAGCAAAAG GCCAGGAACC GTAAAAAGGC CGCGTTGCTG |
| | GCGTTTTTCC ATAGGCTCCG CCCCCCTGAC GAGCATCACA |
| | AAAATCGACG CTCAAGTCAG AGGTGGCGAA ACCCGACAGG |
| | ACTATAAAGA TACCAGGCGT TTCCCCCTGG AAGCTCCCTC |
| | GTGCGCTCTC CTGTTCCGAC CCTGCCGCTT ACCGGATACC |
| | TGTCCGCCTT TCTCCCTTCG GGAAGCGTGG CGCTTTCTCA |
| | TAGCTCACGC TGTAGGTATC TCAGTTCGGT GTAGGTCGTT |
| | CGCTCCAAGC TGGGCTGTGT GCACGAACCC CCCGTTCAGC |
| | CCGACCGCTG CGCCTTATCC GGTAACTATC GTCTTGAGTC |
| | CAACCCGGTA AGACACGACT TATCGCCACT GGCAGCAGCC |
| | ACTGGTAACA GGATTAGCAG AGCGAGGTAT GTAGGCGGTG |
| | CTACAGAGTT CTTGAAGTGG TGGCCTAACT ACGGCTACAC |
| | TAGAAGGACA GTATTTGGTA TCTGCGCTCT GCTGAAGCCA |
| | GTTACCTTCG GAAAAAGAGT TGGTAGCTCT TGATCCGGCA |
| | AACAAACCAC CGCTGGTAGC GGTGGTTTTT TTGTTTGCAA |
| | GCAGCAGATT ACGCGCAGAA AAAAAGGATC TCAAGAAGAT |
| | CCTTTGATCT TTTCTACGGG GTCTGACGCT CAGTGGAACG |
| | AAAACTCACG TTAAGGGATT TTGGTCATGA GATTATCAAA |
| | AAGGATCTTC ACCTAGATCC TTTTAAATTA AAAATGAAGT |
| | TTTAAATCAA TCTAAAGTAT ATATGAGTAA ACTTGGTCTG |
| | ACAGTTACCA ATGCTTAATC AGTGAGGCAC CTATCTCAGC |
| | GATCTGTCTA TTTCGTTCAT CCATAGTTGC CTGACTCCCC |
| | GTCGTGTAGA TAACTACGAT ACGGGAGGGC TTACCATCTG |
| | GCCCCAGTGC TGCAATGATA CCGCGAGACC CACGCTCACC |
| | GGCTCCAGAT TTATCAGCAA TAAACCAGCC AGCCGGAAGG |
| | GCCGAGCGCA GAAGTGGTCC TGCAACTTTA TCCGCCTCCA |
| | TCCAGTCTAT TAATTGTTGC CGGGAAGCTA GAGTAAGTAG |
| | TTCGCCAGTT AATAGTTTGC GCAACGTTGT TGCCATTGCT |
| | ACAGGCATCG TGGTGTCACG CTCGTCGTTT GGTATGGCTT |
| | CATTCAGCTC CGGTTCCCAA CGATCAAGGC GAGTTACATG |
| | ATCCCCCATG TTGTGCAAAA AAGCGGTTAG CTCCTTCGGT |
| | CCTCCGATCG TTGTCAGAAG TAAGTTGGCC GCAGTGTTAT |
| | CACTCATGGT TATGGCAGCA CTGCATAATT CTCTTACTGT |
| | CATGCCATCC GTAAGATGCT TTTCTGTGAC TGGTGAGTAC |
| | TCAACCAAGT CATTCTGAGA ATAGTGTATG CGGCGACCGA |
| | GTTGCTCTTG CCCGGCGTCA ATACGGGATA ATACCGCGCC |
| | ACATAGCAGA ACTTTAAAAG TGCTCATCAT TGGAAAACGT |
| | TCTTCGGGGC GAAAACTCTC AAGGATCTTA CCGCTGTTGA |
| | GATCCAGTTC GATGTAACCC ACTCGTGCAC CCAACTGATC |
| | TTCAGCATCT TTTACTTTCA CCAGCGTTTC TGGGTGAGCA |
| | AAAACAGGAA GGCAAAATGC CGCAAAAAAG |
| | GGAATAAGGG CGACACGGAA ATGTTGAATA CTCATACTCT |
| | TCCTTTTTCA ATATTATTGA AGCATTTATC AGGGTTATTG |
| | TCTCATGAGC GGATACATAT TTGAATGTAT TTAGAAAAAT |
| | AAACAAATAG GGGTTCCGCG CACATTTCCC CGAAAAGTGC |
| | CACCTGACGT CTAAGAAACC ATTATTATCA TGACATTAAC |
| | CTATAAAAAT AGGCGTATCA CGAGGCCCTT TCGTCTCGCG |
| | CGTTTCGGTG ATGACGGTGA AAACCTCTGA CACATGCAGC |
| | TCCCGGAGAC GGTCACAGCT TGTCTGTAAG CGGATGCCGG |
| | GAGCAGACAA GCCCGTCAGG GCGCGTCAGC GGGTGTTGGC |
| | GGGTGTCGGG GCTGGCTTAA CTATGCGGCA TCAGAGCAGA |
| | TTGTACTGAG AGTGCACCAT ATGCGGTGTG AAATACCGCA |
| | CAGATGCGTA AGGAGAAAAT ACCGCATCAG GCGCCATTCG |
| | CCATTCAGGC TGCGCAACTG TTGGGAAGGG CGATCGGTGC |
| | GGGCCTCTTC GCTATTACGC CAGCTGGCGA AAGGGGGATG |
| | TGCTGCAAGG CGATTAAGTT GGGTAACGCC AGGGTTTTCC |
| | CAGTCACGAC GTTGTAAAAC GACGGCGCAA GGAATGGTGC |
| | ATGCAAGGAG ATGGCGCCCA ACAGTCCCCC GGCCACGGGG |
| | CCTGCCACCA TACCCACGCC GAAACAAGCG CTCATGAGCC |
| | CGAAGTGGCG AGCCCGATCT TCCCCATCGG TGATGTCGGC |
| | GATATAGGCG CCAGCAACCG CACCTGTGGC GCCGGTGATG |
| | CCGGCCACGA TGCGTCCGGC GTAGAGGCGA TTAGTCCAAT |
| | TTGTTAAAGA CAGGATATCA GTGGTCCAGG CTCTAGTTTT |
| | GACTCAACAA TATCACCAGC TGAAGCCTAT AGAGTACGAG |
| | CCATAGATAA AATAAAAGAT TTTATTTAGT CTCCAGAAAA |
| | AGGGGGG |
| | (SEQ ID NO: 26) |
| MSCV_PGK-2A- | AATGAAAGAC CCCACCTGTA GGTTTGGCAA GCTAGCTTAA |
| GFP vector | GTAACGCCAT TTTGCAAGGC ATGGAAAATA CATAACTGAG |
| (FIG. 20, | AATAGAGAAG TTCAGATCAA GGTTAGGAAC AGAGAGACAG |
| bottom) | CAGAATATGG GCCAAACAGG ATATCTGTGG TAAGCAGTTC |
| | CTGCCCCGGC TCAGGGCCAA GAACAGATGG TCCCCAGATG |
| | CGGTCCCGCC CTCAGCAGTT TCTAGAGAAC CATCAGATGT |
| | TTCCAGGGTG CCCCAAGGAC CTGAAATGAC CCTGTGCCTT |
| | ATTTGAACTA ACCAATCAGT TCGCTTCTCG CTTCTGTTCG |
| | CGCGCTTCTG CTCCCCGAGC TCAATAAAAG AGCCCACAAC |
| | CCCTCACTCG GCGCGCCAGT CCTCCGATAG ACTGCGTCGC |
| | CCGGGTACCC GTATTCCCAA TAAAGCCTCT TGCTGTTTGC |
| | ATCCGAATCG TGGACTCGCT GATCCTTGGG AGGGTCTCCT |
| | CAGATTGATT GACTGCCCAC CTCGGGGGTC TTTCATTTGG |
| | AGGTTCCACC GAGATTTGGA GACCCCTGCC CAGGGACCAC |
| | CGACCCCCCC GCCGGGAGGT AAGCTGGCCA GCGGTCGTTT |
| | CGTGTCTGTC TCTGTCTTTG TGCGTGTTTG TGCCGGCATC |
| | TAATGTTTGC GCCTGCGTCT GTACTAGTTA GCTAACTAGC |
| | TCTGTATCTG GCGGACCCGT GGTGGAACTG ACGAGTTCTG |
| | AACACCCGGC CGCAACCCTG GGAGACGTCC CAGGGACTTT |
| | GGGGGCCGTT TTTGTGGCCC GACCTGAGGA AGGGAGTCGA |
| | TGTGGAATCC GACCCCGTCA GGATATGTGG TTCTGGTAGG |
| | AGACGAGAAC CTAAAACAGT TCCCGCCTCC GTCTGAATTT |
| | TTGCTTTCGG TTTGGAACCG AAGCCGCGCG TCTTGTCTGC |
| | TGCAGCGCTG CAGCATCGTT CTGTGTTGTC TCTGTCTGAC |
| | TGTGTTTCTG TATTTGTCTG AAAATTAGGG CCAGACTGTT |
| | ACCACTCCCT TAAGTTTGAC CTTAGGTCAC TGGAAAGATG |
| | TCGAGCGGAT CGCTCACAAC CAGTCGGTAG ATGTCAAGAA |
| | GAGACGTTGG GTTACCTTCT GCTCTGCAGA ATGGCCAACC |
| | TTTAACGTCG GATGGCCGCG AGACGGCACC TTTAACCGAG |
| | ACCTCATCAC CCAGGTTAAG ATCAAGGTCT TTTCACCTGG |
| | CCCGCATGGA CACCCAGACC AGGTCCCCTA CATCGTGACC |
| | TGGGAAGCCT TGGCTTTTGA CCCCCCTCCC TGGGTCAAGC |
| | CCTTTGTACA CCCTAAGCCT CCGCCTCCTC TTCCTCCATC |
| | CGCCCCGTCT CTCCCCCTTG AACCTCCTCG TTCGACCCCG |
| | CCTCGATCCT CCCTTTATCC AGCCCTCACT CCTTCTCTAG |
| | GCGCCGGAAT TAGATCTGGT GATAACGAAT TCTACCGGGT |
| | AGGTGAGGCG CTTTTCCCAA GGCAGTCTGG AGCATGCGCT |
| | TTAGCAGCCC CGCTGGGCAC TTGGCGCTAC ACAAGTGGCC |
| | TCTGGCCTCG CACACATTCC ACATCCACCG GTAGGCGCCA |
| | ACCGGCTCCG TTCTTTGGTG GCCCCTTCGC GCCACCTTCT |
| | ACTCCTCCCC TAGTCAGGAA GTTCCCCCCC GCCCCGCAGC |
| | TCGCGTCGTG CAGGACGTGA CAAATGGAAG TAGCACGTCT |
| | CACTAGTCTC GTGCAGATGG ACAGCACCGC TGAGCAATGG |
| | AAGCGGGTAG GCCTTTGGGG CAGCGGCCAA TAGCAGCTTT |
| | GCTCCTTCGC TTTCTGGGCT CAGAGGCTGG GAAGGGGTGG |
| | GTCCGGGGGC GGGCTCAGGG GCGGGCTCAG GGGCGGGGCG |
| | GGCGCCCGAA GGTCCTCCGG AGGCCCGGCA TTCTGCACGC |
| | TTCAAAAGCG CACGTCTGCC GCGCTGTTCT CCTCTTCCTC |
| | ATCTCCGGGC CTTTCGACCT GCAGCCCAAG CTAGGACCGC |
| | GGCCGCACTG GCCGCCACCA TGGGATCCGG CTCCGGAGAG |
| | GGCCGCGGTA GCCTCCTGAC CTGCGGGGAC GTGGAGGAGA |
| | ACCCCGGCCC TATGGTGAGC AAGGGCGAGG AGCTGTTCAC |
| | CGGGGTGGTG CCCATCCTGG TCGAGCTGGA CGGCGACGTA |
| | AACGGCCACA AGTTCAGCGT GTCCGGCGAG GGCGAGGGCG |
| | ATGCCACCTA CGGCAAGCTG ACCCTGAAGT TCATCTGCAC |
| | CACCGGCAAG CTGCCCGTGC CCTGGCCCAC CCTCGTGACC |
| | ACCTTCACCT ACGGCGTGCA GTGCTTCAGC CGCTACCCCG |
| | ACCACATGAA GCAGCACGAC TTCTTCAAGT CCGCCATGCC |
| | CGAAGGCTAC GTCCAGGAGC GCACCATCTC TTTCAAGGAC |
| | GACGGCAACT ACAAGACCCG CGCCGAGGTG AAGTTCGAGG |
| | GCGACACCCT GGTGAACCGC ATCGAGCTGA AGGGCATCGA |
| | CTTCAAGGAG GACGGCAACA TCCTGGGGCA CAAGCTGGAG |
| | TACAACTACA ACAGCCACAA CGTCTATATC ACGGCCGACA |
| | AGCAGAAGAA CGGCATCAAG GCTAACTTCA AGATCCGCCA |
| | CAACATCGAG GACGGCAGCG TGCAGCTCGC CGACCACTAC |
| | CAGCAGAACA CCCCCATCGG CGACGGCCCC GTGCTGCTGC |
| | CCGACAACCA CTACCTGAGC ACCCAGTCCG CCCTGAGCAA |
| | AGACCCCAAC GAGAAGCGCG ATCACATGGT CCTGCTGGAG |
| | TTCGTGACCG CCGCCGGGAT CACTCTCGGC ATGGACGAGC |
| | TGTACAAGTG ACGCCCGCCC CACGACCCGC AGCGCCCGAC |
| | CGAAAGGAGC GCACGACCCC ATGCATATAA TTCGATAATC |
| | AACCTCTGGA TTACAAAATT TGTGAAAGAT TGACTGGTAT |
| | TCTTAACTAT GTTGCTCCTT TTACGCTATG TGGATACGCT |
| | GCTTTAATGC CTTTGTATCA TGCTATTGCT TCCCGTATGG |
| | CTTTCATTTT CTCCTCCTTG TATAAATCCT GGTTGCTGTC |
| | TCTTTATGAG GAGTTGTGGC CCGTTGTCAG GCAACGTGGC |
| | GTGGTGTGCA CTGTGTTTGC TGACGCAACC CCCACTGGTT |
| | GGGGCATTGC CACCACCTGT CAGCTCCTTT CCGGGACTTT |
| | CGCTTTCCCC CTCCCTATTG CCACGGCGGA ACTCATCGCC |
| | GCCTGCCTTG CCCGCTGCTG GACAGGGGCT CGGCTGTTGG |
| | GCACTGACAA TTCCGTGGTG TTGTCGGGGA AATCATCGTC |
| | CTTTCCTTGG CTGCTCGCCT GTGTTGCCAC CTGGATTCTG |
| | CGCGGGACGT CCTTCTGCTA CGTCCCTTCG GCCCTCAATC |
| | CAGCGGACCT TCCTTCCCGC GGCCTGCTGC CGGCTCTGCG |
| | GCCTCTTCCG CGTCTTCGCC TTCGCCCTCA GACGAGTCGG |
| | ATCTCCCTTT GGGCCGCCTC CCCGCATCGG GAATTATCGA |
| | TAAAATAAAA GATTTTATTT AGTCTCCAGA AAAAGGGGGG |
| | AATGAAAGAC CCCACCTGTA GGTTTGGCAA GCTAGCTTAA |
| | GTAACGCCAT TTTGCAAGGC ATGGAAAATA CATAACTGAG |
| | AATAGAGAAG TTCAGATCAA GGTTAGGAAC AGAGAGACAG |
| | CAGAATATGG GCCAAACAGG ATATCTGTGG TAAGCAGTTC |
| | CTGCCCCGGC TCAGGGCCAA GAACAGATGG TCCCCAGATG |
| | CGGTCCCGCC CTCAGCAGTT TCTAGAGAAC CATCAGATGT |
| | TTCCAGGGTG CCCCAAGGAC CTGAAATGAC CCTGTGCCTT |
| | ATTTGAACTA ACCAATCAGT TCGCTTCTCG CTTCTGTTCG |
| | CGCGCTTCTG CTCCCCGAGC TCAATAAAAG AGCCCACAAC |
| | CCCTCACTCG GCGCGCCAGT CCTCCGATAG ACTGCGTCGC |
| | CCGGGTACCC GTGTATCCAA TAAACCCTCT TGCAGTTGCA |
| | TCCGACTTGT GGTCTCGCTG TTCCTTGGGA GGGTCTCCTC |
| | TGAGTGATTG ACTACCCGTC AGCGGGGGTC TTTCATGGGT |
| | AACAGTTTCT TGAAGTTGGA GAACAACATT CTGAGGGTAG |
| | GAGTCGAATA TTAAGTAATC CTGACTCAAT TAGCCACTGT |
| | TTTGAATCCA CATACTCCAA TACTCCTGAA ATAGTTCATT |
| | ATGGACAGCG CAGAAGAGCT GGGGAGAATT GTGAAATTGT |
| | TATCCGCTCA CAATTCCACA CAACATACGA GCCGGAAGCA |
| | TAAAGTGTAA AGCCTGGGGT GCCTAATGAG TGAGCTAACT |
| | CACATTAATT GCGTTGCGCT CACTGCCCGC TTTCCAGTCG |
| | GGAAACCTGT CGTGCCAGCT GCATTAATGA ATCGGCCAAC |
| | GCGCGGGGAG AGGCGGTTTG CGTATTGGGC GCTCTTCCGC |
| | TTCCTCGCTC ACTGACTCGC TGCGCTCGGT CGTTCGGCTG |
| | CGGCGAGCGG TATCAGCTCA CTCAAAGGCG GTAATACGGT |
| | TATCCACAGA ATCAGGGGAT AACGCAGGAA AGAACATGTG |
| | AGCAAAAGGC CAGCAAAAGG CCAGGAACCG TAAAAAGGCC |
| | GCGTTGCTGG CGTTTTTCCA TAGGCTCCGC CCCCCTGACG |
| | AGCATCACAA AAATCGACGC TCAAGTCAGA GGTGGCGAAA |
| | CCCGACAGGA CTATAAAGAT ACCAGGCGTT TCCCCCTGGA |
| | AGCTCCCTCG TGCGCTCTCC TGTTCCGACC CTGCCGCTTA |
| | CCGGATACCT GTCCGCCTTT CTCCCTTCGG GAAGCGTGGC |
| | GCTTTCTCAT AGCTCACGCT GTAGGTATCT CAGTTCGGTG |
| | TAGGTCGTTC GCTCCAAGCT GGGCTGTGTG CACGAACCCC |
| | CCGTTCAGCC CGACCGCTGC GCCTTATCCG GTAACTATCG |
| | TCTTGAGTCC AACCCGGTAA GACACGACTT ATCGCCACTG |
| | GCAGCAGCCA CTGGTAACAG GATTAGCAGA GCGAGGTATG |
| | TAGGCGGTGC TACAGAGTTC TTGAAGTGGT GGCCTAACTA |
| | CGGCTACACT AGAAGGACAG TATTTGGTAT CTGCGCTCTG |
| | CTGAAGCCAG TTACCTTCGG AAAAAGAGTT GGTAGCTCTT |
| | GATCCGGCAA ACAAACCACC GCTGGTAGCG GTGGTTTTTT |
| | TGTTTGCAAG CAGCAGATTA CGCGCAGAAA AAAAGGATCT |
| | CAAGAAGATC CTTTGATCTT TTCTACGGGG TCTGACGCTC |
| | AGTGGAACGA AAACTCACGT TAAGGGATTT TGGTCATGAG |
| | ATTATCAAAA AGGATCTTCA CCTAGATCCT TTTAAATTAA |
| | AAATGAAGTT TTAAATCAAT CTAAAGTATA TATGAGTAAA |
| | CTTGGTCTGA CAGTTACCAA TGCTTAATCA GTGAGGCACC |
| | TATCTCAGCG ATCTGTCTAT TTCGTTCATC CATAGTTGCC |
| | TGACTCCCCG TCGTGTAGAT AACTACGATA CGGGAGGGCT |
| | TACCATCTGG CCCCAGTGCT GCAATGATAC CGCGAGACCC |
| | ACGCTCACCG GCTCCAGATT TATCAGCAAT AAACCAGCCA |
| | GCCGGAAGGG CCGAGCGCAG AAGTGGTCCT GCAACTTTAT |
| | CCGCCTCCAT CCAGTCTATT AATTGTTGCC GGGAAGCTAG |
| | AGTAAGTAGT TCGCCAGTTA ATAGTTTGCG CAACGTTGTT |
| | GCCATTGCTA CAGGCATCGT GGTGTCACGC TCGTCGTTTG |
| | GTATGGCTTC ATTCAGCTCC GGTTCCCAAC GATCAAGGCG |
| | AGTTACATGA TCCCCCATGT TGTGCAAAAA AGCGGTTAGC |
| | TCCTTCGGTC CTCCGATCGT TGTCAGAAGT AAGTTGGCCG |
| | CAGTGTTATC ACTCATGGTT ATGGCAGCAC TGCATAATTC |
| | TCTTACTGTC ATGCCATCCG TAAGATGCTT TTCTGTGACT |
| | GGTGAGTACT CAACCAAGTC ATTCTGAGAA TAGTGTATGC |
| | GGCGACCGAG TTGCTCTTGC CCGGCGTCAA TACGGGATAA |
| | TACCGCGCCA CATAGCAGAA CTTTAAAAGT GCTCATCATT |
| | GGAAAACGTT CTTCGGGGCG AAAACTCTCA AGGATCTTAC |
| | CGCTGTTGAG ATCCAGTTCG ATGTAACCCA CTCGTGCACC |
| | CAACTGATCT TCAGCATCTT TTACTTTCAC CAGCGTTTCT |
| | GGGTGAGCAA AAACAGGAAG GCAAAATGCC |
| | GCAAAAAAGG GAATAAGGGC GACACGGAAA TGTTGAATAC |
| | TCATACTCTT CCTTTTTCAA TATTATTGAA GCATTTATCA |
| | GGGTTATTGT CTCATGAGCG GATACATATT TGAATGTATT |
| | TAGAAAAATA AACAAATAGG GGTTCCGCGC ACATTTCCCC |
| | GAAAAGTGCC ACCTGACGTC TAAGAAACCA TTATTATCAT |
| | GACATTAACC TATAAAAATA GGCGTATCAC GAGGCCCTTT |
| | CGTCTCGCGC GTTTCGGTGA TGACGGTGAA AACCTCTGAC |
| | ACATGCAGCT CCCGGAGACG GTCACAGCTT GTCTGTAAGC |
| | GGATGCCGGG AGCAGACAAG CCCGTCAGGG CGCGTCAGCG |
| | GGTGTTGGCG GGTGTCGGGG CTGGCTTAAC TATGCGGCAT |
| | CAGAGCAGAT TGTACTGAGA GTGCACCATA TGCGGTGTGA |
| | AATACCGCAC AGATGCGTAA GGAGAAAATA CCGCATCAGG |
| | CGCCATTCGC CATTCAGGCT GCGCAACTGT TGGGAAGGGC |
| | GATCGGTGCG GGCCTCTTCG CTATTACGCC AGCTGGCGAA |
| | AGGGGGATGT GCTGCAAGGC GATTAAGTTG GGTAACGCCA |
| | GGGTTTTCCC AGTCACGACG TTGTAAAACG ACGGCGCAAG |
| | GAATGGTGCA TGCAAGGAGA TGGCGCCCAA CAGTCCCCCG |
| | GCCACGGGGC CTGCCACCAT ACCCACGCCG AAACAAGCGC |
| | TCATGAGCCC GAAGTGGCGA GCCCGATCTT CCCCATCGGT |
| | GATGTCGGCG ATATAGGCGC CAGCAACCGC ACCTGTGGCG |
| | CCGGTGATGC CGGCCACGAT GCGTCCGGCG TAGAGGCGAT |
| | TAGTCCAATT TGTTAAAGAC AGGATATCAG TGGTCCAGGC |
| | TCTAGTTTTG ACTCAACAAT ATCACCAGCT GAAGCCTATA |
| | GAGTACGAGC CATAGATAAA ATAAAAGATT TTATTTAGTC |
| | TCCAGAAAAA GGGGGG |
| | (SEQ ID NO: 27) |
| MSCV_PGK-cdt- | AATGAAAGAC CCCACCTGTA GGTTTGGCAA GCTAGCTTAA |
| 1-2A-mCherry | GTAACGCCAT TTTGCAAGGC ATGGAAAATA CATAACTGAG |
| (FIG. 21, top) | AATAGAGAAG TTCAGATCAA GGTTAGGAAC AGAGAGACAG |
| | CAGAATATGG GCCAAACAGG ATATCTGTGG TAAGCAGTTC |
| | CTGCCCCGGC TCAGGGCCAA GAACAGATGG TCCCCAGATG |
| | CGGTCCCGCC CTCAGCAGTT TCTAGAGAAC CATCAGATGT |
| | TTCCAGGGTG CCCCAAGGAC CTGAAATGAC CCTGTGCCTT |
| | ATTTGAACTA ACCAATCAGT TCGCTTCTCG CTTCTGTTCG |
| | CGCGCTTCTG CTCCCCGAGC TCAATAAAAG AGCCCACAAC |
| | CCCTCACTCG GCGCGCCAGT CCTCCGATAG ACTGCGTCGC |
| | CCGGGTACCC GTATTCCCAA TAAAGCCTCT TGCTGTTTGC |
| | ATCCGAATCG TGGACTCGCT GATCCTTGGG AGGGTCTCCT |
| | CAGATTGATT GACTGCCCAC CTCGGGGGTC TTTCATTTGG |
| | AGGTTCCACC GAGATTTGGA GACCCCTGCC CAGGGACCAC |
| | CGACCCCCCC GCCGGGAGGT AAGCTGGCCA GCGGTCGTTT |
| | CGTGTCTGTC TCTGTCTTTG TGCGTGTTTG TGCCGGCATC |
| | TAATGTTTGC GCCTGCGTCT GTACTAGTTA GCTAACTAGC |
| | TCTGTATCTG GCGGACCCGT GGTGGAACTG ACGAGTTCTG |
| | AACACCCGGC CGCAACCCTG GGAGACGTCC CAGGGACTTT |
| | GGGGGCCGTT TTTGTGGCCC GACCTGAGGA AGGGAGTCGA |
| | TGTGGAATCC GACCCCGTCA GGATATGTGG TTCTGGTAGG |
| | AGACGAGAAC CTAAAACAGT TCCCGCCTCC GTCTGAATTT |
| | TTGCTTTCGG TTTGGAACCG AAGCCGCGCG TCTTGTCTGC |
| | TGCAGCGCTG CAGCATCGTT CTGTGTTGTC TCTGTCTGAC |
| | TGTGTTTCTG TATTTGTCTG AAAATTAGGG CCAGACTGTT |
| | ACCACTCCCT TAAGTTTGAC CTTAGGTCAC TGGAAAGATG |
| | TCGAGCGGAT CGCTCACAAC CAGTCGGTAG ATGTCAAGAA |
| | GAGACGTTGG GTTACCTTCT GCTCTGCAGA ATGGCCAACC |
| | TTTAACGTCG GATGGCCGCG AGACGGCACC TTTAACCGAG |
| | ACCTCATCAC CCAGGTTAAG ATCAAGGTCT TTTCACCTGG |
| | CCCGCATGGA CACCCAGACC AGGTCCCCTA CATCGTGACC |
| | TGGGAAGCCT TGGCTTTTGA CCCCCCTCCC TGGGTCAAGC |
| | CCTTTGTACA CCCTAAGCCT CCGCCTCCTC TTCCTCCATC |
| | CGCCCCGTCT CTCCCCCTTG AACCTCCTCG TTCGACCCCG |
| | CCTCGATCCT CCCTTTATCC AGCCCTCACT CCTTCTCTAG |
| | GCGCCGGAAT TAGATCTGGT GATAACGAAT TCTACCGGGT |
| | AGGTGAGGCG CTTTTCCCAA GGCAGTCTGG AGCATGCGCT |
| | TTAGCAGCCC CGCTGGGCAC TTGGCGCTAC ACAAGTGGCC |
| | TCTGGCCTCG CACACATTCC ACATCCACCG GTAGGCGCCA |
| | ACCGGCTCCG TTCTTTGGTG GCCCCTTCGC GCCACCTTCT |
| | ACTCCTCCCC TAGTCAGGAA GTTCCCCCCC GCCCCGCAGC |
| | TCGCGTCGTG CAGGACGTGA CAAATGGAAG TAGCACGTCT |
| | CACTAGTCTC GTGCAGATGG ACAGCACCGC TGAGCAATGG |
| | AAGCGGGTAG GCCTTTGGGG CAGCGGCCAA TAGCAGCTTT |
| | GCTCCTTCGC TTTCTGGGCT CAGAGGCTGG GAAGGGGTGG |
| | GTCCGGGGGC GGGCTCAGGG GCGGGCTCAG GGGCGGGGCG |
| | GGCGCCCGAA GGTCCTCCGG AGGCCCGGCA TTCTGCACGC |
| | TTCAAAAGCG CACGTCTGCC GCGCTGTTCT CCTCTTCCTC |
| | ATCTCCGGGC CTTTCGACCT GCAGCCCAAG CTAGGACCGC |
| | GCCGCCACCA TGGCGTCATC TCACGGTTCT CACGACGGGG |
| | CCTCCACCGA GAAACATCTC GCTACTCATG ACATCGCTCC |
| | AACACATGAT GCCATAAAGA TCGTGCCCAA GGGTCACGGA |
| | CAGACAGCCA CAAAGCCTGG GGCTCAGGAA AAGGAAGTTA |
| | GAAATGCAGC CCTGTTCGCT GCTATTAAAG AAAGTAACAT |
| | CAAACCGTGG AGTAAGGAAA GCATCCACCT GTATTTCGCA |
| | ATATTTGTGG CTTTCTGCTG CGCCTGTGCC AATGGCTATG |
| | ACGGATCTTT GATGACAGGA ATAATTGCTA TGGACAAGTT |
| | CCAGAACCAG TTCCACACTG GGGACACCGG CCCCAAAGTC |
| | TCCGTGATCT TTTCTTTATA CACCGTTGGT GCTATGGTAG |
| | GTGCCCCCTT TGCTGCGATA CTGAGTGACA GATTTGGTAG |
| | GAAGAAAGGT ATGTTTATTG GGGGCATTTT TATCATAGTC |
| | GGGTCTATTA TTGTGGCATC CTCCAGCAAA CTGGCTCAAT |
| | TTGTCGTGGG GCGGTTCGTA TTGGGCCTGG GGATTGCTAT |
| | TATGACAGTT GCAGCACCTG CATACAGCAT TGAGATCGCT |
| | CCGCCACACT GGCGGGGACG ATGTACAGGA TTCTACAACT |
| | GTGGGTGGTT TGGAGGCTCC ATCCCAGCCG CCTGCATCAC |
| | CTATGGCTGC TACTTCATCA AGAGCAACTG GAGCTGGCGC |
| | ATCCCCCTCA TCCTCCAAGC CTTCACCTGC CTGATTGTTA |
| | TGTCAAGCGT CTTCTTTCTC CCTGAGTCAC CACGCTTCCT |
| | GTTTGCCAAC GGGCGTGATG CAGAGGCCGT AGCCTTTCTG |
| | GTGAAATACC ACGGGAACGG AGACCCAAAT TCAAAACTTG |
| | TGCTGCTCGA GACAGAAGAA ATGCGTGACG GCATCAGGAC |
| | AGATGGTGTT GATAAAGTGT GGTGGGACTA CCGGCCTCTT |
| | TTTATGACGC ACTCCGGACG CTGGCGAATG GCACAGGTAT |
| | TGATGATCTC CATTTTCGGG CAATTCTCTG GAAACGGACT |
| | AGGATATTTT AACACAGTCA TCTTTAAGAA TATTGGAGTC |
| | ACATCAACCA GTCAGCAGTT GGCGTATAAC ATTCTGAACA |
| | GCGTTATTTC AGCGATCGGC GCTTTAACGG CTGTTTCAAT |
| | GACAGATCGA ATGCCCAGGA GAGCTGTGCT TATCATCGGG |
| | ACTTTTATGT GTGCTGCTGC GCTGGCCACG AATAGTGGCC |
| | TGTCAGCCAC TTTGGATAAG CAGACCCAGC GTGGTACTCA |
| | GATCAACCTC AACCAGGGTA TGAATGAGCA GGACGCCAAG |
| | GACAACGCCT ATCTGCACGT GGACAGCAAC TATGCTAAAG |
| | GCGCGTTGGC AGCCTACTTT CTCTTCAATG TCATCTTCAG |
| | CTTTACCTAC ACACCTCTGC AGGGCGTGAT TCCTACAGAA |
| | GCTTTAGAAA CCACCATCCG AGGCAAAGGA CTCGCTTTGT |
| | CTGGTTTCAT AGTGAATGCT ATGGGATTTA TCAATCAGTT |
| | TGCAGGGCCC ATTGCACTTC ACAACATCGG CTACAAGTAC |
| | ATCTTCGTCT TTGTTGGCTG GGATCTTATT GAAACTGTGG |
| | CCTGGTACTT CTTCGGAGTG GAGTCTCAAG GTCGGACTCT |
| | AGAACAGCTG GAGTGGGTGT ATGACCAGCC AAACCCAGTG |
| | AAGGCATCGC TGAAAGTAGA GAAGGTGGTG GTACAAGCGG |
| | ACGGTCATGT CAGTGAAGCA ATAGTCGCAT ACCCATACGA |
| | TGTTCCAGAT TACGCTGGAT CCGGCTCCGG AGAGGGCCGC |
| | GGTAGCCTCC TGACCTGCGG GGACGTGGAG GAGAACCCCG |
| | GCCCTATGGT GAGCAAGGGC GAGGAGGATA ACATGGCCAT |
| | CATCAAGGAG TTCATGCGCT TCAAGGTGCA CATGGAGGGC |
| | TCCGTGAACG GCCACGAGTT CGAGATCGAG GGCGAGGGCG |
| | AGGGCCGCCC CTACGAGGGC ACCCAGACCG CCAAGCTGAA |
| | GGTGACCAAG GGTGGCCCCC TGCCCTTCGC CTGGGACATC |
| | CTGTCCCCTC AGTTCATGTA CGGCTCCAAG GCCTACGTGA |
| | AGCACCCCGC CGACATCCCC GACTACTTGA AGCTGTCCTT |
| | CCCCGAGGGC TTCAAGTGGG AGCGCGTGAT GAACTTCGAG |
| | GACGGCGGCG TGGTGACCGT GACCCAGGAC TCCTCCCTGC |
| | AGGACGGCGA GTTCATCTAC AAGGTGAAGC TGCGCGGCAC |
| | CAACTTCCCC TCCGACGGCC CCGTAATGCA GAAGAAGACC |
| | ATGGGCTGGG AGGCCTCCTC CGAGCGGATG TACCCCGAGG |
| | ACGGCGCCCT GAAGGGCGAG ATCAAGCAGA GGCTGAAGCT |
| | GAAGGACGGC GGCCACTACG ACGCTGAGGT CAAGACCACC |
| | TACAAGGCCA AGAAGCCCGT GCAGCTGCCC GGCGCCTACA |
| | ACGTCAACAT CAAGTTGGAC ATCACCTCCC ACAACGAGGA |
| | CTACACCATC GTGGAACAGT ACGAACGCGC CGAGGGCCGC |
| | CACTCCACCG GCGGCATGGA CGAGCTGTAC AAGTGACGCC |
| | CGCCCCACGA CCCGCAGCGC CCGACCGAAA GGAGCGCACG |
| | ACCCCATGCA TATAATTCGA TAATCAACCT CTGGATTACA |
| | AAATTTGTGA AAGATTGACT GGTATTCTTA ACTATGTTGC |
| | TCCTTTTACG CTATGTGGAT ACGCTGCTTT AATGCCTTTG |
| | TATCATGCTA TTGCTTCCCG TATGGCTTTC ATTTTCTCCT |
| | CCTTGTATAA ATCCTGGTTG CTGTCTCTTT ATGAGGAGTT |
| | GTGGCCCGTT GTCAGGCAAC GTGGCGTGGT GTGCACTGTG |
| | TTTGCTGACG CAACCCCCAC TGGTTGGGGC ATTGCCACCA |
| | CCTGTCAGCT CCTTTCCGGG ACTTTCGCTT TCCCCCTCCC |
| | TATTGCCACG GCGGAACTCA TCGCCGCCTG CCTTGCCCGC |
| | TGCTGGACAG GGGCTCGGCT GTTGGGCACT GACAATTCCG |
| | TGGTGTTGTC GGGGAAATCA TCGTCCTTTC CTTGGCTGCT |
| | CGCCTGTGTT GCCACCTGGA TTCTGCGCGG GACGTCCTTC |
| | TGCTACGTCC CTTCGGCCCT CAATCCAGCG GACCTTCCTT |
| | CCCGCGGCCT GCTGCCGGCT CTGCGGCCTC TTCCGCGTCT |
| | TCGCCTTCGC CCTCAGACGA GTCGGATCTC CCTTTGGGCC |
| | GCCTCCCCGC ATCGGGAATT ATCGATAAAA TAAAAGATTT |
| | TATTTAGTCT CCAGAAAAAG GGGGGAATGA AAGACCCCAC |
| | CTGTAGGTTT GGCAAGCTAG CTTAAGTAAC GCCATTTTGC |
| | AAGGCATGGA AAATACATAA CTGAGAATAG AGAAGTTCAG |
| | ATCAAGGTTA GGAACAGAGA GACAGCAGAA TATGGGCCAA |
| | ACAGGATATC TGTGGTAAGC AGTTCCTGCC CCGGCTCAGG |
| | GCCAAGAACA GATGGTCCCC AGATGCGGTC CCGCCCTCAG |
| | CAGTTTCTAG AGAACCATCA GATGTTTCCA GGGTGCCCCA |
| | AGGACCTGAA ATGACCCTGT GCCTTATTTG AACTAACCAA |
| | TCAGTTCGCT TCTCGCTTCT GTTCGCGCGC TTCTGCTCCC |
| | CGAGCTCAAT AAAAGAGCCC ACAACCCCTC ACTCGGCGCG |
| | CCAGTCCTCC GATAGACTGC GTCGCCCGGG TACCCGTGTA |
| | TCCAATAAAC CCTCTTGCAG TTGCATCCGA CTTGTGGTCT |
| | CGCTGTTCCT TGGGAGGGTC TCCTCTGAGT GATTGACTAC |
| | CCGTCAGCGG GGGTCTTTCA TGGGTAACAG TTTCTTGAAG |
| | TTGGAGAACA ACATTCTGAG GGTAGGAGTC GAATATTAAG |
| | TAATCCTGAC TCAATTAGCC ACTGTTTTGA ATCCACATAC |
| | TCCAATACTC CTGAAATAGT TCATTATGGA CAGCGCAGAA |
| | AGAGCTGGGG AGAATTGTGA AATTGTTATC CGCTCACAAT |
| | TCCACACAAC ATACGAGCCG GAAGCATAAA GTGTAAAGCC |
| | TGGGGTGCCT AATGAGTGAG CTAACTCACA TTAATTGCGT |
| | TGCGCTCACT GCCCGCTTTC CAGTCGGGAA ACCTGTCGTG |
| | CCAGCTGCAT TAATGAATCG GCCAACGCGC GGGGAGAGGC |
| | GGTTTGCGTA TTGGGCGCTC TTCCGCTTCC TCGCTCACTG |
| | ACTCGCTGCG CTCGGTCGTT CGGCTGCGGC GAGCGGTATC |
| | AGCTCACTCA AAGGCGGTAA TACGGTTATC CACAGAATCA |
| | GGGGATAACG CAGGAAAGAA CATGTGAGCA AAAGGCCAGC |
| | AAAAGGCCAG GAACCGTAAA AAGGCCGCGT TGCTGGCGTT |
| | TTTCCATAGG CTCCGCCCCC CTGACGAGCA TCACAAAAAT |
| | CGACGCTCAA GTGAGAGGTG GCGAAACCCG ACAGGACTAT |
| | AAAGATACCA GGCGTTTCCC CCTGGAAGCT CCCTCGTGCG |
| | CTCTCCTGTT CCGACCCTGC CGCTTACCGG ATACCTGTCC |
| | GCCTTTCTCC CTTCGGGAAG CGTGGCGCTT TCTCATAGCT |
| | CACGCTGTAG GTATCTCAGT TCGGTGTAGG TCGTTCGCTC |
| | CAAGCTGGGC TGTGTGCACG AACCCCCCGT TCAGCCCGAC |
| | CGCTGCGCCT TATCCGGTAA CTATCGTCTT GAGTCCAACC |
| | CGGTAAGACA CGACTTATCG CCACTGGCAG CAGCCACTGG |
| | TAACAGGATT AGCAGAGCGA GGTATGTAGG CGGTGCTACA |
| | GAGTTCTTGA AGTGGTGGCC TAACTACGGC TACACTAGAA |
| | GGACAGTATT TGGTATCTGC GCTCTGCTGA AGCCAGTTAC |
| | CTTCGGAAAA AGAGTTGGTA GCTCTTGATC CGGCAAACAA |
| | ACCACCGCTG GTAGCGGTGG TTTTTTTGTT TGCAAGCAGC |
| | AGATTACGCG CAGAAAAAAA GGATCTCAAG AAGATCCTTT |
| | GATCTTTTCT ACGGGGTCTG ACGCTCAGTG GAACTAAAAC |
| | TCACGTTAAG GGATTTTGGT CATGAGATTA TCAAAAAGGA |
| | TCTTCACCTA GATCCTTTTA AATTAAAAAT GAAGTTTTAA |
| | ATCAATCTAA AGTATATATG AGTAAACTTG GTCTGACAGT |
| | TACCAATGCT TAATCAGTGA GGCACCTATC TCAGCGATCT |
| | GTCTATTTCG TTCATCCATA GTTGCCTGAC TCCCCGTCGT |
| | GTAGATAACT ACGATACGGG AGGGCTTACC ATCTGGCCCC |
| | AGTGCTGCAA TGATACCGCG AGACCCACGC TCACCGGCTC |
| | CAGATTTATC AGCAATAAAC CAGCCAGCCG GAAGGGCCGA |
| | GCGCAGAAGT GGTCCTGCAA CTTTATCCGC CTCCATCCAG |
| | TCTATTAATT GTTGCCGGGA AGCTAGAGTA AGTAGTTCGC |
| | CAGTTAATAG TTTGCGCAAC GTTGTTGCCA TTGCTACAGG |
| | CATCGTGGTG TCACGCTCGT CGTTTGGTAT GGCTTCATTC |
| | AGCTCCGGTT CCCAACGATC AAGGCGAGTT ACATGATCCC |
| | CCATGTTGTG CAAAAAAGCG GTTAGCTCCT TCGGTCCTCC |
| | GATCGTTGTA AGAAGTAAGT TGGCCGCAGT GTTATCACTC |
| | ATGGTTATGG CAGCACTGCA TAATTCTCTT ACTGTCATGC |
| | CATCCGTAAG ATGCTTTTCT GTGACTGGTG AGTACTCAAC |
| | CAAGTCATTC TGAGAATAGT GTATGCGGCG ACCGAGTTGC |
| | TCTTGCCCGG CGTCAATACG GGATAATACC GCGCCACATA |
| | GCAGAACTTT AAAAGTGCTC ATCATTGGAA AACGTTCTTC |
| | GGGGCGAAAA CTCTCAAGGA TCTTACCGCT GTTGAGATCC |
| | AGTTCGATGT AACCCACTCG TGCACCCAAC TGATCTTCAG |
| | CATCTTTTAC TTTCACCAGC GTTTCTGGGT GAGCAAAAAC |
| | AGGAAGGCAA AATGCCGCAA AAAAGGGAAT |
| | AAGGGCGACA CGGAAATGTT GAATACTCAT ACTCTTCCTT |
| | TTTCAATATT ATTGAAGCAT TTATCAGGGT TATTGTCTCA |
| | TGAGCGGATA CATATTTGAA TGTATTTAGA AAAATAAACA |
| | AATAGGGGTT CCGCGCACAT TTCCCCGAAA AGTGCCACCT |
| | GACGTCTAAG AAACCATTAT TATCATGACA TTAACCTATA |
| | AAAATAGGCG TATCACGAGG CCCTTTCGTC TCGCGCGTTT |
| | CGGTGATGAC GGTGAAAACC TCTGACACAT GCAGCTCCCG |
| | GAGACGGTCA CAGCTTGTCT GTAAGCGGAT GCCGGGAGCA |
| | GACAAGCCCG TCAGGGCGCG TCAGCGGGTG TTGGCGGGTG |
| | TCGGGGCTGG CTTAACTATG CGGCATCAGA GCAGATTGTA |
| | CTGAGAGTGC ACCATATGCG GTGTGAAATA CCGCACAGAT |
| | GCGTAAGGAG AAAATACCGC ATCAGGCGCC ATTCGCCATT |
| | CAGGCTGCGC AACTGTTGGG AAGGGCGATC GGTGCGGGCC |
| | TCTTCGCTAT TACGCCAGCT GGCGAAAGGG GGATGTGCTG |
| | CAAGGCGATT AAGTTGGGTA ACGCCAGGGT TTTCCCAGTC |
| | ACGACGTTGT AAAACGACGG CGCAAGGAAT GGTGCATGCA |
| | AGGAGATGGC GCCCAACAGT CCCCCGGCCA CGGGGCCTGC |
| | CACCATACCC ACGCCGAAAC AAGCGCTCAT GAGCCCGAAG |
| | TGGCGAGCCC GATCTTCCCC ATCGGTGATG TCGGCGATAT |
| | AGGCGCCAGC AACCGCACCT GTGGCGCCGG TGATGCCGGC |
| | CACGATGCGT CCGGCGTAGA GGCGATTAGT CCAATTTGTT |
| | AAAGACAGGA TATCAGTGGT CCAGGCTCTA GTTTTGACTC |
| | AACAATATCA CCAGCTGAAG CCTATAGAGT ACGAGCCATA |
| | GATAAAATAA AAGATTTTAT TTAGTCTCCA GAAAAAGGGG |
| | GG |
| | (SEQ ID NO: 28) |
| MSCV_PGK-gh1- | AATGAAAGAC CCCACCTGTA GGTTTGGCAA GCTAGCTTAA |
| 1-2A-GFP | GTAACGCCAT TTTGCAAGGC ATGGAAAATA CATAACTGAG |
| (FIG. 21, | AATAGAGAAG TTCAGATCAA GGTTAGGAAC AGAGAGACAG |
| bottom) | CAGAATATGG GCCAAACAGG ATATCTGTGG TAAGCAGTTC |
| | CTGCCCCGGC TCAGGGCCAA GAACAGATGG TCCCCAGATG |
| | CGGTCCCGCC CTCAGCAGTT TCTAGAGAAC CATCAGATGT |
| | TTCCAGGGTG CCCCAAGGAC CTGAAATGAC CCTGTGCCTT |
| | ATTTGAACTA ACCAATCAGT TCGCTTCTCG CTTCTGTTCG |
| | CGCGCTTCTG CTCCCCGAGC TCAATAAAAG AGCCCACAAC |
| | CCCTCACTCG GCGCGCCAGT CCTCCGATAG ACTGCGTCGC |
| | CCGGGTACCC GTATTCCCAA TAAAGCCTCT TGCTGTTTGC |
| | ATCCGAATCG TGGACTCGCT GATCCTTGGG AGGGTCTCCT |
| | CAGATTGATT GACTGCCCAC CTCGGGGGTC TTTCATTTGG |
| | AGGTTCCACC GAGATTTGGA GACCCCTGCC CAGGGACCAC |
| | CGACCCCCCC GCCGGGAGGT AAGCTGGCCA GCGGTCGTTT |
| | CGTGTCTGTC TCTGTCTTTG TGCGTGTTTG TGCCGGCATC |
| | TAATGTTTGC GCCTGCGTCT GTACTAGTTA GCTAACTAGC |
| | TCTGTATCTG GCGGACCCGT GGTGGAACTG ACGAGTTCTG |
| | AACACCCGGC CGCAACCCTG GGAGACGTCC CAGGGACTTT |
| | GGGGGCCGTT TTTGTGGCCC GACCTGAGGA AGGGAGTCGA |
| | TGTGGAATCC GACCCCGTCA GGATATGTGG TTCTGGTAGG |
| | AGACGAGAAC CTAAAACAGT TCCCGCCTCC GTCTGAATTT |
| | TTGCTTTCGG TTTGGAACCG AAGCCGCGCG TCTTGTCTGC |
| | TGCAGCGCTG CAGCATCGTT CTGTGTTGTC TCTGTCTGAC |
| | TGTGTTTCTG TATTTGTCTG AAAATTAGGG CCAGACTGTT |
| | ACCACTCCCT TAAGTTTGAC CTTAGGTCAC TGGAAAGATG |
| | TCGAGCGGAT CGCTCACAAC CAGTCGGTAG ATGTCAAGAA |
| | GAGACGTTGG GTTACCTTCT GCTCTGCAGA ATGGCCAACC |
| | TTTAACGTCG GATGGCCGCG AGACGGCACC TTTAACCGAG |
| | ACCTCATCAC CCAGGTTAAG ATCAAGGTCT TTTCACCTGG |
| | CCCGCATGGA CACCCAGACC AGGTCCCCTA CATCGTGACC |
| | TGGGAAGCCT TGGCTTTTGA CCCCCCTCCC TGGGTCAAGC |
| | CCTTTGTACA CCCTAAGCCT CCGCCTCCTC TTCCTCCATC |
| | CGCCCCGTCT CTCCCCCTTG AACCTCCTCG TTCGACCCCG |
| | CCTCGATCCT CCCTTTATCC AGCCCTCACT CCTTCTCTAG |
| | GCGCCGGAAT TAGATCTGGT GATAACGAAT TCTACCGGGT |
| | AGGTGAGGCG CTTTTCCCAA GGCAGTCTGG AGCATGCGCT |
| | TTAGCAGCCC CGCTGGGCAC TTGGCGCTAC ACAAGTGGCC |
| | TCTGGCCTCG CACACATTCC ACATCCACCG GTAGGCGCCA |
| | ACCGGCTCCG TTCTTTGGTG GCCCCTTCGC GCCACCTTCT |
| | ACTCCTCCCC TAGTCAGGAA GTTCCCCCCC GCCCCGCAGC |
| | TCGCGTCGTG CAGGACGTGA CAAATGGAAG TAGCACGTCT |
| | CACTAGTCTC GTGCAGATGG ACAGCACCGC TGAGCAATGG |
| | AAGCGGGTAG GCCTTTGGGG CAGCGGCCAA TAGCAGCTTT |
| | GCTCCTTCGC TTTCTGGGCT CAGAGGCTGG GAAGGGGTGG |
| | GTCCGGGGGC GGGCTCAGGG GCGGGCTCAG GGGCGGGGCG |
| | GGCGCCCGAA GGTCCTCCGG AGGCCCGGCA TTCTGCACGC |
| | TTCAAAAGCG CACGTCTGCC GCGCTGTTCT CCTCTTCCTC |
| | ATCTCCGGGC CTTTCGACCT GCAGCCCAAG CTAGGACCGC |
| | GCCGCCACCA TGGCGTACCC ATACGATGTT CCAGATTACG |
| | CTTCCTTGCC CAAGGATTTT CTGTGGGGGT TTGCCACAGC |
| | TGCCTATCAA ATTGAGGGCG CTATTCACGC AGATGGAAGA |
| | GGACCATCCA TTTGGGACAC ATTTTGCAAC ATCCCTGGCA |
| | AGATAGCAGA CGGATCTAGC GGTGCCGTGG CTTGCGACTC |
| | ATACAACAGA ACTAAAGAGG ATATTGACCT CCTGAAGAGC |
| | TTGGGCGCAA CAGCATACAG GTTTAGTATT TCATGGAGCA |
| | GAATCATCCC AGTAGGAGGC AGAAACGACC CTATTAACCA |
| | GAAGGGTATA GATCACTACG TTAAGTTTGT GGATGATCTG |
| | CTTGAGGCAG GTATCACCCC ATTTATTACC CTCTTTCATT |
| | GGGATTTGCC TGATGGTCTC GATAAGCGCT ATGGCGGGCT |
| | CTTGAATCGG GAGGAGTTCC CTCTGGACTT CGAGCATTAC |
| | GCTAGGACTA TGTTCAAGGC TATACCAAAA TGTAAGCATT |
| | GGATCACTTT CAACGAACCC TGGTGCTCCT CAATCCTCGG |
| | ATACAACTCA GGATATTTTG CTCCAGGACA CACTTCTGAC |
| | AGAACAAAAA GTCCAGTAGG CGATAGCGCC CGCGAGCCCT |
| | GGATAGTTGG CCATAATCTG TTGATCGCAC ATGGGCGAGC |
| | TGTCAAAGTT TATCGGGAAG ATTTCAAGCC TACACAGGGA |
| | GGCGAAATTG GCATCACCCT GAACGGGGAC GCCACCCTGC |
| | CCTGGGACCC AGAGGACCCT CTCGATGTCG AGGCCTGCGA |
| | TCGCAAGATA GAGTTTGCAA TTTCATGGTT TGCTGATCCC |
| | ATTTATTTTG GAAAGTACCC TGACTCCATG AGAAAGCAGC |
| | TGGGTGACAG GCTTCCAGAG TTCACACCTG AAGAAGTTGC |
| | TCTTGTCAAG GGATCCAACG ATTTCTACGG TATGAATCAT |
| | TATACAGCTA ACTATATCAA ACATAAAAAA GGTGTTCCAC |
| | CCGAGGACGA TTTTTTGGGT AATCTCGAAA CCTTGTTTTA |
| | TAACAAAAAG GGAAACTGTA TAGGCCCAGA GACCCAGAGT |
| | TTCTGGCTCC GACCCCATGC TCAAGGGTTC CGCGACCTCC |
| | TGAATTGGTT GTCCAAGCGA TACGGCTATC CTAAGATTTA |
| | TGTGACAGAG AACGGTACTT CATTGAAGGG CGAGAATGCA |
| | ATGCCTTTGA AGCAAATTGT AGAAGATGAT TTCCGCGTTA |
| | AGTACTTTAA TGACTATGTA AATGCTATGG CTAAGGCACA |
| | CTCCGAAGAT GGAGTTAATG TCAAAGGATA CCTCGCTTGG |
| | TCTCTTATGG ATAATTTCGA GTGGGCAGAA GGCTATGAGA |
| | CTAGATTCGG TGTGACATAT GTGGATTACG AGAACGATCA |
| | GAAGCGCTAT CCCAAGAAAT CAGCCAAATC CCTCAAACCA |
| | TTGTTTGATT CATTGATTAA GAAAGACGGA TCCGGCTCCG |
| | GAGAGGGCCG CGGTAGCCTC CTGACCTGCG GGGACGTGGA |
| | GGAGAACCCC GGCCCTATGG TGAGCAAGGG CGAGGAGCTG |
| | TTCACCGGGG TGGTGCCCAT CCTGGTCGAG CTGGACGGCG |
| | ACGTAAACGG CCACAAGTTC AGCGTGTCCG GCGAGGGCGA |
| | GGGCGATGCC ACCTACGGCA AGCTGACCCT GAAGTTCATC |
| | TGCACCACCG GCAAGCTGCC CGTGCCCTGG CCCACCCTCG |
| | TGACCACCTT CACCTACGGC GTGCAGTGCT TCAGCCGCTA |
| | CCCCGACCAC ATGAAGCAGC ACGACTTCTT CAAGTCCGCC |
| | ATGCCCGAAG GCTACGTCCA GGAGCGCACC ATCTCTTTCA |
| | AGGACGACGG CAACTACAAG ACCCGCGCCG AGGTGAAGTT |
| | CGAGGGCGAC ACCCTGGTGA ACCGCATCGA GCTGAAGGGC |
| | ATCGACTTCA AGGAGGACGG CAACATCCTG GGGCACAAGC |
| | TGGAGTACAA CTACAACAGC CACAACGTCT ATATCACGGC |
| | CGACAAGCAG AAGAACGGCA TCAAGGCTAA CTTCAAGATC |
| | CGCCACAACA TCGAGGACGG CAGCGTGCAG CTCGCCGACC |
| | ACTACCAGCA GAACACCCCC ATCGGCGACG GCCCCGTGCT |
| | GCTGCCCGAC AACCACTACC TGAGCACCCA GTCCGCCCTG |
| | AGCAAAGACC CCAACGAGAA GCGCGATCAC ATGGTCCTGC |
| | TGGAGTTCGT GACCGCCGCC GGGATCACTC TCGGCATGGA |
| | CGAGCTGTAC AAGTGACGCC CGCCCCACGA CCCGCAGCGC |
| | CCGACCGAAA GGAGCGCACG ACCCCATGCA TCGATAATTC |
| | CCGATAATCA ACCTCTGGAT TACAAAATTT GTGAAAGATT |
| | GACTGGTATT CTTAACTATG TTGCTCCTTT TACGCTATGT |
| | GGATACGCTG CTTTAATGCC TTTGTATCAT GCTATTGCTT |
| | CCCGTATGGC TTTCATTTTC TCCTCCTTGT ATAAATCCTG |
| | GTTGCTGTCT CTTTATGAGG AGTTGTGGCC CGTTGTCAGG |
| | CAACGTGGCG TGGTGTGCAC TGTGTTTGCT GACGCAACCC |
| | CCACTGGTTG GGGCATTGCC ACCACCTGTC AGCTCCTTTC |
| | CGGGACTTTC GCTTTCCCCC TCCCTATTGC CACGGCGGAA |
| | CTCATCGCCG CCTGCCTTGC CCGCTGCTGG ACAGGGGCTC |
| | GGCTGTTGGG CACTGACAAT TCCGTGGTGT TGTCGGGGAA |
| | ATCATCGTCC TTTCCTTGGC TGCTCGCCTG TGTTGCCACC |
| | TGGATTCTGC GCGGGACGTC CTTCTGCTAC GTCCCTTCGG |
| | CCCTCAATCC AGCGGACCTT CCTTCCCGCG GCCTGCTGCC |
| | GGCTCTGCGG CCTCTTCCGC GTCTTCGCCT TCGCCCTCAG |
| | ACGAGTCGGA TCTCCCTTTG GGCCGCCTCC CCGCATCGGG |
| | AATTATCGAT AAAATAAAAG ATTTTATTTA GTCTCCAGAA |
| | AAAGGGGGGA ATGAAAGACC CCACCTGTAG GTTTGGCAAG |
| | CTAGCTTAAG TAACGCCATT TTGCAAGGCA TGGAAAATAC |
| | ATAACTGAGA ATAGAGAAGT TCAGATCAAG GTTAGGAACA |
| | GAGAGACAGC AGAATATGGG CCAAACAGGA TATCTGTGGT |
| | AAGCAGTTCC TGCCCCGGCT CAGGGCCAAG AACAGATGGT |
| | CCCCAGATGC GGTCCCGCCC TCAGCAGTTT CTAGAGAACC |
| | ATCAGATGTT TCCAGGGTGC CCCAAGGACC TGAAATGACC |
| | CTGTGCCTTA TTTGAACTAA CCAATCAGTT CGCTTCTCGC |
| | TTCTGTTCGC GCGCTTCTGC TCCCCGAGCT CAATAAAAGA |
| | GCCCACAACC CCTCACTCGG CGCGCCAGTC CTCCGATAGA |
| | CTGCGTCGCC CGGGTACCCG TGTATCCAAT AAACCCTCTT |
| | GCAGTTGCAT CCGACTTGTG GTCTCGCTGT TCCTTGGGAG |
| | GGTCTCCTCT GAGTGATTGA CTACCCGTCA GCGGGGGTCT |
| | TTCATGGGTA ACAGTTTCTT GAAGTTGGAG AACAACATTC |
| | TGAGGGTAGG AGTCGAATAT TAAGTAATCC TGACTCAATT |
| | AGCCACTGTT TTGAATCCAC ATACTCCAAT ACTCCTGAAA |
| | TAGTTCATTA TGGACAGCGC AGAAGAGCTG GGGAGAATTG |
| | TGAAATTGTT ATCCGCTCAC AATTCCACAC AACATACGAG |
| | CCGGAAGCAT AAAGTGTAAA GCCTGGGGTG CCTAATGAGT |
| | GAGCTAACTC ACATTAATTG CGTTGCGCTC ACTGCCCGCT |
| | TTCCAGTCGG GAAACCTGTC GTGCCAGCTG CATTAATGAA |
| | TCGGCCAACG CGCGGGGAGA GGCGGTTTGC GTATTGGGCG |
| | CTCTTCCGCT TCCTCGCTCA CTGACTCGCT GCGCTCGGTC |
| | GTTCGGCTGC GGCGAGCGGT ATCAGCTCAC TCAAAGGCGG |
| | TAATACGGTT ATCCACAGAA TCAGGGGATA ACGCAGGAAA |
| | GAACATGTGA GCAAAAGGCC AGCAAAAGGC CAGGAACCGT |
| | AAAAAGGCCG CGTTGCTGGC GTTTTTCCAT AGGCTCCGCC |
| | CCCCTGACGA GCATCACAAA AATCGACGCT CAAGTCAGAG |
| | GTGGCGAAAC CCGACAGGAC TATAAAGATA CCAGGCGTTT |
| | CCCCCTGGAA GCTCCCTCGT GCGCTCTCCT GTTCCGACCC |
| | TGCCGCTTAC CGGATACCTG TCCGCCTTTC TCCCTTCGGG |
| | AAGCGTGGCG CTTTCTCATA GCTCACGCTG TAGGTATCTC |
| | AGTTCGGTGT AGGTCGTTCG CTCCAAGCTG GGCTGTGTGC |
| | ACGAACCCCC CGTTCAGCCC GACCGCTGCG CCTTATCCGG |
| | TAACTATCGT CTTGAGTCCA ACCCGGTAAG ACACGACTTA |
| | TCGCCACTGG CAGCAGCCAC TGGTAACAGG ATTAGCAGAG |
| | CGAGGTATGT AGGCGGTGCT ACAGAGTTCT TGAAGTGGTG |
| | GCCTAACTAC GGCTACACTA GAAGGACAGT ATTTGGTATC |
| | TGCGCTCTGC TGAAGCCAGT TACCTTCGGA AAAAGAGTTG |
| | GTAGCTCTTG ATCCGGCAAA CAAACCACCG CTGGTAGCGG |
| | TGGTTTTTTT GTTTGCAAGC AGCAGATTAC GCGCAGAAAA |
| | AAAGGATCTC AAGAAGATCC TTTGATCTTT TCTACGGGGT |
| | CTGACGCTCA GTGGAACGAA AACTCACGTT AAGGGATTTT |
| | GGTCATGAGA TTATCAAAAA GGATCTTCAC CTAGATCCTT |
| | TTAAATTAAA AATGAAGTTT TAAATCAATC TAAAGTATAT |
| | ATGAGTAAAC TTGGTCTGAC AGTTACCAAT GCTTAATCAG |
| | TGAGGCACCT ATCTCAGCGA TCTGTCTATT TCGTTCATCC |
| | ATAGTTGCCT GACTCCCCGT CGTGTAGATA ACTACGATAC |
| | GGGAGGGCTT ACCATCTGGC CCCAGTGCTG CAATGATACC |
| | GCGAGACCCA CGCTCACCGG CTCCAGATTT ATCAGCAATA |
| | AACCAGCCAG CCGGAAGGGC CGAGCGCAGA AGTGGTCCTG |
| | CAACTTTATC CGCCTCCATC CAGTCTATTA ATTGTTGCCG |
| | GGAAGCTAGA GTAAGTAGTT CGCCAGTTAA TAGTTTGCGC |
| | AACGTTGTTG CCATTGCTAC AGGCATCGTG GTGTCACGCT |
| | CGTCGTTTGG TATGGCTTCA TTCAGCTCCG GTTCCCAACG |
| | ATCAAGGCGA GTTACATGAT CCCCCATGTT GTGCAAAAAA |
| | GCGGTTAGCT CCTTCGGTCC TCCGATCGTT GTCAGAAGTA |
| | AGTTGGCCGC AGTGTTATCA CTCATGGTTA TGGCAGCACT |
| | GCATAATTCT CTTACTGTCA TGCCATCCGT AAGATGCTTT |
| | TCTGTGACTG GTGAGTACTC AACCAAGTCA TTCTGAGAAT |
| | AGTGTATGCG GCGACCGAGT TGCTCTTGCC CGGCGTCAAT |
| | ACGGGATAAT ACCGCGCCAC ATAGCAGAAC TTTAAAAGTG |
| | CTCATCATTG GAAAACGTTC TTCGGGGCGA AAACTCTCAA |
| | GGATCTTACC GCTGTTGAGA TCCAGTTCGA TGTAACCCAC |
| | TCGTGCACCC AACTGATCTT CAGCATCTTT TACTTTCACC |
| | AGCGTTTCTG GGTGAGCAAA AACAGGAAGG CAAAATGCCG |
| | CAAAAAAGGG AATAAGGGCG ACACGGAAAT GTTGAATACT |
| | CATACTCTTC CTTTTTCAAT ATTATTGAAG CATTTATCAG |
| | GGTTATTGTC TCATGAGCGG ATACATATTT GAATGTATTT |
| | AGAAAAATAA ACAAATAGGG GTTCCGCGCA CATTTCCCCG |
| | AAAAGTGCCA CCTGACGTCT AAGAAACCAT TATTATCATG |
| | ACATTAACCT ATAAAAATAG GCGTATCACG AGGCCCTTTC |
| | GTCTCGCGCG TTTCGGTGAT GACGGTGAAA ACCTCTGACA |
| | CATGCAGCTC CCGGAGACGG TCACAGCTTG TCTGTAAGCG |
| | GATGCCGGGA GCAGACAAGC CCGTCAGGGC GCGTCAGCGG |
| | GTGTTGGCGG GTGTCGGGGC TGGCTTAACT ATGCGGCATC |
| | AGAGCAGATT GTACTGAGAG TGCACCATAT GCGGTGTGAA |
| | ATACCGCACA GATGCGTAAG GAGAAAATAC CGCATCAGGC |
| | GCCATTCGCC ATTCAGGCTG CGCAACTGTT GGGAAGGGCG |
| | ATCGGTGCGG GCCTCTTCGC TATTACGCCA GCTGGCGAAA |
| | GGGGGATGTG CTGCAAGGCG ATTAAGTTGG GTAACGCCAG |
| | GGTTTTCCCA GTCACGACGT TGTAAAACGA CGGCGCAAGG |
| | AATGGTGCAT GCAAGGAGAT GGCGCCCAAC AGTCCCCCGG |
| | CCACGGGGCC TGCCACCATA CCCACGCCGA AACAAGCGCT |
| | CATGAGCCCG AAGTGGCGAG CCCGATCTTC CCCATCGGTG |
| | ATGTCGGCGA TATAGGCGCC AGCAACCGCA CCTGTGGCGC |
| | CGGTGATGCC GGCCACGATG CGTCCGGCGT AGAGGCGATT |
| | AGTCCAATTT GTTAAAGACA GGATATCAGT GGTCCAGGCT |
| | CTAGTTTTGA CTCAACAATA TCACCAGCTG AAGCCTATAG |
| | AGTACGAGCC ATAGATAAAA TAAAAGATTT TATTTAGTCT |
| | CCAGAAAAAG GGGGG |
| | (SEQ ID NO: 29) |
| Backbone | ATGAAAGACCCCACCTGTAGGTTTGGCAAGCTAGCTTAAGTA |
| sequence for | ACGCCATTTTGCAAGGCATGGAAAATACATAACTGAGAATAG |
| MSCV_PGK-2A- | AGAAGTTCAGATCAAGGTTAGGAACAGAGAGACAGCAGAAT |
| mCherry vector | ATGGGCCAAACAGGATATCTGTGGTAAGCAGTTCCTGCCCCG |
| | GCTCAGGGCCAAGAACAGATGGTCCCCAGATGCGGTCCCGCC |
| | CTCAGCAGTTTCTAGAGAACCATCAGATGTTTCCAGGGTGCC |
| | CCAAGGACCTGAAATGACCCTGTGCCTTATTTGAACTAACCA |
| | ATCAGTTCGCTTCTCGCTTCTGTTCGCGCGCTTCTGCTCCCCG |
| | AGCTCAATAAAAGAGCCCACAACCCCTCACTCGGCGCGCCAG |
| | TCCTCCGATAGACTGCGTCGCCCGGGTACCCGTGTATCCAAT |
| | AAACCCTCTTGCAGTTGCATCCGACTTGTGGTCTCGCTGTTCC |
| | TTGGGAGGGTCTCCTCTGAGTGATTGACTACCCGTCAGCGGG |
| | GGTCTTTCATGGGTAACAGTTTCTTGAAGTTGGAGAACAACA |
| | TTCTGAGGGTAGGAGTCGAATATTAAGTAATCCTGACTCAAT |
| | TAGCCACTGTTTTGAATCCACATACTCCAATACTCCTGAAATA |
| | GTTCATTATGGACAGCGCAGAAGAGCTGGGGAGAATTGTGAA |
| | ATTGTTATCCGCTCACAATTCCACACAACATACGAGCCGGAA |
| | GCATAAAGTGTAAAGCCTGGGGTGCCTAATGAGTGAGCTAAC |
| | TCACATTAATTGCGTTGCGCTCACTGCCCGCTTTCCAGTCGGG |
| | AAACCTGTCGTGCCAGCTGCATTAATGAATCGGCCAACGCGC |
| | GGGGAGAGGCGGTTTGCGTATTGGGCGCTCTTCCGCTTCCTC |
| | GCTCACTGACTCGCTGCGCTCGGTCGTTCGGCTGCGGCGAGC |
| | GGTATCAGCTCACTCAAAGGCGGTAATACGGTTATCCACAGA |
| | ATCAGGGGATAACGCAGGAAAGAACATGTGAGCAAAAGGCC |
| | AGCAAAAGGCCAGGAACCGTAAAAAGGCCGCGTTGCTGGCG |
| | TTTTTCCATAGGCTCCGCCCCCCTGACGAGCATCACAAAAATC |
| | GACGCTCAAGTCAGAGGTGGCGAAACCCGACAGGACTATAA |
| | AGATACCAGGCGTTTCCCCCTGGAAGCTCCCTCGTGCGCTCTC |
| | CTGTTCCGACCCTGCCGCTTACCGGATACCTGTCCGCCTTTCT |
| | CCCTTCGGGAAGCGTGGCGCTTTCTCATAGCTCACGCTGTAG |
| | GTATCTCAGTTCGGTGTAGGTCGTTCGCTCCAAGCTGGGCTGT |
| | GTGCACGAACCCCCCGTTCAGCCCGACCGCTGCGCCTTATCC |
| | GGTAACTATCGTCTTGAGTCCAACCCGGTAAGACACGACTTA |
| | TCGCCACTGGCAGCAGCCACTGGTAACAGGATTAGCAGAGCG |
| | AGGTATGTAGGCGGTGCTACAGAGTTCTTGAAGTGGTGGCCT |
| | AACTACGGCTACACTAGAAGGACAGTATTTGGTATCTGCGCT |
| | CTGCTGAAGCCAGTTACCTTCGGAAAAAGAGTTGGTAGCTCT |
| | TGATCCGGCAAACAAACCACCGCTGGTAGCGGTGGTTTTTTT |
| | GTTTGCAAGCAGCAGATTACGCGCAGAAAAAAAGGATCTCA |
| | AGAAGATCCTTTGATCTTTTCTACGGGGTCTGACGCTCAGTGG |
| | AACGAAAACTCACGTTAAGGGATTTTGGTCATGAGATTATCA |
| | AAAAGGATCTTCACCTAGATCCTTTTAAATTAAAAATGAAGT |
| | TTTAAATCAATCTAAAGTATATATGAGTAAACTTGGTCTGAC |
| | AGTTACCAATGCTTAATCAGTGAGGCACCTATCTCAGCGATC |
| | TGTCTATTTCGTTCATCCATAGTTGCCTGACTCCCCGTCGTGT |
| | AGATAACTACGATACGGGAGGGCTTACCATCTGGCCCCAGTG |
| | CTGCAATGATACCGCGAGACCCACGCTCACCGGCTCCAGATT |
| | TATCAGCAATAAACCAGCCAGCCGGAAGGGCCGAGCGCAGA |
| | AGTGGTCCTGCAACTTTATCCGCCTCCATCCAGTCTATTAATT |
| | GTTGCCGGGAAGCTAGAGTAAGTAGTTCGCCAGTTAATAGTT |
| | TGCGCAACGTTGTTGCCATTGCTACAGGCATCGTGGTGTCAC |
| | GCTCGTCGTTTGGTATGGCTTCATTCAGCTCCGGTTCCCAACG |
| | ATCAAGGCGAGTTACATGATCCCCCATGTTGTGCAAAAAAGC |
| | GGTTAGCTCCTTCGGTCCTCCGATCGTTGTCAGAAGTAAGTTG |
| | GCCGCAGTGTTATCACTCATGGTTATGGCAGCACTGCATAATT |
| | CTCTTACTGTCATGCCATCCGTAAGATGCTTTTCTGTGACTGG |
| | TGAGTACTCAACCAAGTCATTCTGAGAATAGTGTATGCGGCG |
| | ACCGAGTTGCTCTTGCCCGGCGTCAATACGGGATAATACCGC |
| | GCCACATAGCAGAACTTTAAAAGTGCTCATCATTGGAAAACG |
| | TTCTTCGGGGCGAAAACTCTCAAGGATCTTACCGCTGTTGAG |
| | ATCCAGTTCGATGTAACCCACTCGTGCACCCAACTGATCTTCA |
| | GCATCTTTTACTTTCACCAGCGTTTCTGGGTGAGCAAAAACAG |
| | GAAGGCAAAATGCCGCAAAAAAGGGAATAAGGGCGACACGG |
| | AAATGTTGAATACTCATACTCTTCCTTTTTCAATATTATTGAA |
| | GCATTTATCAGGGTTATTGTCTCATGAGCGGATACATATTTGA |
| | ATGTATTTAGAAAAATAAACAAATAGGGGTTCCGCGCACATT |
| | TCCCCGAAAAGTGCCACCTGACGTCTAAGAAACCATTATTAT |
| | CATGACATTAACCTATAAAAATAGGCGTATCACGAGGCCCTT |
| | TCGTCTCGCGCGTTTCGGTGATGACGGTGAAAACCTCTGACA |
| | CATGCAGCTCCCGGAGACGGTCACAGCTTGTCTGTAAGCGGA |
| | TGCCGGGAGCAGACAAGCCCGTCAGGGCGCGTCAGCGGGTG |
| | TTGGCGGGTGTCGGGGCTGGCTTAACTATGCGGCATCAGAGC |
| | AGATTGTACTGAGAGTGCACCATATGCGGTGTGAAATACCGC |
| | ACAGATGCGTAAGGAGAAAATACCGCATCAGGCGCCATTCGC |
| | CATTCAGGCTGCGCAACTGTTGGGAAGGGCGATCGGTGCGGG |
| | CCTCTTCGCTATTACGCCAGCTGGCGAAAGGGGGATGTGCTG |
| | CAAGGCGATTAAGTTGGGTAACGCCAGGGTTTTCCCAGTCAC |
| | GACGTTGTAAAACGACGGCGCAAGGAATGGTGCATGCAAGG |
| | AGATGGCGCCCAACAGTCCCCCGGCCACGGGGCCTGCCACCA |
| | TACCCACGCCGAAACAAGCGCTCATGAGCCCGAAGTGGCGA |
| | GCCCGATCTTCCCCATCGGTGATGTCGGCGATATAGGCGCCA |
| | GCAACCGCACCTGTGGCGCCGGTGATGCCGGCCACGATGCGT |
| | CCGGCGTAGAGGCGATTAGTCCAATTTGTTAAAGACAGGATA |
| | TCAGTGGTCCAGGCTCTAGTTTTGACTCAACAATATCACCAGC |
| | TGAAGCCTATAGAGTACGAGCCATAGATAAAATAAAAGATTT |
| | TATTTAGTCTCCAGAAAAAGGGGGGAATGAAAGACCCCACCT |
| | GTAGGTTTGGCAAGCTAGCTTAAGTAACGCCATTTTGCAAGG |
| | CATGGAAAATACATAACTGAGAATAGAGAAGTTCAGATCAA |
| | GGTTAGGAACAGAGAGACAGCAGAATATGGGCCAAACAGGA |
| | TATCTGTGGTAAGCAGTTCCTGCCCCGGCTCAGGGCCAAGAA |
| | CAGATGGTCCCCAGATGCGGTCCCGCCCTCAGCAGTTTCTAG |
| | AGAACCATCAGATGTTTCCAGGGTGCCCCAAGGACCTGAAAT |
| | GACCCTGTGCCTTATTTGAACTAACCAATCAGTTCGCTTCTCG |
| | CTTCTGTTCGCGCGCTTCTGCTCCCCGAGCTCAATAAAAGAGC |
| | CCACAACCCCTCACTCGGCGCGCCAGTCCTCCGATAGACTGC |
| | GTCGCCCGGGTACCCGTATTCCCAATAAAGCCTCTTGCTGTTT |
| | GCATCCGAATCGTGGACTCGCTGATCCTTGGGAGGGTCTCCT |
| | CAGATTGATTGACTGCCCACCTCGGGGGTCTTTCATTTGGAGG |
| | TTCCACCGAGATTTGGAGACCCCTGCCCAGGGACCACCGACC |
| | CCCCCGCCGGGAGGTAAGCTGGCCAGCGGTCGTTTCGTGTCT |
| | GTCTCTGTCTTTGTGCGTGTTTGTGCCGGCATCTAATGTTTGC |
| | GCCTGCGTCTGTACTAGTTAGCTAACTAGCTCTGTATCTGGCG |
| | GACCCGTGGTGGAACTGACGAGTTCTGAACACCCGGCCGCAA |
| | CCCTGGGAGACGTCCCAGGGACTTTGGGGGCCGTTTTTGTGG |
| | CCCGACCTGAGGAAGGGAGTCGATGTGGAATCCGACCCCGTC |
| | AGGATATGTGGTTCTGGTAGGAGACGAGAACCTAAAACAGTT |
| | CCCGCCTCCGTCTGAATTTTTGCTTTCGGTTTGGAACCGAAGC |
| | CGCGCGTCTTGTCTGCTGCAGCGCTGCAGCATCGTTCTGTGTT |
| | GTCTCTGTCTGACTGTGTTTCTGTATTTGTCTGAAAATTAGGG |
| | CCAGACTGTTACCACTCCCTTAAGTTTGACCTTAGGTCACTGG |
| | AAAGATGTCGAGCGGATCGCTCACAACCAGTCGGTAGATGTC |
| | AAGAAGAGACGTTGGGTTACCTTCTGCTCTGCAGAATGGCCA |
| | ACCTTTAACGTCGGATGGCCGCGAGACGGCACCTTTAACCGA |
| | GACCTCATCACCCAGGTTAAGATCAAGGTCTTTTCACCTGGCC |
| | CGCATGGACACCCAGACCAGGTCCCCTACATCGTGACCTGGG |
| | AAGCCTTGGCTTTTGACCCCCCTCCCTGGGTCAAGCCCTTTGT |
| | ACACCCTAAGCCTCCGCCTCCTCTTCCTCCATCCGCCCCGTCT |
| | CTCCCCCTTGAACCTCCTCGTTCGACCCCGCCTCGATCCTCCC |
| | TTTATCCAGCCCTCACTCCTTCTCTAGGCGCCGGAATTAGATC |
| | TGGTGATAACGAATTCTACCGGGTAGGTGAGGCGCTTTTCCC |
| | AAGGCAGTCTGGAGCATGCGCTTTAGCAGCCCCGCTGGGCAC |
| | TTGGCGCTACACAAGTGGCCTCTGGCCTCGCACACATTCCAC |
| | ATCCACCGGTAGGCGCCAACCGGCTCCGTTCTTTGGTGGCCC |
| | CTTCGCGCCACCTTCTACTCCTCCCCTAGTCAGGAAGTTCCCC |
| | CCCGCCCCGCAGCTCGCGTCGTGCAGGACGTGACAAATGGAA |
| | GTAGCACGTCTCACTAGTCTCGTGCAGATGGACAGCACCGCT |
| | GAGCAATGGAAGCGGGTAGGCCTTTGGGGCAGCGGCCAATA |
| | GCAGCTTTGCTCCTTCGCTTTCTGGGCTCAGAGGCTGGGAAG |
| | GGGTGGGTCCGGGGGGGGCTCAGGGGGGGGCTCAGGGGCG |
| | GGGCGGGCGCCCGAAGGTCCTCCGGAGGCCCGGCATTCTGCA |
| | CGCTTCAAAAGCGCACGTCTGCCGCGCTGTTCTCCTCTTCCTC |
| | ATCTCCGGGCCTTTCG |
| | (SEQ ID NO: 42) |
| Backbone | ATGAAAGACCCCACCTGTAGGTTTGGCAAGCTAGCTTAAGTA |
| sequence for | ACGCCATTTTGCAAGGCATGGAAAATACATAACTGAGAATAG |
| MSCV_PGK-2A- | AGAAGTTCAGATCAAGGTTAGGAACAGAGAGACAGCAGAAT |
| GFP vector | ATGGGCCAAACAGGATATCTGTGGTAAGCAGTTCCTGCCCCG |
| | GCTCAGGGCCAAGAACAGATGGTCCCCAGATGCGGTCCCGCC |
| | CTCAGCAGTTTCTAGAGAACCATCAGATGTTTCCAGGGTGCC |
| | CCAAGGACCTGAAATGACCCTGTGCCTTATTTGAACTAACCA |
| | ATCAGTTCGCTTCTCGCTTCTGTTCGCGCGCTTCTGCTCCCCG |
| | AGCTCAATAAAAGAGCCCACAACCCCTCACTCGGCGCGCCAG |
| | TCCTCCGATAGACTGCGTCGCCCGGGTACCCGTGTATCCAAT |
| | AAACCCTCTTGCAGTTGCATCCGACTTGTGGTCTCGCTGTTCC |
| | TTGGGAGGGTCTCCTCTGAGTGATTGACTACCCGTCAGCGGG |
| | GGTCTTTCATGGGTAACAGTTTCTTGAAGTTGGAGAACAACA |
| | TTCTGAGGGTAGGAGTCGAATATTAAGTAATCCTGACTCAAT |
| | TAGCCACTGTTTTGAATCCACATACTCCAATACTCCTGAAATA |
| | GTTCATTATGGACAGCGCAGAAGAGCTGGGGAGAATTGTGAA |
| | ATTGTTATCCGCTCACAATTCCACACAACATACGAGCCGGAA |
| | GCATAAAGTGTAAAGCCTGGGGTGCCTAATGAGTGAGCTAAC |
| | TCACATTAATTGCGTTGCGCTCACTGCCCGCTTTCCAGTCGGG |
| | AAACCTGTCGTGCCAGCTGCATTAATGAATCGGCCAACGCGC |
| | GGGGAGAGGCGGTTTGCGTATTGGGCGCTCTTCCGCTTCCTC |
| | GCTCACTGACTCGCTGCGCTCGGTCGTTCGGCTGCGGCGAGC |
| | GGTATCAGCTCACTCAAAGGCGGTAATACGGTTATCCACAGA |
| | ATCAGGGGATAACGCAGGAAAGAACATGTGAGCAAAAGGCC |
| | AGCAAAAGGCCAGGAACCGTAAAAAGGCCGCGTTGCTGGCG |
| | TTTTTCCATAGGCTCCGCCCCCCTGACGAGCATCACAAAAATC |
| | GACGCTCAAGTCAGAGGTGGCGAAACCCGACAGGACTATAA |
| | AGATACCAGGCGTTTCCCCCTGGAAGCTCCCTCGTGCGCTCTC |
| | CTGTTCCGACCCTGCCGCTTACCGGATACCTGTCCGCCTTTCT |
| | CCCTTCGGGAAGCGTGGCGCTTTCTCATAGCTCACGCTGTAG |
| | GTATCTCAGTTCGGTGTAGGTCGTTCGCTCCAAGCTGGGCTGT |
| | GTGCACGAACCCCCCGTTCAGCCCGACCGCTGCGCCTTATCC |
| | GGTAACTATCGTCTTGAGTCCAACCCGGTAAGACACGACTTA |
| | TCGCCACTGGCAGCAGCCACTGGTAACAGGATTAGCAGAGCG |
| | AGGTATGTAGGCGGTGCTACAGAGTTCTTGAAGTGGTGGCCT |
| | AACTACGGCTACACTAGAAGGACAGTATTTGGTATCTGCGCT |
| | CTGCTGAAGCCAGTTACCTTCGGAAAAAGAGTTGGTAGCTCT |
| | TGATCCGGCAAACAAACCACCGCTGGTAGCGGTGGTTTTTTT |
| | GTTTGCAAGCAGCAGATTACGCGCAGAAAAAAAGGATCTCA |
| | AGAAGATCCTTTGATCTTTTCTACGGGGTCTGACGCTCAGTGG |
| | AACGAAAACTCACGTTAAGGGATTTTGGTCATGAGATTATCA |
| | AAAAGGATCTTCACCTAGATCCTTTTAAATTAAAAATGAAGT |
| | TTTAAATCAATCTAAAGTATATATGAGTAAACTTGGTCTGAC |
| | AGTTACCAATGCTTAATCAGTGAGGCACCTATCTCAGCGATC |
| | TGTCTATTTCGTTCATCCATAGTTGCCTGACTCCCCGTCGTGT |
| | AGATAACTACGATACGGGAGGGCTTACCATCTGGCCCCAGTG |
| | CTGCAATGATACCGCGAGACCCACGCTCACCGGCTCCAGATT |
| | TATCAGCAATAAACCAGCCAGCCGGAAGGGCCGAGCGCAGA |
| | AGTGGTCCTGCAACTTTATCCGCCTCCATCCAGTCTATTAATT |
| | GTTGCCGGGAAGCTAGAGTAAGTAGTTCGCCAGTTAATAGTT |
| | TGCGCAACGTTGTTGCCATTGCTACAGGCATCGTGGTGTCAC |
| | GCTCGTCGTTTGGTATGGCTTCATTCAGCTCCGGTTCCCAACG |
| | ATCAAGGCGAGTTACATGATCCCCCATGTTGTGCAAAAAAGC |
| | GGTTAGCTCCTTCGGTCCTCCGATCGTTGTCAGAAGTAAGTTG |
| | GCCGCAGTGTTATCACTCATGGTTATGGCAGCACTGCATAATT |
| | CTCTTACTGTCATGCCATCCGTAAGATGCTTTTCTGTGACTGG |
| | TGAGTACTCAACCAAGTCATTCTGAGAATAGTGTATGCGGCG |
| | ACCGAGTTGCTCTTGCCCGGCGTCAATACGGGATAATACCGC |
| | GCCACATAGCAGAACTTTAAAAGTGCTCATCATTGGAAAACG |
| | TTCTTCGGGGCGAAAACTCTCAAGGATCTTACCGCTGTTGAG |
| | ATCCAGTTCGATGTAACCCACTCGTGCACCCAACTGATCTTCA |
| | GCATCTTTTACTTTCACCAGCGTTTCTGGGTGAGCAAAAACAG |
| | GAAGGCAAAATGCCGCAAAAAAGGGAATAAGGGCGACACGG |
| | AAATGTTGAATACTCATACTCTTCCTTTTTCAATATTATTGAA |
| | GCATTTATCAGGGTTATTGTCTCATGAGCGGATACATATTTGA |
| | ATGTATTTAGAAAAATAAACAAATAGGGGTTCCGCGCACATT |
| | TCCCCGAAAAGTGCCACCTGACGTCTAAGAAACCATTATTAT |
| | CATGACATTAACCTATAAAAATAGGCGTATCACGAGGCCCTT |
| | TCGTCTCGCGCGTTTCGGTGATGACGGTGAAAACCTCTGACA |
| | CATGCAGCTCCCGGAGACGGTCACAGCTTGTCTGTAAGCGGA |
| | TGCCGGGAGCAGACAAGCCCGTCAGGGCGCGTCAGCGGGTG |
| | TTGGCGGGTGTCGGGGCTGGCTTAACTATGCGGCATCAGAGC |
| | AGATTGTACTGAGAGTGCACCATATGCGGTGTGAAATACCGC |
| | ACAGATGCGTAAGGAGAAAATACCGCATCAGGCGCCATTCGC |
| | CATTCAGGCTGCGCAACTGTTGGGAAGGGCGATCGGTGCGGG |
| | CCTCTTCGCTATTACGCCAGCTGGCGAAAGGGGGATGTGCTG |
| | CAAGGCGATTAAGTTGGGTAACGCCAGGGTTTTCCCAGTCAC |
| | GACGTTGTAAAACGACGGCGCAAGGAATGGTGCATGCAAGG |
| | AGATGGCGCCCAACAGTCCCCCGGCCACGGGGCCTGCCACCA |
| | TACCCACGCCGAAACAAGCGCTCATGAGCCCGAAGTGGCGA |
| | GCCCGATCTTCCCCATCGGTGATGTCGGCGATATAGGCGCCA |
| | GCAACCGCACCTGTGGCGCCGGTGATGCCGGCCACGATGCGT |
| | CCGGCGTAGAGGCGATTAGTCCAATTTGTTAAAGACAGGATA |
| | TCAGTGGTCCAGGCTCTAGTTTTGACTCAACAATATCACCAGC |
| | TGAAGCCTATAGAGTACGAGCCATAGATAAAATAAAAGATTT |
| | TATTTAGTCTCCAGAAAAAGGGGGGAATGAAAGACCCCACCT |
| | GTAGGTTTGGCAAGCTAGCTTAAGTAACGCCATTTTGCAAGG |
| | CATGGAAAATACATAACTGAGAATAGAGAAGTTCAGATCAA |
| | GGTTAGGAACAGAGAGACAGCAGAATATGGGCCAAACAGGA |
| | TATCTGTGGTAAGCAGTTCCTGCCCCGGCTCAGGGCCAAGAA |
| | CAGATGGTCCCCAGATGCGGTCCCGCCCTCAGCAGTTTCTAG |
| | AGAACCATCAGATGTTTCCAGGGTGCCCCAAGGACCTGAAAT |
| | GACCCTGTGCCTTATTTGAACTAACCAATCAGTTCGCTTCTCG |
| | CTTCTGTTCGCGCGCTTCTGCTCCCCGAGCTCAATAAAAGAGC |
| | CCACAACCCCTCACTCGGCGCGCCAGTCCTCCGATAGACTGC |
| | GTCGCCCGGGTACCCGTATTCCCAATAAAGCCTCTTGCTGTTT |
| | GCATCCGAATCGTGGACTCGCTGATCCTTGGGAGGGTCTCCT |
| | CAGATTGATTGACTGCCCACCTCGGGGGTCTTTCATTTGGAGG |
| | TTCCACCGAGATTTGGAGACCCCTGCCCAGGGACCACCGACC |
| | CCCCCGCCGGGAGGTAAGCTGGCCAGCGGTCGTTTCGTGTCT |
| | GTCTCTGTCTTTGTGCGTGTTTGTGCCGGCATCTAATGTTTGC |
| | GCCTGCGTCTGTACTAGTTAGCTAACTAGCTCTGTATCTGGCG |
| | GACCCGTGGTGGAACTGACGAGTTCTGAACACCCGGCCGCAA |
| | CCCTGGGAGACGTCCCAGGGACTTTGGGGGCCGTTTTTGTGG |
| | CCCGACCTGAGGAAGGGAGTCGATGTGGAATCCGACCCCGTC |
| | AGGATATGTGGTTCTGGTAGGAGACGAGAACCTAAAACAGTT |
| | CCCGCCTCCGTCTGAATTTTTGCTTTCGGTTTGGAACCGAAGC |
| | CGCGCGTCTTGTCTGCTGCAGCGCTGCAGCATCGTTCTGTGTT |
| | GTCTCTGTCTGACTGTGTTTCTGTATTTGTCTGAAAATTAGGG |
| | CCAGACTGTTACCACTCCCTTAAGTTTGACCTTAGGTCACTGG |
| | AAAGATGTCGAGCGGATCGCTCACAACCAGTCGGTAGATGTC |
| | AAGAAGAGACGTTGGGTTACCTTCTGCTCTGCAGAATGGCCA |
| | ACCTTTAACGTCGGATGGCCGCGAGACGGCACCTTTAACCGA |
| | GACCTCATCACCCAGGTTAAGATCAAGGTCTTTTCACCTGGCC |
| | CGCATGGACACCCAGACCAGGTCCCCTACATCGTGACCTGGG |
| | AAGCCTTGGCTTTTGACCCCCCTCCCTGGGTCAAGCCCTTTGT |
| | ACACCCTAAGCCTCCGCCTCCTCTTCCTCCATCCGCCCCGTCT |
| | CTCCCCCTTGAACCTCCTCGTTCGACCCCGCCTCGATCCTCCC |
| | TTTATCCAGCCCTCACTCCTTCTCTAGGCGCCGGAATTAGATC |
| | TGGTGATAACGAATTCTACCGGGTAGGTGAGGCGCTTTTCCC |
| | AAGGCAGTCTGGAGCATGCGCTTTAGCAGCCCCGCTGGGCAC |
| | TTGGCGCTACACAAGTGGCCTCTGGCCTCGCACACATTCCAC |
| | ATCCACCGGTAGGCGCCAACCGGCTCCGTTCTTTGGTGGCCC |
| | CTTCGCGCCACCTTCTACTCCTCCCCTAGTCAGGAAGTTCCCC |
| | CCCGCCCCGCAGCTCGCGTCGTGCAGGACGTGACAAATGGAA |
| | GTAGCACGTCTCACTAGTCTCGTGCAGATGGACAGCACCGCT |
| | GAGCAATGGAAGCGGGTAGGCCTTTGGGGCAGCGGCCAATA |
| | GCAGCTTTGCTCCTTCGCTTTCTGGGCTCAGAGGCTGGGAAG |
| | GGGTGGGTCCGGGGGCGGGCTCAGGGGGGGGCTCAGGGGCG |
| | GGGCGGGCGCCCGAAGGTCCTCCGGAGGCCCGGCATTCTGCA |
| | CGCTTCAAAAGCGCACGTCTGCCGCGCTGTTCTCCTCTTCCTC |
| | ATCTCCGGGCCTTTCG |
| | (SEQ ID NO: 43) |

The sequences of the resulting proteins expressed by the MSCV_PGK-cdt-1-2A-mCherry vector (SEQ ID NO: 28; see FIG. 21, top) and by the MSCV_PGK-gh1-1-2A-GFP vector (SEQ ID NO: 29; see FIG. 21, bottom) are shown in Table 3.

TABLE 3
Proteins Expressed by MSCV_
PGK-cdt-1-2A-mCherry Vector and by
MSCV_PGK-gh1-1-2A-GFP Vector.

| Construct | |
|---|---|
| (Corresponding | Sequence |
| FIGURE) | (SEQ ID NO:) |
| Linker | GSGSG |
| (FIG. 20, top | (SEQ ID NO: 30) |
| & bottom; | |
| FIG. 21, top | |
| & bottom) | |
| T2A amino acids | EGRGSLLTCGDVEENPGP |
| (FIG. 20, top | (SEQ ID NO: 31) |
| & bottom; | |
| FIG. 21, top | |
| & bottom) | |
| CDT-1 | MASSHGSHDGASTEKHLATHDIAPT |
| (FIG. 21, top) | HDAIKIVPKGHGQTATKPGAQEKEV |
| | RNAALFAAIKESNIKPWSKESIHLY |
| | FAIFVAFCCACANGYDGSLMTGIIA |
| | MDKFQNQFHTGDTGPKVSVIFSLYT |
| | VGAMVGAPFAAILSDRFGRKKGMFI |
| | GGIFIIVGSIIVASSSKLAQFVVGR |
| | FVLGLGIAIMTVAAPAYSIEIAPPH |
| | WRGRCTGFYNCGWFGGSIPAACITY |
| | GCYFIKSNWSWRIPLILQAFTCLIV |
| | MSSVFFLPESPRFLFANGRDAEAVA |
| | FLVKYHGNGDPNSKLVLLETEEMRD |
| | GIRTDGVDKVWWDYRPLFMTHSGRW |
| | RMAQVLMISIFGQFSGNGLGYFNTV |
| | IFKNIGVTSTSQQLAYNILNSVISA |
| | IGALTAVSMTDRMPRRAVLIIGTFM |
| | CAAALATNSGLSATLDKQTQRGTQI |
| | NLNQGMNEQDAKDNAYLHVDSNYAK |
| | GALAAYFLFNVIFSFTYTPLQGVIP |
| | TEALETTIRGKGLALSGFIVNAMGF |
| | INQFAGPIALHNIGYKYIFVFVGWD |
| | LIETVAWYFFGVESQGRTLEQLEWV |
| | YDQPNPVKASLKVEKVVVQADGHVS |
| | EAIVAYPYDVPDYA |
| | (SEQ ID NO: 32) |
| CDT-1 with linker | MASSHGSHDGASTEKHLATHDIAPT |
| and T2A amino | HDAIKIVPKGHGQTATKPGAQEKEV |
| acids | RNAALFAAIKESNIKPWSKESIHLY |
| (FIG. 21, top) | FAIFVAFCCACANGYDGSLMTGIIA |
| | MDKFQNQFHTGDTGPKVSVIFSLYT |
| | VGAMVGAPFAAILSDRFGRKKGMFI |
| | GGIFIIVGSIIVASSSKLAQFVVGR |
| | FVLGLGIAIMTVAAPAYSIEIAPPH |
| | WRGRCTGFYNCGWFGGSIPAACITY |
| | GCYFIKSNWSWRIPLILQAFTCLIV |
| | MSSVFFLPESPRFLFANGRDAEAVA |
| | FLVKYHGNGDPNSKLVLLETEEMRD |
| | GIRTDGVDKVWWDYRPLFMTHSGRW |
| | RMAQVLMISIFGQFSGNGLGYFNTV |
| | IFKNIGVTSTSQQLAYNILNSVISA |
| | IGALTAVSMTDRMPRRAVLIIGTFM |
| | CAAALATNSGLSATLDKQTQRGTQI |
| | NLNQGMNEQDAKDNAYLHVDSNYAK |
| | GALAAYFLFNVIFSFTYTPLQGVIP |
| | TEALETTIRGKGLALSGFIVNAMGF |
| | INQFAGPIALHNIGYKYIFVFVGWD |
| | LIETVAWYFFGVESQGRTLEQLEWV |
| | YDQPNPVKASLKVEKVVVQADGHVS |
| | EAIVAYPYDVPDYAGSGSGEGRGSL |
| | LTCGDVEENPG |
| | (SEQ ID NO: 33) |
| mCherry | MVSKGEEDNMAIIKEFMRFKVHMEG |
| (FIG. 20, top | SVNGHEFEIEGEGEGRPYEGTQTAK |
| & FIG. 21, | LKVTKGGPLPFAWDILSPQFMYGSK |
| top) | AYVKHPADIPDYLKLSFPEGFKWER |
| | VMNFEDGGVVTVTQDSSLQDGEFIY |
| | KVKLRGTNFPSDGPVMQKKTMGWEA |
| | SSERMYPEDGALKGEIKQRLKLKDG |
| | GHYDAEVKTTYKAKKPVQLPGAYNV |
| | NIKLDITSHNEDYTIVEQYERAEGR |
| | HSTGGMDELYK |
| | (SEQ ID NO: 34) |
| mCherry with T2A | PMVSKGEEDNMAIIKEFMRFKVHME |
| amino acid | GSVNGHEFEIEGEGEGRPYEGTQTA |
| (FIG. 20, top | KLKVTKGGPLPFAWDILSPQFMYGS |
| & FIG. 21, | KAYVKHPADIPDYLKLSFPEGFKWE |
| top) | RVMNFEDGGVVTVTQDSSLQDGEFI |
| | YKVKLRGTNFPSDGPVMQKKTMGWE |
| | ASSERMYPEDGALKGEIKQRLKLKD |
| | GGHYDAEVKTTYKAKKPVQLPGAYN |
| | VNIKLDITSHNEDYTIVEQYERAEG |
| | RHSTGGMDELYK |
| | (SEQ ID NO: 35) |
| CDT-1 with linker, | MASSHGSHDGASTEKHLATHDIAPT |
| T2A amino acids, | HDAIKIVPKGHGQTATKPGAQEKEV |
| and mCherry | RNAALFAAIKESNIKPWSKESIHLY |
| (FIG. 21, top) | FAIFVAFCCACANGYDGSLMTGIIA |
| | MDKFQNQFHTGDTGPKVSVIFSLYT |
| | VGAMVGAPFAAILSDRFGRKKGMFI |
| | GGIFIIVGSIIVASSSKLAQFVVGR |
| | FVLGLGIAIMTVAAPAYSIEIAPPH |
| | WRGRCTGFYNCGWFGGSIPAACITY |
| | GCYFIKSNWSWRIPLILQAFTCLIV |
| | MSSVFFLPESPRFLFANGRDAEAVA |
| | FLVKYHGNGDPNSKLVLLETEEMRD |
| | GIRTDGVDKVWWDYRPLFMTHSGRW |
| | RMAQVLMISIFGQFSGNGLGYFNTV |
| | IFKNIGVTSTSQQLAYNILNSVISA |
| | IGALTAVSMTDRMPRRAVLIIGTFM |
| | CAAALATNSGLSATLDKQTQRGTQI |
| | NLNQGMNEQDAKDNAYLHVDSNYAK |
| | GALAAYFLFNVIFSFTYTPLQGVIP |
| | TEALETTIRGKGLALSGFIVNAMGF |
| | INQFAGPIALHNIGYKYIFVFVGWD |
| | LIETVAWYFFGVESQGRTLEQLEWV |
| | YDQPNPVKASLKVEKVVVQADGHVS |
| | EAIVAYPYDVPDYAGSGSGEGRGSL |
| | LTCGDVEENPGPMVSKGEEDNMAII |
| | KEFMRFKVHMEGSVNGHEFEIEGEG |
| | EGRPYEGTQTAKLKVTKGGPLPFAW |
| | DILSPQFMYGSKAYVKHPADIPDYL |
| | KLSFPEGFKWERVMNFEDGGVVTVT |
| | QDSSLQDGEFIYKVKLRGTNFPSDG |
| | PVMQKKTMGWEASSERMYPEDGALK |
| | GEIKQRLKLKDGGHYDAEVKTTYKA |
| | KKPVQLPGAYNVNIKLDITSHNEDY |
| | TIVEQYERAEGRHSTGGMDELYK |
| | (SEQ ID NO: 36) |
| GH1-1 | MAYPYDVPDYASLPKDFLWGFATAA |
| (FIG. 21, | YQIEGAIHADGRGPSIWDTFCNIPG |
| bottom) | KIADGSSGAVACDSYNRTKEDIDLL |
| | KSLGATAYRFSISWSRIIPVGGRND |
| | PINQKGIDHYVKFVDDLLEAGITPF |
| | ITLFHWDLPDGLDKRYGGLLNREEF |
| | PLDFEHYARTMFKAIPKCKHWITFN |
| | EPWCSSILGYNSGYFAPGHTSDRTK |
| | SPVGDSAREPWIVGHNLLIAHGRAV |
| | KVYREDFKPTQGGEIGITLNGDATL |
| | PWDPEDPLDVEACDRKIEFAISWFA |
| | DPIYFGKYPDSMRKQLGDRLPEFTP |
| | EEVALVKGSNDFYGMNHYTANYIKH |
| | KKGVPPEDDFLGNLETLFYNKKGNC |
| | IGPETQSFWLRPHAQGFRDLLNWLS |
| | KRYGYPKIYVTENGTSLKGENAMPL |
| | KQIVEDDFRVKYFNDYVNAMAKAHS |
| | EDGVNVKGYLAWSLMDNFEWAEGYE |
| | TRFGVTYVDYENDQKRYPKKSAKSL |
| | KPLFDSLIKKD |
| | (SEQ ID NO: 37) |
| GH1-1 with linker | MAYPYDVPDYASLPKDFLWGFATAA |
| and T2A amino | YQIEGAIHADGRGPSIWDTFCNIPG |
| acids | KIADGSSGAVACDSYNRTKEDIDLL |
| (FIG. 21, | KSLGATAYRFSISWSRIIPVGGRND |
| bottom) | PINQKGIDHYVKFVDDLLEAGITPF |
| | ITLFHWDLPDGLDKRYGGLLNREEF |
| | PLDFEHYARTMFKAIPKCKHWITFN |
| | EPWCSSILGYNSGYFAPGHTSDRTK |
| | SPVGDSAREPWIVGHNLLIAHGRAV |
| | KVYREDFKPTQGGEIGITLNGDATL |
| | PWDPEDPLDVEACDRKIEFAISWFA |
| | DPIYFGKYPDSMRKQLGDRLPEFTP |
| | EEVALVKGSNDFYGMNHYTANYIKH |
| | KKGVPPEDDFLGNLETLFYNKKGNC |
| | IGPETQSFWLRPHAQGFRDLLNWLS |
| | KRYGYPKIYVTENGTSLKGENAMPL |
| | KQIVEDDFRVKYFNDYVNAMAKAHS |
| | EDGVNVKGYLAWSLMDNFEWAEGYE |
| | TRFGVTYVDYENDQKRYPKKSAKSL |
| | KPLFDSLIKKDGSGSGEGRGSLLTC |
| | GDVEENPG |
| | (SEQ ID NO: 38) |
| GFP | MVSKGEELFTGVVPILVELDGDVNG |
| (FIG. 20, | HKFSVSGEGEGDATYGKLTLKFICT |
| bottom; FIG. | TGKLPVPWPTLVTTFTYGVQCFSRY |
| 21, bottom) | PDHMKQHDFFKSAMPEGYVQERTIS |
| | FKDDGNYKTRAEVKFEGDTLVNRIE |
| | LKGIDFKEDGNILGHKLEYNYNSHN |
| | VYITADKQKNGIKANFKIRHNIEDG |
| | SVQLADHYQQNTPIGDGPVLLPDNH |
| | YLSTQSALSKDPNEKRDHMVLLEFV |
| | TAAGITLGMDELYK |
| | (SEQ ID NO: 39) |
| GFP with T2A | PMVSKGEELFTGVVPILVELDGDVN |
| amino acid | GHKFSVSGEGEGDATYGKLTLKFIC |
| (FIG. 20, | TTGKLPVPWPTLVTTFTYGVQCFSR |
| bottom; FIG. | YPDHMKQHDFFKSAMPEGYVQERTI |
| 21, bottom) | SFKDDGNYKTRAEVKFEGDTLVNRI |
| | ELKGIDFKEDGNILGHKLEYNYNSH |
| | NVYITADKQKNGIKANFKIRHNIED |
| | GSVQLADHYQQNTPIGDGPVLLPDN |
| | HYLSTQSALSKDPNEKRDHMVLLEF |
| | VTAAGITLGMDELYK |
| | (SEQ ID NO: 40) |
| GH1-1 with linker, | MAYPYDVPDYASLPKDFLWGFATAA |
| T2A amino acids, | YQIEGAIHADGRGPSIWDTFCNIPG |
| and GFP | KIADGSSGAVACDSYNRTKEDIDLL |
| (FIG. 21, | KSLGATAYRFSISWSRIIPVGGRND |
| bottom) | PINQKGIDHYVKFVDDLLEAGITPF |
| | ITLFHWDLPDGLDKRYGGLLNREEF |
| | PLDFEHYARTMFKAIPKCKHWITFN |
| | EPWCSSILGYNSGYFAPGHTSDRTK |
| | SPVGDSAREPWIVGHNLLIAHGRAV |
| | KVYREDFKPTQGGEIGITLNGDATL |
| | PWDPEDPLDVEACDRKIEFAISWFA |
| | DPIYFGKYPDSMRKQLGDRLPEFTP |
| | EEVALVKGSNDFYGMNHYTANYIKH |
| | KKGVPPEDDFLGNLETLFYNKKGNC |
| | IGPETQSFWLRPHAQGFRDLLNWLS |
| | KRYGYPKIYVTENGTSLKGENAMPL |
| | KQIVEDDFRVKYFNDYVNAMAKAHS |
| | EDGVNVKGYLAWSLMDNFEWAEGYE |
| | TRFGVTYVDYENDQKRYPKKSAKSL |
| | KPLFDSLIKKDGSGSGEGRGSLLTC |
| | GDVEENPGPMVSKGEELFTGVVPIL |
| | VELDGDVNGHKFSVSGEGEGDATYG |
| | KLTLKFICTTGKLPVPWPTLVTTFT |
| | YGVQCFSRYPDHMKQHDFFKSAMPE |
| | GYVQERTISFKDDGNYKTRAEVKFE |
| | GDTLVNRIELKGIDFKEDGNILGHK |
| | LEYNYNSHNVYITADKQKNGIKANF |
| | KIRHNIEDGSVQLADHYQQNTPIGD |
| | GPVLLPDNHYLSTQSALSKDPNEKR |
| | DHMVLLEFVTAAGITLGMDELYK |
| | (SEQ ID NO: 41) |

### Mammalian Cell Culture and Transfection

Human embryonic kidney 293 T (HEK-293T) cells or PLATINUM-E™ (Plat-E) cells (CELL BIOLABS™; https://www.cellbiolabs.com/platinum-e-plat-e-retroviral-packaging-cell-line) were maintained in Dulbecco's Modified Eagle's Medium (DMEM) with 10% fetal bovine serum (FBS) and 1% penicillin/streptomycin (pen/strep) at 37C (37° C.) and 5% CO₂ (CO₂) in a hydrated incubator. PLATINUM-E™ (Plat-E) cells (CELL BIOLABS™) are based on the 293T cell line. They exhibit longer stability and produce higher yields of retroviral structure proteins. Plat-E cells contain gag, pol and env genes, allowing retroviral packaging with a single plasmid transfection.

For transfection, 1×10⁶ cells were seeded into a 6-well dish. The following day the cells were transfected using 450 fmol of DNA (approximately 2.5 micrograms [μg]) and Lipofectamine 3000 (Thermo, #L3000001) using the standard protocol (https://www.thermofisher.com/document-connect/document-connect.html?url=https %3A %2F %2Fassets.thermofisher.com %2FTFS-Assets %2FLSG %2Fmanuals %2Flipofectamine3000_protocol.pdf&title=TGlwb2ZlY3Rh bWluZSAzMDAwIFJlYWdlbnQgUHJvdG9jb2wgKEVuZ2xpc2gp). Essentially, cells were seeded to be 70-90% confluent at transfection. LIPOFECTAMINE™ 3000 Reagent in OPTI-MEM™ Medium (2 tubes) was diluted and mixed well. A master mix of DNA was prepared by diluting DNA in Opti-MEM™ Medium, then adding P3000™ Reagent, followed by mixing well. Diluted DNA was added to each tube of Diluted Lipofectamine™ 3000 Reagent (1:1 ratio) and incubated for 10-15 minutes at room temperature (approximately 19C-25C). DNA-lipid complex was added to the cells. The transfected cells were then analyzed or visualized.

### Immunoblotting

Cells were lysed with CST cell lysis buffer (10×) (CELL SIGNALING TECHNOLOGY®, #9803) and spun down at 17,000×g at 4C for 20 minutes to clear precipitates. The resulting supernatant is mixed with BOLT™ 4×LDS Sample Buffer (THERMOFISHER SCIENTIFIC™, #B0007; lithium dodecyl sulfate, pH 8.4) and BOLT™ 10× Sample Reducing Agent (THERMOFISHER SCIENTIFIC™, #B0009), to 1× final concentrations, and incubated at 70C (70° C.) for 10 minutes before being processed with gel electrophoresis on a BOLT™ 4 to 12%, Bis-Tris, 1.0 mm, Mini Protein Gel (THERMOFISHER SCIENTIFIC™, #NW04120BOX). Gel protein was then transferred to a polyvinylidene fluoride (PVDF) membrane using an IBLOT™ 2 Transfer Stack System (THERMOFISHER SCIENTIFIC™, #IB24002). The membrane was blocked by 60 minutes of room temperature incubation in TBS-T+5% BSA (Tris-buffered [tris(hydroxymethyl)aminomethane] saline with Tween-20 [polyoxyethylene (20) sorbitan monolaurate]+5% bovine serum albumin). The membrane was then transferred to overnight, 4C (4° C.) incubation with HA-Tag Rabbit monoclonal antibody (mAb) (CELL SIGNALLING TECHNOLOGY™, #C29F4) diluted 1:1000 in TBS-T+1% BSA. The following morning the membrane was washed 3 times in TBS-T (5 min each), before 60 min room temperature incubation with IRDye® 800CW Donkey anti-Rabbit IgG Secondary Antibody (LI-COR™, #926-32213), diluted 1:10,000 in TBS-T+1% BSA. The membrane was washed 3 times with TBS-T (5 min each) and imaged on a BIO-RAD™ CHEMIDOC™ MP Imaging System (BIO-RAD™).

### Immunocytochemistry

48 hours after transfection, PLATINUM-E™ cells (CELL BIOLABS™) were harvested and spun down onto NUNC™ LAB-TEK™ CHAMBER SLIDE™ SYSTEM (THERMOFISHER SCIENTIFIC™, #177402) slides. Cells were fixed with 2% paraformaldehyde (PFA)/5% sucrose and permeabilized using 1× Intracellular Staining Permeabilization Wash Buffer (BIOLEGEND™, #421002). After permeabilization, cells were blocked overnight at 4C (4° C.) with 5% donkey serum (https://www.sigmaaldrich.com/catalog/product/sigma/d9663?lang=en&region=US&gclid=CjwKCAjwjbCDBhAwEiwAiudBy_sMpY439ksB7ddOSgfnwUSaQrXG7kviZRq15H7 YgGn9gloXG9_rshoCnlsQAvD_BwE; Sigma Aldrich, D9663-10ML). The following morning, cells were incubated with ALEXA FLUOR® 647 anti-HA.11 Epitope Tag Antibody (Clone 16B12) (BIOLEGEND™, #682404) for 1 hr at room temperature before washing three times (5 min each) with 1× perm buffer. Cells were then overlaid with FLUOROMOUNT-G™ Mounting Medium, with DAPI (4′,6-diamidino-2-phenylindole) (THERMOFISHER SCIENTIFIC™, #00-4959-52) and imaged using a NIKON® ECLIPSE™ Ti+YOKOGAWA™ CSU-X1 Confocal Spinning Disc Scanning System.

### T Cell Activation and Transduction

Spleens from BL/6J mice (Jackson Laboratory #000664; https://www.jax.org/strain/000664) were harvested, minced, resuspended in fluorescence-activated cell sorting (FACS) buffer (phosphate buffered saline (PBS)+2% fetal bovine serum (FBS)+1 mM ethylene diamine tetra-acetic acid (EDTA) (THERMOFISHER SCIENTIFIC™, #15575020)), and strained through a 40-micron nylon mesh filter (Millipore Sigma, CLS431750-50EA). The cellular suspension was then centrifuged at 600× g for 10 minutes before isolating CD8+ T cells using standard protocol from an immunomagnetic separation kit (EASYSEP™ Mouse CD8+ T Cell Isolation Kit, STEMCELL™ Technologies, #19853).

For activation, 5×10⁶ stained CD8+ T cells were resuspended in 1 mL of complete T cell medium (RPMI 1640+10% fetal bovine serum (FBS)+1 mM Na Pyruvate+10 mM 4-(2-hydroxyethyl)-1-piperazineethanesulfonic acid (HEPES)+1% penicillin/streptomycin (pen/strep)+0.1% beta-mercaptoethanol) supplemented with 2 ug/mL anti-CD28 clone 37.51 (BIOXCELL™, #BE0015-1) and plated into one well of a 12 well plate coated with 10 micrograms/mL (μg/mL) of anti-CD3e clone 2C11 (BIOXCELL™, #BE0001-1). 24 hours later, plates were lightly spun down at 100× g for 2 minutes, before removing 800 ul of supernatant. 1 mL of T cell media containing concentrated virus was then overlaid, before centrifugation at 800×g for 1 hour at 32C (32° C.). After centrifugation, the plates were placed into a 37C (37° C.), 5% CO₂ (CO₂), humidified incubator for one hour to allow T cells to equilibrate. 1 mL of media containing the concentrated virus was then removed before replacement with 1 mL of complete T cell media containing 2 micrograms/mL (μg/mL) anti-CD28.

### Retrovirus Production

24 hours after PLATINUM-E™ cell (CELL BIOLABS™) transfection, the media was replaced and cells were incubated for another 24 hours, at which point the supernatant was harvested and centrifuged for 5 minutes at 700× g to pellet any cells in suspension. Supernatants were then concentrated by centrifugation at 1000×g for 15 minutes using AMICON™ Ultra-15 Centrifugal Filter Units (MILLIPORE™, #UFC910008). Concentrated virus was brought up to 1 mL using complete T cell media and supplemented with 5 micrograms/mL (μg/mL) Polybrene (SIGMA-ALDRICH™, #TR-1003-G). Fresh (non-refrigerated, non-frozen) virus was consistently used to maintain high viral titers.

### Proliferation Assays

For PLATINUM-E™ cells (CELL BIOLABS™), 48 hours after transfection, the cells were harvested with 0.05% Trypsin-ethylene diamine tetra-acetic acid (EDTA) (THERMOFISHER SCIENTIFIC™), pelleted by centrifugation at 350×g for 5 minutes (min), and resuspended at a concentration of 1×10⁶ cells/mL in phosphate buffered saline (PBS)+0.1% bovine serum albumin (BSA) with 5 micromolar (μM) CELLTRACE™ Violet (THERMOFISHER SCIENTIFIC™, #C34571). Cell suspensions were incubated for 15 minutes at 37C (37° C.) before the reaction was quenched with 5× volume of complete T cell media (see above) containing 10% fetal bovine serum (FBS; THERMOFISHER SCIENTIFIC™ #A3382001; https://www.thermofisher.com/order/catalog/product/26140079 #/26140079). Cells were again pelleted, and resuspended in 2 mL basal media (THERMOFISHER SCIENTIFIC™ #A1443001) containing 10% dialyzed fetal bovine serum (FBS) (THERMOFISHER SCIENTIFIC™, #A3382001) and 1% penicillin/streptomycin (pen/strep). 500 microliters (μL) of the cell suspension were then plated in triplicate into a 12-well plate. Next, 500 microliters (μL) of 2× metabolic assay media was overlaid, which comprised the basal medium+glucose at 10 mM or 200 micromolar (μM) or the basal medium+cellobiose (SIGMA™, #22150) at 10 mM. Cells were then incubated at 37C (37° C.) and 5% CO₂ (CO₂) in a humidified chamber for 48 hours before harvesting for flow cytometric analysis. Basal medium was composed of DMEM without glucose and glutamine and with 10% dialyzed FBS (THERMOFISHER SCIENTIFIC™, #A338200) and 1% penicillin/streptomycin. In some instances, events with a particular forward and side scatter (“alive cells”) were selected for further analysis. Within this population, events that were GFP and mCherry positive (viable GFP+ mCherry+) were selected for further analysis.

For T cells (sourced from spleens of BL/6J mice, as discussed above), CELLTRACE™ Violet staining took place immediately before plating for activation and followed the same protocol. T cells were stained at this stage because of their uniform size and status in the cell cycle, allowing for an enhanced capacity to track cell generations. T cells were plated into metabolic assay media (basal media=AGILENT™, #103576-100) 24 hours after transduction and allowed to grow for 48 hours before analysis on a flow cytometer.

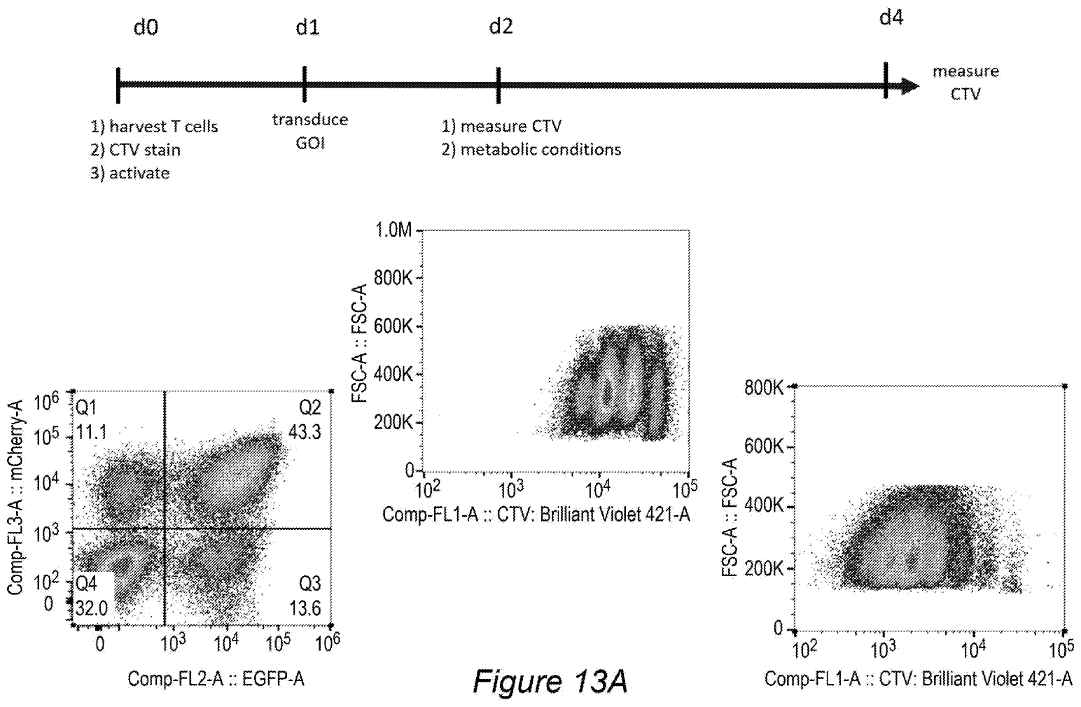

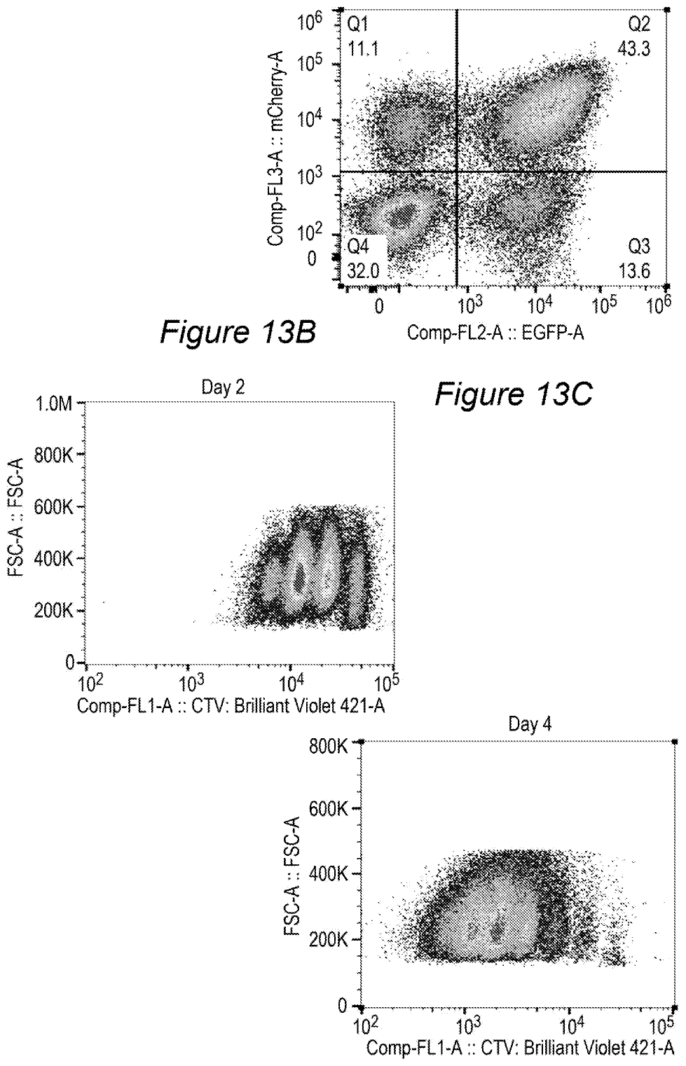

In some instances, with respect to double-transductants (e.g., FIGS. 13A-13D), sorting techniques were not used. Instead, a small aliquot of cells was run on the cytometer to measure transduction efficiency (cells double positive for GFP and mCherry) and to measure the state of CTV signal at the onset of the various metabolic incubations. Otherwise, the assays were carried out on the bulk population of cells.

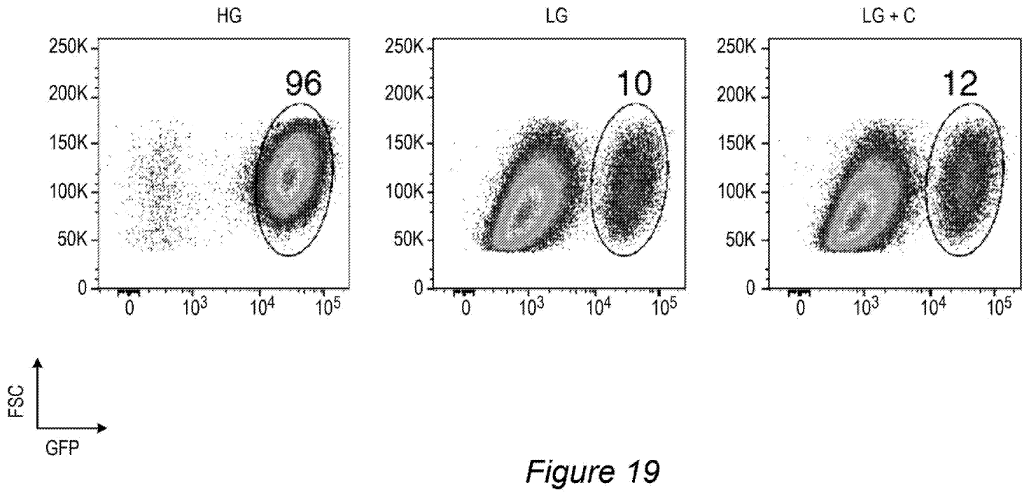

The protocol for proliferation of the B16 melanoma tumor cell line (FIG. 19) constitutively expressing GFP is identical to the other proliferation assays described above, minus the prior transfection with plasmids.

### Flow Cytometry and Cell Sorting

PLATINUM-E™ cells (CELL BIOLABS™) were harvested using trypsinization, centrifuged at 350×g for 5 minutes, and resuspended in fluorescence-activated cell sorting (FACS) buffer (phosphate buffered saline [PBS]+2% fetal bovine serum [FBS]+1 mM ethylene diamine tetra-acetic acid [EDTA]). Cell suspensions were passed through cell strainer into 5 mL tubes (FALCON™, #352235) and then analyzed or processed on a SONY® SH800S cell sorter.

In some instances, with respect to double-transductants (e.g., FIGS. 13A-13D), sorting techniques were not used. Instead, a small aliquot of cells was run on the cytometer to measure transduction efficiency (cells double positive for GFP and mCherry) and to measure the state of CTV signal at the onset of the various metabolic incubations. Otherwise, the assays were carried out on the bulk population of cells.

### Example 14: Construction of Gh1-1 and Cdt-1 Vectors and Expression of Same

Vectors for gene delivery into mouse T cells were designed (FIG. 1) to utilize components of the mouse stem cell virus (MSCV), a retrovirus capable of delivery DNA cargo into a target genome. The MSCV system comprises long terminal repeats (LTRs) that serve both to integrate into host genome and to promote and suppress transcription of DNA cargo. The vector contains a MESV Ψ signal element that facilitates packaging of the viral RNA into capsids particles. The only difference between the vectors is the expression of different fluorescent markers, with GFP or mCherry being constitutively driven by the PGK promoter. When these plasmids are transfected into the PLATINUM-E™ cell line (CELL BIOLABS™), a derivative of HEK-293T cell line, that expresses the gag (group antigens polyprotein), pol (reverse transcriptase polymerase), and env (envelope) viral proteins, infectious viral particles are produced that can be used to transduce primary T cells. The envelope of the virus is ecotropic (i.e., can only infect mouse or rat cells). The MCS_PGK-GFP vector (FIG. 1, top; SEQ ID NO: 7) includes the following elements from the 5′-end: 5′ long terminal repeats (5′ LTR), murine embryonic stem cell virus psi (MESV ψ), multiple cloning site (MCS), mouse phosphoglycerate kinase 1 promoter (PGK promoter), folding reporter green fluorescent protein (frGFP; abbreviated GFP herein) as a marker, and 3′ long terminal repeats (3′ LTR). The MCS_PGK-mCherry vector (FIG. 1, bottom; SEQ ID NO: 8) is identical expect that it utilizes mCherry, a member of the monomeric red fluorescent protein family, as a marker.

CDT-1 and GH1-1 were amended at their N-termini with an HA peptide tag to allow for antibody-mediated analysis of protein expression. FIG. 2 shows schematic maps of vectors for genomic integration of DNA cargo, with the codon optimized gh1-1 gene, expressing BETA-GLUCOSIDASE (GH1-1; Neurospora crassa [strain ATCC 24698/74-OR23-1A/CBS 708.71/DSM 1257/FGSC 987]), with the codon optimized cdt-1 gene, expressing CELLODEXTRIN TRANSPORTER 1 (Neurospora crassa [strain ATCC 24698/74-OR23-1A/CBS 708.71/DSM 1257/FGSC 987]). Each expressed protein was designed to have an N-terminal hemagglutinin tag (HA) on either the GH1-1 or CDT-1 protein. HA-cdt-1 or HA-gh1-1 constructs were inserted into the MCS of the vectors shown in FIG. 1. As a result, gh1-1 (gh1-1-PGK GFP [FIG. 2, above top; SEQ ID NO: 9]) utilizes folding reporter green fluorescent protein (frGFP) as a marker, while the other gene cdt-1 (cdt-1 PGK mCherry [FIG. 2, bottom; SEQ ID NO: II], utilizes mCherry as a marker. Cdt-1 was also placed into the frGFP backbone (cdt-1 PGK frGFP [FIG. 2, below top; SEQ ID NO: 10]).

PLATINUM-E™ cells (CELL BIOLABS™) were cultured and maintained. For transfection, 1×10⁶ cells were seeded into a 6-well dish overnight, then transfected using 450 fmol DNA (approximately 2.5 micrograms [μg]) and LIPOFECTAMINE™ 3000 (THERMOFISHER SCIENTIFIC™, #L3000001) with standard protocols. [00362] 48 hours after transfection, cells were lysed and centrifuged to clear precipitates. The resulting supernatant was mixed with buffer and reducing agent and subjected to gel electrophoresis. Gel proteins were then transferred to a polyvinylidene fluoride (PVDF) membrane, which was blocked with TBS-T+ 5% BSA (Tris-buffered [tris(hydroxymethyl)aminomethane] saline with Tween-20 [polyoxyethylene (20) sorbitan monolaurate]+5% bovine serum albumin). The membrane was then transferred overnight via incubation with HA-Tag Rabbit monoclonal antibody (mAb) (CELL SIGNALLING TECHNOLOGY™, #C29F4) diluted 1:1000 in TBS-T+1% BSA, then subjected to a series of washes before being incubated for 60 mins with IRDye® 800CW Donkey anti-Rabbit IgG Secondary Antibody (LI-COR™, #926-32213), diluted 1:10,000 in TBS-T+1% BSA, followed by additional washes and imaging on a BIO-RAD™ CHEMIDOC™ MP Imaging System (BIO-RAD™).

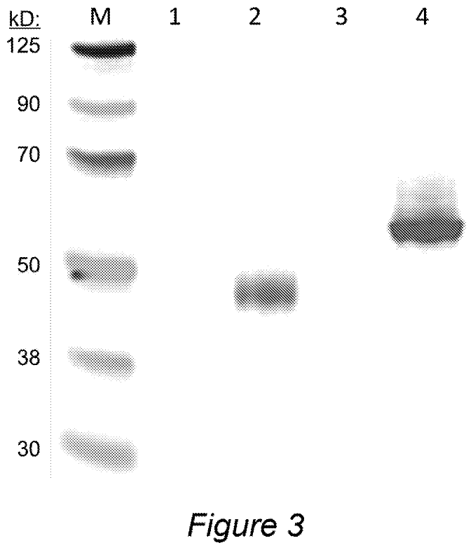

FIG. 3 is a photograph of a PLATINUM-E™ (Plat-E) (CELL BIOLABS™) immunoblot gel ladder (M), MSCV mCherry control (1), MSCV cdt-1 PGK mCherry vector (2), MSCV GFP control (3), and MSCV gh1-1 GFP vector (4). The ladder (M) provides proteins with the sizes (kDa) as indicated on the left of FIG. 3. Immunoblot analysis of single-gene transfectants shows CDT-1 is expressed but migration corresponds to a shifted molecular weight (approximately 45 kDa, with smaller, fainter bands at approximately 36 kDa vs. predicted 64 kD). GH1-1 expression is robust and at the expected molecular weight (predicted 55 kD).

To enrich the fraction of CDT-1 found in the plasma membrane, the HA-tag was transferred to the C-terminus (FIG. 4, top; SEQ ID NO: 12), based on prior work showing that green fluorescent protein (GFP) fused to the C-terminus does not inhibit protein function (Lian et al. (2014) Biotechnol. Bioeng. 111: 1521-1531). This vector construct includes the following elements from the 5′-end: 5′ LTR, MESV psi (MESV T), cdt-1 with C-terminal HA tag-encoding sequence inserted in the MCS, PGK promoter, mCherry, and 3′ LTR (FIG. 4, top; SEQ ID NO: 12).

Additionally, the C-terminus was amended with an endoplasmic reticulum export signal (ERES) (FIG. 4, bottom; SEQ ID NO: 13), which results in protein localization to the plasma membrane (PM), even for proteins not normally found there (Stockklausner et al. (2001) FEBS Lett. 493: 129-133). This vector construct includes the following elements from the 5′-end: 5′ LTR MESV psi (MESV ψ), cdt-1 with HA tag (HA tag-encoding sequence expressed C-terminal to the cdt-1) and discrete endoplasmic reticulum export signal-encoding sequence (ERES), PGK promoter, mCherry, and 3′ LTR (FIG. 4, bottom; SEQ ID NO: 13).

### Example 15: Mammalian Expression of Cellodextrin Transporter and β-Glucosidase

and Optimization Thereof [00366] 48 hours after transfection, PLATINUM-E™ cells (CELL BIOLABS™) were harvested and spun down onto slides. Cells were fixed and permeabilized. After permeabilization, cells were blocked overnight at 4C (4° C.) with 5% donkey serum (Sigma Aldrich, D9663-10ML). The following morning, cells were incubated with ALEXA FLUOR® 647 anti-HA.11 Epitope Tag Antibody (Clone 16B12) (BIOLEGEND™, #682404) for 1 hr at room temperature before washing three times (5 min each) with 1× perm buffer. Cells were then overlaid with FLUOROMOUNT-G™ Mounting Medium, with DAPI (4′,6-diamidino-2-phenylindole) (THERMOFISHER SCIENTIFIC™, #00-4959-52) and imaged using a NIKON® ECLIPSE™ Ti+YOKOGAWA™ CSU-X1 Confocal Spinning Disc Scanning System.

FIG. 5 is a series of confocal micrographs showing PLATINUM-E™ immunocytochemistry of PLATINUM-E™ cells (CELL BIOLABS™) transfected with the vector constructs as shown (MSCV GFP control [top row; see FIG. 1—top for vector; SEQ ID NO: 7]; MSCV gh1-1 GFP [middle row; see FIG. 2—below top for vector; SEQ ID NO: 10]; MSCV HA-cdt-1 GFP [N-terminal HA tag] [bottom row; see FIG. 2—above top for vector; SEQ ID NO: 9]), then detected as indicated with, left to right: green fluorescent protein (GFP); 2-(4-amidinophenyl)-1H-indole-6-carboxamidine (4′,6-diamidino-2-phenylindole; DAPI); GFP+DAPI; hemagglutinin (HA); HA+DAPI. Detection of the HA tag indicates gh1-1 and cdt-1 protein expression. However, while cdt-1 seems to localize to plasma membrane, it is also present diffusely in cytosol.

To enrich the fraction of CDT-1 found in the plasma membrane, the HA-tag was transferred to the C-terminus (FIG. 4, top; SEQ ID NO: 12), as described above. Additionally, the C-terminus was amended with an endoplasmic reticulum export signal (ERES) (FIG. 4, bottom; SEQ ID NO: 13), which results in protein localization to the plasma membrane (PM), even for proteins not normally found there, as described above. PLATINUM-E™ cells (CELL BIOLABS™) were transfected, harvested, and spun down onto slides, followed by fixing, permeabilizing, incubating with ALEXA FLUOR® 647 anti-HA.11 Epitope Tag Antibody (Clone 16B12) (BIOLEGEND™, #682404), and mounting as described.

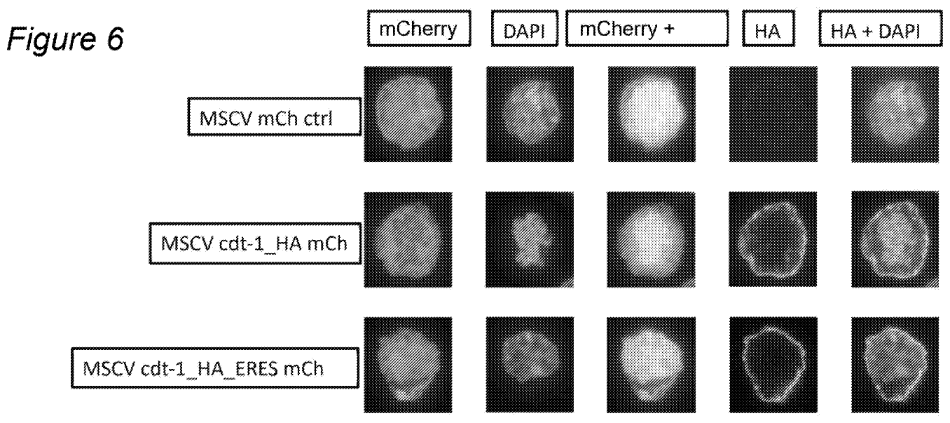

FIG. 6 is a series of confocal micrographs showing PLATINUM-E™ immunocytochemistry of PLATINUM-E™ cells (CELL BIOLABS™) transfected with the constructs as shown (MSCV mCherry control [top row; see FIG. 1—bottom for vector; SEQ ID NO: 8]; MSCV cdt-1 HA [HA tag C-terminal to cdt-1 protein] [middle row; see FIG. 4—top for vector; SEQ ID NO: 12]; MSCV cdt-1 HA ERES mCherry [bottom row; see FIG. 4—bottom for vector; SEQ ID NO: 13]), then detected as indicated with, left to right: mCherry; DAPI; mCherry+DAPI; HA; HA+DAPI. Compared with the results in FIG. 5, FIG. 6 demonstrates that cdt with C-terminal amendments (C-terminal HA or C-terminal HA ERES) shows much more discrete localization only in plasma membrane.

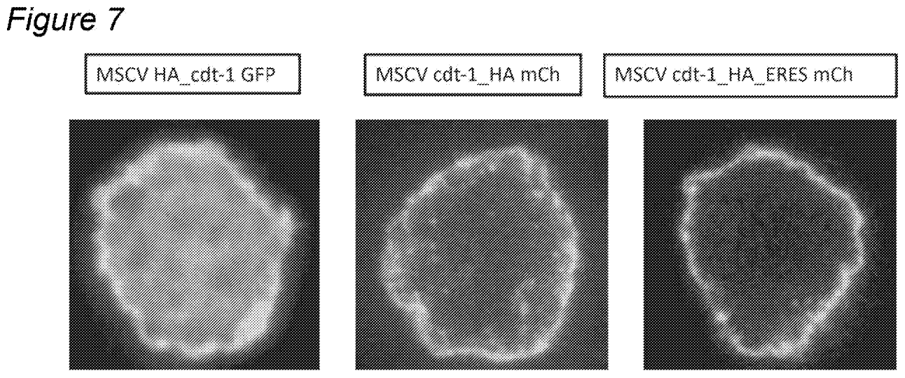

FIG. 7 compares selected electron micrographs of FIG. 5 and FIG. 6 showing PLATINUM-E™ immunocytochemistry of HA detection in PLATINUM-E™ cells (CELL BIOLABS™) transfected with the constructs as shown (MSCV HA cdt-1 GFP [N-terminal HA tag] [left; see FIG. 2—above top for vector; SEQ ID NO: 9]; MSCV cdt-1 HA mCherry [C-terminal HA tag] [center; see FIG. 4—top for vector; SEQ ID NO: 12]; MSCV cdt-1 HA ERES mCherry [C-terminal HA tag+ERES] [right; see FIG. 4—bottom for vector; SEQ ID NO: 13]), demonstrating that the addition of an HA tag and ERES peptide motif to the C-terminus of protein results in best protein localization (right).

The combination of a C-terminal HA-tag and ERES improved the localization of CDT-1 to the PM. These data show the feasibility and impact of manipulating the translational and post-translational aspects of these proteins.

### Example 16: Assessment of Mammalian Cellobiose Metabolism and Proliferation with MSCV-GH1-1-GFP Vector and MSCV-CDT-1-mCherry Vector

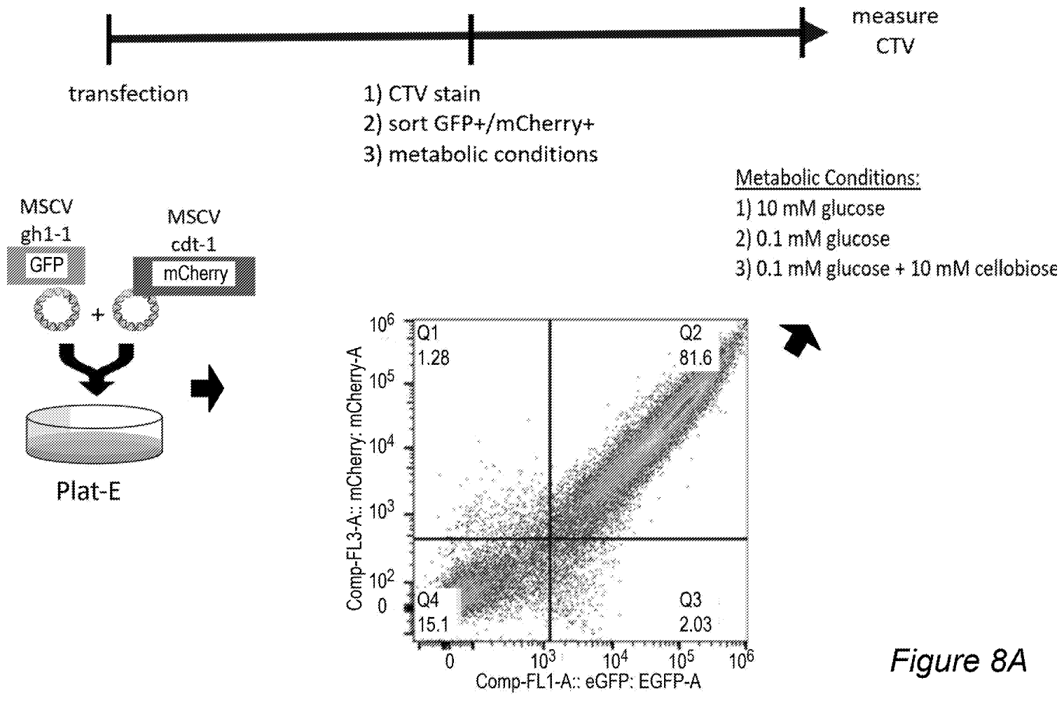

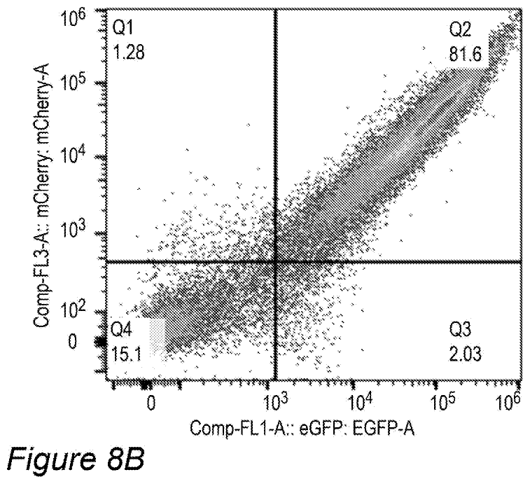

CDT-1 and GH1-1 were codon optimized and cloned into mouse stem cell virus (MSCV) plasmids as described. These constructs facilitated ecotropic retrovirus production when transfected into PLATINUM-E™ cells (CELL BIOLABS™). To study effectiveness, functional experiments were performed and results were obtained measuring cell proliferation with a combination of cellobiose and low glucose, as compared to high glucose and low glucose controls (FIG. 8A—top).

PLATINUM-E™ cells (CELL BIOLABS™) were transfected with plasmids of interest, plated in metabolic conditions, and their proliferation monitored. 48 hours after transfection, the cells were harvested, pelleted by centrifugation, and resuspended at a concentration of 1×10⁶ cells/mL in phosphate buffered saline (PBS)+0.1% bovine serum albumin (BSA) with 5 micromolar (μM) CELLTRACE™ Violet (CTV) (THERMOFISHER SCIENTIFIC™, #C34571) (FIG. 8A—top). Transfection took place on Day 0 (d0). PLATINUM-E™ cells were transfected with MSCV gh1-1 GFP vector (see FIG. 2—top below for vector) and MSCV cdt-1 mCherry vector (see FIG. 2 bottom for vector), as represented by the schematic (FIG. 8A—bottom left). On Day 2 (d2), cells were stained with CELLTRACE™ VIOLET using the CELLTRACE™ VIOLET Cell Proliferation Kit (CTV; THERMOFISHER™ SCIENTIFIC C34557, then sorted via fluorescence-activated cell sorting (FACS) for GFP+/mCherry+ cells (FIG. 8A—bottom center graph,), and then plated in metabolic conditions as shown on the schematic (FIG. 8A—bottom right). Metabolic conditions were: 10 millimolar (mM) glucose (high glucose); 0.1 mM glucose (low glucose); or 0.1 mM glucose (low glucose)+10 mM cellobiose. On Day 4 (d4), cells were harvested, and proliferation under each metabolic condition was measured as a function of CTV. Cells were then incubated at 37C (37° C.) and 5% CO₂ (CO₂) in a humidified chamber for 48 hours before harvesting for flow cytometric analysis (FIGS. 8A-8B).

FIG. 8B, an expanded view of the corresponding graph in FIG. 8A bottom center, is a logarithmic graph depicting the results of flow cytometric measurement of the GFP (X-axis) and mCherry (y-axis) fluorescent signals of PLATINUM-E™ cells post-transfection. By looking at Quadrant 2 it can be seen that 81% of the cells are double-positive, and it was this population that was sorted for proliferation analysis.

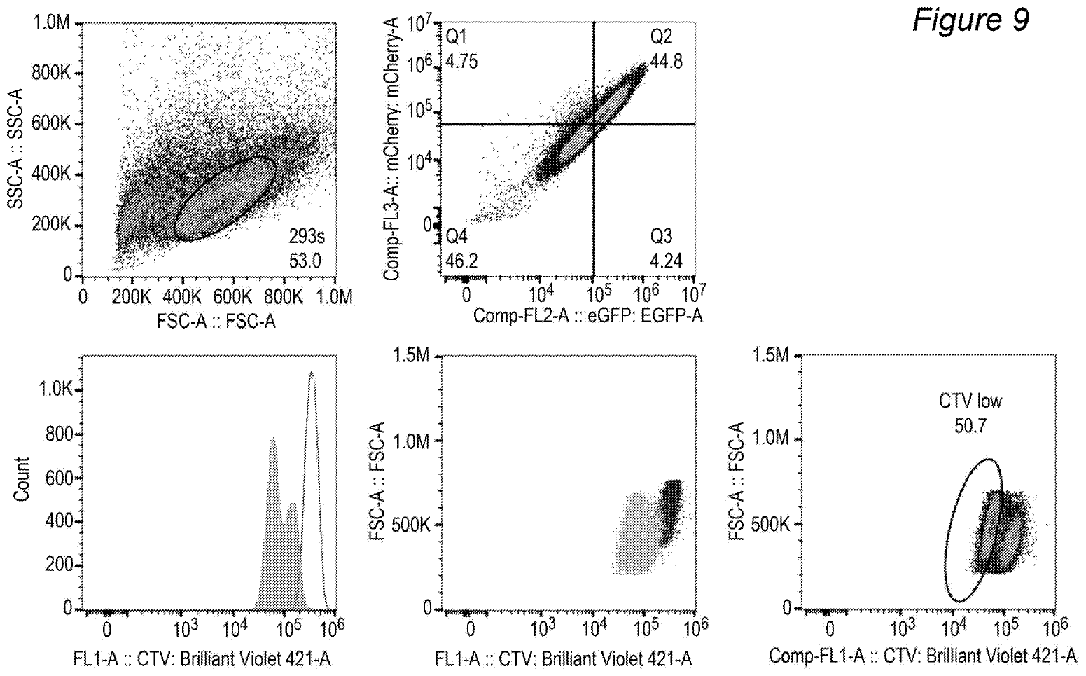

The results of the PLATINUM-E™ (CELL BIOLABS™) cell proliferation experiment of FIGS. 8A-B were further analyzed in a series of graphs (FIG. 9) depicting the analysis pipeline used to determine which cells underwent division. Forward (x-axis) and side (y-axis) scatter were used to determine viable cells (FIG. 9—top left, gating on “size”). Within this population, the cells expressing the highest levels of GFP (x-axis) and mCherry (y-axis) were gated for further analysis (FIG. 9—top center, gating on “GFP+ mCherry+”). Within the GFP+ mCherry+ population, a histogram projection displays the level of CELLTRACE™ Violet fluorescent signal (FIG. 9—bottom left). This analysis is transformed by changing the y-axis from counts to forward scatter (FIG. 9—bottom center). Finally, the discrete population of cells that has had the fluorescent signal diluted in half, which is considered the population of cells that has undergone division, was gated and quantified. (FIG. 9—bottom right, “% divided”).

### Example 17: Assessment of Mammalian Cellobiose Metabolism and Proliferation with MSCV-GH1-1-GFP Vector and MSCV-CDT-1-HA-IDTv1-mCherry Vector or MSCV-GH1-1-GFP Vector and MSCV-CDT-1-HA-ERES-mCherry Vector

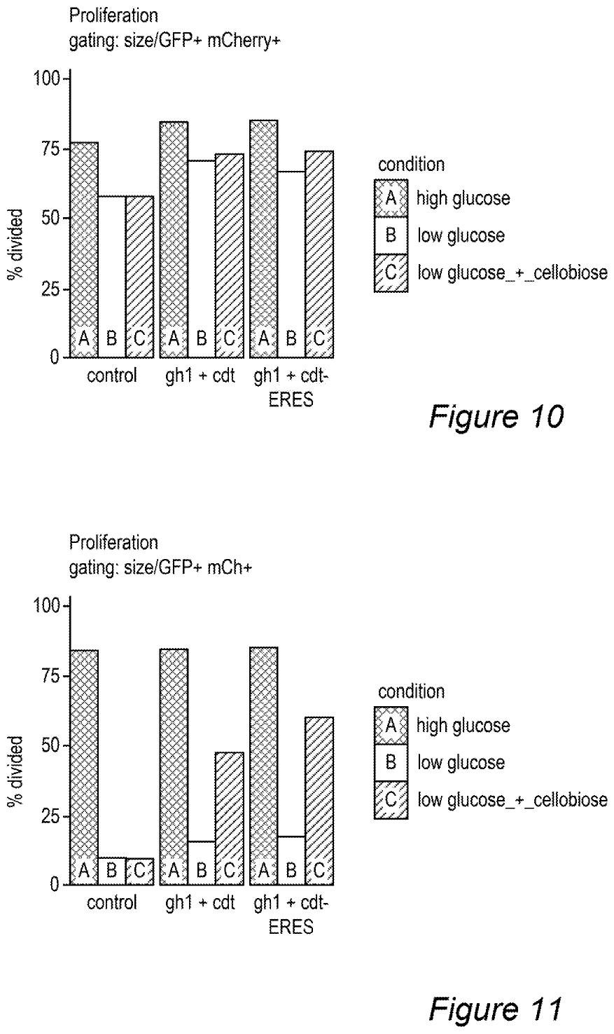

The results of a cell proliferation study in PLATINUM-E™ cells (CELL BIOLABS™) that follows the pipeline illustrated in FIG. 8A above. FIG. 10 shows the results of the percentage of the viable, GFP+ mCherry+ cells that underwent division when incubated in each of the three metabolic conditions (high glucose [gray]; low glucose [blue]; low glucose+cellobiose [green]) for PLATINUM-E™ cells transfected with the control vectors (left trio, FIG. 1—top and bottom), PLATINUM-E™ cells transfected with HA-GH1-1-GFP and CDT-1-HA-IDTv1 vectors (center trio, FIG. 2—below top+FIG. 4—top), and PLATINUM-E™ cells transfected with HA-GH1-1-GFP and CDT-1-HA-ERES vectors (right trio, FIG. 2—below top+FIG. 4—bottom). A slightly higher fraction of cells co-transfected with cdt-1 and gh1-1 undergo cell division in the presence of cellobiose compared to control.

### Example 18: Assessment of Mammalian Cellobiose Metabolism and Proliferation in Reduced Glutamine and Serum Protein Conditions

A second cell proliferation study was conducted. The proliferation experiment was repeated with different basal metabolic conditions, with the base media containing 5× less dialyzed FBS and 10× less D-glutamine (with new final concentrations of 2% and 200 uM respectively). FIG. 11 shows the results of the percentage of the viable, GFP+ mCherry+ cells that underwent division when incubated in the three metabolic conditions (high glucose [gray]; low glucose [blue]; low glucose+cellobiose [green]) for PLATINUM-E™ cells (CELL BIOLABS™) transfected with a the control vectors (left trio, FIG. 1 top and bottom [SEQ ID NO: 7 and SEQ ID NO: 8]), PLATINUM-E™ cells transfected with gh1-1 and cdt-1 vectors (center trio, FIG. 2 below top [SEQ ID NO: 10]+FIG. 4 top [SEQ ID NO: 12]), and PLATINUM-E™ cells transfected with gh1-1 and cdt-1-ERES vectors (right trio, FIG. 2 below top [SEQ ID NO: 10]+FIG. 4 bottom [SEQ ID NO: 13]).

Glutamine (and protein found in fetal bovine serum [FBS]) feed heavily into bioenergetic and biosynthetic pathways. In the absence of these alternative resources, the impact of glucose withdrawal (and rescue) became more apparent. Therefore, the ability of cells expressing gh1-1 and cdt-1 to proliferate using cellobiose was more apparent.

### Example 19: Effect of CDT-1 and GH1-1 on Cell Proliferation and Morphology

To assess the effect of CDT-1 and GH1-1 on cell proliferation, PLATINUM-E™ cells (CELL BIOLABS™) were transfected.

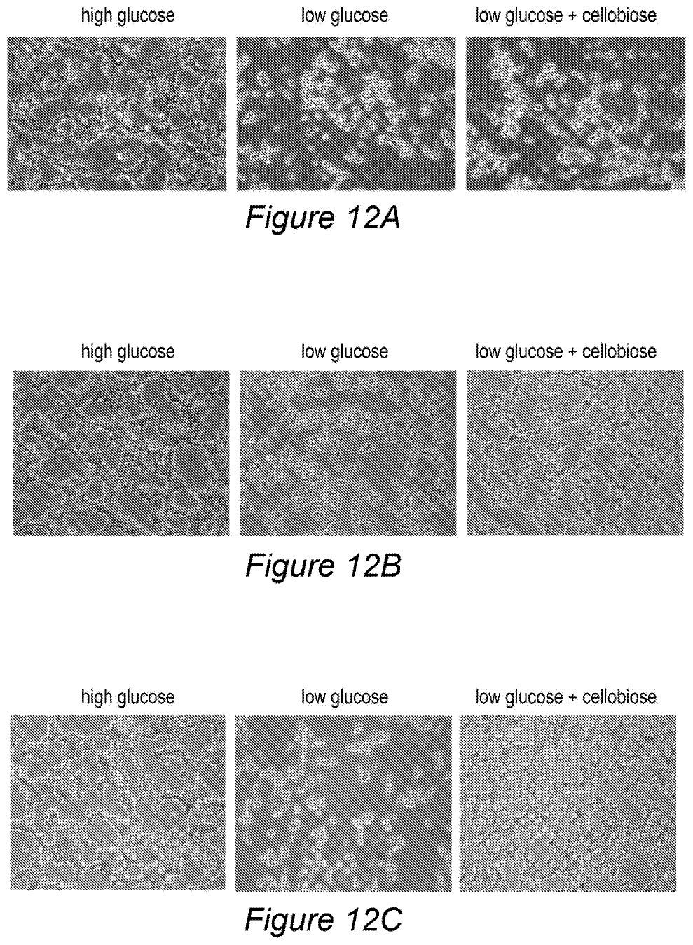

FIGS. 12A-12C are a series of compound light micrographs of PLATINUM-E™ cells (CELL BIOLABS™) in culture. FIG. 12A is a series of compound light micrographs of PLATINUM-E™ cells transfected with both of the parent plasmids [FIG. 1—top and bottom [SEQ ID NO: 7 and SEQ ID NO: 8] under various metabolic conditions (high glucose [left]; low glucose [center]; low glucose+cellobiose [right]). Of note is cell morphology, with cell cultured in high glucose showing a larger size and cellular projections, and adherence to the surface. Cells cultured in low glucose and low glucose+cellobiose display smaller, spherical morphology and exist in suspension or loosely adhered to the cell plate. FIG. 12B is a series of compound light micrographs of PLATINUM-E™ cells transfected with the gh1-1 vector [FIG. 2 below top (SEQ ID NO: 10)] and the cdt-1 vector [FIG. 4 top (SEQ ID NO: 12) under various metabolic conditions (high glucose [left]; low glucose [center]; low glucose+cellobiose [right]). Of note is cell morphology and adherence to the surface, with gh1-1+cdt-1 expressing cells displaying rescued size, projections, and adherence to the culture surface in the presence of low glucose+cellobiose. FIG. 12C is a series of compound light micrographs of PLATINUM-E™ cells transfected with the gh1-1 vector [FIG. 2 below top (SEQ ID NO: 10)] and the cdt-1-ERES vector [FIG. 4 bottom (SEQ ID NO: 12)] under various metabolic conditions (high glucose [left]; low glucose [center]; low glucose+cellobiose [right]).

Of note is cell morphology and adherence to the surface, again with gh1-1+cdt-1-ERES expressing cells displaying rescued size, projections, and adherence to the culture surface in the presence of low glucose+cellobiose.

### Example 20: Assessment of Mammalian Cellobiose Metabolism and Proliferation in Murine T Cells

Spleens from BL/6J mice were harvested, and CD8+ T cells were isolated using standard protocol from an immunomagnetic separation kit (EASYSEP™ Mouse CD8+ T Cell Isolation Kit, STEMCELL™ Technologies, #19853), as described above.

PLATINUM-E™ cell (CELL BIOLABS™) were transfected and incubated. Supernatant was harvested and centrifuged to pellet any cells in suspension. Supernatants were then concentrated by centrifugation at 1000×g for 15 minutes using AMICON™ Ultra-15 Centrifugal Filter Units (MILLIPORE™, #UFC910008). Concentrated virus was brought up to 1 mL using complete T cell media and supplemented with 5 micrograms/mL (μg/mL) Polybrene (SIGMA-ALDRICH™, #TR-1003-G). Fresh (non-refrigerated, non-frozen) virus was consistently used to maintain high viral titers.

For activation, 5×10⁶ stained CD8+ T cells were resuspended in 1 mL of complete T cell medium (RPMI 1640+10% fetal bovine serum (FBS)+1 mM Na Pyruvate+1% 4-(2-hydroxyethyl)-1-piperazineethanesulfonic acid (HEPES)+1% penicillin/streptomycin (pen/strep)+0.1% beta-mercaptoethanol) supplemented with 2 ug/mL anti-CD28 clone 37.51 (BIOXCELL™, #BE0015-1) and plated into one well of a 12 well plate coated with 10 micrograms/mL (μg/mL) of anti-CD3e clone 2C11 (BIOXCELL™, #BE0001-1). 24 hours later, plates were lightly spun down at 100× g for 2 minutes, before removing 800 ul of supernatant. 1 mL of T cell media containing concentrated virus was then overlaid, before centrifugation at 800×g for 1 hour at 32C (32° C.), as described above. After centrifugation, the plates were placed into a 37C (37° C.), 5% CO₂ (CO₂), humidified incubator to allow T cells to equilibrate. 1 mL of media containing the concentrated virus was then removed before replacement with 1 mL of complete T cell media containing 2 micrograms/mL (μg/mL) anti-CD28.

For T cells (sourced from BL/6J splenocytes), CELLTRACE™ Violet (CTV) staining took place immediately before plating for activation and followed the same protocol used for PLATINUM-E™ cells. T cells were stained at this stage because of their uniform size and status in the cell cycle, allowing for an enhanced capacity to track cell generations. T cells were plated into metabolic assay media (basal media=AGILENT™ #103576-100) 24 hours after transduction and allowed to grow for 48 hours before analysis on a flow cytometer.

FIGS. 13A-13D are a schematic timeline and graphs depicting a T cell functional experimental method and results measuring cell proliferation with a combination of cellobiose and low glucose, as compared to high glucose and low glucose controls. Transduced T cells were assessed for their ability to proliferate with cellobiose. T cells received genetic cargo in the form of MSCV virus and then expressed gh1-1 and cdt-1. They were put into metabolic conditions and then their proliferation assessed. FIG. 13A shows a schematic timeline (top) of the experiment. On Day 0 (d0), T cells were harvested from the spleen of a BL/6J mouse, stained with CELLTRACE™ VIOLET using the CELLTRACE™ VIOLET Cell Proliferation Kit (CTV; THERMOFISHER™ SCIENTIFIC C34557and then activated. On Day 1 (d1), the stained, activated T cells were transduced by spinfection with MSCV virus containing empty control, cdt-1, or gh1-1 genetic cargo [empty controls=FIG. 1—top and bottom (SEQ ID NO: 7 and SEQ ID NO: 8), gh1-1=FIG. 2—below top (SEQ ID NO: 10), cdt-1=FIG. 4 top (SEQ ID NO: 12) or FIG. 4 bottom (SEQ ID NO: 13)]. On Day 2 (d2), measured for CTV, and plated in metabolic conditions. On Day 4 (d4) proliferation under each metabolic condition was measured as a function of CTV. FIG. 13B is an enlarged view of the graph of FIG. 13A (bottom left) depicting mCherry (y-axis) and GPF (x-axis) fluorescent signal of T cells one day post-transduction, demonstrating that a significant fraction of T cells is successfully co-transduced, based on the percentage of cells that are double-positive for mCherry and GFP. FIG. 13C is an enlarged view of the graph of FIG. 13A (bottom center) depicting forward scatter (y-axis) and CELLTRACE™ VIOLET (x-axis) fluorescent signal on Day 2. Day 2 fluorescence (left) is measured to establish a baseline signal before cells are plated into metabolic conditions and allowed to continue to proliferate. Dilution of the signal over successive cellular generations can be seen and is used to assess proliferation.

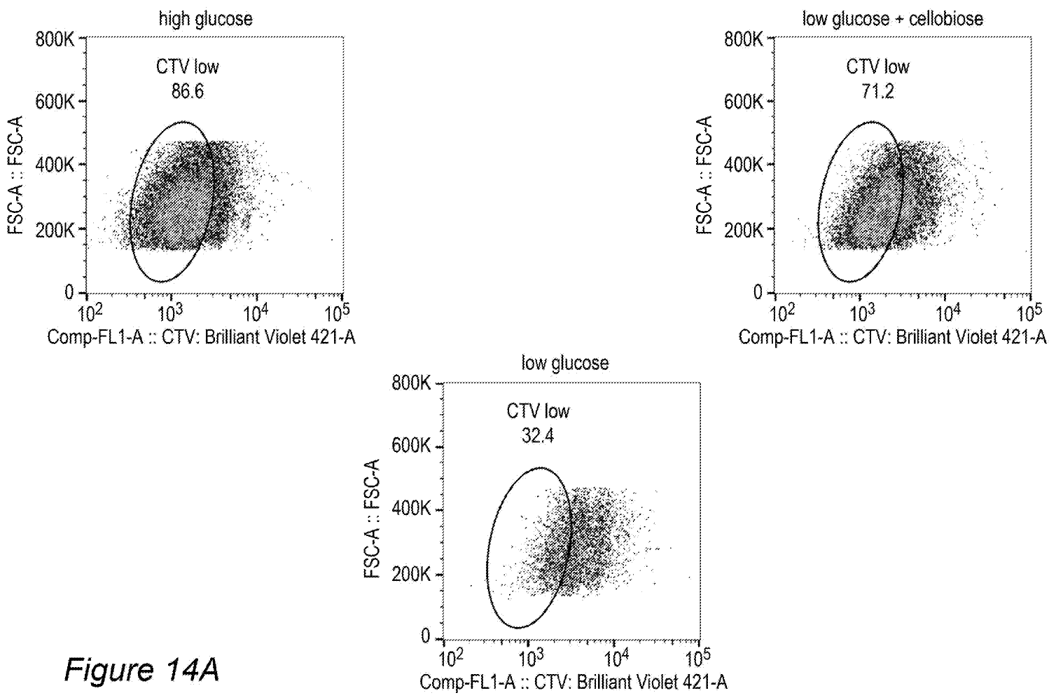

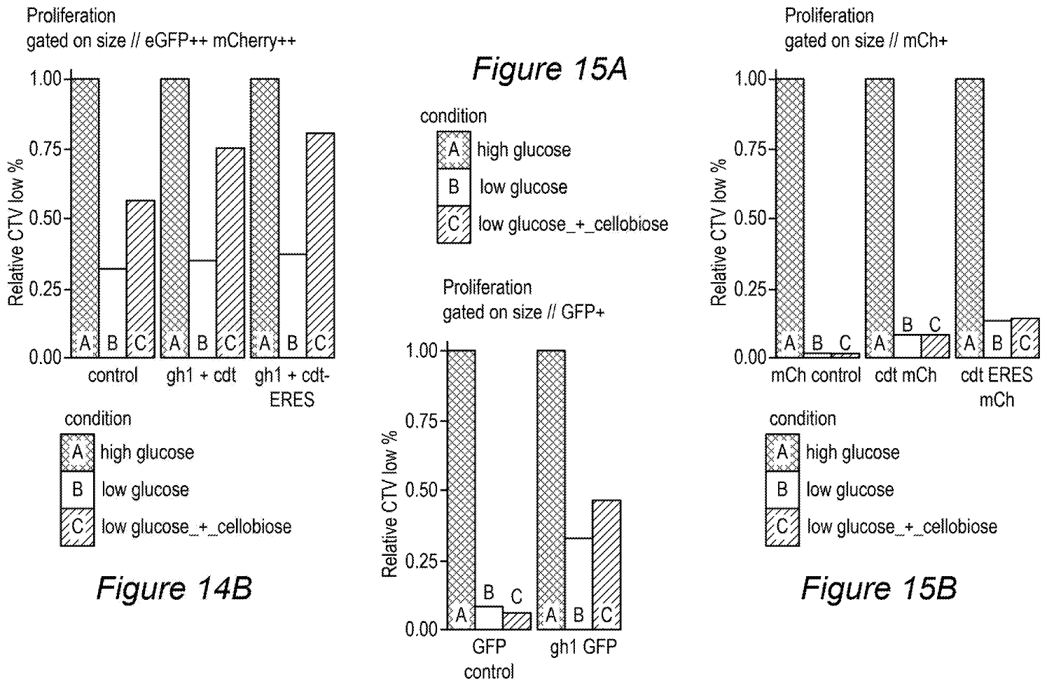

FIGS. 14A-14B are graphs depicting the results of the T cell proliferation experiment of FIGS. 13A-13C. FIG. 14A is a series of graphs depicting forward scatter (y-axis) and CELLTRACE™ VIOLET (x-axis) fluorescent signal of transduced T cells incubated for two days in high glucose, low glucose, or low glucose+cellobiose metabolic conditions. The percentage of the cells that have divided 4 or more times have been quantified and annotated as “CTV low”. FIG. 14B is a bar graph depicting the results of a T cell proliferation study and shows the results of the relative CTV low % with respect to each of the three samples (T cells transduced with a control virus [left trio]; T cells transduced with gh1-1 and cdt-1 virus [center trio]; T cells transduced with gh1-1 and cdt-1-ERES virus [right trio]) with respect to each of the three metabolic conditions (high glucose [gray, left bar of each trio], low glucose [green, center bar of each trio], and low glucose+cellobiose [blue, right bar of each trio]).

### Example 21: Assessment of Expression of Single-Gene Control Transfections on Mammalian Cellobiose Metabolism and Proliferation

To confirm whether transfection of both cdt-1 and gh1-1 plasmids is required, a PLATINUM-E™ cell (CELL BIOLABS™) proliferation study following single-gene control transfections was performed. With respect to a single-gene gh1-1 vector, PLATINUM-E™ cells were transfected with a single control vector (FIG. 1—top [SEQ ID NO: 7]) or gh1-1 GFP vector (FIG. 2—below top [SEQ ID NO: 10]). Cell proliferation was measured with respect to each of the three metabolic conditions (high glucose, low glucose, and low glucose+cellobiose). With respect to single-gene cdt-1 vectors, the experiment was repeated with PLATINUM-E™ cells transfected with a single control vector (FIG. 1—bottom [SEQ ID NO: 8]); cdt mCherry vector (FIG. 4—top [SEQ ID NO: 12]); or cdt-ERES mCherry vector (FIG. 4—bottom [SEQ ID NO: 13])) with respect to each of the three metabolic conditions (high glucose, low glucose, and low glucose+cellobiose).

FIG. 15A shows the results of the relative CELLTRACE™ Violet (THERMOFISHER SCIENTIFIC™, #C34571; CTV) low % with respect to PLATINUM-E™ cells transfected with a single control vector (FIG. 1—top [SEQ ID NO: 7]) [left trio] or gh1-1 GFP vector (FIG. 2—below top [SEQ ID NO: 10]) [right trio] with respect to each of the three metabolic conditions (high glucose [gray, left bar of each trio], low glucose [green, center bar of each trio], and low glucose+cellobiose [blue, right bar of each trio]).

FIG. 15B shows the results of the relative CTV low % with respect to PLATINUM-E™ cells transfected with a single control vector (FIG. 1—bottom [SEQ ID NO: 8]) [left trio]; cdt mCherry vector (FIG. 4—top [SEQ ID NO: 12]) [center trio]; or cdt-ERES mCherry vector (FIG. 4—bottom [SEQ ID NO: 13]) [right trio]) with respect to each of the three metabolic conditions (high glucose [gray, left bar of each trio], low glucose [green, center bar of each trio], and low glucose+cellobiose [blue, right bar of each trio]).

These data indicate that expression of a single gene, either cdt-1 or gh1-1, is not sufficient to rescue proliferation with cellobiose.

### Example 22: Construction of Additional cdt-1 Vectors and Expression of Same

To explore the effects of codon optimization of cdt-1 vectors further, additional codon-optimized cdt-1 vectors were constructed. As shown in Table 1 and Table 2, the amino acid sequence for cellodextrin transport-1 (cdt-1; https://www.genome.jp/dbget-bin/www_bget?ncr:NCU00801) from Neurospora crassa was codon-optimized twice (two versions) using the IDT Codon Optimization Tool (https://www.idtdna.com/pages/tools/codon-optimization-tool). It was also codon-optimized using the BLUE HERON™ BioTech Codon Optimization Tool (https://www.blueheronbio.com/codon-optimization/?gclid=CjwKCAjw9MuCBhBUEiwAbDZ-7jQJqeOS6NfjW40raaApv_wPSBk6kTzS7V3D1CxiQifvAfUBvJ_6hhoCttEQAvD_BwE) (EUROFINS GENOMICS™), or the OPTIMUM GENE™ BioTech Codon Optimization Tool (https://www.genscript.com/codon-opt.html?src=google&gclid=CjwKCAjw9MuCBhBUEiwAbDZ-7sdh1Ve2q8emWgomPW4wxh9pigffndWQJefv7ay19-rB-s919Rbp9BoCt7oQAvD_BwE) (GENSCRIPT®). Codon-optimized cdt-1 sequences were then synthesized as GBLOCKS™ Gene Fragments by INTEGRATED DNA TECHNOLOGIES™ (IDT). Each of the resultant double-stranded DNA fragments was cloned into MSCV MCS PGK-mCherry vector. The linearized plasmid was then combined with the cdt-1 gBlock (GBLOCKS™ Gene Fragment, INTEGRATED DNA TECHNOLOGIES™) and NEBUILDER® HiFi DNA Assembly Master Mix (NEW ENGLAND BIOLABS®, #E2621S) before transformation into NEB® 5-alpha Competent E. coli (High Efficiency) (NEW ENGLAND BIOLABS®, #C2987H). Additional iterations of the cdt-1 plasmid followed the same protocol, but with the HA-tag and ERES signal addended to the C-terminus.

FIGS. 16A-16B are schematic maps and expression analysis of MSCV vectors that were constructed to contain different codon optimized variants of the cdt-1 gene, each with an HA-tag sequence amended to the C-terminus. In FIG. 16A, the top vector is the same as in FIG. 4 [above] (SEQ ID NO: 12). The second vector (SEQ ID NO: 14) from the top includes a different cdt-1 DNA sequence generated by the IDT codon optimization tool, which is the same tool used to generate the cdt-1 sequence in the top vector. This tool is not deterministic and results in different outputs each time a sequence is entered. The third vector (SEQ ID NO: 15) from the top is a third cdt-1 DNA sequence generated using a codon optimization tool from BLUE HERON™ BIOTECH, and the bottom vector (SEQ ID NO: 16) is a fourth cdt-1 DNA sequence generated using a codon optimization tool from GENSCRIPT™ BIOTECH. FIG. 16B shows flow cytometric analysis of transfected PLATINUM-E™ cells (CELL BIOLABS™) stained with anti HA-tag antibody. These graphs depict the anti-HA tag signal within the mCherry+ positive populations, or the populations that were successfully transfected. The percent of the parent population is displayed (or the percent of mCherry+ cells that have a detectable HA-tag signal) as well as the mean fluorescence intensity (MFI) of the HA-tag signal within the entire mCherry+ population. These results indicate that the GENSCRIPT™ codon optimized variant [FIG. 16B—far right; SEQ ID NO: 16] results in the highest percentage of mCherry+ cells with a detectable HA-tag signal as well as the highest MFI, showing a 7-10 fold increase over the other variants.

### Example 23: Assessment of Mammalian Cellobiose Metabolism and Proliferation with Additional Codon-Optimized cdt-1 Vector

An additional PLATINUM-E™ cell (CELL BIOLABS™) proliferation experiment, using the codon optimized cdt-1 variant from GENSCRIPT™, (FIG. 16A—vector 4; SEQ ID NO: 16) was performed and compared to other vectors with optimized variants of cdt-1.

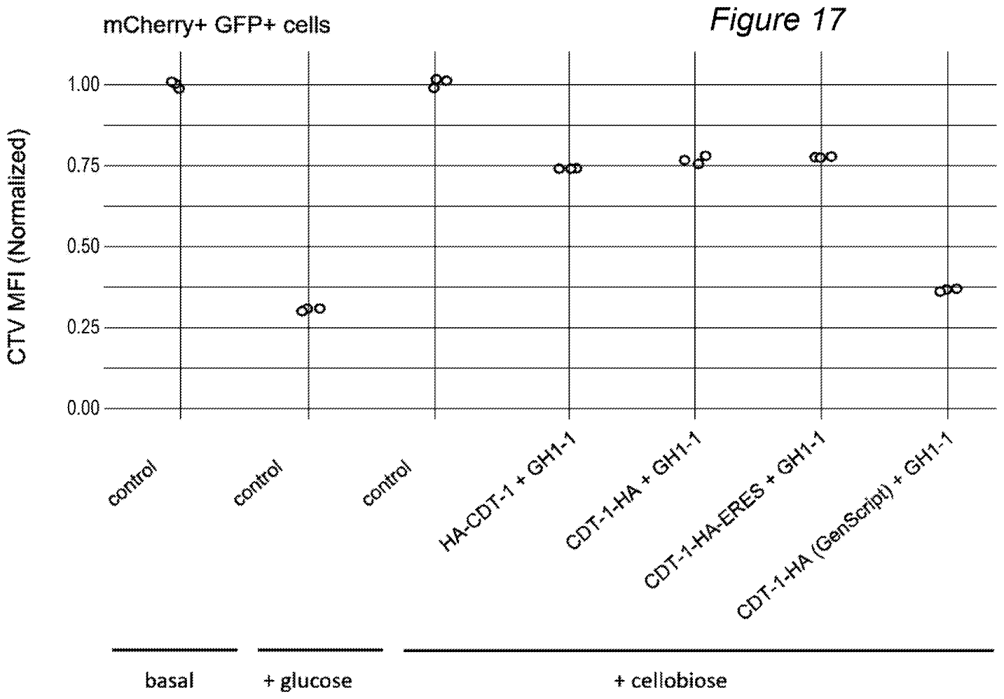

FIG. 17 shows the results of another PLATINUM-E™ cell (CELL BIOLABS™) proliferation experiment, using the new codon optimized cdt-1 variant from GenScript, compared to the previously constructed variants. The results display the CELLTRACE™ VIOLET signal of the GFP+ mCherry+ positive cells transfected with the various constructs (control [FIG. 1—top and bottom together (SEQ ID NO: 7 and SEQ ID NO: 8)], the remaining conditions use the gh1-1 vector [FIG. 2—below top (SEQ ID NO: 10)] together with cdt-1 [from FIG. 2—bottom (SEQ ID NO: 11) or FIG. 4—top (SEQ ID NO: 12) or FIG. 4—bottom (SEQ ID NO: 13) or FIG. 16A—bottom (SEQ ID NO: 16)]); and incubated in a basal condition, basal condition plus glucose, or basal condition plus cellobiose. The signals are normalized to the control (EV) cells in the basal condition.

These results indicate that the cells co-transfected with the construct containing gh1-1 and the GENSCRIP™ cdt-1 gene can proliferate using cellobiose at a comparable level to control cells growing in glucose, suggesting that total expression of cdt-1 is an important factor for utility of cellobiose as a fuel source.

### Example 24: Assessment of Mammalian Cellobiose Metabolism and Proliferation in Primary T Cells Expressing CDT-1 and GH1-1

The data set from the experimental results depicted in FIGS. 13A-13C and FIGS. 14A-14B was subjected to further analysis.

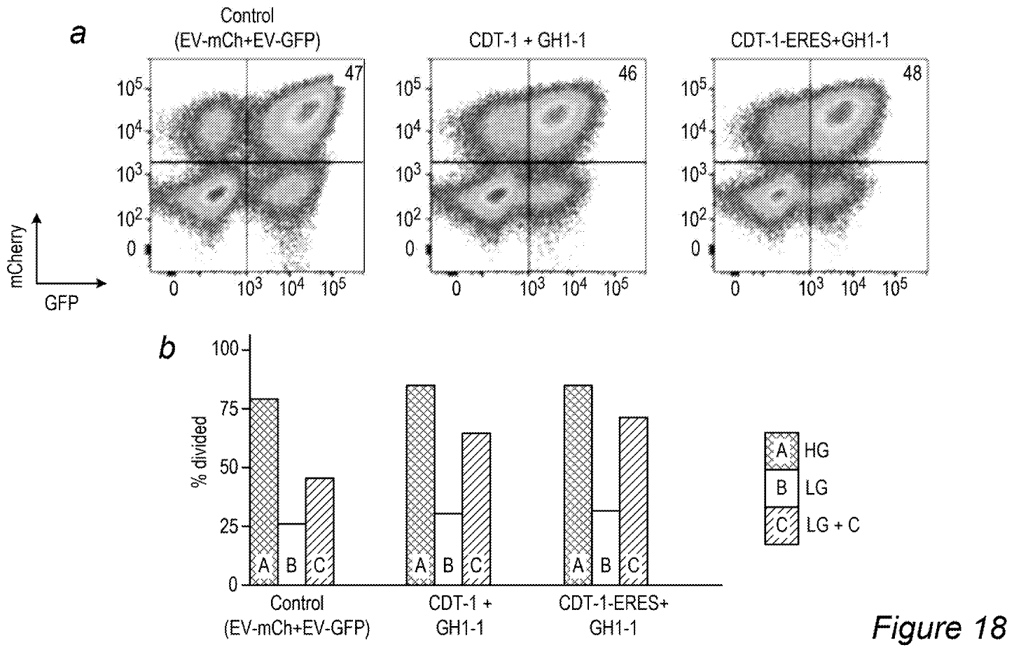

FIG. 18A shows flow cytometric analysis of T cells co-transduced with MSCV (Control [EV-mCh+EV-GFP; left]; CDT-1+GH1-1 [center]; CDT-1-ERES+GH1-1 [right]), resulting in dual expression of mCherry and GFP in approximately 50% of the population. CELLTRACE™ VIOLET-stained CG-T cells were incubated in high glucose (HG), low glucose (LG), and low glucose+cellobiose (LG+C) conditions for 48 hr, after which their fluorescent signals were measured. CG-T cells showed a boost in proliferation when cellobiose was added to the low glucose environment.

FIG. 18B is a series of bar graphs showing the results of FIG. 18A (Control [EV-mCh+EV-GFP; right]; CDT-1+GH1-1 [center]; CDT-1-ERES+GH1-1 [right]) with respect to HG [left of each trio], LG [center of each trio], and LG+C [right of each trio]. In mCh+ GFP+ cells, cellobiose (+C) rescued T-cell proliferation in starvation conditions (low glucose, LG), approaching the high glucose (HG) state.

The analysis was slightly changed (gating), and the plot in FIG. 18B shows absolute percentages rather than relative. Just an alternative way of looking at the same data set. Notably, there was a minor increase in wild-type (WT) T-cell proliferation in the low glucose environment when cellobiose was added, suggesting perhaps spontaneous or enzymatic hydrolysis, but the increase in proliferation of WT T-cells did not match the increase in proliferation of CG-T cells.

### Example 25: Assessment of Mammalian Cellobiose Metabolism and Proliferation in Primary T Cells Expressing CDT-1 and GH1-1

In order to study the effects of cellobiose on tumors, the high glucose (HG), low glucose (LG), and low glucose+cellobiose (LG+C) in vitro studies of FIGS. 18A-18B were repeated using a B16 melanoma cell line constitutively expressing GFP. By using GFP positivity as a proxy for cell viability, the data demonstrated that cellobiose did not provide B16 melanoma any survival advantage in low glucose environments.

As shown in FIG. 19, The flow cytometric analysis demonstrated that cellobiose is inert to tumors. Cellobiose (+C) did not promote B16 melanoma tumor survival in starvation (low glucose, LG) conditions. The tumors did not derive a benefit from cellobiose.

### Example 26: Construction of Additional cdt-1 and gh1-1 Vectors and Expression of Same

In order to increase the expression of the transgenic genes in primary T cells, the viral delivery vector was redesigned. As shown in FIG. 20 (top and bottom; SEQ ID NO: 26 and SEQ ID NO: 27) and FIG. 21 (top and bottom; SEQ ID NO: 28 and SEQ ID NO: 29) and in Table 2 and Table 3, the transgenes, cdt-1 and gh1-1, encoding cdt-1 (SEQ ID NO:32) and gh1-1 (SEQ ID NO:37) were relocated under the control of the strong, constitutive promoter PGK. The 3′ ends of the genes were modified to contain a T2A ribosomal skipping sequence that was then followed in-frame by either the mCherry or GFP coding sequence. This design allows for (1) enhanced expression of the transgene and (2) coupling of the fluorescent protein markers to cells actively expressing the transgene. Additionally, the Woodchuck hepatitis virus post-transcriptional regulatory element (WPRE) was addended downstream from the transgene. WPRE has been shown to increase transcript stability and leads to enhanced protein expression on transcripts where it is present. This new vector design is referred to herein as MSCV GENERATION 2 for the MSCV_PGK-cdt-1-2A-mCherry vector (SEQ ID NO: 28) and MSCV_PGK-gh1-1-2A-GFP vector (SEQ ID NO: 29).

### Example 27: Assessment of Mammalian Cellobiose Metabolism and Proliferation in Primary T Cells Expressing CDT-1 and GH1-1

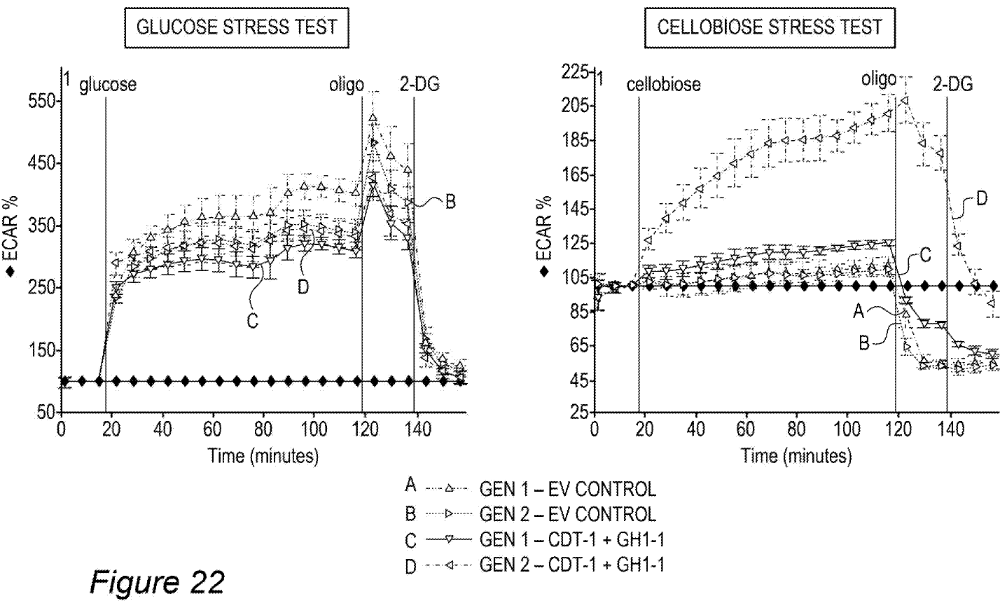

In order to assess the enhanced functionality of the new vector design, the first-generation transgene constructs (SEQ ID NO: 12 and SEQ ID NO: 10; FIG. 4/FIG. 16A and FIG. 2, respectively), the second-generation transgene constructs (SEQ ID NO: 28 and SEQ ID NO: 29; FIG. 22), the first-generation empty vectors (SEQ ID NO: 7 and SEQ ID NO: 8; FIG. 1), or the second-generation empty vectors (SEQ ID NO: 26 and SEQ ID NO: 27; FIG. 21) were transfected in pairs into PLATINUM-E™ cells.

After two days, the transfected cells were assayed on a SEAHORSE EXTRACELLULAR FLUX ANALYZER™ (AGILENT™) to measure extracellular acidification rates (ECAR) using either glucose or cellobiose as a primary carbon source. [00406] 80,000 PLATINUM-E™ cells were suspended in a basal media (media containing no glucose or cellobiose) and plated onto SEAHORSE XF96 CELL CULTURE™ Microplates (AGILENT™, 101085-004) that had previously been coated with 100 ug/mL poly-D-lysine (THERMOFISHER SCIENTIFIC™, A3890401). The basal media consisted of SEAHORSE XF BASE MEDIUM™ (AGILENT™, 103334-100) with a pH adjustment to pH 7.4 and a supplementation of 2 mM glutamine (THERMOFISHER SCIENTIFIC™, 25030081). For the glucose stress test, 100 mM glucose (SIGMA™ G5767) was loaded into the first port of the SEAHORSE EXTRACELLULAR FLUX CARTRIDGE™ (AGILENT™, 102416-100), 1 uM oligomycin was loaded into the second port (TOCRIS™, 4110), and 100 mM 2-deoxyglucose (SIGMA™, D6134) was loaded into the third port. The plates were then assayed using a SEAHORSE XFE96 ANALYZER™ (AGILENT™). For the cellobiose stress test, 50 mM cellobiose was loaded into the first port instead of glucose, followed by oligomycin in the second port, and 2-deoxyglucose in the third port.

The ECAR of cells in basal media (no glucose or cellobiose) was first measured to obtain a baseline rate. Next, glucose (Glucose Stress Test) or cellobiose (Cellobiose Stress Test) was injected into each well, and the relative increase in ECAR was measured.

Oligomycin and 2-deoxyglucose were also sequentially injected into the wells. Oligomycin blocks ATP synthase and measures maximal glycolytic capacity and 2-deoxyglucose competes with glucose as a substrate for hexokinase and measures the portion of ECAR that is attributable to glycolysis.

As shown in FIG. 22, left panel, when glucose was injected into the wells, all four conditions responded with a rapid increase in ECAR, with the rates increasing and plateauing between 250-350% over basal ECAR. In contrast, as shown in FIG. 22, right panel, when cellobiose was injected into each well, only the cells transfected with the MSCV GENERATION 2 transgene vectors (SEQ ID NO: 28 and SEQ ID NO: 29) showed an increase in ECAR, indicating that the transgene expression level was enhanced sufficiently to allow for the consumption of cellobiose to be measured in this format. (The black line that repeats in value at 100% represents the normalized value from measurement 3, to which all other measurements are compared [y-axis is percent change in ECAR value relative to ECAR at measurement 3].)

While certain features of the nanoliposomes, microparticles, and methods of use thereof have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A bioengineered cell modified to metabolize a xenobiotic fuel, the xenobiotic fuel not metabolized by a corresponding unmodified cell, the bioengineered cell comprising:
(a) at least one foreign nucleic acid encoding at least one transporter protein or a functional fragment thereof for transport of the xenobiotic fuel into the bioengineered cell;
(b) at least one foreign nucleic acid encoding at least one protein or a functional fragment thereof for enabling the metabolizing of the xenobiotic fuel in the bioengineered cell; or
(c) a combination of (a) and (b).

2. The bioengineered cell of claim 1, wherein:
(a) the xenobiotic fuel comprises cellobiose;
(b) the transporter protein comprises a cellodextrin transporter protein or a functional fragment thereof;
(c) the protein for enabling the metabolizing of the xenobiotic fuel comprises a beta-glucosidase protein or a functional fragment thereof or a cellobiose phosphorylase protein or a functional fragment thereof.

3. The bioengineered cell of claim 1 or claim 2, wherein the sequence of the nucleic acid encoding the transporter protein or a functional fragment thereof or the sequence of the nucleic acid encoding a protein or a functional fragment thereof for enabling the metabolizing of the xenobiotic fuel in the bioengineered cell is codon-optimized for the bioengineered cell.

4. The bioengineered cell of claim 2 or claim 3, wherein:
(a) the nucleic acid sequence encoding the cellodextrin transporter protein or a functional fragment thereof encodes a protein at least 90% identical to SEQ ID NO: 32 or SEQ ID NO: 3; or
(b) the nucleic acid sequence encoding the beta-glucosidase protein or a functional fragment thereof encodes a protein at least 90% identical to SEQ ID NO: 37 or SEQ ID NO: 6.

5. The bioengineered cell of claim 4, wherein:
(a) the nucleic acid sequence encoding the cellodextrin transporter protein or a functional fragment thereof is at least 90% identical to SEQ ID NO: 1, SEQ ID NO:2, SEQ ID NO: 17, SEQ ID NO: 18, SEQ ID NO: 19, or SEQ ID NO: 28; or
(b) the nucleic acid sequence encoding the beta-glucosidase protein or a functional fragment thereof is at least 90% identical to SEQ ID NO: 4, SEQ ID NO: 5, or SEQ ID NO: 29.

6. The bioengineered cell of any one of claims 2-4, further comprising a nucleic acid sequence comprising a Woodchuck hepatitis virus post-transcriptional regulatory element (WPRE) operably linked to the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof.

7. The bioengineered cell of any one of claims 2-5, the cellodextrin transporter protein or functional fragment thereof operably linked to a signal peptide, the signal peptide translocating the cellodextrin transporter protein or functional fragment thereof to the membrane of the bioengineered cell.

8. The bioengineered cell of claim 6, the signal peptide comprising an endoplasmic reticulum export signal (ERES).

9. The bioengineered cell of any one of claims 2-7, further comprising a hemagglutinin (HA) tag operably linked to the cellodextrin transporter protein or functional fragment thereof, the beta-glucosidase protein or functional fragment thereof, or the cellobiose phosphorylase protein or functional fragment thereof.

10. The bioengineered cell of any one of claims 2-7, further comprising a 2A ribosomal skipping peptide operably linked to the cellodextrin transporter protein or a functional fragment thereof, the beta-glucosidase protein or a functional fragment thereof, or the cellobiose phosphorylase protein or a functional fragment thereof.

11. The bioengineered cell of any one of claims 1-10, the vector comprising a retroviral vector, a viral vector, or a plasmid vector.

12. The bioengineered cell of any one of claims 1-11, comprising:
(a) a vector comprising a nucleic acid sequence encoding the cellodextrin transporter protein or a functional fragment thereof, the nucleic acid sequence at least 90% identical to SEQ ID NO: 28; or
(b) a vector comprising a nucleic acid sequence encoding the cellodextrin transporter protein or a functional fragment thereof, the nucleic acid sequence at least 90% identical to SEQ ID NO: 29.

13. The bioengineered cell of any one of claims 1-12, wherein the bioengineered cell is a bioengineered immune cell.

14. The bioengineered cell of claim 13, wherein the bioengineered immune cell is a mammalian cell or an avian cell.

15. The bioengineered cell of any one of claims 1-14, wherein the bioengineered cell is a bioengineered immune cell comprising a T-cell, a regulatory T-cell (Treg), a B-cell, a dendritic cell, a macrophage, an M1 polarized macrophage, a B cell receptor (BCR)-stimulated B cell, a tumor-infiltrating lymphocyte (TIL), or a natural killer cell (NK).

16. The bioengineered cell of claim 15, the bioengineered immune cell comprising a chimeric antigen receptor (CAR)-T cell, a CAR-B cell, a CAR-T regulatory cell (CAR Treg), or a T-cell engineered to alter the specificity of the T-cell receptor (TCR).

17. The bioengineered cell of any one of claims 1-12, wherein the bioengineered cell is a stromal cell, a neuron, or a cardiac cell.

18. A method of modulating an immune response at a focus of interest in a subject in need thereof, the method comprising: administering a xenobiotic fuel-enabled bioengineered immune cell to said subject said bioengineered immune cell comprising:
(a) at least one vector comprising at least one nucleic acid encoding at least one transporter protein or a functional fragment thereof for transport of the xenobiotic fuel into the bioengineered immune cell;
(b) at least one vector comprising at least one nucleic acid encoding at least one protein or a functional fragment thereof for enabling the metabolizing of the xenobiotic fuel in the bioengineered immune cell; or
(c) a combination of (a) and (b);
administering the xenobiotic fuel to said subject; wherein said modulating the immune response comprises stimulating said immune response or suppressing said immune response.

19. The method of claim 18, wherein administering the xenobiotic fuel-enabled bioengineered immune cell to said subject comprises administering the xenobiotic fuel-enabled bioengineered immune cell on or adjacent to said focus of interest; or administering the xenobiotic fuel to said subject comprises implanting a scaffold comprising releasable xenobiotic fuel on, adjacent to, or near said focus of interest.

20. The method of claim 18-19, wherein:
(a) the xenobiotic fuel comprises cellobiose;
(b) the transporter protein comprises a cellodextrin transporter protein or a functional fragment thereof;
(c) the protein for enabling the metabolizing of the xenobiotic fuel comprises a beta-glucosidase protein or a functional fragment thereof or a cellobiose phosphorylase protein or a functional fragment thereof.

21. The method of any one of claims 18-20 wherein the sequence of the nucleic acid encoding the transporter protein or a functional fragment thereof or the sequence of the nucleic acid encoding a protein or a functional fragment thereof for enabling the metabolizing of the xenobiotic fuel in the bioengineered immune cell is codon-optimized for the bioengineered immune cell.

22. The method of claim 20 or claim 21, wherein:
(a) the nucleic acid sequence encoding the cellodextrin transporter protein or a functional fragment thereof encodes a protein at least 90% identical to SEQ ID NO: 32 or SEQ ID NO: 3; or
(b) the nucleic acid sequence encoding the beta-glucosidase protein or a functional fragment thereof encodes a protein at least 90% identical to SEQ ID NO: 37 or SEQ ID NO: 6.

23. The method of any one of claims 19-22, the nucleic acid further comprising a Woodchuck hepatitis virus post-transcriptional regulatory element (WPRE) operably linked to the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof.

24. The method of any one of claims 20-23, the cellodextrin transporter protein or functional fragment thereof operably linked to a signal peptide, the signal peptide translocating the cellodextrin transporter protein or functional fragment thereof to the membrane of the bioengineered immune cell.

25. The method of claim 24, the signal peptide comprising an endoplasmic reticulum export signal (ERES).

26. The method any one of claims 20-25, further comprising a hemagglutinin (HA) tag operably linked to the cellodextrin transporter protein or functional fragment thereof, the beta-glucosidase protein or functional fragment thereof, or the cellobiose phosphorylase protein or functional fragment thereof.

27. The method of any one of claims 20-26, further comprising a 2A ribosomal skipping peptide operably linked to the cellodextrin transporter protein or a functional fragment thereof, the beta-glucosidase protein or a functional fragment thereof, or the cellobiose phosphorylase protein or a functional fragment thereof.

28. The method of any one of claims 20-27, the vector comprising a retroviral vector, a viral vector, or a plasmid vector.

29. The method of any one of claims 18-28, wherein the xenobiotic fuel-enabled bioengineered immune cell comprises:
(a) a vector comprising a nucleic acid sequence encoding the cellodextrin transporter protein or a functional fragment thereof, the nucleic acid sequence at least 90% identical to SEQ ID NO: 28; or
(b) a vector comprising a nucleic acid sequence encoding the cellodextrin transporter protein or a functional fragment thereof, the nucleic acid sequence at least 90% identical to SEQ ID NO: 29.

30. The method of any one of claims 18-29, wherein the bioengineered immune cell is a mammalian cell or an avian cell.

31. The method of any one of claims 18-30, wherein modulating the immune response comprises stimulating the immune response and wherein the bioengineered immune cell comprising a T-cell, a chimeric antigen receptor (CAR)-T cell, a T cell engineered to alter the specificity of the T-cell receptor (TCR), a B-cell, a CAR-B cell, a dendritic cell, a macrophage, an M1 polarized macrophage, a B cell receptor (BCR)-stimulated B cell, a tumor-infiltrating lymphocyte (TIL), or a natural killer cell (NK).

32. The method of claim 31, wherein said bioengineered immune cell comprises a T-cell or a CAR-T cell and said modulating the immune response comprises increasing proliferation of cytotoxic T cells, increasing proliferation of helper T cells, maintaining the population of helper T cells at the site of said tumor, activating cytotoxic T cells at the site of said solid tumor or infection, or any combination thereof.

33. The method of claim 31, wherein said bioengineered immune cell comprises a B-cell or a CAR-B cell and said modulating the immune response comprises increasing production of antibodies from the B-cell or CAR-B cell.

34. The method of any one of claims 18-33, wherein modulating the immune response comprises suppressing the immune response and wherein the bioengineered immune cell comprises a regulatory T cell (Treg), a chimeric antigen receptor (CAR)-Treg, or a T-cell engineered to alter the specificity of the T-cell receptor (TCR).

35. The method of claim 34, wherein said bioengineered immune cell comprises a Treg cell or a CAR-Treg cell and said modulating the immune response comprises decreasing proliferation of cytotoxic T cells; decreasing proliferation of helper T cells; suppressing cytotoxic T cells at the site of said focus of interest; or any combination thereof.

36. The method of any one of claims 18-35, wherein the focus of interest comprises a solid tumor.

37. The method of claim 36, wherein said solid tumor comprises a cancerous, pre-cancerous, or non-cancerous tumor.

38. The method of claim 36 or claim 37, wherein said solid tumor comprises a tumor comprising a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodenroglioma, a schwannoma, a meningioma, a melanoma, a neuroblastoma, or a retinoblastoma.

39. The method of any one of claims 36-38, further comprising reducing the size of the solid tumor, eliminating said solid tumor, slowing the growth of the solid tumor, or prolonging survival of said subject, or any combination thereof.

40. The method of any one of claims 18-30 and 34-35, wherein said focus of interest comprises:
(a) an autoimmune-targeted or symptomatic focus of an autoimmune disease;
(b) a reactive focus of an allergic reaction or hypersensitivity reaction;
(c) a focus of infection or symptoms of a localized infection or infectious disease;
(d) an injury or a site of chronic damage;
(e) a surgical site;
(f) a site of a transplanted organ, tissue, or cell; or
(g) a site of blood clot causing or at risk for causing a myocardial infarction, ischemic stroke, or pulmonary embolism.

41. The method of claim 40, wherein said modulating the immune response:
(a) reduces or eliminates inflammation or another symptom of said autoimmune-targeted or symptomatic focus of said autoimmune disease, prolongs survival of said subject, or any combination thereof;
(b) reduces or eliminates inflammation or another symptom of allergic reaction or hypersensitivity reaction at said reactive focus of said allergic reaction or hypersensitivity reaction, prolongs survival of said subject, or any combination thereof;
(c) reduces or eliminates infection or symptoms at said focus of infection or symptoms of said localized infection or infectious disease, prolongs survival of said subject, or any combination thereof;
(d) reduces, eliminates, inhibits or prevents structural, organ, tissue, or cell damage, inflammation, infection, or another symptom at said site of injury or said site of chronic damage, improves structural, organ, tissue, or cell function at said site of injury or said site of chronic damage, improves mobility of said subject, prolongs survival of said subject, or any combination thereof;
(e) reduces, eliminates, inhibits, or prevents structural, organ, tissue, or cell damage, inflammation, infection, or another symptom at said surgical site, improves structural, organ, tissue, or cell function at said surgical site, improves mobility of said subject, prolongs survival of said subject, or any combination thereof;
(f) reduces, eliminates, inhibits or prevents transplanted organ, tissue, or cell damage or rejection, inflammation, infection or another symptom at said transplant site, improves mobility of said subject, prolongs survival of said transplanted organ, tissue, or cell, prolongs survival of said subject, or any combination thereof; or
(g) reduces or eliminates said blood clot causing or at risk for causing said myocardial infarction, said ischemic stroke, or said pulmonary embolism in said subject, improves function or survival of a heart, brain, or lung organ, tissue, or cell in said subject, reduces damage to a heart, brain, or lung organ, tissue, or cell in said subject, prolongs survival of a heart, brain, or lung organ, tissue, or cell in said subject, prolongs survival of said subject, or any combination thereof.

42. A method of modulating an immune response at the site of a solid tumor or infection, said method comprising: administering a cellobiose-enabled bioengineered T cell to said subject adjacent to a solid tumor or infection, said cellobiose-enabled bioengineered T cell comprising a vector comprising a nucleic acid encoding a cellodextrin transporter protein or a functional fragment thereof and a vector comprising a nucleic acid encoding a beta-glucosidase protein or a functional fragment thereof; and administering cellobiose to said subject or implanting a scaffold that releases cellobiose adjacent to said solid tumor or infection, said modulating the immune response comprising increasing proliferation of cytotoxic T cells; increasing proliferation of helper T cells; maintaining the population of helper T cells at the site of said tumor; activating cytotoxic T cells at the site of said solid tumor or infection; or any combination thereof.

43. A method of modulating an immune response at the site of a solid tumor or infection, said method comprising: administering a cellobiose-enabled bioengineered B cell to said subject adjacent to a solid tumor or infection, said cellobiose-enabled bioengineered B cell comprising a vector comprising a nucleic acid encoding a cellodextrin transporter protein or a functional fragment thereof and a vector comprising a nucleic acid encoding a beta-glucosidase protein or a functional fragment thereof; and administering cellobiose to said subject or implanting a scaffold that releases cellobiose adjacent to said solid tumor or infection, said modulating the immune response comprising increasing production of antibodies from the B cell; increasing isotype switching; increasing affinity maturation; or any combination thereof.

44. A method of modulating an immune response at a focus of interest of an autoimmune disease, an allergic reaction, a localized infection or an infectious disease, an injury or other damage, a transplant or other surgical site, or a symptom thereof, or a combination thereof, in a subject in need thereof, comprising administering to said subject a bioengineered T regulatory (Treg) cell, adjacent to said focus of interest, said cellobiose-enabled bioengineered Treg cell comprising a vector comprising a nucleic acid encoding a cellodextrin transporter protein or a functional fragment thereof and a vector comprising a nucleic acid encoding a beta-glucosidase protein or a functional fragment thereof; and administering cellobiose to said subject or implanting a scaffold that release said cellobiose adjacent to said focus of interest; wherein said regulating the immune response comprises decreasing proliferation of cytotoxic T cells; decreasing proliferation of helper T cells; suppressing cytotoxic T cells at the site of said focus of interest; or any combination thereof.

45. A vector comprising at least one nucleic acid sequence encoding at least one protein for modifying a bioengineered cell to enable metabolism of a xenobiotic fuel in the cell, the xenobiotic fuel not metabolized by a corresponding unmodified cell, the vector comprising:
(a) a promoter, the promoter operably linked to (i) a nucleic acid encoding a transporter protein or a functional fragment thereof for transport of the xenobiotic fuel into the bioengineered cell; (ii) a nucleic acid encoding a protein or a functional fragment thereof for enabling the metabolizing of the xenobiotic fuel in the bioengineered cell; or (iii) a combination of (i) and (ii); and
(b) a selective marker.

46. The vector of claim 45, wherein:
(a) the transporter protein or functional fragment thereof comprises a cellodextrin transporter protein or a functional fragment thereof; or
(b) the protein or functional fragment thereof for enabling the metabolizing of the xenobiotic fuel in the bioengineered cell comprises a beta-glucosidase protein or a functional fragment thereof or a cellobiose phosphorylase protein or a functional fragment thereof.

47. The vector of claim 45 or claim 46, wherein the sequence of the nucleic acid encoding the transporter protein or a functional fragment thereof or the sequence of the nucleic acid encoding a protein or a functional fragment thereof for enabling the metabolizing of the xenobiotic fuel in the bioengineered cell is codon-optimized for the bioengineered cell.

48. The vector of claim 46 or claim 47, wherein:
(a) the nucleic acid sequence encoding the cellodextrin transporter protein or a functional fragment thereof encodes a protein at least 90% identical to SEQ ID NO: 32 or SEQ ID NO: 3; or
(b) the nucleic acid sequence encoding the beta-glucosidase protein or a functional fragment thereof encodes a protein at least 90% identical to SEQ ID NO: 37 or SEQ ID NO: 6.

49. The vector of claim 48, wherein:
(a) the nucleic acid sequence encoding the cellodextrin transporter protein or a functional fragment thereof is at least 90% identical to SEQ ID NO: 1, SEQ ID NO: 2, SEQ ID NO: 17, SEQ ID NO: 18, or SEQ ID NO: 19; or
(b) the nucleic acid sequence encoding the beta-glucosidase protein or a functional fragment thereof is at least 90% identical to SEQ ID NO: 4 or SEQ ID NO: 5.

50. The vector of any one of claims 46-49, further comprising a nucleic acid sequence comprising a Woodchuck hepatitis virus post-transcriptional regulatory element (WPRE) operably linked to the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof.

51. The vector of claim 50, wherein the WPRE is downstream of the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof.

52. The vector of any one of claims 46-51, the nucleic acid sequence encoding the cellodextrin transporter protein or functional fragment thereof operably linked to a nucleic acid sequence encoding a signal peptide, the signal peptide translocating the cellodextrin transporter protein or functional fragment thereof to the membrane of the bioengineered cell.

53. The vector of claim 52, the signal peptide comprising an endoplasmic reticulum export signal (ERES)-encoding sequence.

54. The vector of claim 53, wherein the ERES-encoding sequence is C-terminal to the cellodextrin transporter protein or functional fragment thereof.

55. The vector of any one of claims 46-54, further comprising a nucleic acid sequence encoding a hemagglutinin (HA) tag operably linked to the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof.

56. The vector of claim 55, wherein the HA tag is C-terminal to the cellodextrin transporter protein or functional fragment thereof, is C-terminal to the beta-glucosidase protein or functional fragment thereof, or is C-terminal to the cellobiose phosphorylase protein or functional fragment thereof.

57. The vector of any one of claims 46-56, further comprising a nucleic acid sequence encoding a 2A ribosomal skipping peptide operably linked to the nucleic acid sequence encoding the cellodextrin transporter protein or a functional fragment thereof, the nucleic acid sequence encoding the beta-glucosidase protein or a functional fragment thereof, or the nucleic acid sequence encoding the cellobiose phosphorylase protein or a functional fragment thereof.

58. The vector of claim 57, wherein the 2A ribosomal skipping peptide is C-terminal to the cellodextrin transporter protein or functional fragment thereof, is C-terminal to the beta-glucosidase protein or functional fragment thereof, or is C-terminal to the cellobiose phosphorylase protein or functional fragment thereof.

59. The vector of claim 57 or claim 58, wherein the 2A ribosomal skipping peptide is a T2A ribosomal skipping peptide.

60. The vector of any one of claims 45-60, the vector comprising a retroviral vector, a viral vector, or a plasmid vector.

61. The vector of any one of claims 45-60, wherein:
(a) the vector has a nucleic acid sequence at least 90% identical to SEQ ID NO: 28 and comprising a nucleic acid sequence encoding the cellodextrin transporter protein or a functional fragment thereof at least 90% identical to SEQ ID NO: 32;
(b) the vector has a nucleic acid sequence at least 90% identical to SEQ ID NO: 29 and comprising a nucleic acid sequence encoding the beta-glucosidase protein or a functional fragment thereof at least 90% identical to SEQ ID NO: 37;
(c) the vector has a nucleic acid sequence at least 90% identical to SEQ ID NO: 10 and comprising a nucleic acid sequence encoding the beta-glucosidase protein or a functional fragment thereof at least 90% identical to SEQ ID NO: 5; or
(d) the vector has a nucleic acid sequence at least 90% identical to SEQ ID NO: 16, SEQ ID NO: 13, SEQ ID NO: 15, SEQ ID NO: 14, SEQ ID NO: 12, SEQ ID NO: 11 or SEQ ID NO: 9 and comprising a nucleic acid sequence encoding the cellodextrin transporter protein or a functional fragment thereof at least 90% identical to SEQ ID NO: 3.

62. The vector of any one of claims 45-61, wherein the xenobiotic-enabled bioengineered cell is a xenobiotic-enabled bioengineered immune cell.

63. A method of making a xenobiotic-enabled bioengineered cell, modified to metabolize a xenobiotic fuel, the xenobiotic fuel not metabolized by a corresponding unmodified cell, the method comprising:
(a) selecting a xenobiotic fuel;
(b) selecting a transporter protein or functional fragment thereof for transport of the xenobiotic fuel and obtaining a nucleic acid sequence encoding the same;
(c) selecting a protein or functional fragment thereof for enabling the metabolizing of the xenobiotic fuel and obtaining a nucleic acid sequence encoding the same;
(d) providing (i) a vector comprising a promoter, the promoter operably linked to a nucleic acid encoding a transporter protein or a functional fragment thereof for transport of the xenobiotic fuel into the bioengineered cell, and a selective marker; and (ii) a vector comprising a promoter, the promoter operably linked to a nucleic acid encoding a protein or a functional fragment thereof for metabolizing the xenobiotic fuel in the bioengineered cell, and a selective marker;
(e) isolating a cell of interest from a subject;
(f) transfecting or transducing the cell of interest with (i) the vector comprising a nucleic acid encoding a transporter protein or a functional fragment thereof for transport of the xenobiotic fuel into the bioengineered cell; and (ii) the vector comprising a nucleic acid encoding a protein or a functional fragment thereof for enabling the metabolizing of the xenobiotic fuel in the bioengineered cell.

64. The method of claim 63, wherein:
(a) the xenobiotic fuel comprises cellobiose;
(b) the transporter protein comprises a cellodextrin transporter protein or a functional fragment thereof;
(c) the protein for enabling the metabolizing of the xenobiotic fuel comprises a beta-glucosidase protein or a functional fragment thereof or a cellobiose phosphorylase protein or a functional fragment thereof.

65. The method of claim 64, wherein the protein for enabling the metabolizing of the xenobiotic fuel comprises a beta-glucosidase protein.

66. The method of any one of claims 63-65, further comprising codon-optimizing the nucleic acid of step (b) and the nucleic acid of step (c) with reference to codon usage in the bioengineered cell.

67. The method of any one of claims 64-66, wherein:
(a) the nucleic acid sequence encoding the cellodextrin transporter protein or a functional fragment thereof encodes a protein at least 90% identical to SEQ ID NO: 32 or SEQ ID NO: 3; or
(b) the nucleic acid sequence encoding the beta-glucosidase protein or a functional fragment thereof encodes a protein at least 90% identical to SEQ ID NO: 37 or SEQ ID NO: 6.

68. The method of claim 67, wherein:
(a) the nucleic acid sequence encoding the cellodextrin transporter protein or a functional fragment thereof is at least 90% identical to SEQ ID NO: 1, SEQ ID NO: 2, SEQ ID NO: 17, SEQ ID NO: 18, or SEQ ID NO: 19; or
(b) the nucleic acid sequence encoding the beta-glucosidase protein or a functional fragment thereof is at least 90% identical to SEQ ID NO: 4 or SEQ ID NO: 5.

69. The method of any one of claims 63-66, the cell of interest comprising an immune cell, and the bioengineered cell comprising a bioengineered immune cell.


SEQUENCE LISTING (43 sequences)

SEQ ID NO: 1 - DNA, 1740 bp, Neurospora crassa
atgtcgtctcacggctcccatgacggggccagcaccgagaagcatcttgctactcatgacattgcgcccacccacgacgccatcaagatagtgcccaagggccatggccagacagccacaaagcccggtgcccaagagaaggaggtccgcaacgccgccctatttgcggccatcaaggagtccaatatcaagccctggagcaaggagtccatccacctctatttcgccatcttcgtcgccttttgttgtgcatgcgccaacggttacgatggttcactcatgaccggaatcatcgctatggacaagttccagaaccaattccacactggtgacactggtcctaaagtctctgtcatcttttctctctataccgttggtgccatggttggagctcccttcgctgctatcctctctgatcgttttggccgtaagaagggcatgttcatcggtggtatctttatcattgtcggctccattattgttgctagctcctccaagctcgctcagtttgtcgttggccgcttcgttcttggcctcggtatcgccatcatgaccgttgctgccccggcctactccatcgaaatcgcccctcctcactggcgcggccgctgcactggcttctacaactgcggttggttcggaggttcgattcctgccgcctgcatcacctatggctgctacttcattaagagcaactggtcatggcgtatccccttgatccttcaggctttcacgtgccttatcgtcatgtcctccgtcttcttcctcccagaatcccctcgcttcctatttgccaacggccgcgacgctgaggctgttgcctttcttgtcaagtatcacggcaacggcgatcccaattccaagctggtgttgctcgagactgaggagatgagggacggtatcaggaccgacggtgtcgacaaggtctggtgggattaccgcccgctcttcatgacccacagcggccgctggcgcatggcccaggtgctcatgatctccatctttggccagttctccggcaacggtctcggttacttcaataccgtcatcttcaagaacattggtgtcaccagcacctcccaacagctcgcctacaacatcctcaactccgtcatctccgctatcggtgccttgaccgccgtctccatgactgatcgtatgccccgccgcgcggtgctcattatcggtaccttcatgtgcgccgctgctcttgccaccaactcgggtctttcggctactctcgacaagcagactcaaagaggcacgcaaatcaacctgaaccagggtatgaacgagcaggatgccaaggacaacgcctacctccacgtcgacagcaactacgccaagggtgccctggccgcttacttcctcttcaacgtcatcttctccttcacctacactcccctccagggtgttattcccaccgaggctctcgagaccaccatccgtggcaagggtcttgccctttccggcttcattgtcaacgccatgggcttcatcaaccagttcgctggccccatcgctctccacaacattggctacaagtacatctttgtctttgtcggctgggatcttatcgagaccgtcgcttggtacttctttggtgtcgaatcccaaggccgtaccctcgagcagctcgaatgggtctacgaccagcccaaccccgtcaaggcctccctaaaagtcgaaaaggtcgtcgtccaggccgacggccatgtgtccgaagctatcgttgcttag

SEQ ID NO: 2 - DNA, 1737 bp, Artificial Sequence
  cdt-1 (optimized DNA, IDTTM, version 1; IDT v1)
atgtcttcccacggttcacacgacggagctagtactgaaaagcatctggccacccacgacatagcccccacacatgatgcaatcaagatagtcccaaaagggcatggacagactgcaactaaacccggtgcacaagagaaggaagtcagaaatgcagccctgtttgctgcaataaaagagtccaatataaaaccttggtcaaaggagtccattcacttgtatttcgccatctttgtagccttctgttgtgcctgcgctaatgggtatgacggaagtcttatgacagggataattgcaatggacaagttccagaaccagttccacactggagacacaggtcccaaagtcagcgttattttttcactctacaccgtaggtgctatggtaggggctccatttgcagcaatcctcagtgatcgattcggacgaaaaaaaggcatgttcataggcgggatctttatcatagtgggctccatcattgtagcctcttcctcaaaattggcacaatttgtggtcggtcgcttcgttctcgggctgggtatagccatcatgaccgtcgcagctccagcatattcaatagagatcgccccaccccattggcggggtcgctgcaccggcttctacaactgcgggtggttcggcgggtcaatcccagccgcttgtataacttatgggtgctattttattaaatcaaattggtcatggcgaatcccactcatactgcaagcttttacctgtcttattgtcatgagctcagtcttcttcttgccagaatctcctcggtttttgttcgccaatggaagggatgctgaagctgtcgccttcctggtcaagtatcacggaaacggagacccaaactctaaattggttctgttggagaccgaggaaatgcgagacggaatccggacagatggggttgacaaggtatggtgggattataggccactgttcatgactcactccgggcgctggcgcatggcccaggtattgatgatttcaattttcgggcaatttagtggcaatggacttggatacttcaatactgtcatcttcaaaaacatcggcgtcactagcacctcacagcagctcgcctacaatatactcaacagcgttatatctgctattggtgcactcaccgctgtgtctatgacagacagaatgcccaggcgcgcagttctcataataggcacttttatgtgcgctgctgctctggcaactaacagtgggctcagtgctactcttgataaacaaactcagagagggacccagattaaccttaaccaagggatgaatgagcaggatgcaaaagataacgcataccttcacgtggattcaaactatgctaagggcgctctggctgcatatttcctctttaatgtaatttttagcttcacatatacccctcttcaaggtgtcatccccaccgaggccctggaaaccaccattcgggggaagggtctcgctctgtcaggattcattgtaaatgccatgggctttatcaatcaatttgccggcccaatagccttgcacaatatcggatataaatatatctttgtatttgtcggttgggatttgatagaaacagttgcatggtactttttcggagttgaatcccagggcagaaccttggaacaactggaatgggtgtacgaccaacctaatccagtgaaagcaagtctcaaggtcgagaaagtcgtagttcaagccgacggccatgtgagtgaagccatcgtggcc

SEQ ID NO: 3 - protein, 579 aa, Neurospora crassa
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 4 - DNA, 1431 bp, Neurospora crassa
atgtctcttcctaaggatttcctctggggcttcgctactgcggcctatcagattgagggtgctatccacgccgacggccgtggcccctctatctgggatactttctgcaacattcccggtaaaatcgccgacggcagctctggtgccgtcgcctgcgactcttacaaccgcaccaaggaggacattgacctcctcaagtctctcggcgccaccgcctaccgcttctccatctcctggtctcgcatcatccccgttggtggtcgcaacgaccccatcaaccagaagggcatcgaccactatgtcaagtttgtcgatgacctgctcgaggctggtattaccccctttatcaccctcttccactgggatcttcccgatggtctcgacaagcgctacggcggtcttctgaaccgtgaagagttccccctcgactttgagcactacgcccgcactatgttcaaggccattcccaagtgcaagcactggatcaccttcaacgagccctggtgcagctccatcctcggctacaactcgggctactttgcccctggccacacctccgaccgtaccaagtcacccgttggtgacagcgctcgcgagccctggatcgtcggccataacctgctcatcgctcacgggcgtgccgtcaaggtgtaccgagaagacttcaagcccacgcagggcggcgagatcggtatcaccttgaacggcgacgccactcttccctgggatccagaggaccccttggacgtcgaggcgtgcgaccgcaagattgagttcgccatcagctggttcgcagaccccatctactttggaaagtaccccgactcgatgcgcaaacagctcggtgaccggctgcccgagtttacgcccgaggaggtggcgcttgtcaagggttccaacgacttctacggcatgaaccactacacagccaactacatcaagcacaagaagggcgtccctcccgaggacgacttcctcggcaacctcgagacgctcttctacaacaagaagggtaactgcatcgggcccgagacccagtcgttctggctccggccgcacgcccagggcttccgcgacctgctcaactggctcagcaagcgctacggataccccaagatctacgtgaccgagaacgggaccagtctcaagggcgagaacgccatgccgctcaagcaaattgtcgaggacgacttccgcgtcaagtacttcaacgactacgtcaacgccatggccaaggcgcatagcgaggacggcgtcaacgtcaagggatatcttgcctggagcttgatggacaactttgagtgggccgagggctatgagacgcggttcggcgttacctatgtcgactatgagaacgaccagaagaggtaccccaagaagagcgccaagagcttgaagccgctctttgactctttgatcaagaaggactaa

SEQ ID NO: 5 - DNA, 1425 bp, Artificial Sequence
  gh1-1 (optimized DNA)
tccttgcccaaggattttctgtgggggtttgccacagctgcctatcaaattgagggcgctattcacgcagatggaagaggaccatccatttgggacacattttgcaacatccctggcaagatagcagacggatctagcggtgccgtggcttgcgactcatacaacagaactaaagaggatattgacctcctgaagagcttgggcgcaacagcatacaggtttagtatttcatggagcagaatcatcccagtaggaggcagaaacgaccctattaaccagaagggtatagatcactacgttaagtttgtggatgatctgcttgaggcaggtatcaccccatttattaccctctttcattgggatttgcctgatggtctcgataagcgctatggcgggctcttgaatcgggaggagttccctctggacttcgagcattacgctaggactatgttcaaggctataccaaaatgtaagcattggatcactttcaacgaaccctggtgctcctcaatcctcggatacaactcaggatattttgctccaggacacacttctgacagaacaaaaagtccagtaggcgatagcgcccgcgagccctggatagttggccataatctgttgatcgcacatgggcgagctgtcaaagtttatcgggaagatttcaagcctacacagggaggcgaaattggcatcaccctgaacggggacgccaccctgccctgggacccagaggaccctctcgatgtcgaggcctgcgatcgcaagatagagtttgcaatttcatggtttgctgatcccatttattttggaaagtaccctgactccatgagaaagcagctgggtgacaggcttccagagttcacacctgaagaagttgctcttgtcaagggatccaacgatttctacggtatgaatcattatacagctaactatatcaaacataaaaaaggtgttccacccgaggacgattttttgggtaatctcgaaaccttgttttataacaaaaagggaaactgtataggcccagagacccagagtttctggctccgaccccatgctcaagggttccgcgacctcctgaattggttgtccaagcgatacggctatcctaagatttatgtgacagagaacggtacttcattgaagggcgagaatgcaatgcctttgaagcaaattgtagaagatgatttccgcgttaagtactttaatgactatgtaaatgctatggctaaggcacactccgaagatggagttaatgtcaaaggatacctcgcttggtctcttatggataatttcgagtgggcagaaggctatgagactagattcggtgtgacatatgtggattacgagaacgatcagaagcgctatcccaagaaatcagccaaatccctcaaaccattgtttgattcattgattaagaaagac

SEQ ID NO: 6 - protein, 476 aa, Neurospora crassa
MSLPKDFLWGFATAAYQIEGAIHADGRGPSIWDTFCNIPGKIADGSSGAVACDSYNRTKEDIDLLKSLGATAYRFSISWSRIIPVGGRNDPINQKGIDHYVKFVDDLLEAGITPFITLFHWDLPDGLDKRYGGLLNREEFPLDFEHYARTMFKAIPKCKHWITFNEPWCSSILGYNSGYFAPGHTSDRTKSPVGDSAREPWIVGHNLLIAHGRAVKVYREDFKPTQGGEIGITLNGDATLPWDPEDPLDVEACDRKIEFAISWFADPIYFGKYPDSMRKQLGDRLPEFTPEEVALVKGSNDFYGMNHYTANYIKHKKGVPPEDDFLGNLETLFYNKKGNCIGPETQSFWLRPHAQGFRDLLNWLSKRYGYPKIYVTENGTSLKGENAMPLKQIVEDDFRVKYFNDYVNAMAKAHSEDGVNVKGYLAWSLMDNFEWAEGYETRFGVTYVDYENDQKRYPKKSAKSLKPLFDSLIKKD

SEQ ID NO: 7 - DNA, 6484 bp, Artificial Sequence
  MSCV-MCS-PGK-GFP vector
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctctcgaggttatcacaagtttgtacaaaaaagcaggcttcgaaggagatagaaccaattctctaaggaaatacttaaccatggtcgactggatccggtaccgaattcgcggccgcactcgagatatctagacccagctttcttgtacaaagtggtgataacgaattctaccgggtaggtgaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatctctttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcacggccgacaagcagaagaacggcatcaaggctaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 8 - DNA, 6475 bp, Artificial Sequence
  MSCV-MCS-PGK-mCherry vector
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctctcgaggttatcacaagtttgtacaaaaaagcaggcttcgaaggagatagaaccaattctctaaggaaatacttaaccatggtcgactggatccggtaccgaattcgcggccgcactcgagatatctagacccagctttcttgtacaaagtggtgataacgaattctaccgggtaggtgaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 9 - DNA, 8106 bp, Artificial Sequence
  MSCV-HA-CDT-1-PGK-GFP vector
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagccgccaccatggcgtacccatacgatgttccagattacgcttcttcccacggttcacacgacggagctagtactgaaaagcatctggccacccacgacatagcccccacacatgatgcaatcaagatagtcccaaaagggcatggacagactgcaactaaacccggtgcacaagagaaggaagtcagaaatgcagccctgtttgctgcaataaaagagtccaatataaaaccttggtcaaaggagtccattcacttgtatttcgccatctttgtagccttctgttgtgcctgcgctaatgggtatgacggaagtcttatgacagggataattgcaatggacaagttccagaaccagttccacactggagacacaggtcccaaagtcagcgttattttttcactctacaccgtaggtgctatggtaggggctccatttgcagcaatcctcagtgatcgattcggacgaaaaaaaggcatgttcataggcgggatctttatcatagtgggctccatcattgtagcctcttcctcaaaattggcacaatttgtggtcggtcgcttcgttctcgggctgggtatagccatcatgaccgtcgcagctccagcatattcaatagagatcgccccaccccattggcggggtcgctgcaccggcttctacaactgcgggtggttcggcgggtcaatcccagccgcttgtataacttatgggtgctattttattaaatcaaattggtcatggcgaatcccactcatactgcaagcttttacctgtcttattgtcatgagctcagtcttcttcttgccagaatctcctcggtttttgttcgccaatggaagggatgctgaagctgtcgccttcctggtcaagtatcacggaaacggagacccaaactctaaattggttctgttggagaccgaggaaatgcgagacggaatccggacagatggggttgacaaggtatggtgggattataggccactgttcatgactcactccgggcgctggcgcatggcccaggtattgatgatttcaattttcgggcaatttagtggcaatggacttggatacttcaatactgtcatcttcaaaaacatcggcgtcactagcacctcacagcagctcgcctacaatatactcaacagcgttatatctgctattggtgcactcaccgctgtgtctatgacagacagaatgcccaggcgcgcagttctcataataggcacttttatgtgcgctgctgctctggcaactaacagtgggctcagtgctactcttgataaacaaactcagagagggacccagattaaccttaaccaagggatgaatgagcaggatgcaaaagataacgcataccttcacgtggattcaaactatgctaagggcgctctggctgcatatttcctctttaatgtaatttttagcttcacatatacccctcttcaaggtgtcatccccaccgaggccctggaaaccaccattcgggggaagggtctcgctctgtcaggattcattgtaaatgccatgggctttatcaatcaatttgccggcccaatagccttgcacaatatcggatataaatatatctttgtatttgtcggttgggatttgatagaaacagttgcatggtactttttcggagttgaatcccagggcagaaccttggaacaactggaatgggtgtacgaccaacctaatccagtgaaagcaagtctcaaggtcgagaaagtcgtagttcaagccgacggccatgtgagtgaagccatcgtggcctgataagatctgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatctctttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcacggccgacaagcagaagaacggcatcaaggctaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 10 - DNA, 7797 bp, Artificial Sequence
  MSCV-HA-GH1-1-PGK-GFP vector
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagccgccaccatggcgtacccatacgatgttccagattacgcttccttgcccaaggattttctgtgggggtttgccacagctgcctatcaaattgagggcgctattcacgcagatggaagaggaccatccatttgggacacattttgcaacatccctggcaagatagcagacggatctagcggtgccgtggcttgcgactcatacaacagaactaaagaggatattgacctcctgaagagcttgggcgcaacagcatacaggtttagtatttcatggagcagaatcatcccagtaggaggcagaaacgaccctattaaccagaagggtatagatcactacgttaagtttgtggatgatctgcttgaggcaggtatcaccccatttattaccctctttcattgggatttgcctgatggtctcgataagcgctatggcgggctcttgaatcgggaggagttccctctggacttcgagcattacgctaggactatgttcaaggctataccaaaatgtaagcattggatcactttcaacgaaccctggtgctcctcaatcctcggatacaactcaggatattttgctccaggacacacttctgacagaacaaaaagtccagtaggcgatagcgcccgcgagccctggatagttggccataatctgttgatcgcacatgggcgagctgtcaaagtttatcgggaagatttcaagcctacacagggaggcgaaattggcatcaccctgaacggggacgccaccctgccctgggacccagaggaccctctcgatgtcgaggcctgcgatcgcaagatagagtttgcaatttcatggtttgctgatcccatttattttggaaagtaccctgactccatgagaaagcagctgggtgacaggcttccagagttcacacctgaagaagttgctcttgtcaagggatccaacgatttctacggtatgaatcattatacagctaactatatcaaacataaaaaaggtgttccacccgaggacgattttttgggtaatctcgaaaccttgttttataacaaaaagggaaactgtataggcccagagacccagagtttctggctccgaccccatgctcaagggttccgcgacctcctgaattggttgtccaagcgatacggctatcctaagatttatgtgacagagaacggtacttcattgaagggcgagaatgcaatgcctttgaagcaaattgtagaagatgatttccgcgttaagtactttaatgactatgtaaatgctatggctaaggcacactccgaagatggagttaatgtcaaaggatacctcgcttggtctcttatggataatttcgagtgggcagaaggctatgagactagattcggtgtgacatatgtggattacgagaacgatcagaagcgctatcccaagaaatcagccaaatccctcaaaccattgtttgattcattgattaagaaagactgataagatctgaattctaccgggtaggtgaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatctctttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcacggccgacaagcagaagaacggcatcaaggctaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgaatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 11 - DNA, 8097 bp, Artificial Sequence
  MSCV-HA-CDT-1-PGK-mCherry vector
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagccgccaccatggcgtacccatacgatgttccagattacgcttcttcccacggttcacacgacggagctagtactgaaaagcatctggccacccacgacatagcccccacacatgatgcaatcaagatagtcccaaaagggcatggacagactgcaactaaacccggtgcacaagagaaggaagtcagaaatgcagccctgtttgctgcaataaaagagtccaatataaaaccttggtcaaaggagtccattcacttgtatttcgccatctttgtagccttctgttgtgcctgcgctaatgggtatgacggaagtcttatgacagggataattgcaatggacaagttccagaaccagttccacactggagacacaggtcccaaagtcagcgttattttttcactctacaccgtaggtgctatggtaggggctccatttgcagcaatcctcagtgatcgattcggacgaaaaaaaggcatgttcataggcgggatctttatcatagtgggctccatcattgtagcctcttcctcaaaattggcacaatttgtggtcggtcgcttcgttctcgggctgggtatagccatcatgaccgtcgcagctccagcatattcaatagagatcgccccaccccattggcggggtcgctgcaccggcttctacaactgcgggtggttcggcgggtcaatcccagccgcttgtataacttatgggtgctattttattaaatcaaattggtcatggcgaatcccactcatactgcaagcttttacctgtcttattgtcatgagctcagtcttcttcttgccagaatctcctcggtttttgttcgccaatggaagggatgctgaagctgtcgccttcctggtcaagtatcacggaaacggagacccaaactctaaattggttctgttggagaccgaggaaatgcgagacggaatccggacagatggggttgacaaggtatggtgggattataggccactgttcatgactcactccgggcgctggcgcatggcccaggtattgatgatttcaattttcgggcaatttagtggcaatggacttggatacttcaatactgtcatcttcaaaaacatcggcgtcactagcacctcacagcagctcgcctacaatatactcaacagcgttatatctgctattggtgcactcaccgctgtgtctatgacagacagaatgcccaggcgcgcagttctcataataggcacttttatgtgcgctgctgctctggcaactaacagtgggctcagtgctactcttgataaacaaactcagagagggacccagattaaccttaaccaagggatgaatgagcaggatgcaaaagataacgcataccttcacgtggattcaaactatgctaagggcgctctggctgcatatttcctctttaatgtaatttttagcttcacatatacccctcttcaaggtgtcatccccaccgaggccctggaaaccaccattcgggggaagggtctcgctctgtcaggattcattgtaaatgccatgggctttatcaatcaatttgccggcccaatagccttgcacaatatcggatataaatatatctttgtatttgtcggttgggatttgatagaaacagttgcatggtactttttcggagttgaatcccagggcagaaccttggaacaactggaatgggtgtacgaccaacctaatccagtgaaagcaagtctcaaggtcgagaaagtcgtagttcaagccgacggccatgtgagtgaagccatcgtggcctgataagatctgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 12 - DNA, 8085 bp, Artificial Sequence
  MSCV-CDT-1-HA-IDTTM-v1-PGK-mCherry vector
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagccgccaccatgtcttcccacggttcacacgacggagctagtactgaaaagcatctggccacccacgacatagcccccacacatgatgcaatcaagatagtcccaaaagggcatggacagactgcaactaaacccggtgcacaagagaaggaagtcagaaatgcagccctgtttgctgcaataaaagagtccaatataaaaccttggtcaaaggagtccattcacttgtatttcgccatctttgtagccttctgttgtgcctgcgctaatgggtatgacggaagtcttatgacagggataattgcaatggacaagttccagaaccagttccacactggagacacaggtcccaaagtcagcgttattttttcactctacaccgtaggtgctatggtaggggctccatttgcagcaatcctcagtgatcgattcggacgaaaaaaaggcatgttcataggcgggatctttatcatagtgggctccatcattgtagcctcttcctcaaaattggcacaatttgtggtcggtcgcttcgttctcgggctgggtatagccatcatgaccgtcgcagctccagcatattcaatagagatcgccccaccccattggcggggtcgctgcaccggcttctacaactgcgggtggttcggcgggtcaatcccagccgcttgtataacttatgggtgctattttattaaatcaaattggtcatggcgaatcccactcatactgcaagcttttacctgtcttattgtcatgagctcagtcttcttcttgccagaatctcctcggtttttgttcgccaatggaagggatgctgaagctgtcgccttcctggtcaagtatcacggaaacggagacccaaactctaaattggttctgttggagaccgaggaaatgcgagacggaatccggacagatggggttgacaaggtatggtgggattataggccactgttcatgactcactccgggcgctggcgcatggcccaggtattgatgatttcaattttcgggcaatttagtggcaatggacttggatacttcaatactgtcatcttcaaaaacatcggcgtcactagcacctcacagcagctcgcctacaatatactcaacagcgttatatctgctattggtgcactcaccgctgtgtctatgacagacagaatgcccaggcgcgcagttctcataataggcacttttatgtgcgctgctgctctggcaactaacagtgggctcagtgctactcttgataaacaaactcagagagggacccagattaaccttaaccaagggatgaatgagcaggatgcaaaagataacgcataccttcacgtggattcaaactatgctaagggcgctctggctgcatatttcctctttaatgtaatttttagcttcacatatacccctcttcaaggtgtcatccccaccgaggccctggaaaccaccattcgggggaagggtctcgctctgtcaggattcattgtaaatgccatgggctttatcaatcaatttgccggcccaatagccttgcacaatatcggatataaatatatctttgtatttgtcggttgggatttgatagaaacagttgcatggtactttttcggagttgaatcccagggcagaaccttggaacaactggaatgggtgtacgaccaacctaatccagtgaaagcaagtctcaaggtcgagaaagtcgtagttcaagccgacggccatgtgagtgaagccatcgtggcctacccatacgatgttccagattacgcttgaaattctaccgggtaggtgaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 13 - DNA, 8085 bp, Artificial; identical to SEQ ID NO: 12
  MSCV-CDT-1-HA-IDTTM-v1-PGK-mCherry vector

SEQ ID NO: 14 - DNA, 8097 bp, Artificial Sequence
  MSCV-CDT-1-HA-IDTTM-v2-PGK-mCherry vector
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagccgccaccatggcgtcttcccacggttcacacgacggagctagtactgaaaagcatctggccacccacgacatagcccccacacatgatgcaatcaagatagtcccaaaagggcacggacagactgcaactaaacccggtgcacaagagaaggaagtcagaaatgcagccctgtttgctgcaataaaagagtccaatataaaaccttggtcaaaggagtccattcacttgtatttcgccatctttgtagccttctgttgtgcctgcgctaatgggtatgacggatctttgatgacagggataattgctatggacaagttccagaaccagttccacactggagacacaggtcccaaagtcagcgttattttttcactctacaccgtaggtgctatggtaggggctccatttgcagcaatcctcagtgatcgattcggacgaaaaaaaggtatgtttataggcgggatctttatcatagtgggctccatcattgtagcctcttcctcaaaattggcacaatttgtggtcggtcgcttcgttctcgggctgggtatagctattatgacagtcgcagctccagcatattcaatagagatcgccccaccccattggcggggtcgctgcaccggcttctacaactgcgggtggttcggcgggtcaatcccagccgcttgtataacttatgggtgctattttattaaatcaaattggtcctggcgaatcccactcatactgcaagcttttacctgtcttattgttatgtcatcagtcttcttcttgccagaatctcctcggtttttgttcgccaatggaagggatgctgaagctgtcgccttcctggtcaagtatcacggaaacggagacccaaactctaaattggttctgttggagaccgaagaaatgcgtgacggaatccggacagatggggttgacaaggtctggtgggattataggccactgtttatgactcactccgggcgctggcgaatggcacaggtattgatgatttcaattttcgggcaatttagtggcaacggacttggatacttcaatactgtcatcttcaaaaacatcggcgtcactagcacctcacagcagctcgcctacaatatactcaacagcgttatatctgctattggtgcactcaccgctgtgtcaatgacagatcgaatgcccaggcgcgcagttctcataataggcacttttatgtgcgctgctgctctggcaactaacagtgggctcagtgctactcttgataaacaaactcagagagggacccagattaaccttaaccaaggtatgaatgagcaggatgcaaaagataacgcataccttcacgtggattcaaactatgctaagggcgctctggctgcatatttcctctttaatgtaatttttagcttcacatatacccctcttcaaggtgtcatccccaccgaggccctggaaaccaccattcgggggaagggtctcgctctgtcaggattcattgtaaatgctatgggatttatcaatcaatttgccggcccaatagccttgcacaatatcggatataaatatatctttgtatttgtcggttgggatttgatagaaacagttgcatggtactttttcggagttgaatcccagggcagaaccttggaacaactggagtgggtgtacgaccaacctaatccagtgaaagcaagtctcaaggtcgagaaagtcgtagttcaagccgacggccatgtgagtgaagccatcgtggcctacccatacgatgttccagattacgcttgataagatctgaattctaccgggtaggtgaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 15 - DNA, 8097 bp, Artificial Sequence
  MSCV-CDT-1-HA-BLUEHERONTM-PGK-mCherry vector
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagccgccaccatggcgtctagtcacggaagtcacgacggcgctagcaccgaaaagcacctggccactcacgatattgcccctacccacgacgctatcaagatcgtacccaaaggtcacgggcagactgctactaagcccggagcgcaggaaaaagaggtgcgcaacgctgcccttttcgcagctatcaaggaaagtaatattaaaccgtggagtaaggagagtatccatctctatttcgctatcttcgtagctttctgctgtgcgtgcgccaacgggtatgacggatctttgatgacaggaatcattgctatggacaaattccagaatcagttccatacaggagacacaggtcccaaggtcagtgttatattttctctgtacacagtcggtgctatggtaggtgcccccttcgctgctattctgtccgaccgcttcggacggaaaaaaggtatgtttatcgggggaatttttatcattgtgggcagcattatcgtggcaagttcaagcaaactggctcaattcgttgttggcaggttcgtcctgggactgggtatcgctattatgacagtcgcagctcccgcttattctatcgaaatcgcaccaccgcactggagaggacgctgcactggtttttataactgcggctggtttggcggcagcatcccggcggcatgcatcacctatggctgctattttatcaagtccaactggagctggcgaatccccttgatcctccaggccttcacttgtctcattgttatgtcatctgttttttttctccctgagtcccctagatttcttttcgccaacggtagagacgctgaggctgttgccttcctggtaaagtaccacggcaacggcgaccccaactccaaactcgtgctgctggagactgaagaaatgcgtgacgggattcggaccgacggggtcgacaaggtctggtgggactatcgccctctttttatgacccatagtgggcggtggcgaatggcacaggtattgatgatctctatctttgggcaattctctgggaacggacttggttactttaacaccgttatctttaaaaacatcggggtcacttcaacctctcagcaattggcgtataacattctgaactccgtcatcagcgcaatcggggcactgacagcggtctcaatgactgatcgaatgcctcgcagagcggtgcttatcatcggaacttttatgtgcgctgctgccttggccactaacagcggcctttccgcgactttggataaacaaacacagcggggtacgcagattaacctcaatcagggtatgaacgaacaagatgctaaagacaatgcgtatttgcacgtcgatagcaattacgctaagggtgctttggccgcctatttcctgttcaacgtgatttttagcttcacgtacactcctctgcagggtgttattccaaccgaggcactcgaaaccacgatccgaggcaagggactggcactcagcggctttatcgtgaacgctatgggattcattaatcagtttgctggccctattgctctgcacaacattgggtacaagtacatcttcgttttcgtgggctgggacctcatcgaaactgtggcgtggtatttcttcggagtggagagtcaggggcgaacgctggaacagctcgaatgggtgtatgatcaacccaatcctgtaaaagcaagtctgaaggtggagaaagttgtggtgcaggctgatggacacgtgtctgaagccatcgtggcgtacccatacgatgttccagattacgcttgataagatctgaattctaccgggtaggtgaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 16 - DNA, 8097 bp, Artificial
  MSCV-CDT-1-HA-GENSCRIPTTM-mCherry vector
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagccgccaccatggcgtcatctcacggttctcacgacggggcctccaccgagaaacatctcgctactcatgacatcgctccaacacatgatgccataaagatcgtgcccaagggtcacggacagacagccacaaagcctggggctcaggaaaaggaagttagaaatgcagccctgttcgctgctattaaagaaagtaacatcaaaccgtggagtaaggaaagcatccacctgtatttcgcaatatttgtggctttctgctgcgcctgtgccaatggctatgacggatctttgatgacaggaataattgctatggacaagttccagaaccagttccacactggggacaccggccccaaagtctccgtgatcttttctttatacaccgttggtgctatggtaggtgccccctttgctgcgatactgagtgacagatttggtaggaagaaaggtatgtttattgggggcatttttatcatagtcgggtctattattgtggcatcctccagcaaactggctcaatttgtcgtggggcggttcgtattgggcctggggattgctattatgacagttgcagcacctgcatacagcattgagatcgctccgccacactggcggggacgatgtacaggattctacaactgtgggtggtttggaggctccatcccagccgcctgcatcacctatggctgctacttcatcaagagcaactggagctggcgcatccccctcatcctccaagccttcacctgcctgattgttatgtcaagcgtcttctttctccctgagtcaccacgcttcctgtttgccaacgggcgtgatgcagaggccgtagcctttctggtgaaataccacgggaacggagacccaaattcaaaacttgtgctgctcgagacagaagaaatgcgtgacggcatcaggacagatggtgttgataaagtgtggtgggactaccggcctctttttatgacgcactccggacgctggcgaatggcacaggtattgatgatctccattttcgggcaattctctggaaacggactaggatattttaacacagtcatctttaagaatattggagtcacatcaaccagtcagcagttggcgtataacattctgaacagcgttatttcagcgatcggcgctttaacggctgtttcaatgacagatcgaatgcccaggagagctgtgcttatcatcgggacttttatgtgtgctgctgcgctggccacgaatagtggcctgtcagccactttggataagcagacccagcgtggtactcagatcaacctcaaccagggtatgaatgagcaggacgccaaggacaacgcctatctgcacgtggacagcaactatgctaaaggcgcgttggcagcctactttctcttcaatgtcatcttcagctttacctacacacctctgcagggcgtgattcctacagaagctttagaaaccaccatccgaggcaaaggactcgctttgtctggtttcatagtgaatgctatgggatttatcaatcagtttgcagggcccattgcacttcacaacatcggctacaagtacatcttcgtctttgttggctgggatcttattgaaactgtggcctggtacttcttcggagtggagtctcaaggtcggactctagaacagctggagtgggtgtatgaccagccaaacccagtgaaggcatcgctgaaagtagagaaggtggtggtacaagcggacggtcatgtcagtgaagcaatagtcgcatacccatacgatgttccagattacgcttgataagatctgaattctaccgggtaggtgaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 17 - DNA, 1734 bp, Artificial Sequence
  cdt-1 (optimized DNA, IDTTM, version 2; IDT v2)
tcttcccacggttcacacgacggagctagtactgaaaagcatctggccacccacgacatagcccccacacatgatgcaatcaagatagtcccaaaagggcacggacagactgcaactaaacccggtgcacaagagaaggaagtcagaaatgcagccctgtttgctgcaataaaagagtccaatataaaaccttggtcaaaggagtccattcacttgtatttcgccatctttgtagccttctgttgtgcctgcgctaatgggtatgacggatctttgatgacagggataattgctatggacaagttccagaaccagttccacactggagacacaggtcccaaagtcagcgttattttttcactctacaccgtaggtgctatggtaggggctccatttgcagcaatcctcagtgatcgattcggacgaaaaaaaggtatgtttataggcgggatctttatcatagtgggctccatcattgtagcctcttcctcaaaattggcacaatttgtggtcggtcgcttcgttctcgggctgggtatagctattatgacagtcgcagctccagcatattcaatagagatcgccccaccccattggcggggtcgctgcaccggcttctacaactgcgggtggttcggcgggtcaatcccagccgcttgtataacttatgggtgctattttattaaatcaaattggtcctggcgaatcccactcatactgcaagcttttacctgtcttattgttatgtcatcagtcttcttcttgccagaatctcctcggtttttgttcgccaatggaagggatgctgaagctgtcgccttcctggtcaagtatcacggaaacggagacccaaactctaaattggttctgttggagaccgaagaaatgcgtgacggaatccggacagatggggttgacaaggtctggtgggattataggccactgtttatgactcactccgggcgctggcgaatggcacaggtattgatgatttcaattttcgggcaatttagtggcaacggacttggatacttcaatactgtcatcttcaaaaacatcggcgtcactagcacctcacagcagctcgcctacaatatactcaacagcgttatatctgctattggtgcactcaccgctgtgtcaatgacagatcgaatgcccaggcgcgcagttctcataataggcacttttatgtgcgctgctgctctggcaactaacagtgggctcagtgctactcttgataaacaaactcagagagggacccagattaaccttaaccaaggtatgaatgagcaggatgcaaaagataacgcataccttcacgtggattcaaactatgctaagggcgctctggctgcatatttcctctttaatgtaatttttagcttcacatatacccctcttcaaggtgtcatccccaccgaggccctggaaaccaccattcgggggaagggtctcgctctgtcaggattcattgtaaatgctatgggatttatcaatcaatttgccggcccaatagccttgcacaatatcggatataaatatatctttgtatttgtcggttgggatttgatagaaacagttgcatggtactttttcggagttgaatcccagggcagaaccttggaacaactggagtgggtgtacgaccaacctaatccagtgaaagcaagtctcaaggtcgagaaagtcgtagttcaagccgacggccatgtgagtgaagccatcgtggcc

SEQ ID NO: 18 - DNA, 1734 bp, Artificial Sequence
  cdt-1 (optimized DNA, BLUE HERONTM; BlueHeron)
tctagtcacggaagtcacgacggcgctagcaccgaaaagcacctggccactcacgatattgcccctacccacgacgctatcaagatcgtacccaaaggtcacgggcagactgctactaagcccggagcgcaggaaaaagaggtgcgcaacgctgcccttttcgcagctatcaaggaaagtaatattaaaccgtggagtaaggagagtatccatctctatttcgctatcttcgtagctttctgctgtgcgtgcgccaacgggtatgacggatctttgatgacaggaatcattgctatggacaaattccagaatcagttccatacaggagacacaggtcccaaggtcagtgttatattttctctgtacacagtcggtgctatggtaggtgcccccttcgctgctattctgtccgaccgcttcggacggaaaaaaggtatgtttatcgggggaatttttatcattgtgggcagcattatcgtggcaagttcaagcaaactggctcaattcgttgttggcaggttcgtcctgggactgggtatcgctattatgacagtcgcagctcccgcttattctatcgaaatcgcaccaccgcactggagaggacgctgcactggtttttataactgcggctggtttggcggcagcatcccggcggcatgcatcacctatggctgctattttatcaagtccaactggagctggcgaatccccttgatcctccaggccttcacttgtctcattgttatgtcatctgttttttttctccctgagtcccctagatttcttttcgccaacggtagagacgctgaggctgttgccttcctggtaaagtaccacggcaacggcgaccccaactccaaactcgtgctgctggagactgaagaaatgcgtgacgggattcggaccgacggggtcgacaaggtctggtgggactatcgccctctttttatgacccatagtgggcggtggcgaatggcacaggtattgatgatctctatctttgggcaattctctgggaacggacttggttactttaacaccgttatctttaaaaacatcggggtcacttcaacctctcagcaattggcgtataacattctgaactccgtcatcagcgcaatcggggcactgacagcggtctcaatgactgatcgaatgcctcgcagagcggtgcttatcatcggaacttttatgtgcgctgctgccttggccactaacagcggcctttccgcgactttggataaacaaacacagcggggtacgcagattaacctcaatcagggtatgaacgaacaagatgctaaagacaatgcgtatttgcacgtcgatagcaattacgctaagggtgctttggccgcctatttcctgttcaacgtgatttttagcttcacgtacactcctctgcagggtgttattccaaccgaggcactcgaaaccacgatccgaggcaagggactggcactcagcggctttatcgtgaacgctatgggattcattaatcagtttgctggccctattgctctgcacaacattgggtacaagtacatcttcgttttcgtgggctgggacctcatcgaaactgtggcgtggtatttcttcggagtggagagtcaggggcgaacgctggaacagctcgaatgggtgtatgatcaacccaatcctgtaaaagcaagtctgaaggtggagaaagttgtggtgcaggctgatggacacgtgtctgaagccatcgtggcg

SEQ ID NO: 19 - DNA, 1734 bp, Artificial Sequence
  cdt-1 (optimized DNA, GENSCRIPTTM; GenScript)
tcatctcacggttctcacgacggggcctccaccgagaaacatctcgctactcatgacatcgctccaacacatgatgccataaagatcgtgcccaagggtcacggacagacagccacaaagcctggggctcaggaaaaggaagttagaaatgcagccctgttcgctgctattaaagaaagtaacatcaaaccgtggagtaaggaaagcatccacctgtatttcgcaatatttgtggctttctgctgcgcctgtgccaatggctatgacggatctttgatgacaggaataattgctatggacaagttccagaaccagttccacactggggacaccggccccaaagtctccgtgatcttttctttatacaccgttggtgctatggtaggtgccccctttgctgcgatactgagtgacagatttggtaggaagaaaggtatgtttattgggggcatttttatcatagtcgggtctattattgtggcatcctccagcaaactggctcaatttgtcgtggggcggttcgtattgggcctggggattgctattatgacagttgcagcacctgcatacagcattgagatcgctccgccacactggcggggacgatgtacaggattctacaactgtgggtggtttggaggctccatcccagccgcctgcatcacctatggctgctacttcatcaagagcaactggagctggcgcatccccctcatcctccaagccttcacctgcctgattgttatgtcaagcgtcttctttctccctgagtcaccacgcttcctgtttgccaacgggcgtgatgcagaggccgtagcctttctggtgaaataccacgggaacggagacccaaattcaaaacttgtgctgctcgagacagaagaaatgcgtgacggcatcaggacagatggtgttgataaagtgtggtgggactaccggcctctttttatgacgcactccggacgctggcgaatggcacaggtattgatgatctccattttcgggcaattctctggaaacggactaggatattttaacacagtcatctttaagaatattggagtcacatcaaccagtcagcagttggcgtataacattctgaacagcgttatttcagcgatcggcgctttaacggctgtttcaatgacagatcgaatgcccaggagagctgtgcttatcatcgggacttttatgtgtgctgctgcgctggccacgaatagtggcctgtcagccactttggataagcagacccagcgtggtactcagatcaacctcaaccagggtatgaatgagcaggacgccaaggacaacgcctatctgcacgtggacagcaactatgctaaaggcgcgttggcagcctactttctcttcaatgtcatcttcagctttacctacacacctctgcagggcgtgattcctacagaagctttagaaaccaccatccgaggcaaaggactcgctttgtctggtttcatagtgaatgctatgggatttatcaatcagtttgcagggcccattgcacttcacaacatcggctacaagtacatcttcgtctttgttggctgggatcttattgaaactgtggcctggtacttcttcggagtggagtctcaaggtcggactctagaacagctggagtgggtgtatgaccagccaaacccagtgaaggcatcgctgaaagtagagaaggtggtggtacaagcggacggtcatgtcagtgaagcaatagtcgca

SEQ ID NO: 20 - DNA, 27 bp, Artificial Sequence
  HA tag
tacccatacgatgttccagattacgct

SEQ ID NO: 21 - DNA, 25 bp, Artificial Sequence
  5′-overlap (5′-3′)
actccttctctaggcgccggaatta

SEQ ID NO: 22 - DNA, 25 bp, Artificial Sequence
  3′-overlap (5′-3′)
actccttctctaggcgccggaatta

SEQ ID NO: 23 - DNA, 25 bp, Artificial Sequence
  ERES signal
tcgcggagtggatgggccatcttaa

SEQ ID NO: 24 - DNA, 18 bp, Artificial Sequence
  ERES signal
ttctgctacgagaatgaa

SEQ ID NO: 25 - protein, 6 aa, Artificial Sequence
  ERES signal
FCYENE

SEQ ID NO: 26 - DNA, 7077 bp, Artificial Sequence
  MSCV_PGK-2A-mCherry Vector
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctggtgataacgaattctaccgggtaggtgaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccgcggccgcactggccgccaccatgggatccggctccggagagggccgcggtagcctcctgacctgcggggacgtggaggagaaccccggccctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgacgcccgccccacgacccgcagcgcccgaccgaaaggagcgcacgaccccatgcatataattcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 27 - DNA, 7086 bp, Artificial Sequence
  MSCV_PGK-2A-GFP Vector
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctggtgataacgaattctaccgggtaggtgaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccgcggccgcactggccgccaccatgggatccggctccggagagggccgcggtagcctcctgacctgcggggacgtggaggagaaccccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatctctttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcacggccgacaagcagaagaacggcatcaaggctaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacgcccgccccacgacccgcagcgcccgaccgaaaggagcgcacgaccccatgcatataattcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 28 - DNA, 8832 bp, Artificial Sequence
  MSCV_PGK-cdt-1-2A-mCherry Vector
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctggtgataacgaattctaccgggtaggtgaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccgcgccgccaccatggcgtcatctcacggttctcacgacggggcctccaccgagaaacatctcgctactcatgacatcgctccaacacatgatgccataaagatcgtgcccaagggtcacggacagacagccacaaagcctggggctcaggaaaaggaagttagaaatgcagccctgttcgctgctattaaagaaagtaacatcaaaccgtggagtaaggaaagcatccacctgtatttcgcaatatttgtggctttctgctgcgcctgtgccaatggctatgacggatctttgatgacaggaataattgctatggacaagttccagaaccagttccacactggggacaccggccccaaagtctccgtgatcttttctttatacaccgttggtgctatggtaggtgccccctttgctgcgatactgagtgacagatttggtaggaagaaaggtatgtttattgggggcatttttatcatagtcgggtctattattgtggcatcctccagcaaactggctcaatttgtcgtggggcggttcgtattgggcctggggattgctattatgacagttgcagcacctgcatacagcattgagatcgctccgccacactggcggggacgatgtacaggattctacaactgtgggtggtttggaggctccatcccagccgcctgcatcacctatggctgctacttcatcaagagcaactggagctggcgcatccccctcatcctccaagccttcacctgcctgattgttatgtcaagcgtcttctttctccctgagtcaccacgcttcctgtttgccaacgggcgtgatgcagaggccgtagcctttctggtgaaataccacgggaacggagacccaaattcaaaacttgtgctgctcgagacagaagaaatgcgtgacggcatcaggacagatggtgttgataaagtgtggtgggactaccggcctctttttatgacgcactccggacgctggcgaatggcacaggtattgatgatctccattttcgggcaattctctggaaacggactaggatattttaacacagtcatctttaagaatattggagtcacatcaaccagtcagcagttggcgtataacattctgaacagcgttatttcagcgatcggcgctttaacggctgtttcaatgacagatcgaatgcccaggagagctgtgcttatcatcgggacttttatgtgtgctgctgcgctggccacgaatagtggcctgtcagccactttggataagcagacccagcgtggtactcagatcaacctcaaccagggtatgaatgagcaggacgccaaggacaacgcctatctgcacgtggacagcaactatgctaaaggcgcgttggcagcctactttctcttcaatgtcatcttcagctttacctacacacctctgcagggcgtgattcctacagaagctttagaaaccaccatccgaggcaaaggactcgctttgtctggtttcatagtgaatgctatgggatttatcaatcagtttgcagggcccattgcacttcacaacatcggctacaagtacatcttcgtctttgttggctgggatcttattgaaactgtggcctggtacttcttcggagtggagtctcaaggtcggactctagaacagctggagtgggtgtatgaccagccaaacccagtgaaggcatcgctgaaagtagagaaggtggtggtacaagcggacggtcatgtcagtgaagcaatagtcgcatacccatacgatgttccagattacgctggatccggctccggagagggccgcggtagcctcctgacctgcggggacgtggaggagaaccccggccctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgacgcccgccccacgacccgcagcgcccgaccgaaaggagcgcacgaccccatgcatataattcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtgagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaactaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtaagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 29 - DNA, 8535 bp, Artificial Sequence
  MSCV_PGK-gh1-1-2A-GFP Vector
aatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctggtgataacgaattctaccgggtaggtgaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagctaggaccgcgccgccaccatggcgtacccatacgatgttccagattacgcttccttgcccaaggattttctgtgggggtttgccacagctgcctatcaaattgagggcgctattcacgcagatggaagaggaccatccatttgggacacattttgcaacatccctggcaagatagcagacggatctagcggtgccgtggcttgcgactcatacaacagaactaaagaggatattgacctcctgaagagcttgggcgcaacagcatacaggtttagtatttcatggagcagaatcatcccagtaggaggcagaaacgaccctattaaccagaagggtatagatcactacgttaagtttgtggatgatctgcttgaggcaggtatcaccccatttattaccctctttcattgggatttgcctgatggtctcgataagcgctatggcgggctcttgaatcgggaggagttccctctggacttcgagcattacgctaggactatgttcaaggctataccaaaatgtaagcattggatcactttcaacgaaccctggtgctcctcaatcctcggatacaactcaggatattttgctccaggacacacttctgacagaacaaaaagtccagtaggcgatagcgcccgcgagccctggatagttggccataatctgttgatcgcacatgggcgagctgtcaaagtttatcgggaagatttcaagcctacacagggaggcgaaattggcatcaccctgaacggggacgccaccctgccctgggacccagaggaccctctcgatgtcgaggcctgcgatcgcaagatagagtttgcaatttcatggtttgctgatcccatttattttggaaagtaccctgactccatgagaaagcagctgggtgacaggcttccagagttcacacctgaagaagttgctcttgtcaagggatccaacgatttctacggtatgaatcattatacagctaactatatcaaacataaaaaaggtgttccacccgaggacgattttttgggtaatctcgaaaccttgttttataacaaaaagggaaactgtataggcccagagacccagagtttctggctccgaccccatgctcaagggttccgcgacctcctgaattggttgtccaagcgatacggctatcctaagatttatgtgacagagaacggtacttcattgaagggcgagaatgcaatgcctttgaagcaaattgtagaagatgatttccgcgttaagtactttaatgactatgtaaatgctatggctaaggcacactccgaagatggagttaatgtcaaaggatacctcgcttggtctcttatggataatttcgagtgggcagaaggctatgagactagattcggtgtgacatatgtggattacgagaacgatcagaagcgctatcccaagaaatcagccaaatccctcaaaccattgtttgattcattgattaagaaagacggatccggctccggagagggccgcggtagcctcctgacctgcggggacgtggaggagaaccccggccctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatctctttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcacggccgacaagcagaagaacggcatcaaggctaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgacgcccgccccacgacccgcagcgcccgaccgaaaggagcgcacgaccccatgcatcgataattcccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaagggggg

SEQ ID NO: 30 - protein, 5 aa, Artificial Sequence
  Linker
GSGSG

SEQ ID NO: 31 - protein, 18 aa, Artificial Sequence
  T2A amino acids
EGRGSLLTCGDVEENPGP

SEQ ID NO: 32 - protein, 589 aa, Artificial Sequence
  CDT-1
MASSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVAYPYDVPDYA

SEQ ID NO: 33 - protein, 611 aa, Artificial Sequence
  CDT-1 with linker and T2A amino acids
MASSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVAYPYDVPDYAGSGSGEGRGSLLTCGDVEENPG

SEQ ID NO: 34 - protein, 236 aa, Artificial Sequence
  mCherry
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 35 - protein, 237 aa, Artificial Sequence
  mCherry with T2A amino acid
PMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 36 - protein, 848 aa, Artificial Sequence
  CDT-1 with linker, T2A amino acids, and mCherry
MASSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVAYPYDVPDYAGSGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 37 - protein, 486 aa, Artificial Sequence
  GH1-1
MAYPYDVPDYASLPKDFLWGFATAAYQIEGAIHADGRGPSIWDTFCNIPGKIADGSSGAVACDSYNRTKEDIDLLKSLGATAYRFSISWSRIIPVGGRNDPINQKGIDHYVKFVDDLLEAGITPFITLFHWDLPDGLDKRYGGLLNREEFPLDFEHYARTMFKAIPKCKHWITFNEPWCSSILGYNSGYFAPGHTSDRTKSPVGDSAREPWIVGHNLLIAHGRAVKVYREDFKPTQGGEIGITLNGDATLPWDPEDPLDVEACDRKIEFAISWFADPIYFGKYPDSMRKQLGDRLPEFTPEEVALVKGSNDFYGMNHYTANYIKHKKGVPPEDDFLGNLETLFYNKKGNCIGPETQSFWLRPHAQGFRDLLNWLSKRYGYPKIYVTENGTSLKGENAMPLKQIVEDDFRVKYFNDYVNAMAKAHSEDGVNVKGYLAWSLMDNFEWAEGYETRFGVTYVDYENDQKRYPKKSAKSLKPLFDSLIKKD

SEQ ID NO: 38 - protein, 508 aa, Artificial Sequence
  GH1-1 with linker and T2A amino acids
MAYPYDVPDYASLPKDFLWGFATAAYQIEGAIHADGRGPSIWDTFCNIPGKIADGSSGAVACDSYNRTKEDIDLLKSLGATAYRFSISWSRIIPVGGRNDPINQKGIDHYVKFVDDLLEAGITPFITLFHWDLPDGLDKRYGGLLNREEFPLDFEHYARTMFKAIPKCKHWITFNEPWCSSILGYNSGYFAPGHTSDRTKSPVGDSAREPWIVGHNLLIAHGRAVKVYREDFKPTQGGEIGITLNGDATLPWDPEDPLDVEACDRKIEFAISWFADPIYFGKYPDSMRKQLGDRLPEFTPEEVALVKGSNDFYGMNHYTANYIKHKKGVPPEDDFLGNLETLFYNKKGNCIGPETQSFWLRPHAQGFRDLLNWLSKRYGYPKIYVTENGTSLKGENAMPLKQIVEDDFRVKYFNDYVNAMAKAHSEDGVNVKGYLAWSLMDNFEWAEGYETRFGVTYVDYENDQKRYPKKSAKSLKPLFDSLIKKDGSGSGEGRGSLLTCGDVEENPG

SEQ ID NO: 39 - protein, 239 aa, Artificial Sequence
  GFP
MVSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 40 - protein, 240 aa, Artificial Sequence
  GFP with T2A amino acid
PMVSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 41 - protein, 748 aa, Artificial Sequence
  GH1-1 with linker, T2A amino acids, and GFP
MAYPYDVPDYASLPKDFLWGFATAAYQIEGAIHADGRGPSIWDTFCNIPGKIADGSSGAVACDSYNRTKEDIDLLKSLGATAYRFSISWSRIIPVGGRNDPINQKGIDHYVKFVDDLLEAGITPFITLFHWDLPDGLDKRYGGLLNREEFPLDFEHYARTMFKAIPKCKHWITFNEPWCSSILGYNSGYFAPGHTSDRTKSPVGDSAREPWIVGHNLLIAHGRAVKVYREDFKPTQGGEIGITLNGDATLPWDPEDPLDVEACDRKIEFAISWFADPIYFGKYPDSMRKQLGDRLPEFTPEEVALVKGSNDFYGMNHYTANYIKHKKGVPPEDDFLGNLETLFYNKKGNCIGPETQSFWLRPHAQGFRDLLNWLSKRYGYPKIYVTENGTSLKGENAMPLKQIVEDDFRVKYFNDYVNAMAKAHSEDGVNVKGYLAWSLMDNFEWAEGYETRFGVTYVDYENDQKRYPKKSAKSLKPLFDSLIKKDGSGSGEGRGSLLTCGDVEENPGPMVSKGEELFTGVVPILVELDGDVNGHKFSVSGEGEGDATYGKLTLKFICTTGKLPVPWPTLVTTFTYGVQCFSRYPDHMKQHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 42 - DNA, 5541 bp, Artificial Sequence
  Backbone sequence for MSCV_PGK-2A-mCherry vector
atgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctggtgataacgaattctaccgggtaggtgaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 43 - DNA, 5541 bp, Artificial Sequence; identical to SEQ ID NO: 42
  Backbone sequence for MSCV_PGK-2A-GFP vector